(19) United States
(12) Patent Application Publication
(10) Pub. No.: US 2025/0346897 A1
(43) Pub. Date: Nov. 13, 2025

(54) COMPOSITIONS AND METHODS FOR TREATMENT OF MICROSATELLITE DNA EXPANSION DISORDERS

(71) Applicant: Atalanta Therapeutics, Inc., Boston, MA (US)

(72) Inventors: Corrie GALLANT-BEHM, Wellesley, MA (US); Matthew HASSLER, Boston, MA (US); Daniel CURTIS, Belmont, MA (US); Bruno Miguel Da Cruz GODINHO, Reading, MA (US)

(21) Appl. No.: 18/866,188

(22) PCT Filed: May 16, 2023
(86) PCT No.: PCT/US2023/067039

§ 371 Date: Nov. 15, 2024

Related U.S. Application Data
(60) Provisional application No. 63/342,552, filed on May 16, 2022.

(51) Int. Cl. C12N 15/113; A61K 31/713

(52) U.S. Cl. CPC C12N 15/113; A61K 31/713; C12N 2310/14; C12N 2310/315; C12N 2310/321; C12N 2310/322; C12N 2310/52; C12N 2320/30

(57) ABSTRACT

The present disclosure provides single- or double-stranded interfering RNA molecules (e.g., siRNA) that target a MutS Homolog 3 (MSH3) gene. The interfering RNA molecules may contain specific patterns of nucleoside modifications and internucleoside linkage modifications, as pharmaceutical compositions including the same. The siRNA molecules may be branched siRNA molecules, such as di-branched, tri-branched, or tetra-branched siRNA molecules. The disclosed siRNA molecules may further feature a 5′ phosphorus stabilizing moiety and/or a hydrophobic moiety. Additionally, the disclosure provides methods for delivering the siRNA molecule of the disclosure to the central nervous system of a subject, such as a subject identified as having Huntington's Disease.

### SEQUENCE LISTING

The instant application contains a Sequence Listing which has been submitted electronically in XML format and is hereby incorporated by reference in its entirety. Said XML copy, created on May 15, 2023, is named “51436-034WO2_Sequence_Listing_5_15_23” and is 1,089,536 bytes in size.

### TECHNICAL FIELD

This disclosure relates to small interfering RNA (siRNA) molecules, and compositions containing the same, that target RNA transcripts (e.g., mRNA) of a MutS Homolog 3 (MSH3) gene. The disclosure further describes methods for silencing of MSH3 and the treatment of diseases that may benefit from the silencing of MSH3 (e.g., diseases caused by pathological microsatellite DNA expansions, such as Huntington's Disease and myotonic dystrophy type 1, among others) by delivering MSH3-targeting siRNA molecules to a target tissue of a subject in need.

### BACKGROUND

MSH3 (MutS Homolog 3) encodes a protein that is important in the DNA mismatch repair system that may play a role in the age of onset and rate of progression of diseases caused by pathological microsatellite DNA expansions, including Huntington's Disease and myotonic dystrophy type 1. Recent studies show that individuals with a loss of function MSH3 mutation have delayed onset of Huntington's disease compared to individuals with normal forms of the gene. Currently, there are no treatments that can alter the course of Huntington's Disease. Accordingly, there is a need for therapeutics capable of selectively diminishing MSH3 activity in a manner that provides effective treatment for Huntington's Disease or other MSH3-related diseases or disorders.

### SUMMARY OF THE INVENTION

The present disclosure provides compositions and methods for reduction of MutS homolog 3 (MSH3) expression by way of small interfering RNA (siRNA)-mediated silencing of MSH3 transcripts. The compositions and methods provide the benefit of exhibiting high selectivity toward MSH3 over other genes.

The siRNA molecules of the disclosure can be used to silence the MSH3 gene, thereby preventing the translation of the corresponding mRNA transcript and reducing MSH3 protein expression. This reduction of MSH3 levels thus prevents disease onset or progression, as lengthening of DNA microsatellite repeats to pathological lengths depends on MSH3 function. The siRNA molecules of the disclosure can be delivered directly to a subject in need of MSH3 silencing by way of, for example, injection intrathecally, intracerebroventricularly, intrastriatally, intraparenchymally, intra-cisterna magna injection, such as by catheterization, intravenous injection, subcutaneous injection, or intramuscular injection.

In an aspect, the disclosure provides an siRNA molecule containing an antisense strand and sense strand having complementarity to the antisense strand. The antisense strand has complementarity sufficient to hybridize to a region within an MSH3 mRNA transcript having the nucleic acid sequence of any one of SEQ ID NOs: 1-408. In some embodiments, the antisense strand has complementarity sufficient to hybridize to a region within an MSH3 mRNA transcript having the nucleic acid sequence of any one of SEQ ID NOs: 5, 17-20, 33, 42, 44, 102, 103, 105, 106, 108, 109, 113, 126, 129, 130, 158, 161, 166, 177, 183, 193, 194, 196, 197, 202, 209, 217, 231, 271, 273, 291, 294, 305, 307, 317, 318, and 362. In some embodiments, the antisense strand has complementarity sufficient to hybridize to a region within an MSH3 mRNA transcript having the nucleic acid sequence of any one of SEQ ID NOs: 5, 17-20, 42, 44, 126, 129, 130, 158, 177, 183, 193, 194, 196, 197, 202, 209, 231, 271, 273, 291, 294, 305, 307, 317, 318, and 362. In some embodiments, the antisense strand has complementarity sufficient to hybridize to a region within an MSH3 mRNA transcript having the nucleic acid sequence of any one of SEQ ID NOs 1-24, 40-84, 100, 118-139, 173-181, 193-205, 220-222, 240-242, 249-308, 319-330, 336-377, 384-386, and 398-408. In some embodiments, the antisense strand has complementarity sufficient to hybridize to a region within an MSH3 mRNA transcript having the nucleic acid sequence of any one of SEQ ID NOs: 18-20, 102, 105, 106, 108, 109, 113, 183, 194, and 196. In some embodiments, the antisense strand has complementarity sufficient to hybridize to a region within an MSH3 mRNA transcript having the nucleic acid sequence of any one of SEQ ID NOs: 18-20, 183, 194, and 196.

The antisense strand may be, for example, from 10 to 50 nucleotides in length (e.g., from 10 to 45 nucleotides in length, from 10 to 40 nucleotides in length, from 10 to 35 nucleotides in length, from 10 to 30 nucleotides in length, from 10 to 29 nucleotides in length, from 10 to 28 nucleotides in length, from 10 to 27 nucleotides in length, from 10 to 26 nucleotides in length, from 10 to 25 nucleotides in length, from 10 to 24 nucleotides in length, from 10 to 23 nucleotides in length, from 10 to 22 nucleotides in length, from 10 to 21 nucleotides in length, or from 10 to 20 nucleotides in length).

In some embodiments, the antisense strand is 10 nucleotides in length, 11 nucleotides in length, 12 nucleotides in length, 13 nucleotides in length, 14 nucleotides in length, 15 nucleotides in length, 16 nucleotides in length, 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, 29 nucleotides in length, 30 nucleotides in length, or more.

In some embodiments, the antisense strand has at least 70% complementarity to the region within the MSH3 mRNA transcript having the nucleic acid sequence of any one of SEQ ID NOs: 1-408. In some embodiments, the region within the MSH3 mRNA transcript has the nucleic acid sequence of any one of SEQ ID NOs: 5, 17-20, 33, 42, 44, 102, 103, 105, 106, 108, 109, 113, 126, 129, 130, 158, 161, 166, 177, 183, 193, 194, 196, 197, 202, 209, 217, 231, 271, 273, 291, 294, 305, 307, 317, 318, and 362. In some embodiments, the region within the MSH3 mRNA transcript has the nucleic acid sequence of any one of SEQ ID NOs: 5, 17-20, 42, 44, 126, 129, 130, 158, 177, 183, 193, 194, 196, 197, 202, 209, 231, 271, 273, 291, 294, 305, 307, 317, 318, and 362. In some embodiments, the region within the MSH3 transcript has the nucleic acid sequence of any one of SEQ ID NOs: 1-24, 40-84, 100, 118-139, 173-181, 193-205, 220-222, 240-242, 249-308, 319-330, 336-377, 384-386, and 398-408. In some embodiments, the region within the MSH3 transcript has the nucleic acid sequence of any one of SEQ ID NOs: 18-20, 102, 105, 106, 108, 109, 113, 183, 194, and 196. In some embodiments, the region within the MSH3 transcript has the nucleic acid sequence of any one of SEQ ID NOs: 18-20, 183, 194, and 196.

In some embodiments, the antisense strand has at least 75% complementarity to the region within the MSH3 mRNA transcript having the nucleic acid sequence of any one of SEQ ID NOs: 1-408. For example, the antisense strand may have at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to the region within the MSH3 mRNA transcript having the nucleic acid sequence of any one of SEQ ID NOs: 1-408. In some embodiments, the region within the MSH3 mRNA transcript has the nucleic acid sequence of any one of SEQ ID NOs: 5, 17-20, 33, 42, 44, 102, 103, 105, 106, 108, 109, 113, 126, 129, 130, 158, 161, 166, 177, 183, 193, 194, 196, 197, 202, 209, 217, 231, 271, 273, 291, 294, 305, 307, 317, 318, and 362. In some embodiments, the region within the MSH3 mRNA transcript has the nucleic acid sequence of any one of SEQ ID NOs: 5, 17-20, 42, 44, 126, 129, 130, 158, 177, 183, 193, 194, 196, 197, 202, 209, 231, 271, 273, 291, 294, 305, 307, 317, 318, and 362. In some embodiments, the region within the MSH3 transcript has the nucleic acid sequence of any one of SEQ ID NOs: 1-24, 40-84, 100, 118-139, 173-181, 193-205, 220-222, 240-242, 249-308, 319-330, 336-377, 384-386, and 398-408. In some embodiments, the region within the MSH3 transcript has the nucleic acid sequence of any one of SEQ ID NOs: 18-20, 102, 105, 106, 108, 109, 113, 183, 194, and 196. In some embodiments, the region within the MSH3 transcript has the nucleic acid sequence of any one of SEQ ID NOs: 18-20, 183, 194, and 196.

In some embodiments, the antisense strand has at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 contiguous nucleotides that are fully complementary to a contiguous polynucleotide segment of equal length within the region of the MSH3 mRNA transcript having the nucleic acid sequence of any one of SEQ ID NOs: 1-408. In some embodiments, the region of the MSH3 mRNA transcript has the nucleic acid sequence of any one of SEQ ID NOs: 5, 17-20, 33, 42, 44, 102, 103, 105, 106, 108, 109, 113, 126, 129, 130, 158, 161, 166, 177, 183, 193, 194, 196, 197, 202, 209, 217, 231, 271, 273, 291, 294, 305, 307, 317, 318, and 362. In some embodiments, the region of the MSH3 mRNA transcript has the nucleic acid sequence of any one of SEQ ID NOs: 5, 17-20, 42, 44, 126, 129, 130, 158, 177, 183, 193, 194, 196, 197, 202, 209, 231, 271, 273, 291, 294, 305, 307, 317, 318, and 362. In some embodiments, the region of the MSH3 mRNA transcript has the nucleic acid sequence of any one of SEQ ID NOs: 1-24, 40-84, 100, 118-139, 173-181, 193-205, 220-222, 240-242, 249-308, 319-330, 336-377, 384-386, and 398-408. In some embodiments, the region of the MSH3 mRNA transcript has the nucleic acid sequence of any one of SEQ ID NOs: 18-20, 102, 105, 106, 108, 109, 113, 183, 194, and 196. In some embodiments, the region of the MSH3 mRNA transcript has the nucleic acid sequence of any one of SEQ ID NOs: 18-20, 183, 194, and 196.

In some embodiments, the antisense strand has from 10 to 30 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that are fully complementary to a contiguous polynucleotide segment of equal length within the region of the MSH3 mRNA transcript having the nucleic acid sequence of any one of SEQ ID NOs: 1-408. In some embodiments, the region of the MSH3 mRNA transcript has the nucleic acid sequence of any one of SEQ ID NOs: 5, 17-20, 33, 42, 44, 102, 103, 105, 106, 108, 109, 113, 126, 129, 130, 158, 161, 166, 177, 183, 193, 194, 196, 197, 202, 209, 217, 231, 271, 273, 291, 294, 305, 307, 317, 318, and 362. In some embodiments, the region of the MSH3 mRNA transcript has the nucleic acid sequence of any one of SEQ ID NOs: 5, 17-20, 42, 44, 126, 129, 130, 158, 177, 183, 193, 194, 196, 197, 202, 209, 231, 271, 273, 291, 294, 305, 307, 317, 318, and 362. In some embodiments, the region of the MSH3 mRNA transcript has the nucleic acid sequence of any one of SEQ ID NOs: 1-24, 40-84, 100, 118-139, 173-181, 193-205, 220-222, 240-242, 249-308, 319-330, 336-377, 384-386, and 398-408. In some embodiments, the region of the MSH3 mRNA transcript has the nucleic acid sequence of any one of SEQ ID NOs: 18-20, 102, 105, 106, 108, 109, 113, 183, 194, and 196. In some embodiments, the region of the MSH3 mRNA transcript has the nucleic acid sequence of any one of SEQ ID NOs: 18-20, 183, 194, and 196.

In some embodiments, the antisense strand has from 12 to 30 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that are fully complementary to a contiguous polynucleotide segment of equal length within the region of the MSH3 mRNA transcript having the nucleic acid sequence of any one of SEQ ID NOs: 1-408. In some embodiments, the region of the MSH3 mRNA transcript has the nucleic acid sequence of any one of SEQ ID NOs: 5, 17-20, 33, 42, 44, 102, 103, 105, 106, 108, 109, 113, 126, 129, 130, 158, 161, 166, 177, 183, 193, 194, 196, 197, 202, 209, 217, 231, 271, 273, 291, 294, 305, 307, 317, 318, and 362. In some embodiments, the region of the MSH3 mRNA transcript has the nucleic acid sequence of any one of SEQ ID NOs: 5, 17-20, 42, 44, 126, 129, 130, 158, 177, 183, 193, 194, 196, 197, 202, 209, 231, 271, 273, 291, 294, 305, 307, 317, 318, and 362. In some embodiments, the region of the MSH3 mRNA transcript has the nucleic acid sequence of any one of SEQ ID NOs: 1-24, 40-84, 100, 118-139, 173-181, 193-205, 220-222, 240-242, 249-308, 319-330, 336-377, 384-386, and 398-408. In some embodiments, the region of the MSH3 mRNA transcript has the nucleic acid sequence of any one of SEQ ID NOs: 18-20, 102, 105, 106, 108, 109, 113, 183, 194, and 196. In some embodiments, the region of the MSH3 mRNA transcript has the nucleic acid sequence of any one of SEQ ID NOs: 18-20, 183, 194, and 196.

In some embodiments, the antisense strand has from 15 to 30 contiguous nucleotides (e.g., 15, 16, 17, 18, 19, 20, 21, 22, 23, 24, 25, 26, 27, 28, 29, or 30 contiguous nucleotides) that are fully complementary to a contiguous polynucleotide segment of equal length within the region of the MSH3 mRNA transcript having the nucleic acid sequence of any one of SEQ ID NOs: 1-408. In some embodiments, the region of the MSH3 mRNA transcript has the nucleic acid sequence of any one of SEQ ID NOs: 5, 17-20, 33, 42, 44, 102, 103, 105, 106, 108, 109, 113, 126, 129, 130, 158, 161, 166, 177, 183, 193, 194, 196, 197, 202, 209, 217, 231, 271, 273, 291, 294, 305, 307, 317, 318, and 362. In some embodiments, the region of the MSH3 mRNA transcript has the nucleic acid sequence of any one of SEQ ID NOs: 5, 17-20, 42, 44, 126, 129, 130, 158, 177, 183, 193, 194, 196, 197, 202, 209, 231, 271, 273, 291, 294, 305, 307, 317, 318, and 362. In some embodiments, the region of the MSH3 mRNA transcript has the nucleic acid sequence of any one of SEQ ID NOs: 1-24, 40-84, 100, 118-139, 173-181, 193-205, 220-222, 240-242, 249-308, 319-330, 336-377, 384-386, and 398-408. In some embodiments, the region of the MSH3 mRNA transcript has the nucleic acid sequence of any one of SEQ ID NOs: 18-20, 102, 105, 106, 108, 109, 113, 183, 194, and 196. In some embodiments, the region of the MSH3 mRNA transcript has the nucleic acid sequence of any one of SEQ ID NOs: 18-20, 183, 194, and 196.

In some embodiments, the antisense strand has from 18 to 30 contiguous nucleotides (e.g., 18, 19, 20, 21, 22, 23, 24, 25, 26, 27, 28, 29, or 30 contiguous nucleotides) that are fully complementary to a contiguous polynucleotide segment of equal length within the region of the MSH3 RNA transcript having the nucleic acid sequence of any one of SEQ ID NOs: 1-408. In some embodiments, the region of the MSH3 mRNA transcript has the nucleic acid sequence of any one of SEQ ID NOs: 5, 17-20, 33, 42, 44, 102, 103, 105, 106, 108, 109, 113, 126, 129, 130, 158, 161, 166, 177, 183, 193, 194, 196, 197, 202, 209, 217, 231, 271, 273, 291, 294, 305, 307, 317, 318, and 362. In some embodiments, the region of the MSH3 mRNA transcript has the nucleic acid sequence of any one of SEQ ID NOs: 5, 17-20, 42, 44, 126, 129, 130, 158, 177, 183, 193, 194, 196, 197, 202, 209, 231, 271, 273, 291, 294, 305, 307, 317, 318, and 362. In some embodiments, the region of the MSH3 mRNA transcript has the nucleic acid sequence of any one of SEQ ID NOs: 1-24, 40-84, 100, 118-139, 173-181, 193-205, 220-222, 240-242, 249-308, 319-330, 336-377, 384-386, and 398-408. In some embodiments, the region of the MSH3 mRNA transcript has the nucleic acid sequence of any one of SEQ ID NOs: 18-20, 102, 105, 106, 108, 109, 113, 183, 194, and 196. In some embodiments, the region of the MSH3 mRNA transcript has the nucleic acid sequence of any one of SEQ ID NOs: 18-20, 183, 194, and 196.

In some embodiments, the antisense strand has from 18 to 25 contiguous nucleotides (e.g., 18, 19, 20, 21, 22, 23, 24, or 25 contiguous nucleotides) that are fully complementary to a contiguous polynucleotide segment of equal length within the region of the MSH3 mRNA transcript having the nucleic acid sequence of any one of SEQ ID Nos: 1-408. In some embodiments, the region of the MSH3 mRNA transcript has the nucleic acid sequence of any one of SEQ ID NOs: 5, 17-20, 33, 42, 44, 102, 103, 105, 106, 108, 109, 113, 126, 129, 130, 158, 161, 166, 177, 183, 193, 194, 196, 197, 202, 209, 217, 231, 271, 273, 291, 294, 305, 307, 317, 318, and 362. In some embodiments, the region of the MSH3 mRNA transcript has the nucleic acid sequence of any one of SEQ ID NOs: 5, 17-20, 42, 44, 126, 129, 130, 158, 177, 183, 193, 194, 196, 197, 202, 209, 231, 271, 273, 291, 294, 305, 307, 317, 318, and 362. In some embodiments, the region of the MSH3 mRNA transcript has the nucleic acid sequence of any one of SEQ ID NOs: 1-24, 40-84, 100, 118-139, 173-181, 193-205, 220-222, 240-242, 249-308, 319-330, 336-377, 384-386, and 398-408. In some embodiments, the region of the MSH3 mRNA transcript has the nucleic acid sequence of any one of SEQ ID NOs: 18-20, 102, 105, 106, 108, 109, 113, 183, 194, and 196. In some embodiments, the region of the MSH3 mRNA transcript has the nucleic acid sequence of any one of SEQ ID NOs: 18-20, 183, 194, and 196.

In some embodiments, the antisense strand has from 18 to 21 contiguous nucleotides (e.g., 18, 19, 20, or 21 contiguous nucleotides) that are fully complementary to a contiguous polynucleotide segment of equal length within the region of the MSH3 mRNA transcript having the nucleic acid sequence of any one of SEQ ID NOs: 1-408. In some embodiments, the region of the MSH3 mRNA transcript has the nucleic acid sequence of any one of SEQ ID NOs: 5, 17-20, 33, 42, 44, 102, 103, 105, 106, 108, 109, 113, 126, 129, 130, 158, 161, 166, 177, 183, 193, 194, 196, 197, 202, 209, 217, 231, 271, 273, 291, 294, 305, 307, 317, 318, and 362. In some embodiments, the region of the MSH3 mRNA transcript has the nucleic acid sequence of any one of SEQ ID NOs: 5, 17-20, 42, 44, 126, 129, 130, 158, 177, 183, 193, 194, 196, 197, 202, 209, 231, 271, 273, 291, 294, 305, 307, 317, 318, and 362. In some embodiments, the region of the MSH3 mRNA transcript has the nucleic acid sequence of any one of SEQ ID NOs: 1-24, 40-84, 100, 118-139, 173-181, 193-205, 220-222, 240-242, 249-308, 319-330, 336-377, 384-386, and 398-408. In some embodiments, the region of the MSH3 mRNA transcript has the nucleic acid sequence of any one of SEQ ID NOs: 18-20, 102, 105, 106, 108, 109, 113, 183, 194, and 196. In some embodiments, the region of the MSH3 mRNA transcript has the nucleic acid sequence of any one of SEQ ID NOs: 18-20, 183, 194, and 196.

In some embodiments, the antisense strand has 21 contiguous nucleotides that are fully complementary to a contiguous polynucleotide segment of equal length within the region of the MSH3 mRNA transcript having the nucleic acid sequence of any one of SEQ ID NOs: 1-408. In some embodiments, the region of the MSH3 mRNA transcript has the nucleic acid sequence of any one of SEQ ID NOs: 5, 17-20, 33, 42, 44, 102, 103, 105, 106, 108, 109, 113, 126, 129, 130, 158, 161, 166, 177, 183, 193, 194, 196, 197, 202, 209, 217, 231, 271, 273, 291, 294, 305, 307, 317, 318, and 362. In some embodiments, the region of the MSH3 mRNA transcript has the nucleic acid sequence of any one of SEQ ID NOs: 5, 17-20, 42, 44, 126, 129, 130, 158, 177, 183, 193, 194, 196, 197, 202, 209, 231, 271, 273, 291, 294, 305, 307, 317, 318, and 362. In some embodiments, the region of the MSH3 mRNA transcript has the nucleic acid sequence of any one of SEQ ID NOs: 1-24, 40-84, 100, 118-139, 173-181, 193-205, 220-222, 240-242, 249-308, 319-330, 336-377, 384-386, and 398-408. In some embodiments, the region of the MSH3 mRNA transcript has the nucleic acid sequence of any one of SEQ ID NOs: 18-20, 102, 105, 106, 108, 109, 113, 183, 194, and 196. In some embodiments, the region of the MSH3 mRNA transcript has the nucleic acid sequence of any one of SEQ ID NOs: 18-20, 183, 194, and 196.

In some embodiments, the antisense strand has 9 or fewer nucleotide mismatches relative to a region of 21 contiguous nucleobases of the MSH3 RNA transcript having the nucleic acid sequence of any one of SEQ ID NOs: 1-408, optionally wherein the antisense strand contains 8 or fewer, 7 or fewer, 6 or fewer, 5 or fewer, 4 or fewer, 3 or fewer, 2 or fewer, or only 1 mismatch relative to the region of the MSH3 mRNA transcript having the nucleic acid sequence of any one of SEQ ID NOs: 1-408. In some embodiments, the region of the MSH3 mRNA transcript has the nucleic acid sequence of any one of SEQ ID NOs: 5, 17-20, 33, 42, 44, 102, 103, 105, 106, 108, 109, 113, 126, 129, 130, 158, 161, 166, 177, 183, 193, 194, 196, 197, 202, 209, 217, 231, 271, 273, 291, 294, 305, 307, 317, 318, and 362. In some embodiments, the region of the MSH3 mRNA transcript has the nucleic acid sequence of any one of SEQ ID NOs: 5, 17-20, 42, 44, 126, 129, 130, 158, 177, 183, 193, 194, 196, 197, 202, 209, 231, 271, 273, 291, 294, 305, 307, 317, 318, and 362. In some embodiments, the region of the MSH3 mRNA transcript has the nucleic acid sequence of any one of SEQ ID NOs: 1-24, 40-84, 100, 118-139, 173-181, 193-205, 220-222, 240-242, 249-308, 319-330, 336-377, 384-386, and 398-408. In some embodiments, the region of the MSH3 mRNA transcript has the nucleic acid sequence of any one of SEQ ID NOs: 18-20, 102, 105, 106, 108, 109, 113, 183, 194, and 196. In some embodiments, the region of the MSH3 mRNA transcript has the nucleic acid sequence of any one of SEQ ID NOs: 18-20, 183, 194, and 196.

In some embodiments, the antisense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 817-1224. In some embodiments, the antisense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 821, 833-836, 849, 858, 860, 918, 919, 921, 922, 924, 925, 929, 942, 945, 946, 974, 977, 982, 993, 999, 1009, 1010, 1012, 1013, 1018, 1025, 1033, 1047, 1087, 1089, 1107, 1110, 1121, 1123, 1133, and 1134. In some embodiments, the antisense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 821, 833-836, 858, 860, 942, 945, 946, 974, 993, 999, 1009, 1010, 1012, 1013, 1018, 1025, 1047, 1087, 1089, 1107, 1110, 1121, 1123, 1133, and 1134. In some embodiments, the antisense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 817-840, 856-900, 916, 934-955, 989-997, 1009-1021, 1036-1038, 1056-1058, 1065-1124, 1135-1146, 1152-1193, 1200-1202, and 1214-1224. In some embodiments, the antisense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 834-836, 918, 921, 922, 924, 925, 999, 1010, and 1012. In some embodiments, the antisense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 834-836, 999, 1010, and 1012.

In some embodiments, the antisense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 817-1224. In some embodiments, the antisense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 821, 833-836, 849, 858, 860, 918, 919, 921, 922, 924, 925, 929, 942, 945, 946, 974, 977, 982, 993, 999, 1009, 1010, 1012, 1013, 1018, 1025, 1033, 1047, 1087, 1089, 1107, 1110, 1121, 1123, 1133, and 1134. In some embodiments, the antisense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 821, 833-836, 858, 860, 942, 945, 946, 974, 993, 999, 1009, 1010, 1012, 1013, 1018, 1025, 1047, 1087, 1089, 1107, 1110, 1121, 1123, 1133, and 1134. In some embodiments, the antisense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 817-840, 856-900, 916, 934-955, 989-997, 1009-1021, 1036-1038, 1056-1058, 1065-1124, 1135-1146, 1152-1193, 1200-1202, and 1214-1224. In some embodiments, the antisense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 834-836, 918, 921, 922, 924, 925, 999, 1010, and 1012. In some embodiments, the antisense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 834-836, 999, 1010, and 1012.

In some embodiments, the antisense strand has a nucleic acid sequence that is at least 95% identical (e.g., 95%, 96%, 97%, 98%, 99%, or 100% identical) to the nucleic acid sequence of any one of SEQ ID NOs: 817-1224, optionally wherein the antisense strand has a nucleic acid sequence that is at least 96%, 97%, 98%, or 99% identical to the nucleic acid sequence of any one of SEQ ID NOs: 817-1224. In some embodiments, the antisense strand has a nucleic acid sequence that is at least 95% identical (e.g., 95%, 96%, 97%, 98%, 99%, or 100% identical) to the nucleic acid sequence of any one of SEQ ID NOs: 821, 833-836, 849, 858, 860, 918, 919, 921, 922, 924, 925, 929, 942, 945, 946, 974, 977, 982, 993, 999, 1009, 1010, 1012, 1013, 1018, 1025, 1033, 1047, 1087, 1089, 1107, 1110, 1121, 1123, 1133, and 1134. In some embodiments, the antisense strand has a nucleic acid sequence that is at least 95% identical (e.g., 95%, 96%, 97%, 98%, 99%, or 100% identical) to the nucleic acid sequence of any one of SEQ ID NOs: 821, 833-836, 858, 860, 942, 945, 946, 974, 993, 999, 1009, 1010, 1012, 1013, 1018, 1025, 1047, 1087, 1089, 1107, 1110, 1121, 1123, 1133, and 1134. In some embodiments, the antisense strand has a nucleic acid sequence that is at least 95% identical (e.g., 95%, 96%, 97%, 98%, 99%, or 100% identical) to the nucleic acid sequence of any one of SEQ ID NOs: 817-840, 856-900, 916, 934-955, 989-997, 1009-1021, 1036-1038, 1056-1058, 1065-1124, 1135-1146, 1152-1193, 1200-1202, and 1214-1224. In some embodiments, the antisense strand has a nucleic acid sequence that is at least 95% identical (e.g., 95%, 96%, 97%, 98%, 99%, or 100% identical) to the nucleic acid sequence of any one of SEQ ID NOs: 834-836, 918, 921, 922, 924, 925, 999, 1010, and 1012. In some embodiments, the antisense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 834-836, 999, 1010, and 1012.

In some embodiments, the antisense strand has the nucleic acid sequence of any one of SEQ ID NOs: 817-1224. In some embodiments, the antisense strand has the nucleic acid sequence of any one of SEQ ID NOs: 821, 833-836, 849, 858, 860, 918, 919, 921, 922, 924, 925, 929, 942, 945, 946, 974, 977, 982, 993, 999, 1009, 1010, 1012, 1013, 1018, 1025, 1033, 1047, 1087, 1089, 1107, 1110, 1121, 1123, 1133, and 1134. In some embodiments the antisense strand has the nucleic acid sequence of any one of SEQ ID NOs: 821, 833-836, 858, 860, 942, 945, 946, 974, 993, 999, 1009, 1010, 1012, 1013, 1018, 1025, 1047, 1087, 1089, 1107, 1110, 1121, 1123, 1133, and 1134. In some embodiments, the antisense strand has the nucleic acid sequence of any one of SEQ ID NOs: 817-840, 856-900, 916, 934-955, 989-997, 1009-1021, 1036-1038, 1056-1058, 1065-1124, 1135-1146, 1152-1193, 1200-1202, and 1214-1224. In some embodiments, the antisense strand has the nucleic acid sequence of any one of SEQ ID NOs: 834-836, 918, 921, 922, 924, 925, 999, 1010, and 1012. In some embodiments, the antisense strand has the nucleic acid of any one of SEQ ID NOs: 834-836, 999, 1010, and 1012.

In some embodiments, the sense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 409-816. In some embodiments, the sense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 413, 425-428, 441, 450, 452, 510, 511, 513, 514, 516, 517, 521, 534, 537, 538, 566, 569, 574, 585, 591, 601, 602, 604, 605, 610, 617, 625, 639, 679, 681, 699, 702, 713, 715, 725, 726, and 770. In some embodiments, the sense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 413, 425-428, 450, 452, 534, 537, 538, 566, 585, 591, 601, 602, 604, 605, 610, 617, 639, 679, 681, 699, 702, 713, 715, 725, 726, and 770. In some embodiments, the sense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 409-432, 448-492, 508, 526-547, 581-589, 601-613, 628-630, 648-650, 657-716, 727-738, 744-785, 792-794, and 806-816. In some embodiments, the sense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 426-428, 510, 513, 514, 516, 517, 521, 591, 602, and 604. In some embodiments, the sense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 426-428, 591, 602, and 604.

In some embodiments, the sense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 409-816. In some embodiments, the sense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 413, 425-428, 441, 450, 452, 510, 511, 513, 514, 516, 517, 521, 534, 537, 538, 566, 569, 574, 585, 591, 601, 602, 604, 605, 610, 617, 625, 639, 679, 681, 699, 702, 713, 715, 725, 726, and 770. In some embodiments, the sense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 413, 425-428, 450, 452, 534, 537, 538, 566, 585, 591, 601, 602, 604, 605, 610, 617, 639, 679, 681, 699, 702, 713, 715, 725, 726, and 770. In some embodiments, the sense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 409-432, 448-492, 508, 526-547, 581-589, 601-613, 628-630, 648-650, 657-716, 727-738, 744-785, 792-794, and 806-816. In some embodiments, the sense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 426-428, 510, 513, 514, 516, 517, 521, 591, 602, and 604. In some embodiments, the sense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 426-428, 591, 602, and 604.

In some embodiments, the sense strand has a nucleic acid sequence that is at least 95% identical (e.g., 95%, 96%, 97%, 98%, 99%, or 100% identical) to the nucleic acid sequence of any one of SEQ ID NOs: 409-816, optionally wherein the sense strand has a nucleic acid sequence that is at least 96%, 97%, 98%, or 99% identical to the nucleic acid sequence of any one of SEQ ID NOs: 409-816. In some embodiments, the sense strand has a nucleic acid sequence that is at least 95% identical (e.g., 95%, 96%, 97%, 98%, 99%, or 100% identical) to the nucleic acid sequence of any one of SEQ ID NOs: 413, 425-428, 441, 450, 452, 510, 511, 513, 514, 516, 517, 521, 534, 537, 538, 566, 569, 574, 585, 591, 601, 602, 604, 605, 610, 617, 625, 639, 679, 681, 699, 702, 713, 715, 725, 726, and 770. In some embodiments, the sense strand has a nucleic acid sequence that is at least 95% identical (e.g., 95%, 96%, 97%, 98%, 99%, or 100% identical) to the nucleic acid sequence of any one of SEQ ID NOs: 413, 425-428, 450, 452, 534, 537, 538, 566, 585, 591, 601, 602, 604, 605, 610, 617, 639, 679, 681, 699, 702, 713, 715, 725, 726, and 770. In some embodiments, the sense strand has a nucleic acid sequence that is at least 95% identical (e.g., 95%, 96%, 97%, 98%, 99%, or 100% identical) to the nucleic acid sequence of any one of SEQ ID NOs: 409-432, 448-492, 508, 526-547, 581-589, 601-613, 628-630, 648-650, 657-716, 727-738, 744-785, 792-794, and 806-816. In some embodiments, the sense strand has a nucleic acid sequence that is at least 95% identical (e.g., 95%, 96%, 97%, 98%, 99%, or 100% identical) to the nucleic acid sequence of any one of SEQ ID NOs: 426-428, 510, 513, 514, 516, 517, 521, 591, 602, and 604. In some embodiments, the sense strand has a nucleic acid sequence that is at least 95% identical (e.g., 95%, 96%, 97%, 98%, 99%, or 100% identical) to the nucleic acid sequence of any one of SEQ ID NOs: 426-428, 591, 602, and 604.

In some embodiments, the siRNA molecule has a sense strand having the nucleic acid sequence of any one of SEQ ID NOs: 409-816. In some embodiments, the sense strand has a nucleic acid sequence of any one of SEQ ID NOs: 413, 425-428, 441, 450, 452, 510, 511, 513, 514, 516, 517, 521, 534, 537, 538, 566, 569, 574, 585, 591, 601, 602, 604, 605, 610, 617, 625, 639, 679, 681, 699, 702, 713, 715, 725, 726, and 770. In some embodiments, the sense strand has a nucleic acid sequence of any one of SEQ ID NOs: 413, 425-428, 450, 452, 534, 537, 538, 566, 585, 591, 601, 602, 604, 605, 610, 617, 639, 679, 681, 699, 702, 713, 715, 725, 726, and 770. In some embodiments, the siRNA molecule has a sense strand having the nucleic acid sequence of any one of SEQ ID NOs: 409-432, 448-492, 508, 526-547, 581-589, 601-613, 628-630, 648-650, 657-716, 727-738, 744-785, 792-794, and 806-816. In some embodiments, the sense strand has the nucleic acid sequence of any one of SEQ ID NOs: 426-428, 510, 513, 514, 516, 517, 521, 591, 602, and 604. In some embodiments, the sense strand has the nucleic acid sequence of any one of SEQ ID NOs: 426-428, 591, 602, and 604.

In some embodiments, the antisense strand has a structure represented by Formula I, wherein Formula I is, in the 5′-to-3′ direction:

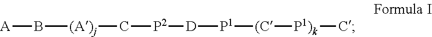

wherein A is represented by the formula C—P¹-D-P¹
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, the antisense strand has a structure represented by Formula A1, wherein Formula A1 is, in the 5′-to-3′ direction:

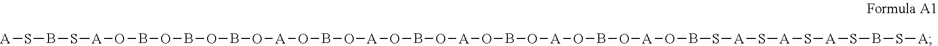

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula II, wherein Formula II is, in the 5′-to-3′ direction:

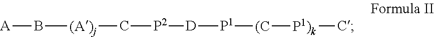

wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, antisense strand has a structure represented by Formula A2, wherein Formula A2 is, in the 5′-to-3′ direction:

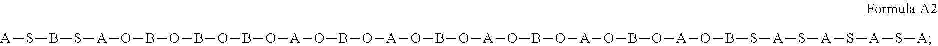

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula III, wherein Formula III is, in the 5′-to-3′ direction:

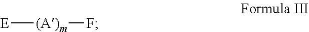

wherein E is represented by the formula (C—P¹)₂;
F is represented by the formula (C—P²)₃-D-P¹—C—P¹—C, (C—P²)₃-D-P²—C—P²—C, (C—P²)₃-D-P¹—C—P¹-D, or (C—P²)₃-D-P²—C—P²-D;
A′, C, D, P¹, and P² are as defined in Formula II; and
m is an integer from 1 to 7 (e.g., 1, 2, 3, 4, 5, 6, or 7).

In some embodiments, the sense strand has a structure represented by Formula S1, wherein Formula S1 is, in the 5′-to-3′ direction:

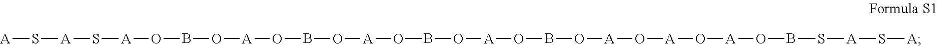

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S2, wherein Formula S2 is, in the 5′-to-3′ direction:

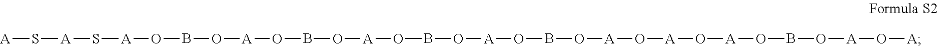

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S3, wherein Formula S3 is, in the 5′-to-3′ direction:

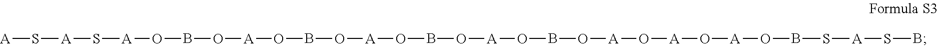

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S4, wherein Formula S4 is, in the 5′-to-3′ direction:

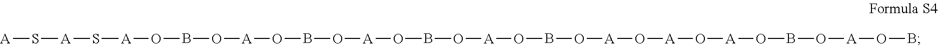

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula IV, wherein Formula IV is, in the 5′-to-3′ direction:

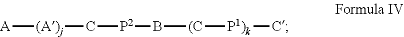

wherein A is represented by the formula C—P¹-D-P¹;
each A′ is represented by the formula C—P²-D-P²;
B is represented by the formula D-P¹—C—P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, the antisense strand has a structure represented by Formula A3, wherein Formula A3 is, in the 5′-to-3′ direction:

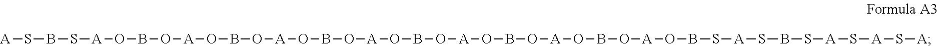

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula V, wherein Formula V is, in the 5′-to-3′ direction:

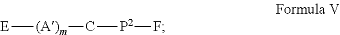

wherein E is represented by the formula (C—P¹)₂;
F is represented by the formula D-P¹—C—P¹—C, D-P²—C—P²—C, D-P¹—C—P¹-D, or D-P²—C—P²-D;
A′, C, D, P¹ and P² are as defined in Formula IV; and
m is an integer from 1 to 7 (e.g., 1, 2, 3, 4, 5, 6, or 7).

In some embodiments, the sense strand has a structure represented by Formula S5, wherein Formula S5 is, in the 5′-to-3′ direction:

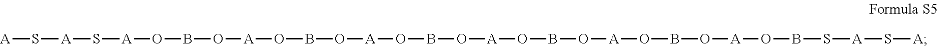

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S6, wherein Formula S6 is, in the 5′-to-3′ direction:

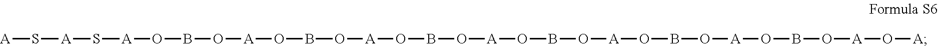

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S7, wherein Formula S7 is, in the 5′-to-3′ direction:

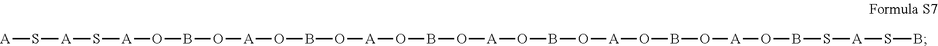

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S8, wherein Formula S8 is, in the 5′-to-3′ direction:

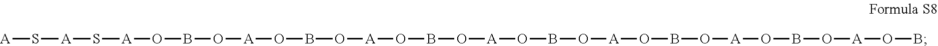

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula VI, wherein Formula VI is, in the 5′-to-3′ direction:

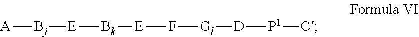

wherein A is represented by the formula C—P¹-D-P¹;
each B is represented by the formula C—P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²—C—P²;
F is represented by the formula D-P¹—C—P¹;
each G is represented by the formula C—P¹;
each P¹ is a phosphorothioate internucleoside linkage;
each P² is a phosphodiester internucleoside linkage;
j is an integer from 1 to 7 (e.g., 1, 2, 3, 4, 5, 6, or 7);
k is an integer from 1 to 7 (e.g., 1, 2, 3, 4, 5, 6, or 7); and
l is an integer from 1 to 7 (e.g., 1, 2, 3, 4, 5, 6, or 7).

In some embodiments, the antisense strand has a structure represented by Formula A4, wherein Formula A4 is, in the 5′-to-3′ direction:

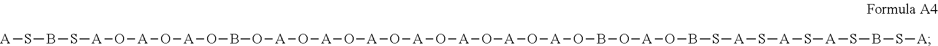

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula VII, wherein Formula VII is, in the 5′-to-3′ direction:

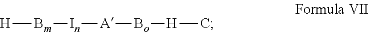

wherein A′ is represented by the formula C—P²-D-P²;
each H is represented by the formula (C—P¹)₂;
each I is represented by the formula (D-P²);
B, C, D, P¹ and P² are as defined in Formula VI;
m is an integer from 1 to 7 (e.g., 1, 2, 3, 4, 5, 6, or 7);
n is an integer from 1 to 7 (e.g., 1, 2, 3, 4, 5, 6, or 7); and
o is an integer from 1 to 7 (e.g., 1, 2, 3, 4, 5, 6, or 7).

In some embodiments, the sense strand has a structure represented by Formula S9, wherein Formula S9 is, in the 5′-to-3′ direction:

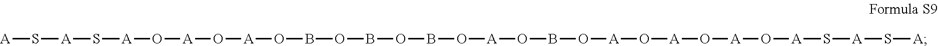

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand also has a 5′ phosphorus stabilizing moiety at the 5′ end of the antisense strand.

In some embodiments, the sense strand also has a 5′ phosphorus stabilizing moiety at the 5′ end of the sense strand.

In some embodiments, each 5′ phosphorus stabilizing moiety is, independently, represented by any one of Formulas IX, XX, XI, XII, XIII, XIV, XV, or XVI:

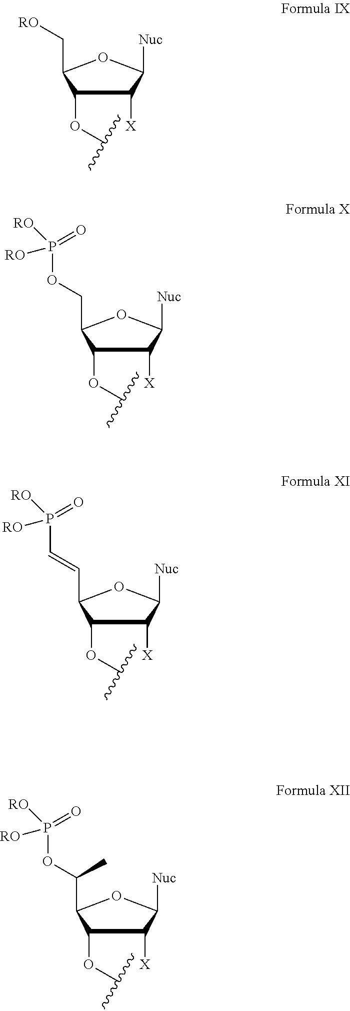

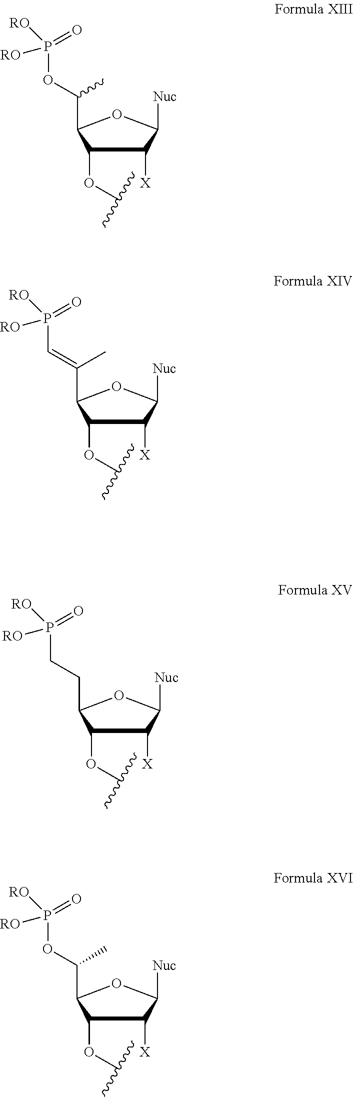

wherein Nuc represents a nucleobase selected from the group consisting of adenine, uracil, guanine, thymine, and cytosine, and R represents an optionally substituted alkyl, optionally substituted alkenyl, optionally substituted alkynyl, phenyl, benzyl, hydroxy, or hydrogen.

In some embodiments, the nucleobase is an adenine, uracil, guanine, thymine, or cytosine.

In some embodiments, the 5′ phosphorus stabilizing moiety is (E)-vinylphosphonate represented by Formula XI.

In some embodiments, the siRNA molecule also has a hydrophobic moiety at the 5′ or the 3′ end of the siRNA molecule.

In some embodiments, the hydrophobic moiety is selected from a group consisting of cholesterol, vitamin D, or tocopherol.

In some embodiments, the length of the sense strand is between 12 and 30 nucleotides (e.g., 12, 13, 14, 15, 16, 17, 18, 19, 20, 21, 22, 23, 24, 25, 26, 27, 28, 29, or 30 nucleotides).

In some embodiments, the siRNA molecule is a branched siRNA molecule.

In some embodiments, the branched siRNA molecule is di-branched, tri-branched, or tetra-branched.

In some embodiments, the siRNA molecule is di-branched, optionally wherein the di-branched siRNA molecule is represented by any one of Formulas XVII, XVIII, or XIX:

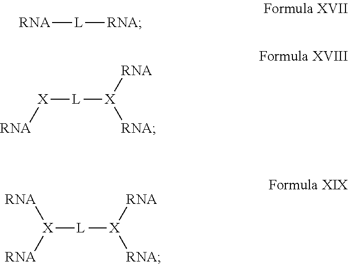

wherein each RNA is, independently, an siRNA molecule, L is a linker, and each X, independently, represents a branch point moiety.

In some embodiments, the siRNA molecule is tri-branched, optionally wherein the tri-branched siRNA molecule is represented by any one of Formulas XX, XXI, XXII, or XXIII:

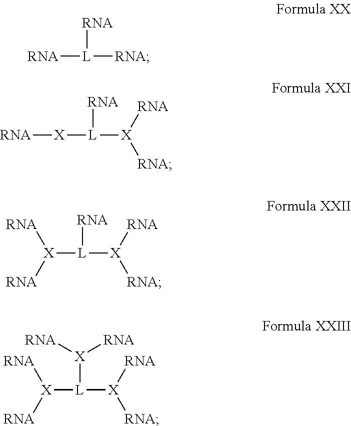

wherein each RNA is, independently, an siRNA molecule, L is a linker, and each X, independently, represents a branch point moiety.

In some embodiments, the siRNA molecule is tetra-branched, optionally wherein the tetra-branched siRNA molecule is represented by any one of Formulas XXIV, XXV, XXVI, XXVII, or XXVIII:

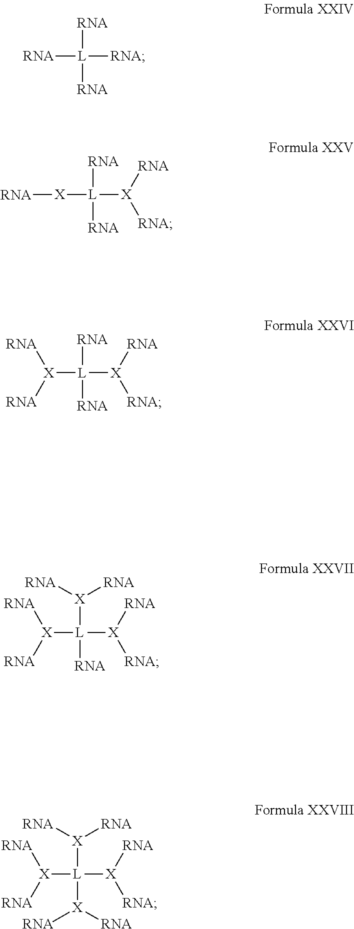

wherein each RNA is, independently, an siRNA molecule, L is a linker, and each X, independently, represents a branch point moiety.

In some embodiments, the linker is selected from a group consisting of one or more contiguous subunits of an ethylene glycol, alkyl, carbohydrate, block copolymer, peptide, RNA, and DNA.

In some embodiments, the one or more contiguous subunits is 2 to 20 contiguous subunits (e.g., 2, 3, 4, 5, 6, 7, 8, 9, 10, 11, 12, 13, 14, 15, 16, 17, 18, 19, or 20 contiguous subunits).

In some embodiments of any of the siRNA molecules described herein, the antisense strand has complementarity sufficient to hybridize to a region within an MSH3 mRNA transcript that is within the open reading frame of the MSH3 mRNA transcript. In some embodiments, the antisense strand has complementarity sufficient to hybridize to a region within an MSH3 mRNA transcript that is within the 3-untranslated region.

In some embodiments of any of the siRNA molecules described herein, the antisense strand has complementarity sufficient to hybridize to a region within exon 1 of an MSH3 mRNA transcript. In some embodiments, the antisense strand has complementarity sufficient to hybridize to a region within an MSH3 mRNA transcript having the nucleic acid sequence of any one of SEQ ID NOs: 1-4.

In some embodiments of any of the siRNA molecules described herein, the antisense strand has complementarity sufficient to hybridize to a region within exon 2 of an MSH3 mRNA transcript. In some embodiments, the antisense strand has complementarity sufficient to hybridize to a region within an MSH3 mRNA transcript having the nucleic acid sequence of any one of SEQ ID NOs: 5-20. In some embodiments, the antisense strand has complementarity sufficient to hybridize to a region within an MSH3 mRNA transcript having the nucleic acid sequence of any one of SEQ ID NOs: 5-15.

In some embodiments of any of the siRNA molecules described herein, the antisense strand has complementarity sufficient to hybridize to a region within exon 3 of an MSH3 mRNA transcript. In some embodiments, the antisense strand has complementarity sufficient to hybridize to a region within an MSH3 mRNA transcript having the nucleic acid sequence of any one of SEQ ID NOs: 16-47. In some embodiments, the antisense strand has complementarity sufficient to hybridize to a region within an MSH3 mRNA transcript having the nucleic acid sequence of any one of SEQ ID NOs: 21-47.

In some embodiments of any of the siRNA molecules described herein, the antisense strand has complementarity sufficient to hybridize to a region within exon 4 of an MSH3 mRNA transcript. In some embodiments, the antisense strand has complementarity sufficient to hybridize to a region within an MSH3 mRNA transcript having the nucleic acid sequence of any one of SEQ ID NOs: 48-84.

In some embodiments of any of the siRNA molecules described herein, the antisense strand has complementarity sufficient to hybridize to a region within exon 5 of an MSH3 mRNA transcript. In some embodiments, the antisense strand has complementarity sufficient to hybridize to a region within an MSH3 mRNA transcript having the nucleic acid sequence of any one of SEQ ID NOs: 85-103. In some embodiments, the antisense strand has complementarity sufficient to hybridize to a region within an MSH3 mRNA transcript having the nucleic acid sequence of any one of SEQ ID NOs: 85-100.

In some embodiments of any of the siRNA molecules described herein, the antisense strand has complementarity sufficient to hybridize to a region within exon 6 of an MSH3 mRNA transcript. In some embodiments, the antisense strand has complementarity sufficient to hybridize to a region within an MSH3 mRNA transcript having the nucleic acid sequence of any one of SEQ ID NOs: 101-120. In some embodiments, the antisense strand has complementarity sufficient to hybridize to a region within an MSH3 mRNA transcript having the nucleic acid sequence of any one of SEQ ID NOs: 104-120.

In some embodiments of any of the siRNA molecules described herein, the antisense strand has complementarity sufficient to hybridize to a region within exon 7 of an MSH3 mRNA transcript. In some embodiments, the antisense strand has complementarity sufficient to hybridize to a region within an MSH3 mRNA transcript having the nucleic acid sequence of any one of SEQ ID NOs: 121-133.

In some embodiments of any of the siRNA molecules described herein, the antisense strand has complementarity sufficient to hybridize to a region within exon 8 of an MSH3 mRNA transcript. In some embodiments, the antisense strand has complementarity sufficient to hybridize to a region within an MSH3 mRNA transcript having the nucleic acid sequence of any one of SEQ ID NOs: 134-139.

In some embodiments of any of the siRNA molecules described herein, the antisense strand has complementarity sufficient to hybridize to a region within exon 9 of an MSH3 mRNA transcript. In some embodiments, the antisense strand has complementarity sufficient to hybridize to a region within an MSH3 mRNA transcript having the nucleic acid sequence of any one of SEQ ID NOs: 140-162. In some embodiments, the antisense strand has complementarity sufficient to hybridize to a region within an MSH3 mRNA transcript having the nucleic acid sequence of any one of SEQ ID NOs: 140-154.

In some embodiments of any of the siRNA molecules described herein, the antisense strand has complementarity sufficient to hybridize to a region within exon 10 of an MSH3 mRNA transcript. In some embodiments, the antisense strand has complementarity sufficient to hybridize to a region within an MSH3 mRNA transcript having the nucleic acid sequence of any one of SEQ ID NOs: 155-187. In some embodiments, the antisense strand has complementarity sufficient to hybridize to a region within an MSH3 mRNA transcript having the nucleic acid sequence of any one of SEQ ID NOs: 163-182.

In some embodiments of any of the siRNA molecules described herein, the antisense strand has complementarity sufficient to hybridize to a region within exon 11 of an MSH3 mRNA transcript. In some embodiments, the antisense strand has complementarity sufficient to hybridize to a region within an MSH3 mRNA transcript having the nucleic acid sequence of any one of SEQ ID NOs: 183-200. In some embodiments, the antisense strand has complementarity sufficient to hybridize to a region within an MSH3 mRNA transcript having the nucleic acid sequence of any one of SEQ ID NOs: 188-192.

In some embodiments of any of the siRNA molecules described herein, the antisense strand has complementarity sufficient to hybridize to a region within exon 12 of an MSH3 mRNA transcript. In some embodiments, the antisense strand has complementarity sufficient to hybridize to a region within an MSH3 mRNA transcript having the nucleic acid sequence of any one of SEQ ID NOs: 193-205.

In some embodiments of any of the siRNA molecules described herein, the antisense strand has complementarity sufficient to hybridize to a region within exon 13 of an MSH3 mRNA transcript. In some embodiments, the antisense strand has complementarity sufficient to hybridize to a region within an MSH3 mRNA transcript having the nucleic acid sequence of any one of SEQ ID NOs: 206-223. In some embodiments, the antisense strand has complementarity sufficient to hybridize to a region within an MSH3 mRNA transcript having the nucleic acid sequence of any one of SEQ ID NOs: 206-222.

In some embodiments of any of the siRNA molecules described herein, the antisense strand has complementarity sufficient to hybridize to a region within exon 14 of an MSH3 mRNA transcript. In some embodiments, the antisense strand has complementarity sufficient to hybridize to a region within an MSH3 mRNA transcript having the nucleic acid sequence of any one of SEQ ID NOs: 223-242. In some embodiments, the antisense strand has complementarity sufficient to hybridize to a region within an MSH3 mRNA transcript having the nucleic acid sequence of any one of SEQ ID NOs: 224-242.

In some embodiments of any of the siRNA molecules described herein, the antisense strand has complementarity sufficient to hybridize to a region within exon 15 of an MSH3 mRNA transcript. In some embodiments, the antisense strand has complementarity sufficient to hybridize to a region within an MSH3 mRNA transcript having the nucleic acid sequence of any one of SEQ ID NOs: 243-262. In some embodiments, the antisense strand has complementarity sufficient to hybridize to a region within an MSH3 mRNA transcript having the nucleic acid sequence of any one of SEQ ID NOs: 243-255.

In some embodiments of any of the siRNA molecules described herein, the antisense strand has complementarity sufficient to hybridize to a region within exon 16 of an MSH3 mRNA transcript. In some embodiments, the antisense strand has complementarity sufficient to hybridize to a region within an MSH3 mRNA transcript having the nucleic acid sequence of any one of SEQ ID NOs: 256-276. In some embodiments, the antisense strand has complementarity sufficient to hybridize to a region within an MSH3 mRNA transcript having the nucleic acid sequence of any one of SEQ ID NOs: 263-268

In some embodiments of any of the siRNA molecules described herein, the antisense strand has complementarity sufficient to hybridize to a region within exon 17 of an MSH3 mRNA transcript. In some embodiments, the antisense strand has complementarity sufficient to hybridize to a region within an MSH3 mRNA transcript having the nucleic acid sequence of any one of SEQ ID NOs: 269-296. In some embodiments, the antisense strand has complementarity sufficient to hybridize to a region within an MSH3 mRNA transcript having the nucleic acid sequence of any one of SEQ ID NOs: 277-289.

In some embodiments of any of the siRNA molecules described herein, the antisense strand has complementarity sufficient to hybridize to a region within exon 18 of an MSH3 mRNA transcript. In some embodiments, the antisense strand has complementarity sufficient to hybridize to a region within an MSH3 mRNA transcript having the nucleic acid sequence of any one of SEQ ID NOs: 290-302. In some embodiments, the antisense strand has complementarity sufficient to hybridize to a region within an MSH3 mRNA transcript having the nucleic acid sequence of any one of SEQ ID NOs: 297-302.

In some embodiments of any of the siRNA molecules described herein, the antisense strand has complementarity sufficient to hybridize to a region within exon 19 of an MSH3 mRNA transcript. In some embodiments, the antisense strand has complementarity sufficient to hybridize to a region within an MSH3 mRNA transcript having the nucleic acid sequence of any one of SEQ ID NOs: 303-304.

In some embodiments of any of the siRNA molecules described herein, the antisense strand has complementarity sufficient to hybridize to a region within exon 20 of an MSH3 mRNA transcript. In some embodiments, the antisense strand has complementarity sufficient to hybridize to a region within an MSH3 mRNA transcript having the nucleic acid sequence of any one of SEQ ID NOs: 303-324. In some embodiments, the antisense strand has complementarity sufficient to hybridize to a region within an MSH3 mRNA transcript having the nucleic acid sequence of any one of SEQ ID NOs: 305-318.

In some embodiments of any of the siRNA molecules described herein, the antisense strand has complementarity sufficient to hybridize to a region within exon 21 of an MSH3 mRNA transcript. In some embodiments, the antisense strand has complementarity sufficient to hybridize to a region within an MSH3 mRNA transcript having the nucleic acid sequence of any one of SEQ ID NOs: 319-339. In some embodiments, the antisense strand has complementarity sufficient to hybridize to a region within an MSH3 mRNA transcript having the nucleic acid sequence of any one of SEQ ID NOs: 325-336.

In some embodiments of any of the siRNA molecules described herein, the antisense strand has complementarity sufficient to hybridize to a region within exon 22 of an MSH3 mRNA transcript. In some embodiments, the antisense strand has complementarity sufficient to hybridize to a region within an MSH3 mRNA transcript having the nucleic acid sequence of any one of SEQ ID NOs: 337-344. In some embodiments, the antisense strand has complementarity sufficient to hybridize to a region within an MSH3 mRNA transcript having the nucleic acid sequence of any one of SEQ ID NOs: 340-344.

In some embodiments of any of the siRNA molecules described herein, the antisense strand has complementarity sufficient to hybridize to a region within exon 23 of an MSH3 mRNA transcript. In some embodiments, the antisense strand has complementarity sufficient to hybridize to a region within an MSH3 mRNA transcript having the nucleic acid sequence of any one of SEQ ID NOs: 345-358.

In some embodiments of any of the siRNA molecules described herein, the antisense strand has complementarity sufficient to hybridize to a region within exon 24 of an MSH3 mRNA transcript. In some embodiments, the antisense strand has complementarity sufficient to hybridize to a region within an MSH3 mRNA transcript having the nucleic acid sequence of any one of SEQ ID NOs: 359-408.

In a further aspect, the disclosure provides a pharmaceutical composition containing an siRNA molecule of any of the preceding aspects or embodiments of the disclosure, and a pharmaceutically acceptable excipient, carrier, or diluent.

In a further aspect, the disclosure provides a method of delivering an siRNA molecule to a subject diagnosed as having a microsatellite repeat expansion disorder by administering a therapeutically effective amount of the siRNA molecule or a pharmaceutical composition of any of the preceding aspects or embodiments of the disclosure to the subject. In some embodiments, the microsatellite repeat expansion disease is Huntington's Disease. In some embodiments, the microsatellite repeat expansion disease is a spinocerebellar ataxia. In some embodiments, the microsatellite repeat expansion disease is a Fragile X syndrome. In some embodiments, the microsatellite repeat expansion disease is a myotonic dystrophy.

In a further aspect, the disclosure provides a method of treating a microsatellite repeat expansion disorder in a subject in need thereof by administering a therapeutically effective amount of an siRNA molecule or a pharmaceutical composition of any of the preceding aspects or embodiments of the disclosure to the subject. In some embodiments, the microsatellite repeat expansion disease is Huntington's disease.

In another aspect, the disclosure provides a method of reducing MSH3 expression in a subject in need thereof by administering a therapeutically effective amount of an siRNA or pharmaceutical composition of any of the preceding aspects or embodiments of the disclosure.

In some embodiments, the siRNA molecule or the pharmaceutical composition is administered to the subject by way of intracerebroventricular, intrastriatal, intraparenchymal or intrathecal injection. In some embodiments, the siRNA molecule or the pharmaceutical composition is administered to the subject by way of intravenous, intramuscular, or subcutaneous injection.

In some embodiments, the subject is a human.

In another aspect, the disclosure provides a kit containing an siRNA molecule or pharmaceutical composition of any of the preceding aspects or embodiments of the disclosure, and a package insert that instructs a user of the kit to perform the method of any of the preceding aspects or embodiments of the disclosure.

### BRIEF DESCRIPTION OF THE FIGURES

FIG. 1 is a graph showing reduction in MSH3 protein in FVB mice that were administered siRNA molecules of the disclosure. The siRNA identifiers are shown in Table 6, below. Protein was quantified in the motor cortex, 28 days following di-siRNA treatment at 1 nmol or 5 nmol dose levels.

FIG. 2 is a graph showing reduction in MSH3 protein in FVB mice that were administered siRNA molecules of the disclosure. The siRNA identifiers are shown in Table 6, below. Protein was quantified in the striatum, 28 days following di-siRNA treatment at 1 nmol or 5 nmol dose levels.

FIG. 3 is a graph showing reduction in MSH3 protein in FVB mice that were administered siRNA molecules of the disclosure. The siRNA identifiers are shown in Table 6, below. Protein was quantified in the hippocampus, 28 days following di-siRNA treatment at 1 nmol or 5 nmol dose levels.

FIG. 4 is a graph showing reduction in MSH3 mRNA in FVB mice that were administered siRNA molecules of the disclosure. The siRNA identifiers are shown in Table 6, below. mRNA was quantified in the motor cortex, 28 days following di-siRNA treatment at 1 nmol or 5 nmol dose levels.

FIG. 5 is a graph showing reduction in MSH3 mRNA in FVB mice that were administered siRNA molecules of the disclosure. The siRNA identifiers are shown in Table 6, below. mRNA was quantified in the striatum, 28 days following di-siRNA treatment at 1 nmol or 5 nmol dose levels.

FIG. 6 is a graph showing reduction in MSH3 mRNA in FVB mice that were administered siRNA molecules of the disclosure. The siRNA identifiers are shown in Table 6, below. mRNA was quantified in the hippocampus, 28 days following di-siRNA treatment at 1 nmol or 5 nmol dose levels.

FIG. 7 is a graph showing reduction in MSH3 protein in FVB mice that were administered siRNA molecules of the disclosure. The siRNA identifiers are shown in Table 6, below. Protein was quantified in the motor cortex, striatum, and hippocampus, 28 days following di-siRNA treatment at a 5 nmol dose level.

FIG. 8 is a graph showing reduction in MSH3 mRNA in FVB mice that were administered siRNA molecules of the disclosure. The siRNA identifiers are shown in Table 6, below. mRNA was quantified in the motor cortex, striatum, and hippocampus, 28 days following di-siRNA treatment at a 5 nmol dose level.

FIG. 9 is a graph showing reduction in MSH3 protein in FVB mice that were administered siRNA molecules of the disclosure. The siRNA identifiers are shown in Table 6, below. Protein was quantified in the motor cortex, striatum, and hippocampus, 28 days following di-siRNA treatment at 0.5 nmol, 1 nmol, 2.5 nmol, 5 nmol, and 20 nmol dose levels.

### DEFINITIONS

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the term “nucleic acids” refers to RNA or DNA molecules consisting of a chain of ribonucleotides or deoxyribonucleotides, respectively.

As used herein, the term “therapeutic nucleic acid” refers to a nucleic acid molecule (e.g., ribonucleic acid) that has partial or complete complementarity to, and interacts with, a disease-associated target mRNA and mediates silencing of expression of the mRNA.

As used herein, the term “carrier nucleic acid” refers to a nucleic acid molecule (e.g., ribonucleic acid) that has sequence complementarity with, and hybridizes with, a therapeutic nucleic acid. As used herein, the term “3′ end” refers to the end of the nucleic acid that contains an unmodified hydroxyl group at the 3′ carbon of the ribose ring.

As used herein, the term “nucleoside” refers to a molecule made up of a heterocyclic base and its sugar.

As used herein, the term “nucleotide” refers to a nucleoside having a phosphate group, or a variant thereof, on its 3′ or 5′ sugar hydroxyl group. Examples of phosphate group variants include, but are not limited to, saturated alkyl phosphonates, unsaturated alkenyl phosphonates, phosphorothioates, and phosphoramidites.

In the context of this disclosure, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring (e.g., modified) portions that function similarly. Such modified or substituted oligonucleotides are often preferred over native forms because of desirable properties such as, for example, enhanced cellular uptake, enhanced affinity for nucleic acid target and increased stability in the presence of nucleases.

As used herein, the term “siRNA” refers to small interfering RNA duplexes that induce the RNA interference (RNAi) pathway. siRNA molecules may vary in length (generally, between 10 and 30 base pairs) and may contain varying degrees of complementarity to their target mRNA. The term “siRNA” includes duplexes of two separate strands, as well as single strands that optionally form hairpin structures including a duplex region.

As used herein, the term “antisense strand” refers to the strand of the siRNA duplex that contains some degree of complementarity to the target gene.

As used herein, the term “sense strand” refers to the strand of the siRNA duplex that contains complementarity to the antisense strand.

The term “interfering RNA molecule” refers to an RNA molecule, such as a small interfering RNA (siRNA), microRNA (miRNA), short hairpin RNA (shRNA), or an antisense oligonucleotide (ASO) that suppresses the endogenous function of a target RNA transcript.

As used herein, the terms “express” and “expression” refer to one or more of the following events: (1) production of an RNA template from a DNA sequence (e.g., by transcription); (2) processing of an RNA transcript (e.g., by splicing, editing, 5′ cap formation, and/or 3′ end processing); and (3) translation of an RNA into a polypeptide or protein. In the context of a gene that encodes a protein product, the terms “gene expression” and the like are used interchangeably with the terms “protein expression” and the like. A change in expression of a gene or protein of interest in a patient can be detected, for example, by: a change in the quantity or concentration of mRNA encoding corresponding protein (as assessed, e.g., using RNA detection procedures described herein or known in the art, such as quantitative polymerase chain reaction (qPCR) and RNA-seq techniques), a change in the quantity or concentration of the corresponding protein (as assessed, e.g., using protein detection methods described herein or known in the art, such as enzyme-linked immunosorbent assays (ELISA), among others), and/or a change in the activity of the corresponding protein (e.g., in the case of an enzyme, as assessed using an enzymatic activity assay described herein or known in the art) in a sample obtained from the patient. As used herein, a cell is considered to “express” a gene or protein of interest if one or more, or all, of the above events can be detected in the cell or in a medium in which the cell resides. For example, a gene or protein of interest is considered to be “expressed” by a cell or population of cells if one can detect (i) production of a corresponding RNA transcript, such as an mRNA template, by the cell or population of cells (e.g., using RNA detection procedures described herein); (ii) processing of the RNA transcript (e.g., splicing, editing, 5′ cap formation, and/or 3′ end processing, such as using RNA detection procedures described herein); (iii) translation of the RNA template into a protein product (e.g., using protein detection procedures described herein); and/or (iv) post-translational modification of the protein product (e.g., using protein detection procedures described herein).

As used herein, the terms “target,” “targeting,” and “targeted,” in the context of the design of an siRNA, refers to generating an antisense strand so as to anneal the antisense strand to a region within the mRNA transcript of interest in a manner that results in a reduction in translation of the mRNA into the protein product.

As used herein, the terms “chemically modified nucleotide,” “nucleotide analog,” “altered nucleotide,” and “modified nucleotide” refer to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function.

As used herein, the term “metabolically stabilized” refers to RNA molecules that contain ribonucleotides that have been chemically modified in order to decrease the rate of metabolism of an RNA molecule that is administered to a subject. Exemplary modifications include 2′-hydroxy to 2′-O-methoxy or 2′-fluoro, and phosphodiester to phosphorothioate.

As used herein, the term “phosphorothioate” refers to a phosphate group of a nucleotide that is modified by substituting one or more of the oxygens of the phosphate group with sulfur.

As used herein, the terms “internucleoside linkage,” “internucleoside bond,” and the like refer to the bonds between nucleosides in a nucleic acid molecule.

As used herein, the term “antagomirs” refers to nucleic acids that can function as inhibitors of miRNA activity.

As used herein, the term “gapmers” refers to chimeric antisense nucleic acids that contain a central block of deoxynucleotide monomers sufficiently long to induce RNase H cleavage. The deoxynucleotide block is flanked by ribonucleotide monomers or ribonucleotide monomers containing modifications.

As used herein, the term “mixmers” refers to nucleic acids that contain a mix of locked nucleic acids (LNAs) and DNA.

As used herein, the term “guide RNAs” refers to nucleic acids that have sequence complementarity to a specific sequence in the genome immediately or 1 base pair upstream of the protospacer adjacent motif (PAM) sequence as used in CRISPR/Cas9 gene editing systems. Alternatively, “guide RNAs” may refer to nucleic acids that have sequence complementarity (e.g., are antisense) to a specific messenger RNA (mRNA) sequence. In this context, a guide RNA may also have sequence complementarity to a “passenger RNA” sequence of equal or shorter length, which is identical or substantially identical to the sequence of mRNA to which the guide RNA hybridizes.

As used herein, the term “branched siRNA” refers to a compound containing two or more double-stranded siRNA molecules covalently bound to one another. Branched siRNA molecules may be “di-branched,” also referred to herein as “di-siRNA,” wherein the siRNA molecule includes 2 siRNA molecules covalently bound to one another, e.g., by way of a linker. Branched siRNA molecules may be “tri-branched,” also referred to herein as “tri-siRNA,” wherein the siRNA molecule includes 3 siRNA molecules covalently bound to one another, e.g., by way of a linker. Branched siRNA molecules may be “tetra-branched,” also referred to herein as “tetra-siRNA,” wherein the siRNA molecule includes 4 siRNA molecules covalently bound to one another, e.g., by way of a linker.

As used herein, the term “branch point moiety” refers to a chemical moiety of a branched siRNA structure of the disclosure that may be covalently linked to a 5′ end or a 3′ end of an antisense strand or a sense strand of an siRNA molecule and which may support the attachment of additional single- or double-stranded siRNA molecules. Non-limiting examples of branch point moieties suitable for use in conjunction with the disclosed methods and compositions include, e.g., phosphoroamidite, tosylated solketal, 1,3-diaminopropanol, pentaerythritol, and any one of the branch point moieties described in U.S. Pat. No. 10,478,503.

The term “phosphate moiety” as used herein, refers to a terminal phosphate group that includes phosphates as well as modified phosphates. The phosphate moiety may be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′) or alkyl where R′ is H, an amino protecting group or unsubstituted or substituted alkyl. In some embodiments, the 5′ and or 3′ terminal group may include from 1 to 3 phosphate moieties that are each, independently, unmodified (di- or tri-phosphates) or modified.

As used herein, the term “5′ phosphorus stabilizing moiety” refers to a terminal phosphate group that includes phosphates as well as modified phosphates (e.g., phosphorothioates, phosphodiesters, phosphonates). The phosphate moiety may be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′), or alkyl where R′ is H, an amino protecting group, or unsubstituted or substituted alkyl. In some embodiments, the 5′ and or 3′ terminal group may include from 1 to 3 phosphate moieties that are each, independently, unmodified (di- or tri-phosphates) or modified.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 10:117-21, 2000; Rusckowski et al., Antisense Nucleic Acid Drug Dev. 10:333-45, 2000; Stein, Antisense Nucleic Acid Drug Dev. 11:317-25, 2001; Vorobjev et al., Antisense Nucleic Acid Drug Dev. 11:77-85, 2001; and U.S. Pat. No. 5,684,143. Certain of the above-referenced modifications (e.g., phosphate group modifications) preferably decrease the rate of hydrolysis of, for example, polynucleotides including said analogs in vivo or in vitro.

As used herein, the term “complementary” refers to two nucleotides that form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software.

“Percent (%) sequence complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows:

$$100 multiplied by ( the fraction X / Y )$$

where X is the number of complementary base pairs in an alignment (e.g., as executed by computer software, such as BLAST) in that program's alignment of A and B, and where Y is the total number of nucleic acids in B. It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

$$100 multiplied by ( the fraction X / Y )$$

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “complementarity sufficient to hybridize,” as used herein, refers to a nucleic acid sequence or a portion thereof that need not be fully complementary (e.g., 100% complementary) to a target region or a nucleic acid sequence or a portion thereof that has one or more nucleotide mismatches relative to the target region but that is still capable of hybridizing to the target region under specified conditions. For example, the nucleic acid may be, e.g., 95% complementary, 90%, complementary, 85% complementary, 80% complementary, 75% complementary, 70% complementary, 65% complementary, 60% complementary, 55% complementary, 50% complementary, or less, but still form sufficient base pairs with the target so as to hybridize across its length. A sequence that has complementarity sufficient to hybridize to a target region or nucleic acid region within a specific exon includes a target region contained entirely within the specific exon or a target region that includes one or more nucleotides within the specific exon but also crosses an exon-exon junction.

“Hybridization” or “annealing” of nucleic acids is achieved when one or more nucleoside residues within a polynucleotide base pairs with one or more complementary nucleosides to form a stable duplex. The base pairing is typically driven by hydrogen bonding events. Hybridization includes Watson-Crick base pairs formed from natural and/or modified nucleobases. The hybridization can also include non-Watson-Crick base pairs, such as wobble base pairs (guanosine-uracil, hypoxanthine-uracil, hypoxanthine-adenine, and hypoxanthine-cytosine) and Hoogsteen base pairs. Nucleic acids need not be 100% complementary to undergo hybridization. For example, one nucleic acid may be, e.g., 95% complementary, 90%, complementary, 85% complementary, 80% complementary, 75% complementary, 70% complementary, 65% complementary, 60% complementary, 55% complementary, 50% complementary, or less, relative to another nucleic acid, but the two nucleic acids may still form sufficient base pairs with one another so as to hybridize.

The “stable duplex” formed upon the annealing/hybridization of one nucleic acid to another is a duplex structure that is not denatured by a stringent wash. Exemplary stringent wash conditions are known in the art and include temperatures of about 5° C. less than the melting temperature of an individual strand of the duplex and low concentrations of monovalent salts, such as monovalent salt concentrations (e.g., NaCl concentrations) of less than 0.2 M (e.g., 0.2 M, 0.19 M, 0.18 M, 0.17 M, 0.16 M, 0.15 M, 0.14 M, 0.13 M, 0.12 M, 0.11 M, 0.1 M, 0.09 M, 0.08 M, 0.07 M, 0.06 M, 0.05 M, 0.04 M, 0.03 M, 0.02 M, 0.01 M, or less).

The term “gene silencing” refers to the suppression of gene expression, e.g., endogenous gene expression of MSH3, which may be mediated through processes that affect transcription and/or through processes that affect post-transcriptional mechanisms. In some embodiments, gene silencing occurs when an RNAi molecule initiates the inhibition or degradation of the mRNA transcribed from a gene of interest in a sequence-specific manner by way of RNA interference, thereby preventing translation of the gene's product.

The phrase “overactive disease driver gene,” as used herein, refers to a gene having increased activity and/or expression that contributes to or causes a disease state in a subject (e.g., a human). The disease state may be caused or exacerbated by the overactive disease driver gene directly or by way of an intermediate gene(s).

As used herein, the term “ethylene glycol chain” refers to a carbon chain with the formula ((CH₂OH)₂).

As used herein, “alkyl” refers to a saturated hydrocarbon group. Alkyl groups may be acyclic or cyclic and contain only C and H when unsubstituted. When an alkyl residue having a specific number of carbons is named, all geometric isomers having that number of carbons are intended to be encompassed and described; thus, for example, “butyl” is meant to include n-butyl, sec-butyl, and iso-butyl. Examples of alkyl include ethyl, propyl, butyl, pentyl, hexyl, heptyl, octyl, cyclopropyl, cyclobutyl, cyclopentyl, cyclohexyl, cycloheptyl, cyclooctyl, and the like. In some embodiments, alkyl may be substituted. Suitable substituents that may be introduced into an alkyl group include, for example, hydroxy, alkoxy, amino, alkylamino, and halo, among others.

As used herein, “alkenyl” refers to an acyclic or cyclic unsaturated hydrocarbon group having at least one site of olefinic unsaturation (i.e., having at least one moiety of the formula C═C). Alkenyl groups contain only C and H when unsubstituted. When an alkenyl residue having a specific number of carbons is named, all geometric isomers having that number of carbons are intended to be encompassed and described; thus, for example, “butenyl” is meant to include n-butenyl, sec-butenyl, and iso-butenyl. Examples of alkenyl include —CH═CH₂, —CH₂—CH═CH₂, and —CH₂—CH═CH—CH═CH₂. In some embodiments, alkenyl may be substituted. Suitable substituents that may be introduced into an alkenyl group include, for example, hydroxy, alkoxy, amino, alkylamino, and halo, among others.

As used herein, “alkynyl” refers to an acyclic or cyclic unsaturated hydrocarbon group having at least one site of acetylenic unsaturation (i.e., having at least one moiety of the formula C═C). Alkynyl groups contain only C and H when unsubstituted. When an alkynyl residue having a specific number of carbons is named, all geometric isomers having that number of carbons are intended to be encompassed and described; thus, for example, “pentynyl” is meant to include n-pentynyl, sec-pentynyl, iso-pentynyl, and tert-pentynyl. Examples of alkynyl include —C═CH and —C═C—CH₃. In some embodiments, alkynyl may be substituted. Suitable substituents that may be introduced into an alkynyl group include, for example, hydroxy, alkoxy, amino, alkylamino, and halo, among others.

As used herein the term “phenyl” denotes a monocyclic arene in which one hydrogen atom from a carbon atom of the ring has been removed. A phenyl group may be unsubstituted or substituted with one or more suitable substituents, wherein the substituent replaces an H of the phenyl group.

As used herein, the term “benzyl” refers to monovalent radical obtained when a hydrogen atom attached to the methyl group of toluene is removed. A benzyl group generally has the formula of phenyl-CH₂—. A benzyl group may be unsubstituted or substituted with one or more suitable substituents. For example, the substituent may replace an H of the phenyl component and/or an H of the methylene (—CH₂—) component.

As used herein, the term “amide” refers to an alkyl, alkenyl, alkynyl, or aromatic group that is attached to an amino-carbonyl functional group.

As used herein, the term “triazole” refers to heterocyclic compounds with the formula (C₂H₃N₃), having a five-membered ring of two carbons and three nitrogens, the positions of which can change resulting in multiple isomers.

As used herein, the term “terminal group” refers to the group at which a carbon chain or nucleic acid ends.

As used herein, an “amino acid” refers to a molecule containing amine and carboxyl functional groups and a side chain specific to the amino acid.

In some embodiments the amino acid is chosen from the group of proteinogenic amino acids. In some embodiments, the amino acid is an L-amino acid or a D-amino acid. In some embodiments, the amino acid is a synthetic amino acid (e.g., a beta-amino acid).

As used herein, the term “lipophilic amino acid” refers to an amino acid including a hydrophobic moiety (e.g., an alkyl chain or an aromatic ring).

As used herein, the term “target of delivery” refers to the organ or part of the body to which it is desired to deliver the branched oligonucleotide compositions.

As used herein, the term “between X and Y” is inclusive of the values of X and Y. For example, “between X and Y” refers to the range of values between the value of X and the value of Y, as well as the value of X and the value of Y.

As used herein, the terms “subject” and “patient” are used interchangeably and refer to an organism, such as a mammal (e.g., a human), that experiences a neurodegenerative disease or disorder (e.g., a microsatellite repeat expansion disorder, such as Huntington's Disease, myotonic dystrophies, or spinocerebellar ataxias) and/or contains a gain-of-function MSH3 variant allele.

As used herein, the term “MSH3” refers to the gene encoding MutS Homolog 3, including any native MSH3 gene from any source. MSH3 is a DNA mismatch repair protein that forms a heterodimer with another mismatch repair protein, MutS Homolog 2 (MSH2) to form the complex MutSp. MutSp corrects insertion/deletion loops and base-base mispairs in microsatellites during DNA synthesis or DNA strand reannealing after transcription. The term encompasses “full-length,” unprocessed MSH3 as well as any form of MSH3 that results from processing in the cell. The term also encompasses naturally occurring variants of MSH3, e.g., splice variants or allelic variants. The nucleic acid sequence of an exemplary MSH3 gene is shown in European Nucleotide Archive (ENA) Accession No. J04810.1. The amino acid sequence of an exemplary protein encoded by an MSH3 gene is shown in UNIPROT™ Accession No. P20585.

As used herein, the terms “microsatellite repeat expansion disorder,” “microsatellite repeat expansion disease,” “nucleotide repeat expansion disorder,” and “nucleotide repeat expansion disease” are used interchangeably to refer to any disease or disorder caused by the instability and expansion of specific microsatellites. “Microsatellites” are coding or non-coding DNA sequences that contain tandem repeats of base pairs. Exemplary microsatellite repeat expansion disorders include, but are not limited to, Fragile X syndrome, Fragile XE syndrome, Fragile X-associated tremor/ataxia syndrome, Fragile X-primary ovarian insufficiency, progressive myoclonic epilepsy type 1/Unverricht-Lundborg disease, spinocerebellar ataxia (SCA) 12, neuronal intranuclear inclusion disease, glutaminase deficiency, Huntington's disease, SCA1, SCA2, SCA3, SCA6, SCA7, SCA17, dentatorubral-pallidoluysian atrophy, spinal-bulbar muscular atrophy, oculopharyngeal muscular dystrophy, Huntington disease-like 2, amyotrophic lateral sclerosis, myotonic dystrophy type 2 (DM2), Friedreich ataxia, Fuchs endothelial corneal dystrophy, SCA10, SCA31, SCA36, SCA37, cerebellar ataxia, neuropathy, and vestibular areflexia syndrome (CANVAS), benign adult familial myoclonic epilepsy, SCA8, and myotonic dystrophy type 1 (DM1). Other microsatellite repeat expansion disorders are discussed in Rodriguez et al., Neurobiology of Disease, 130:104515, 2019, the disclosure of which is incorporated herein by reference.

As used herein, the terms “treat,” “treated,” and “treating” mean both therapeutic treatment and prophylactic or preventative measures wherein the object is to prevent, ameliorate, or slow down (lessen) an undesired physiological condition, disorder, or disease, or obtain beneficial or desired clinical results. Beneficial or desired clinical results include, but are not limited to, a reduction in a patient's reliance on pharmacological treatments; alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical, cognitive, or behavioral (e.g., depressive behavior or apathy) parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the terms “benefit” and “response” are used interchangeably in the context of a subject undergoing therapy for the treatment of, for example, a microsatellite repeat expansion disease, such as Huntington's Disease or myotonic dystrophy type 1. For example, clinical benefits in the context of a subject having Huntington's disease administered an siRNA molecule or siRNA composition of the disclosure include, without limitation, a reduction in involuntary movements, memory lapses, mood swings, or symptoms of anxiety and depression, and/or; a reduction in wild type MSH3 transcripts, mutant MSH3 transcripts, variant MSH3 transcripts, splice isoforms of MSH3 transcripts, and/or overexpressed MSH3 transcripts; and/or an arrest or reduction of the length of the microsatellite repeat in the HTT gene.

### DETAILED DESCRIPTION

The present disclosure provides compositions of small interfering RNA (siRNA) molecules with sequence homology to a MutS Homolog 3 (MSH3) gene and methods for administering said siRNA molecules to a subject. Furthermore, the siRNA molecules described herein may be composed as branched siRNA structures, such as di-branched, tri-branched, and tetra-branched siRNA structures and may further include specific patterns of chemical modifications (e.g., 2′ ribose modifications or internucleoside linkage modifications) to improve resistance against nuclease enzymes, toxicity profile, and physicochemical properties (e.g., thermostability). Small interfering RNA molecules are short, double-stranded RNA molecules. They are capable of mediating RNA interference (RNAi) by degrading mRNA with a complementary nucleotide sequence, thus preventing the translation of the target gene.

The siRNA molecules of the disclosure may exhibit, for example, robust gene-specific suppression of MSH3, relative to other genes in the MutS family (e.g., MSH2, MSH4, MSH5, and MSH6) and other human genes.

The siRNA molecules of the disclosure may feature an antisense strand having a nucleic acid sequence that is complementary to a region of a MSH3 mRNA transcript having the nucleic acid sequence of any one of SEQ ID NOs: 1-408. The degree of complementarity of the antisense strand to the region of the MSH3 mRNA transcript may be sufficient for the antisense strand to anneal over the full length of the region of the MSH3 mRNA transcript. For example, the antisense strand may have a nucleic acid sequence that is at least 60% complementary (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary) to the region of the MSH3 mRNA transcript. In some embodiments, the region of the MSH3 RNA transcript has the sequence of any one of SEQ ID NOs: 5, 17-20, 33, 42, 44, 102, 103, 105, 106, 108, 109, 113, 126, 129, 130, 158, 161, 166, 177, 183, 193, 194, 196, 197, 202, 209, 217, 231, 271, 273, 291, 294, 305, 307, 317, 318, and 362. In some embodiments, the region of the MSH3 RNA transcript has the sequence of any one of SEQ ID NOs: 5, 17-20, 42, 44, 126, 129, 130, 158, 177, 183, 193, 194, 196, 197, 202, 209, 231, 271, 273, 291, 294, 305, 307, 317, 318, and 362.

In some embodiments, the siRNA molecules of the disclosure feature an antisense strand having the nucleic acid sequence of any one of SEQ ID NOs: 817-1224, or a nucleic acid sequence that is at least 60% identical thereto. For example, the siRNA molecules of the disclosure may feature an antisense strand having a nucleic acid sequence that is at least 60% identical (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 817-1224. In some embodiments, the nucleic acid sequence is any one of SEQ ID NOs: 821, 833-836, 849, 858, 860, 918, 919, 921, 922, 924, 925, 929, 942, 945, 946, 974, 977, 982, 993, 999, 1009, 1010, 1012, 1013, 1018, 1025, 1033, 1047, 1087, 1089, 1107, 1110, 1121, 1123, 1133, and 1134. In some embodiments, the nucleic acid sequence is any one of SEQ ID NOs: 821, 833-836, 858, 860, 942, 945, 946, 974, 993, 999, 1009, 1010, 1012, 1013, 1018, 1025, 1047, 1087, 1089, 1107, 1110, 1121, 1123, 1133, and 1134.

In some embodiments, the siRNA molecules of the disclosure feature a sense strand having the nucleic acid sequence ofany one of SEQ ID NOs: 409-816, or a nucleic acid sequence that is at least 60% identical thereto. For example, the siRNA molecules of the disclosure may feature a sense strand having a nucleic acid sequence that is at least 60% identical (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 409-816. In some embodiments, the nucleic acid sequence is any one of SEQ ID NOs: 413, 425-428, 441, 450, 452, 510, 511, 513, 514, 516, 517, 521, 534, 537, 538, 566, 569, 574, 585, 591, 601, 602, 604, 605, 610, 617, 625, 639, 679, 681, 699, 702, 713, 715, 725, 726, and 770. In some embodiments, the nucleic acid sequence is any one of SEQ ID NOs: 413, 425-428, 450, 452, 534, 537, 538, 566, 585, 591, 601, 602, 604, 605, 610, 617, 639, 679, 681, 699, 702, 713, 715, 725, 726, and 770.

Exemplary siRNA molecules of the disclosure are those shown in Table 1A, below. Table 1A summarizes the antisense strands, sense strands, and corresponding regions of an MSH3 mRNA transcript that are targeted by each antisense strand.

TABLE 1A
Nucleotide sequences for gene-specific MSH3-targeting siRNA

| Gene | | Sense | | Anti- | |
|---|---|---|---|---|---|
| region | | SEQ | | sense | |
| SEQ | | ID | Sense | SEQ ID | Antisense |
| ID NO: | Gene Region | NO: | Sequence | NO: | Sequence |
| 1 | GCGAGGCAAGCGGTTTTGA | 409 | GCCGAUUC | 817 | UACUGGAAGA |
| | GCCGATTCTTCCAGTCTACG | | UUCCAGUA | | AUCGGCUCAA |
| | GGAAGC | | | | A |
| 2 | CCGATTCTTCCAGTCTACGG | 410 | GGAAGCCU | 818 | UGGAUUUCAG |
| | GAAGCCTGAAATCCACCTCC | | GAAAUCCA | | GCUUCCCGUA |
| | TCCTC | | | | G |
| 3 | CTTCCAGTCTACGGGAAGCC | 411 | CUGAAAUC | 819 | UGGAGGUGGA |
| | TGAAATCCACCTCCTCCTCC | | CACCUCCA | | UUUCAGGCUU |
| | ACAGG | | | | C |
| 4 | AGTCTACGGGAAGCCTGAAA | 412 | AUCCACCU | 820 | UGAGGAGGAG |
| | TCCACCTCCTCCTCCACAGG | | CCUCCUCA | | GUGGAUUUCA |
| | TGCAG | | | | G |
| 5 | GCCGCACATAGCTACAGAAA | 413 | AUUGACAG | 821 | UCUUUCUUCU |
| | TTGACAGAAGAAAGAAGAGA | | AAGAAAGA | | GUCAAUUUCU |
| | CCATT | | | | G |
| 6 | CCGCACATAGCTACAGAAAT | 414 | UUGACAGA | 822 | UUCUUUCUUC |
| | TGACAGAAGAAAGAAGAGAC | | AGAAAGAA | | UGUCAAUUUC |
| | CATTG | | | | U |
| 7 | CGCACATAGCTACAGAAATT | 415 | UGACAGAA | 823 | UUUCUUUCUU |
| | GACAGAAGAAAGAAGAGAC | | GAAAGAAA | | CUGUCAAUUU |
| | CATTGG | | | | C |
| 8 | GCACATAGCTACAGAAATTG | 416 | GACAGAAG | 824 | UCUUCUUUCU |
| | ACAGAAGAAAGAAGAGACCA | | AAAGAAGA | | UCUGUCAAUU |
| | TTGGA | | | | U |
| 9 | CACATAGCTACAGAAATTGA | 417 | ACAGAAGA | 825 | UUCUUCUUUC |
| | CAGAAGAAAGAAGAGACCAT | | AAGAAGAA | | UUCUGUCAAU |
| | TGGAA | | | | U |
| 10 | ATAGCTACAGAAATTGACAG | 418 | GAAGAAAG | 826 | UGUCUCUUCU |
| | AAGAAAGAAGAGACCATTGG | | AAGAGACA | | UUCUUCUGUC |
| | AAAAT | | | | A |
| 11 | AAGAAAGAAGAGACCATTGG | 419 | GAAAAUGA | 827 | UAGGCCCAUC |
| | AAAATGATGGGCCTGTTAAA | | UGGGCCU | | AUUUUCCAAU |
| | AAGAA | | A | | G |
| 12 | AGAAAGAAGAGACCATTGGA | 420 | AAAAUGAU | 828 | UCAGGCCCAU |
| | AAATGATGGGCCTGTTAAAA | | GGGCCUG | | CAUUUUCCAA |
| | AGAAA | | A | | U |
| 13 | GAAAGAAGAGACCATTGGAA | 421 | AAAUGAUG | 829 | UACAGGCCCA |
| | AATGATGGGCCTGTTAAAAA | | GGCCUGU | | UCAUUUUCCA |
| | GAAAG | | A | | A |
| 14 | AAAGAAGAGACCATTGGAAA | 422 | AAUGAUGG | 830 | UAACAGGCCC |
| | ATGATGGGCCTGTTAAAAAG | | GCCUGUUA | | AUCAUUUUCC |
| | AAAGT | | | | A |
| 15 | AAGAAGAGACCATTGGAAAA | 423 | AUGAUGG | 831 | UUAACAGGCC |
| | TGATGGGCCTGTTAAAAAGA | | GCCUGUUA | | CAUCAUUUUC |
| | AAGTA | | A | | C |
| 16 | GATCTGGGAATGTCTGGCAA | 424 | ACUCUGAG | 832 | UUCUUUGGCU |
| | CTCTGAGCCAAAGAAATGTC | | CCAAAGAA | | CAGAGUUGCC |
| | TGAGG | | | | A |
| 17 | GAATGTCTGGCAACTCTGAG | 425 | GCCAAAGA | 833 | UAGACAUUUC |
| | CCAAAGAAATGTCTGAGGAC | | AAUGUCUA | | UUUGGCUCAG |
| | CAGGA | | | | A |
| 18 | AATGTCTGGCAACTCTGAGC | 426 | CCAAAGAA | 834 | UCAGACAUUU |
| | CAAAGAAATGTCTGAGGACC | | AUGUCUGA | | CUUUGGCUCA |
| | AGGAA | | | | G |
| 19 | ATGTCTGGCAACTCTGAGCC | 427 | CAAAGAAA | 835 | UUCAGACAUU |
| | AAAGAAATGTCTGAGGACCA | | UGUCUGAA | | UCUUUGGCUC |
| | GGAAT | | | | A |
| 20 | TGTCTGGCAACTCTGAGCCA | 428 | AAAGAAAU | 836 | UCUCAGACAU |
| | AAGAAATGTCTGAGGACCAG | | GUCUGAGA | | UUCUUUGGCU |
| | GAATG | | | | C |
| 21 | GTCTGGCAACTCTGAGCCAA | 429 | AAGAAAUG | 837 | UCCUCAGACA |
| | AGAAATGTCTGAGGACCAG | | UCUGAGGA | | UUUCUUUGGC |
| | GAATGT | | | | U |
| 22 | TCTGGCAACTCTGAGCCAAA | 430 | AGAAAUGU | 838 | UUCCUCAGAC |
| | GAAATGTCTGAGGACCAGG | | CUGAGGAA | | AUUUCUUUGG |
| | AATGTT | | | | C |
| 23 | CTGGCAACTCTGAGCCAAAG | 431 | GAAAUGUC | 839 | UGUCCUCAGA |
| | AAATGTCTGAGGACCAGGAA | | UGAGGACA | | CAUUUCUUUG |
| | TGTTT | | | | G |
| 24 | TTGAAAGAATTCTGCTGCGA | 432 | AUUCUGCC | 840 | UGAGGAAGGG |
| | TTCTGCCCTTCCTCAAAGTA | | CUUCCUCA | | CAGAAUCGCA |
| | GAGTC | | | | G |
| 25 | TCCAGACAGAATCTCTGCAG | 433 | GGAGAGAU | 841 | UACUGCAAAU |
| | GAGAGATTTGCAGTTCTGCC | | UUGCAGUA | | CUCUCCUGCA |
| | AAAAT | | | | G |
| 26 | CCAGACAGAATCTCTGCAGG | 434 | GAGAGAUU | 842 | UAACUGCAAA |
| | AGAGATTTGCAGTTCTGCCA | | UGCAGUUA | | UCUCUCCUGC |
| | AAATG | | | | A |
| 27 | CAGACAGAATCTCTGCAGGA | 435 | AGAGAUUU | 843 | UGAACUGCAA |
| | GAGATTTGCAGTTCTGCCAA | | GCAGUUCA | | AUCUCUCCUG |
| | AATGT | | | | C |
| 28 | AGACAGAATCTCTGCAGGAG | 436 | GAGAUUUG | 844 | UAGAACUGCA |
| | AGATTTGCAGTTCTGCCAAA | | CAGUUCUA | | AAUCUCUCCU |
| | ATGTA | | | | G |
| 29 | GACAGAATCTCTGCAGGAGA | 437 | AGAUUUGC | 845 | UCAGAACUGC |
| | GATTTGCAGTTCTGCCAAAA | | AGUUCUGA | | AAAUCUCUCC |
| | TGTAC | | | | U |
| 30 | GCAGGAGAGATTTGCAGTTC | 438 | CUGCCAAA | 846 | UAGUACAUUU |
| | TGCCAAAATGTACTGATTTT | | AUGUACUA | | UGGCAGAACU |
| | GATGA | | | | G |
| 31 | CAGGAGAGATTTGCAGTTCT | 439 | UGCCAAAA | 847 | UCAGUACAUU |
| | GCCAAAATGTACTGATTTTG | | UGUACUGA | | UUGGCAGAAC |
| | ATGAT | | | | U |
| 32 | AGGAGAGATTTGCAGTTCTG | 440 | GCCAAAAU | 848 | UUCAGUACAU |
| | CCAAAATGTACTGATTTTGAT | | GUACUGAA | | UUUGGCAGAA |
| | GATA | | | | C |
| 33 | GGAGAGATTTGCAGTTCTGC | 441 | CCAAAAUG | 849 | UAUCAGUACA |
| | CAAAATGTACTGATTTTGAT | | UACUGAUA | | UUUUGGCAGA |
| | GATAT | | | | A |
| 34 | GAGAGATTTGCAGTTCTGCC | 442 | CAAAAUGU | 850 | UAAUCAGUAC |
| | AAAATGTACTGATTTTGATGA | | ACUGAUUA | | AUUUUGGCAG |
| | TATC | | | | A |
| 35 | AGAGATTTGCAGTTCTGCCA | 443 | AAAAUGUA | 851 | UAAAUCAGUA |
| | AAATGTACTGATTTTGATGAT | | CUGAUUUA | | CAUUUUGGCA |
| | ATCA | | | | G |
| 36 | GAGATTTGCAGTTCTGCCAA | 444 | AAAUGUAC | 852 | UAAAAUCAGU |
| | AATGTACTGATTTTGATGATA | | UGAUUUUA | | ACAUUUUGGC |
| | TCAG | | | | A |
| 37 | GATTTTGATGATATCAGTCTT | 445 | UUCUACAC | 853 | UUCUUUGCGU |
| | CTACACGCAAAGAATGCAGT | | GCAAAGAA | | GUAGAAGACU |
| | TTCT | | | | G |
| 38 | ATTTTGATGATATCAGTCTTC | 446 | UCUACACG | 854 | UUUCUUUGCG |
| | TACACGCAAAGAATGCAGTT | | CAAAGAAA | | UGUAGAAGAC |
| | TCTT | | | | U |
| 39 | ATGATATCAGTCTTCTACAC | 447 | CGCAAAGA | 855 | UACUGCAUUC |
| | GCAAAGAATGCAGTTTCTTC | | AUGCAGUA | | UUUGCGUGUA |
| | TGAAG | | | | G |
| 40 | TGATATCAGTCTTCTACACG | 448 | GCAAAGAA | 856 | UAACUGCAUU |
| | CAAAGAATGCAGTTTCTTCT | | UGCAGUUA | | CUUUGCGUGU |
| | GAAGA | | | | A |
| 41 | GATATCAGTCTTCTACACGC | 449 | CAAAGAAU | 857 | UAAACUGCAU |
| | AAAGAATGCAGTTTCTTCTG | | GCAGUUUA | | UCUUUGCGUG |
| | AAGAT | | | | U |
| 42 | ATATCAGTCTTCTACACGCA | 450 | AAAGAAUG | 858 | UGAAACUGCA |
| | AAGAATGCAGTTTCTTCTGA | | CAGUUUCA | | UUCUUUGCGU |
| | AGATT | | | | G |
| 43 | TATCAGTCTTCTACACGCAA | 451 | AAGAAUGC | 859 | UAGAAACUGC |
| | AGAATGCAGTTTCTTCTGAA | | AGUUUCUA | | AUUCUUUGCG |
| | GATTC | | | | U |
| 44 | ATCAGTCTTCTACACGCAAA | 452 | AGAAUGCA | 860 | UAAGAAACUG |
| | GAATGCAGTTTCTTCTGAAG | | GUUUCUUA | | CAUUCUUUGC |
| | ATTCG | | | | G |
| 45 | TCAGTCTTCTACACGCAAAG | 453 | GAAUGCAG | 861 | UGAAGAAACU |
| | AATGCAGTTTCTTCTGAAGA | | UUUCUUCA | | GCAUUCUUUG |
| | TTCGA | | | | C |
| 46 | ATGCAGTTTCTTCTGAAGAT | 454 | UUCGAAAC | 862 | UAUUUGACGU |
| | TCGAAACGTCAAATTAATCA | | GUCAAAUA | | UUCGAAUCUU |
| | AAAGG | | | | C |
| 47 | TTTCTTCTGAAGATTCGAAA | 455 | ACGUCAAA | 863 | UUGAUUAAUU |
| | CGTCAAATTAATCAAAAGGA | | UUAAUCAA | | UGACGUUUCG |
| | CACAA | | | | A |
| 48 | AAGGACACAACACTTTTTGA | 456 | AUCUCAGU | 864 | UCAAACUGAC |
| | TCTCAGTCAGTTTGGATCAT | | CAGUUUGA | | UGAGAUCAAA |
| | CAAAT | | | | A |
| 49 | CAACACTTTTTGATCTCAGT | 457 | UCAGUUUG | 865 | UGAUGAUCCA |
| | CAGTTTGGATCATCAAATAC | | GAUCAUCA | | AACUGACUGA |
| | AAGTC | | | | G |
| 50 | ACACTTTTTGATCTCAGTCA | 458 | AGUUUGGA | 866 | UUUGAUGAUC |
| | GTTTGGATCATCAAATACAA | | UCAUCAAA | | CAAACUGACU |
| | GTCAT | | | | G |
| 51 | TTTTTGATCTCAGTCAGTTTG | 459 | UGGAUCAU | 867 | UGUAUUUGAU |
| | GATCATCAAATACAAGTCAT | | CAAAUACA | | GAUCCAAACU |
| | GAAA | | | | G |
| 52 | TGATCTCAGTCAGTTTGGAT | 460 | UCAUCAAA | 868 | UACUUGUAUU |
| | CATCAAATACAAGTCATGAA | | UACAAGUA | | UGAUGAUCCA |
| | AATTT | | | | A |
| 53 | ATACAAGTCATGAAAATTTAC | 461 | ACAGAAAA | 869 | UGAAGCAGUU |
| | AGAAAACTGCTTCCAAATCA | | CUGCUUCA | | UUCUGUAAAU |
| | GCTA | | | | U |
| 54 | AGTCATGAAAATTTACAGAA | 462 | AAACUGCU | 870 | UAUUUGGAAG |
| | AACTGCTTCCAAATCAGCTA | | UCCAAAUA | | CAGUUUUCUG |
| | ACAAA | | | | U |
| 55 | GTCATGAAAATTTACAGAAA | 463 | AACUGCUU | 871 | UGAUUUGGAA |
| | ACTGCTTCCAAATCAGCTAA | | CCAAAUCA | | GCAGUUUUCU |
| | CAAAC | | | | G |
| 56 | TCATGAAAATTTACAGAAAA | 464 | ACUGCUUC | 872 | UUGAUUUGGA |
| | CTGCTTCCAAATCAGCTAAC | | CAAAUCAA | | AGCAGUUUUC |
| | AAACG | | | | U |
| 57 | CATGAAAATTTACAGAAAAC | 465 | CUGCUUCC | 873 | UCUGAUUUGG |
| | TGCTTCCAAATCAGCTAACA | | AAAUCAGA | | AAGCAGUUUU |
| | AACGG | | | | C |
| 58 | ATGAAAATTTACAGAAAACT | 466 | UGCUUCCA | 874 | UGCUGAUUUG |
| | GCTTCCAAATCAGCTAACAA | | AAUCAGCA | | GAAGCAGUUU |
| | ACGGT | | | | U |
| 59 | TTTACAGAAAACTGCTTCCA | 467 | AAAUCAGC | 875 | UUUUGUUAGC |
| | AATCAGCTAACAAACGGTCC | | UAACAAAA | | UGAUUUGGAA |
| | AAAAG | | | | G |
| 60 | TTACAGAAAACTGCTTCCAA | 468 | AAUCAGCU | 876 | UGUUUGUUAG |
| | ATCAGCTAACAAACGGTCCA | | AACAAACA | | CUGAUUUGGA |
| | AAAGC | | | | A |
| 61 | TACAGAAAACTGCTTCCAAA | 469 | AUCAGCUA | 877 | UCGUUUGUUA |
| | TCAGCTAACAAACGGTCCAA | | ACAAACGA | | GCUGAUUUGG |
| | AAGCA | | | | A |
| 62 | ACAGAAAACTGCTTCCAAAT | 470 | UCAGCUAA | 878 | UCCGUUUGUU |
| | CAGCTAACAAACGGTCCAAA | | CAAACGGA | | AGCUGAUUUG |
| | AGCAT | | | | G |
| 63 | CAGAAAACTGCTTCCAAATC | 471 | CAGCUAAC | 879 | UACCGUUUGU |
| | AGCTAACAAACGGTCCAAAA | | AAACGGUA | | UAGCUGAUUU |
| | GCATC | | | | G |
| 64 | AGAAAACTGCTTCCAAATCA | 472 | AGCUAACA | 880 | UGACCGUUUG |
| | GCTAACAAACGGTCCAAAAG | | AACGGUCA | | UUAGCUGAUU |
| | CATCT | | | | U |
| 65 | GAAAACTGCTTCCAAATCAG | 473 | GCUAACAA | 881 | UGGACCGUUU |
| | CTAACAAACGGTCCAAAAGC | | ACGGUCCA | | GUUAGCUGAU |
| | ATCTA | | | | U |
| 66 | TGCTTCCAAATCAGCTAACA | 474 | AAACGGUC | 882 | UGCUUUUGGA |
| | AACGGTCCAAAAGCATCTAT | | CAAAAGCA | | CCGUUUGUUA |
| | ACGCC | | | | G |
| 67 | CTTCCAAATCAGCTAACAAA | 475 | ACGGUCCA | 883 | UAUGCUUUUG |
| | CGGTCCAAAAGCATCTATAC | | AAAGCAUA | | GACCGUUUGU |
| | GCCGC | | | | U |
| 68 | AATCAGCTAACAAACGGTCC | 476 | CAAAAGCA | 884 | UGUAUAGAUG |
| | AAAAGCATCTATACGCCGCT | | UCUAUACA | | CUUUUGGACC |
| | AGAAT | | | | G |
| 69 | ATCAGCTAACAAACGGTCCA | 477 | AAAAGCAU | 885 | UCGUAUAGAU |
| | AAAGCATCTATACGCCGCTA | | CUAUACGA | | GCUUUUGGAC |
| | GAATT | | | | C |
| 70 | TCAGCTAACAAACGGTCCAA | 478 | AAAGCAUC | 886 | UGCGUAUAGA |
| | AAGCATCTATACGCCGCTAG | | UAUACGCA | | UGCUUUUGGA |
| | AATTA | | | | C |
| 71 | CAGCTAACAAACGGTCCAAA | 479 | AAGCAUCU | 887 | UGGCGUAUAG |
| | AGCATCTATACGCCGCTAGA | | AUACGCCA | | AUGCUUUUGG |
| | ATTAC | | | | A |
| 72 | ATAGAAATGAAGCAGCAGCA | 480 | ACAAAGAU | 888 | UAAACUGCAU |
| | CAAAGATGCAGTTTTGTGTG | | GCAGUUUA | | CUUUGUGCUG |
| | TGGAA | | | | C |
| 73 | TAGAAATGAAGCAGCAGCAC | 481 | CAAAGAUG | 889 | UAAAACUGCA |
| | AAAGATGCAGTTTTGTGTGT | | CAGUUUUA | | UCUUUGUGCU |
| | GGAAT | | | | G |
| 74 | CAAAGATGCAGTTTTGTGTG | 482 | GUGGAAU | 890 | UAUAUCCACA |
| | TGGAATGTGGATATAAGTAT | | GUGGAUAU | | UUCCACACAC |
| | AGATT | | A | | A |
| 75 | AAAGATGCAGTTTTGTGTGT | 483 | UGGAAUGU | 891 | UUAUAUCCAC |
| | GGAATGTGGATATAAGTATA | | GGAUAUAA | | AUUCCACACA |
| | GATTC | | | | C |
| 76 | AAGATGCAGTTTTGTGTGTG | 484 | GGAAUGU | 892 | UUUAUAUCCA |
| | GAATGTGGATATAAGTATAG | | GGAUAUAA | | CAUUCCACAC |
| | ATTCT | | A | | A |
| 77 | AGATGCAGTTTTGTGTGTGG | 485 | GAAUGUG | 893 | UCUUAUAUCC |
| | AATGTGGATATAAGTATAGA | | GAUAUAAG | | ACAUUCCACA |
| | TTCTT | | A | | C |
| 78 | GATGCAGTTTTGTGTGTGGA | 486 | AAUGUGGA | 894 | UACUUAUAUC |
| | ATGTGGATATAAGTATAGAT | | UAUAAGUA | | CACAUUCCAC |
| | TCTTT | | | | A |
| 79 | ATGCAGTTTTGTGTGTGGAA | 487 | AUGUGGAU | 895 | UUACUUAUAU |
| | TGTGGATATAAGTATAGATT | | AUAAGUAA | | CCACAUUCCA |
| | CTTTG | | | | C |
| 80 | AGTTTTGTGTGTGGAATGTG | 488 | GGAUAUAA | 896 | UUCUAUACUU |
| | GATATAAGTATAGATTCTTTG | | GUAUAGAA | | AUAUCCACAU |
| | GGGA | | | | U |
| 81 | GTTTTGTGTGTGGAATGTGG | 489 | GAUAUAAG | 897 | UAUCUAUACU |
| | ATATAAGTATAGATTCTTTGG | | UAUAGAUA | | UAUAUCCACA |
| | GGAA | | | | U |
| 82 | TTTTGTGTGTGGAATGTGGA | 490 | AUAUAAGU | 898 | UAAUCUAUAC |
| | TATAAGTATAGATTCTTTGG | | AUAGAUUA | | UUAUAUCCAC |
| | GGAAG | | | | A |
| 83 | TTTGTGTGTGGAATGTGGAT | 491 | UAUAAGUA | 899 | UGAAUCUAUA |
| | ATAAGTATAGATTCTTTGGG | | UAGAUUCA | | CUUAUAUCCA |
| | GAAGA | | | | C |
| 84 | TTGTGTGTGGAATGTGGATA | 492 | AUAAGUAU | 900 | UAGAAUCUAU |
| | TAAGTATAGATTCTTTGGGG | | AGAUUCUA | | ACUUAUAUCC |
| | AAGAT | | | | A |
| 85 | CCGAGAGCTCAATATTTATT | 493 | UGCCAUUU | 901 | UGUGAUCUAA |
| | GCCATTTAGATCACAACTTT | | AGAUCACA | | AUGGCAAUAA |
| | ATGAC | | | | A |
| 86 | AGAGCTCAATATTTATTGCC | 494 | CAUUUAGA | 902 | UGUUGUGAUC |
| | ATTTAGATCACAACTTTATGA | | UCACAACA | | UAAAUGGCAA |
| | CAGC | | | | U |
| 87 | GAGCTCAATATTTATTGCCA | 495 | AUUUAGAU | 903 | UAGUUGUGAU |
| | TTTAGATCACAACTTTATGAC | | CACAACUA | | CUAAAUGGCA |
| | AGCA | | | | A |
| 88 | AGCTCAATATTTATTGCCATT | 496 | UUUAGAUC | 904 | UAAGUUGUGA |
| | TAGATCACAACTTTATGACA | | ACAACUUA | | UCUAAAUGGC |
| | GCAA | | | | A |
| 89 | GCTCAATATTTATTGCCATTT | 497 | UUAGAUCA | 905 | UAAAGUUGUG |
| | AGATCACAACTTTATGACAG | | CAACUUUA | | AUCUAAAUGG |
| | CAAG | | | | C |
| 90 | CTCAATATTTATTGCCATTTA | 498 | UAGAUCAC | 906 | UUAAAGUUGU |
| | GATCACAACTTTATGACAGC | | AACUUUAA | | GAUCUAAAUG |
| | AAGT | | | | G |
| 91 | TCAATATTTATTGCCATTTAG | 499 | AGAUCACA | 907 | UAUAAAGUUG |
| | ATCACAACTTTATGACAGCA | | ACUUUAUA | | UGAUCUAAAU |
| | AGTA | | | | G |
| 92 | CAATATTTATTGCCATTTAGA | 500 | GAUCACAA | 908 | UCAUAAAGUU |
| | TCACAACTTTATGACAGCAA | | CUUUAUGA | | GUGAUCUAAA |
| | GTAT | | | | U |
| 93 | ATTTATTGCCATTTAGATCAC | 501 | ACAACUUU | 909 | UCUGUCAUAA |
| | AACTTTATGACAGCAAGTAT | | AUGACAGA | | AGUUGUGAUC |
| | ACCT | | | | U |
| 94 | TTTATTGCCATTTAGATCACA | 502 | CAACUUUA | 910 | UGCUGUCAUA |
| | ACTTTATGACAGCAAGTATA | | UGACAGCA | | AAGUUGUGAU |
| | CCTA | | | | C |
| 95 | TTATTGCCATTTAGATCACAA | 503 | AACUUUAU | 911 | UUGCUGUCAU |
| | CTTTATGACAGCAAGTATAC | | GACAGCAA | | AAAGUUGUGA |
| | CTAC | | | | U |
| 96 | TATTGCCATTTAGATCACAA | 504 | ACUUUAUG | 912 | UUUGCUGUCA |
| | CTTTATGACAGCAAGTATAC | | ACAGCAAA | | UAAAGUUGUG |
| | CTACT | | | | A |
| 97 | ATTGCCATTTAGATCACAAC | 505 | CUUUAUGA | 913 | UCUUGCUGUC |
| | TTTATGACAGCAAGTATACC | | CAGCAAGA | | AUAAAGUUGU |
| | TACTC | | | | G |
| 98 | TTGCCATTTAGATCACAACTT | 506 | UUUAUGAC | 914 | UACUUGCUGU |
| | TATGACAGCAAGTATACCTA | | AGCAAGUA | | CAUAAAGUUG |
| | CTCA | | | | U |
| 99 | TGCCATTTAGATCACAACTTT | 507 | UUAUGACA | 915 | UUACUUGCUG |
| | ATGACAGCAAGTATACCTAC | | GCAAGUAA | | UCAUAAAGUU |
| | TCAC | | | | G |
| 100 | TTTATGACAGCAAGTATACC | 508 | CUACUCAC | 916 | UACAGUCUGU |
| | TACTCACAGACTGTTTGTTC | | AGACUGUA | | GAGUAGGUAU |
| | ATGTA | | | | A |
| 101 | GTGGCAAAAGGATATAAGGT | 509 | UGGGAGU | 917 | UGCUUCACAA |
| | GGGAGTTGTGAAGCAAACT | | UGUGAAGC | | CUCCCACCUU |
| | GAAACT | | A | | A |
| 102 | TGGCAAAAGGATATAAGGTG | 510 | GGGAGUU | 918 | UUGCUUCACA |
| | GGAGTTGTGAAGCAAACTGA | | GUGAAGCA | | ACUCCCACCU |
| | AACTG | | A | | U |
| 103 | GGCAAAAGGATATAAGGTG | 511 | GGAGUUG | 919 | UUUGCUUCAC |
| | GGAGTTGTGAAGCAAACTGA | | UGAAGCAA | | AACUCCCACC |
| | AACTGC | | A | | U |
| 104 | CAAAAGGATATAAGGTGGGA | 512 | AGUUGUGA | 920 | UGUUUGCUUC |
| | GTTGTGAAGCAAACTGAAAC | | AGCAAACA | | ACAACUCCCA |
| | TGCAG | | | | C |
| 105 | AAAAGGATATAAGGTGGGAG | 513 | GUUGUGAA | 921 | UAGUUUGCUU |
| | TTGTGAAGCAAACTGAAACT | | GCAAACUA | | CACAACUCCC |
| | GCAGC | | | | A |
| 106 | AAAGGATATAAGGTGGGAGT | 514 | UUGUGAAG | 922 | UCAGUUUGCU |
| | TGTGAAGCAAACTGAAACTG | | CAAACUGA | | UCACAACUCC |
| | CAGCA | | | | C |
| 107 | AAGGATATAAGGTGGGAGTT | 515 | UGUGAAGC | 923 | UUCAGUUUGC |
| | GTGAAGCAAACTGAAACTGC | | AAACUGAA | | UUCACAACUC |
| | AGCAT | | | | C |
| 108 | AGGATATAAGGTGGGAGTTG | 516 | GUGAAGCA | 924 | UUUCAGUUUG |
| | TGAAGCAAACTGAAACTGCA | | AACUGAAA | | CUUCACAACU |
| | GCATT | | | | C |
| 109 | GGATATAAGGTGGGAGTTGT | 517 | UGAAGCAA | 925 | UUUUCAGUUU |
| | GAAGCAAACTGAAACTGCAG | | ACUGAAAA | | GCUUCACAAC |
| | CATTA | | | | U |
| 110 | GATATAAGGTGGGAGTTGTG | 518 | GAAGCAAA | 926 | UGUUUCAGUU |
| | AAGCAAACTGAAACTGCAGC | | CUGAAACA | | UGCUUCACAA |
| | ATTAA | | | | C |
| 111 | ATATAAGGTGGGAGTTGTGA | 519 | AAGCAAAC | 927 | UAGUUUCAGU |
| | AGCAAACTGAAACTGCAGCA | | UGAAACUA | | UUGCUUCACA |
| | TTAAA | | | | A |
| 112 | TATAAGGTGGGAGTTGTGAA | 520 | AGCAAACU | 928 | UCAGUUUCAG |
| | GCAAACTGAAACTGCAGCAT | | GAAACUGA | | UUUGCUUCAC |
| | TAAAG | | | | A |
| 113 | ATAAGGTGGGAGTTGTGAAG | 521 | GCAAACUG | 929 | UGCAGUUUCA |
| | CAAACTGAAACTGCAGCATT | | AAACUGCA | | GUUUGCUUCA |
| | AAAGG | | | | C |
| 114 | TAAGGTGGGAGTTGTGAAG | 522 | CAAACUGA | 930 | UUGCAGUUUC |
| | CAAACTGAAACTGCAGCATT | | AACUGCAA | | AGUUUGCUUC |
| | AAAGGC | | | | A |
| 115 | AAGGTGGGAGTTGTGAAGC | 523 | AAACUGAA | 931 | UCUGCAGUUU |
| | AAACTGAAACTGCAGCATTA | | ACUGCAGA | | CAGUUUGCUU |
| | AAGGCC | | | | C |
| 116 | AGGTGGGAGTTGTGAAGCA | 524 | AACUGAAA | 932 | UGCUGCAGUU |
| | AACTGAAACTGCAGCATTAA | | CUGCAGCA | | UCAGUUUGCU |
| | AGGCCA | | | | U |
| 117 | CAGCATTAAAGGCCATTGGA | 525 | AGACAACA | 933 | UGAACUUCUG |
| | GACAACAGAAGTTCACTCTT | | GAAGUUCA | | UUGUCUCCAA |
| | TTCCC | | | | U |
| 118 | GAAGTTCACTCTTTTCCCGG | 526 | GAAAUUGA | 934 | UAGGGCAGUC |
| | AAATTGACTGCCCTTTATAC | | CUGCCCUA | | AAUUUCCGGG |
| | AAAAT | | | | A |
| 119 | TTCACTCTTTTCCCGGAAATT | 527 | UUGACUGC | 935 | UAUAAAGGGC |
| | GACTGCCCTTTATACAAAAT | | CCUUUAUA | | AGUCAAUUUC |
| | CTAC | | | | C |
| 120 | TCTTTTCCCGGAAATTGACT | 528 | UGCCCUUU | 936 | UUUUGUAUAA |
| | GCCCTTTATACAAAATCTAC | | AUACAAAA | | AGGGCAGUCA |
| | ACTTA | | | | A |
| 121 | GTGAATCCCCTAATCAAGCT | 529 | UGGAUGAU | 937 | UUUACAGCAU |
| | GGATGATGCTGTAAATGTTG | | GCUGUAAA | | CAUCCAGCUU |
| | ATGAG | | | | G |
| 122 | CTGGATGATGCTGTAAATGT | 530 | UUGAUGAG | 938 | UUCAUUAUCU |
| | TGATGAGATAATGACTGATA | | AUAAUGAA | | CAUCAACAUU |
| | CTTCT | | | | U |
| 123 | TGCTGTAAATGTTGATGAGA | 531 | AUAAUGAC | 939 | UAGUAUCAGU |
| | TAATGACTGATACTTCTACC | | UGAUACUA | | CAUUAUCUCA |
| | AGCTA | | | | U |
| 124 | TTCTACCAGCTATCTTCTGT | 532 | UGCAUCUC | 940 | UAUUUUCAGA |
| | GCATCTCTGAAAATAAGGAA | | UGAAAAUA | | GAUGCACAGA |
| | AATGT | | | | A |
| 125 | TCTACCAGCTATCTTCTGTG | 533 | GCAUCUCU | 941 | UUAUUUUCAG |
| | CATCTCTGAAAATAAGGAAA | | GAAAAUAA | | AGAUGCACAG |
| | ATGTT | | | | A |
| 126 | CTACCAGCTATCTTCTGTGC | 534 | CAUCUCUG | 942 | UUUAUUUUCA |
| | ATCTCTGAAAATAAGGAAAA | | AAAAUAAA | | GAGAUGCACA |
| | TGTTA | | | | G |
| 127 | TACCAGCTATCTTCTGTGCA | 535 | AUCUCUGA | 943 | UCUUAUUUUC |
| | TCTCTGAAAATAAGGAAAAT | | AAAUAAGA | | AGAGAUGCAC |
| | GTTAG | | | | A |
| 128 | ACCAGCTATCTTCTGTGCAT | 536 | UCUCUGAA | 944 | UCCUUAUUUU |
| | CTCTGAAAATAAGGAAAATG | | AAUAAGGA | | CAGAGAUGCA |
| | TTAGG | | | | C |
| 129 | CCAGCTATCTTCTGTGCATC | 537 | CUCUGAAA | 945 | UUCCUUAUUU |
| | TCTGAAAATAAGGAAAATGT | | AUAAGGAA | | UCAGAGAUGC |
| | TAGGG | | | | A |
| 130 | CAGCTATCTTCTGTGCATCT | 538 | UCUGAAAA | 946 | UUUCCUUAUU |
| | CTGAAAATAAGGAAAATGTT | | UAAGGAAA | | UUCAGAGAUG |
| | AGGGA | | | | C |
| 131 | AGCTATCTTCTGTGCATCTC | 539 | CUGAAAAU | 947 | UUUUCCUUAU |
| | TGAAAATAAGGAAAATGTTA | | AAGGAAAA | | UUUCAGAGAU |
| | GGGAC | | | | G |
| 132 | CTATCTTCTGTGCATCTCTG | 540 | GAAAAUAA | 948 | UAUUUUCCUU |
| | AAAATAAGGAAAATGTTAGG | | GGAAAAUA | | AUUUUCAGAG |
| | GACAA | | | | A |
| 133 | TATCTTCTGTGCATCTCTGAA | 541 | AAAAUAAG | 949 | UCAUUUUCCU |
| | AATAAGGAAAATGTTAGGGA | | GAAAAUGA | | UAUUUUCAGA |
| | CAAA | | | | G |
| 134 | CTCGTTCAGAGCTAGAAACC | 542 | CCGGAUG | 950 | UAGGCUUGAC |
| | CGGATGTCAAGCCTGCAGC | | UCAAGCCU | | AUCCGGGUUU |
| | CAGTAG | | A | | C |
| 135 | TCAGAGCTAGAAACCOGGAT | 543 | UGUCAAGC | 951 | UGCUGCAGGC |
| | GTCAAGCCTGCAGCCAGTA | | CUGCAGCA | | UUGACAUCCG |
| | GAGCTG | | | | G |
| 136 | CTAGAAACCCGGATGTCAAG | 544 | GCCUGCA | 952 | UCUACUGGCU |
| | CCTGCAGCCAGTAGAGCTG | | GCCAGUAG | | GCAGGCUUGA |
| | CTGCTT | | A | | C |
| 137 | GTAGAGCTGCTGCTTCCTTC | 545 | CGGCCUU | 953 | UGCUCGGACA |
| | GGCCTTGTCCGAGCAAACA | | GUCCGAG | | AGGCCGAAGG |
| | GAGGCG | | CA | | A |
| 138 | TTCGGCCTTGTCCGAGCAAA | 546 | ACAGAGGC | 954 | UGAUGAGCGC |
| | CAGAGGCGCTCATCCACAG | | GCUCAUCA | | CUCUGUUUGC |
| | AGCCAC | | | | U |
| 139 | AGCAAACAGAGGCGCTCAT | 547 | CCACAGAG | 955 | UGAUGUGGCU |
| | CCACAGAGCCACATCTGTTA | | CCACAUCA | | CUGUGGAUGA |
| | GTGTGC | | | | G |
| 140 | GATGGATAACATTTATTTTGA | 548 | GAAUACAG | 956 | UAGCAUGGCU |
| | ATACAGCCATGCTTTCCAGG | | CCAUGCUA | | GUAUUCAAAA |
| | CAGT | | | | U |
| 141 | ATGGATAACATTTATTTTGAA | 549 | AAUACAGC | 957 | UAAGCAUGGC |
| | TACAGCCATGCTTTCCAGGC | | CAUGCUUA | | UGUAUUCAAA |
| | AGTT | | | | A |
| 142 | TGGATAACATTTATTTTGAAT | 550 | AUACAGCC | 958 | UAAAGCAUGG |
| | ACAGCCATGCTTTCCAGGCA | | AUGCUUUA | | CUGUAUUCAA |
| | GTTA | | | | A |
| 143 | GGATAACATTTATTTTGAATA | 551 | UACAGCCA | 959 | UGAAAGCAUG |
| | CAGCCATGCTTTCCAGGCAG | | UGCUUUCA | | GCUGUAUUCA |
| | TTAC | | | | A |
| 144 | GATAACATTTATTTTGAATAC | 552 | ACAGCCAU | 960 | UGGAAAGCAU |
| | AGCCATGCTTTCCAGGCAGT | | GCUUUCCA | | GGCUGUAUUC |
| | TACA | | | | A |
| 145 | ATAACATTTATTTTGAATACA | 553 | CAGCCAUG | 961 | UUGGAAAGCA |
| | GCCATGCTTTCCAGGCAGTT | | CUUUCCAA | | UGGCUGUAUU |
| | ACAG | | | | C |
| 146 | TAACATTTATTTTGAATACAG | 554 | AGCCAUGC | 962 | UCUGGAAAGC |
| | CCATGCTTTCCAGGCAGTTA | | UUUCCAGA | | AUGGCUGUAU |
| | CAGA | | | | U |
| 147 | TGAATACAGCCATGCTTTCC | 555 | CAGGCAGU | 963 | UCUCUGUAAC |
| | AGGCAGTTACAGAGTTTTAT | | UACAGAGA | | UGCCUGGAAA |
| | GCAAA | | | | G |
| 148 | GAATACAGCCATGCTTTCCA | 556 | AGGCAGUU | 964 | UACUCUGUAA |
| | GGCAGTTACAGAGTTTTATG | | ACAGAGUA | | CUGCCUGGAA |
| | CAAAA | | | | A |
| 149 | AATACAGCCATGCTTTCCAG | 557 | GGCAGUUA | 965 | UAACUCUGUA |
| | GCAGTTACAGAGTTTTATGC | | CAGAGUUA | | ACUGCCUGGA |
| | AAAAG | | | | A |
| 150 | ATACAGCCATGCTTTCCAGG | 558 | GCAGUUAC | 966 | UAAACUCUGU |
| | CAGTTACAGAGTTTTATGCA | | AGAGUUUA | | AACUGCCUGG |
| | AAAGA | | | | A |
| 151 | TACAGCCATGCTTTCCAGGC | 559 | CAGUUACA | 967 | UAAAACUCUG |
| | AGTTACAGAGTTTTATGCAA | | GAGUUUUA | | UAACUGCCUG |
| | AAGAT | | | | G |
| 152 | ACAGCCATGCTTTCCAGGCA | 560 | AGUUACAG | 968 | UUAAAACUCU |
| | GTTACAGAGTTTTATGCAAA | | AGUUUUAA | | GUAACUGCCU |
| | AGATA | | | | G |
| 153 | CAGCCATGCTTTCCAGGCAG | 561 | GUUACAGA | 969 | UAUAAAACUC |
| | TTACAGAGTTTTATGCAAAA | | GUUUUAUA | | UGUAACUGCC |
| | GATAC | | | | U |
| 154 | GGCAGTTACAGAGTTTTATG | 562 | GCAAAAGA | 970 | UAACUGUAUC |
| | CAAAAGATACAGTTGACATC | | UACAGUUA | | UUUUGCAUAA |
| | AAAGG | | | | A |
| 155 | AGTTTTATGCAAAAGATACA | 563 | AGUUGACA | 971 | UCCUUUGAUG |
| | GTTGACATCAAAGGTTCTCA | | UCAAAGGA | | UCAACUGUAU |
| | AATTA | | | | C |
| 156 | GTTTTATGCAAAAGATACAG | 564 | GUUGACAU | 972 | UACCUUUGAU |
| | TTGACATCAAAGGTTCTCAA | | CAAAGGUA | | GUCAACUGUA |
| | ATTAT | | | | U |
| 157 | TTTTATGCAAAAGATACAGTT | 565 | UUGACAUC | 973 | UAACCUUUGA |
| | GACATCAAAGGTTCTCAAAT | | AAAGGUUA | | UGUCAACUGU |
| | TATT | | | | A |
| 158 | TTTATGCAAAAGATACAGTT | 566 | UGACAUCA | 974 | UGAACCUUUG |
| | GACATCAAAGGTTCTCAAAT | | AAGGUUCA | | AUGUCAACUG |
| | TATTT | | | | U |
| 159 | TTATGCAAAAGATACAGTTG | 567 | GACAUCAA | 975 | UAGAACCUUU |
| | ACATCAAAGGTTCTCAAATT | | AGGUUCUA | | GAUGUCAACU |
| | ATTTC | | | | G |
| 160 | TATGCAAAAGATACAGTTGA | 568 | ACAUCAAA | 976 | UGAGAACCUU |
| | CATCAAAGGTTCTCAAATTAT | | GGUUCUCA | | UGAUGUCAAC |
| | TTCT | | | | U |
| 161 | ATGCAAAAGATACAGTTGAC | 569 | CAUCAAAG | 977 | UUGAGAACCU |
| | ATCAAAGGTTCTCAAATTATT | | GUUCUCAA | | UUGAUGUCAA |
| | TCTG | | | | C |
| 162 | TGCAAAAGATACAGTTGACA | 570 | AUCAAAGG | 978 | UUUGAGAACC |
| | TCAAAGGTTCTCAAATTATTT | | UUCUCAAA | | UUUGAUGUCA |
| | CTGG | | | | A |
| 163 | AAAGGTTCTCAAATTATTTCT | 571 | CUGGCAUU | 979 | UAGUUAACAA |
| | GGCATTGTTAACTTAGAGAA | | GUUAACUA | | UGCCAGAAAU |
| | GCCT | | | | A |
| 164 | AAGGTTCTCAAATTATTTCTG | 572 | UGGCAUU | 980 | UAAGUUAACA |
| | GCATTGTTAACTTAGAGAAG | | GUUAACUU | | AUGCCAGAAA |
| | CCTG | | A | | U |
| 165 | AGGTTCTCAAATTATTTCTG | 573 | GGCAUUG | 981 | UUAAGUUAAC |
| | GCATTGTTAACTTAGAGAAG | | UUAACUUA | | AAUGCCAGAA |
| | CCTGT | | A | | A |
| 166 | GGTTCTCAAATTATTTCTGG | 574 | GCAUUGUU | 982 | UCUAAGUUAA |
| | CATTGTTAACTTAGAGAAGC | | AACUUAGA | | CAAUGCCAGA |
| | CTGTG | | | | A |
| 167 | GTTCTCAAATTATTTCTGGCA | 575 | CAUUGUUA | 983 | UUCUAAGUUA |
| | TTGTTAACTTAGAGAAGCCT | | ACUUAGAA | | ACAAUGCCAG |
| | GTGA | | | | A |
| 168 | TTCTCAAATTATTTCTGGCAT | 576 | AUUGUUAA | 984 | UCUCUAAGUU |
| | TGTTAACTTAGAGAAGCCTG | | CUUAGAGA | | AACAAUGCCA |
| | TGAT | | | | G |
| 169 | TCTCAAATTATTTCTGGCATT | 577 | UUGUUAAC | 985 | UUCUCUAAGU |
| | GTTAACTTAGAGAAGCCTGT | | UUAGAGAA | | UAACAAUGCC |
| | GATT | | | | A |
| 170 | ATTTCTGGCATTGTTAACTTA | 578 | UAGAGAAG | 986 | UUCACAGGCU |
| | GAGAAGCCTGTGATTTGCTC | | CCUGUGAA | | UCUCUAAGUU |
| | TTTG | | | | A |
| 171 | TTAGAGAAGCCTGTGATTTG | 579 | GCUCUUU | 987 | UUGGCAGCCA |
| | CTCTTTGGCTGCCATCATAA | | GGCUGCC | | AAGAGCAAAU |
| | AATAC | | AA | | C |
| 172 | GAAGCCTGTGATTTGCTCTT | 580 | UUGGCUG | 988 | UUAUGAUGGC |
| | TGGCTGCCATCATAAAATAC | | CCAUCAUA | | AGCCAAAGAG |
| | CTCAA | | A | | C |
| 173 | GTGATTTGCTCTTTGGCTGC | 581 | CCAUCAUA | 989 | UGGUAUUUUA |
| | CATCATAAAATACCTCAAAG | | AAAUACCA | | UGAUGGCAGC |
| | AATTC | | | | C |
| 174 | GATTTGCTCTTTGGCTGCCA | 582 | AUCAUAAA | 990 | UGAGGUAUUU |
| | TCATAAAATACCTCAAAGAAT | | AUACCUCA | | UAUGAUGGCA |
| | TCAA | | | | G |
| 175 | ATTTGCTCTTTGGCTGCCAT | 583 | UCAUAAAA | 991 | UUGAGGUAUU |
| | CATAAAATACCTCAAAGAATT | | UACCUCAA | | UUAUGAUGGC |
| | CAAC | | | | A |
| 176 | TTTGCTCTTTGGCTGCCATC | 584 | CAUAAAAU | 992 | UUUGAGGUAU |
| | ATAAAATACCTCAAAGAATTC | | ACCUCAAA | | UUUAUGAUGG |
| | AACT | | | | C |
| 177 | TTGCTCTTTGGCTGCCATCA | 585 | AUAAAAUA | 993 | UUUUGAGGUA |
| | TAAAATACCTCAAAGAATTCA | | CCUCAAAA | | UUUUAUGAUG |
| | ACTT | | | | G |
| 178 | TGCTCTTTGGCTGCCATCAT | 586 | UAAAAUAC | 994 | UCUUUGAGGU |
| | AAAATACCTCAAAGAATTCA | | CUCAAAGA | | AUUUUAUGAU |
| | ACTTG | | | | G |
| 179 | GCTCTTTGGCTGCCATCATA | 587 | AAAAUACC | 995 | UUCUUUGAGG |
| | AAATACCTCAAAGAATTCAA | | UCAAAGAA | | UAUUUUAUGA |
| | CTTGG | | | | U |
| 180 | CTCTTTGGCTGCCATCATAA | 588 | AAAUACCU | 996 | UUUCUUUGAG |
| | AATACCTCAAAGAATTCAAC | | CAAAGAAA | | GUAUUUUAUG |
| | TTGGA | | | | A |
| 181 | CATCATAAAATACCTCAAAG | 589 | GAAUUCAA | 997 | UUUCCAAGUU |
| | AATTCAACTTGGAAAAGATG | | CUUGGAAA | | GAAUUCUUUG |
| | CTCTC | | | | A |
| 182 | ATACCTCAAAGAATTCAACTT | 590 | UUGGAAAA | 998 | UGAGCAUCUU |
| | GGAAAAGATGCTCTCCAAAC | | GAUGCUCA | | UUCCAAGUUG |
| | CTGA | | | | A |
| 183 | AGATGCTCTCCAAACCTGAG | 591 | GAAUUUUA | 999 | UAGCUGUUUA |
| | AATTTTAAACAGCTATCAAGT | | AACAGCUA | | AAAUUCUCAG |
| | AAAA | | | | G |
| 184 | GATGCTCTCCAAACCTGAGA | 592 | AAUUUUAA | 1000 | UUAGCUGUUU |
| | ATTTTAAACAGCTATCAAGTA | | ACAGCUAA | | AAAAUUCUCA |
| | AAAT | | | | G |
| 185 | ATGCTCTCCAAACCTGAGAA | 593 | AUUUUAAA | 1001 | UAUAGCUGUU |
| | TTTTAAACAGCTATCAAGTAA | | CAGCUAUA | | UAAAAUUCUC |
| | AATG | | | | A |
| 186 | TGCTCTCCAAACCTGAGAAT | 594 | UUUUAAAC | 1002 | UGAUAGCUGU |
| | TTTAAACAGCTATCAAGTAAA | | AGCUAUCA | | UUAAAAUUCU |
| | ATGG | | | | C |
| 187 | GCTCTCCAAACCTGAGAATT | 595 | UUUAAACA | 1003 | UUGAUAGCUG |
| | TTAAACAGCTATCAAGTAAA | | GCUAUCAA | | UUUAAAAUUC |
| | ATGGA | | | | U |
| 188 | TTTATGACAATTAATGGAACA | 596 | CAACAUUA | 1004 | UGAUUCCUUA |
| | ACATTAAGGAATCTGGAAAT | | AGGAAUCA | | AUGUUGUUCC |
| | CCTA | | | | A |
| 189 | TTATGACAATTAATGGAACA | 597 | AACAUUAA | 1005 | UAGAUUCCUU |
| | ACATTAAGGAATCTGGAAAT | | GGAAUCUA | | AAUGUUGUUC |
| | CCTAC | | | | C |
| 190 | TATGACAATTAATGGAACAA | 598 | ACAUUAAG | 1006 | UCAGAUUCCU |
| | CATTAAGGAATCTGGAAATC | | GAAUCUGA | | UAAUGUUGUU |
| | CTACA | | | | C |
| 191 | ATGACAATTAATGGAACAAC | 599 | CAUUAAGG | 1007 | UCCAGAUUCC |
| | ATTAAGGAATCTGGAAATCC | | AAUCUGGA | | UUAAUGUUGU |
| | TACAG | | | | U |
| 192 | TGACAATTAATGGAACAACA | 600 | AUUAAGGA | 1008 | UUCCAGAUUC |
| | TTAAGGAATCTGGAAATCCT | | AUCUGGAA | | CUUAAUGUUG |
| | ACAGA | | | | U |
| 193 | AGGAATCTGGAAATCCTACA | 601 | AGAAUCAG | 1009 | UUAUCAGUCU |
| | GAATCAGACTGATATGAAAA | | ACUGAUAA | | GAUUCUGUAG |
| | CCAAA | | | | G |
| 194 | GGAATCTGGAAATCCTACAG | 602 | GAAUCAGA | 1010 | UAUAUCAGUC |
| | AATCAGACTGATATGAAAAC | | CUGAUAUA | | UGAUUCUGUA |
| | CAAAG | | | | G |
| 195 | GAATCTGGAAATCCTACAGA | 603 | AAUCAGAC | 1011 | UCAUAUCAGU |
| | ATCAGACTGATATGAAAACC | | UGAUAUGA | | CUGAUUCUGU |
| | AAAGG | | | | A |
| 196 | AATCTGGAAATCCTACAGAA | 604 | AUCAGACU | 1012 | UUCAUAUCAG |
| | TCAGACTGATATGAAAACCA | | GAUAUGAA | | UCUGAUUCUG |
| | AAGGA | | | | U |
| 197 | ATCTGGAAATCCTACAGAAT | 605 | UCAGACUG | 1013 | UUUCAUAUCA |
| | CAGACTGATATGAAAACCAA | | AUAUGAAA | | GUCUGAUUCU |
| | AGGAA | | | | G |
| 198 | TCTGGAAATCCTACAGAATC | 606 | CAGACUGA | 1014 | UUUUCAUAUC |
| | AGACTGATATGAAAACCAAA | | UAUGAAAA | | AGUCUGAUUC |
| | GGAAG | | | | U |
| 199 | TGGAAATCCTACAGAATCAG | 607 | GACUGAUA | 1015 | UGUUUUCAUA |
| | ACTGATATGAAAACCAAAGG | | UGAAAACA | | UCAGUCUGAU |
| | AAGTT | | | | U |
| 200 | AAATCCTACAGAATCAGACT | 608 | UGAUAUGA | 1016 | UUUGGUUUUC |
| | GATATGAAAACCAAAGGAAG | | AAACCAAA | | AUAUCAGUCU |
| | TTTGC | | | | G |
| 201 | CAAAGGAAGTTTGCTGTGGG | 609 | GUUUUAGA | 1017 | UAGUGUGGUC |
| | TTTTAGACCACACTAAAACTT | | CCACACUA | | UAAAACCCAC |
| | CATT | | | | A |
| 202 | AAAGGAAGTTTGCTGTGGGT | 610 | UUUUAGAC | 1018 | UUAGUGUGGU |
| | TTTAGACCACACTAAAACTT | | CACACUAA | | CUAAAACCCA |
| | CATTT | | | | C |
| 203 | AAGGAAGTTTGCTGTGGGTT | 611 | UUUAGACC | 1019 | UUUAGUGUGG |
| | TTAGACCACACTAAAACTTC | | ACACUAAA | | UCUAAAACCC |
| | ATTTG | | | | A |
| 204 | ACCACACTAAAACTTCATTT | 612 | UGGGAGA | 1020 | UAACUUCCGU |
| | GGGAGACGGAAGTTAAAGA | | CGGAAGUU | | CUCCCAAAUG |
| | AGTGGG | | A | | A |
| 205 | TTAAAGAAGTGGGTGACCCA | 613 | AGCCACUC | 1021 | UAUUUAAGGA |
| | GCCACTCCTTAAATTAAGGG | | CUUAAAUA | | GUGGCUGGGU |
| | AAATA | | | | C |
| 206 | GAAATAAATGCCCGGCTTGA | 614 | AUGCUGUA | 1022 | UCUUCCGAUA |
| | TGCTGTATCGGAAGTTCTCC | | UCGGAAGA | | CAGCAUCAAG |
| | ATTCA | | | | C |
| 207 | AAATAAATGCCCGGCTTGAT | 615 | UGCUGUAU | 1023 | UACUUCCGAU |
| | GCTGTATCGGAAGTTCTCCA | | CGGAAGUA | | ACAGCAUCAA |
| | TTCAG | | | | G |
| 208 | AATAAATGCCCGGCTTGATG | 616 | GCUGUAUC | 1024 | UAACUUCCGA |
| | CTGTATCGGAAGTTCTCCAT | | GGAAGUUA | | UACAGCAUCA |
| | TCAGA | | | | A |
| 209 | ATAAATGCCCGGCTTGATGC | 617 | CUGUAUCG | 1025 | UGAACUUCCG |
| | TGTATCGGAAGTTCTCCATT | | GAAGUUCA | | AUACAGCAUC |
| | CAGAA | | | | A |
| 210 | TAAATGCCCGGCTTGATGCT | 618 | UGUAUCG | 1026 | UAGAACUUCC |
| | GTATCGGAAGTTCTCCATTC | | GAAGUUCU | | GAUACAGCAU |
| | AGAAT | | A | | C |
| 211 | AAATGCCCGGCTTGATGCTG | 619 | GUAUCGGA | 1027 | UGAGAACUUC |
| | TATCGGAAGTTCTCCATTCA | | AGUUCUCA | | CGAUACAGCA |
| | GAATC | | | | U |
| 212 | AATGCCCGGCTTGATGCTGT | 620 | UAUCGGAA | 1028 | UGGAGAACUU |
| | ATCGGAAGTTCTCCATTCAG | | GUUCUCCA | | CCGAUACAGC |
| | AATCT | | | | A |
| 213 | CATTCAGAATCTAGTGTGTT | 621 | UUGGUCA | 1029 | UUUUCUAUCU |
| | TGGTCAGATAGAAAATCATC | | GAUAGAAA | | GACCAAACAC |
| | TACGT | | A | | A |
| 214 | TTCAGAATCTAGTGTGTTTG | 622 | GGUCAGAU | 1030 | UAUUUUCUAU |
| | GTCAGATAGAAAATCATCTA | | AGAAAAUA | | CUGACCAAAC |
| | CGTAA | | | | A |
| 215 | TCAGAATCTAGTGTGTTTGG | 623 | GUCAGAUA | 1031 | UGAUUUUCUA |
| | TCAGATAGAAAATCATCTAC | | GAAAAUCA | | UCUGACCAAA |
| | GTAAA | | | | C |
| 216 | CAGAATCTAGTGTGTTTGGT | 624 | UCAGAUAG | 1032 | UUGAUUUUCU |
| | CAGATAGAAAATCATCTACG | | AAAAUCAA | | AUCUGACCAA |
| | TAAAT | | | | A |
| 217 | AGAATCTAGTGTGTTTGGTC | 625 | CAGAUAGA | 1033 | UAUGAUUUUC |
| | AGATAGAAAATCATCTACGT | | AAAUCAUA | | UAUCUGACCA |
| | AAATT | | | | A |
| 218 | GAATCTAGTGTGTTTGGTCA | 626 | AGAUAGAA | 1034 | UGAUGAUUUU |
| | GATAGAAAATCATCTACGTA | | AAUCAUCA | | CUAUCUGACC |
| | AATTG | | | | A |
| 219 | GAAAATCATCTACGTAAATT | 627 | UGCCCGAC | 1035 | UUCUCUAUGU |
| | GCCCGACATAGAGAGGGGA | | AUAGAGAA | | CGGGCAAUUU |
| | CTCTGT | | | | A |
| 220 | GACATAGAGAGGGGACTCT | 628 | GUAGCAUU | 1036 | UUGUGAUAAA |
| | GTAGCATTTATCACAAAAAAT | | UAUCACAA | | UGCUACAGAG |
| | GTTCT | | | | U |
| 22 | ACATAGAGAGGGGACTCTGT | 629 | UAGCAUUU | 1037 | UUUGUGAUAA |
| | AGCATTTATCACAAAAAATGT | | AUCACAAA | | AUGCUACAGA |
| | TCTA | | | | G |
| 222 | CATAGAGAGGGGACTCTGTA | 630 | AGCAUUUA | 1038 | UUUUGUGAUA |
| | GCATTTATCACAAAAAATGTT | | UCACAAAA | | AAUGCUACAG |
| | CTAC | | | | A |
| 223 | ATTTATCACAAAAAATGTTCT | 631 | CUACCCAA | 1039 | UAGAACUCUU |
| | ACCCAAGAGTTCTTCTTGAT | | GAGUUCUA | | GGGUAGAACA |
| | TGTC | | | | U |
| 224 | TTATCACAAAAAATGTTCTAC | 632 | ACCCAAGA | 1040 | UGAAGAACUC |
| | CCAAGAGTTCTTCTTGATTG | | GUUCUUCA | | UUGGGUAGAA |
| | TCAA | | | | C |
| 225 | TATCACAAAAAATGTTCTACC | 633 | CCCAAGAG | 1041 | UAGAAGAACU |
| | CAAGAGTTCTTCTTGATTGT | | UUCUUCUA | | CUUGGGUAGA |
| | CAAA | | | | A |
| 226 | ATCACAAAAAATGTTCTACC | 634 | CCAAGAGU | 1042 | UAAGAAGAAC |
| | CAAGAGTTCTTCTTGATTGT | | UCUUCUUA | | UCUUGGGUAG |
| | CAAAA | | | | A |
| 227 | TCACAAAAAATGTTCTACCC | 635 | CAAGAGUU | 1043 | UCAAGAAGAA |
| | AAGAGTTCTTCTTGATTGTC | | CUUCUUGA | | CUCUUGGGUA |
| | AAAAC | | | | G |
| 228 | CACAAAAAATGTTCTACCCA | 636 | AAGAGUUC | 1044 | UUCAAGAAGA |
| | AGAGTTCTTCTTGATTGTCA | | UUCUUGAA | | ACUCUUGGGU |
| | AAACT | | | | A |
| 229 | ACAAAAAATGTTCTACCCAA | 637 | AGAGUUCU | 1045 | UAUCAAGAAG |
| | GAGTTCTTCTTGATTGTCAA | | UCUUGAUA | | AACUCUUGGG |
| | AACTT | | | | U |
| 230 | TTCTACCCAAGAGTTCTTCTT | 638 | UUGAUUGU | 1046 | UAGUUUUGAC |
| | GATTGTCAAAACTTTATATCA | | CAAAACUA | | AAUCAAGAAG |
| | CCT | | | | A |
| 231 | TTGTCAAAACTTTATATCACC | 639 | CCUAAAGU | 1047 | UAAUUCUGAC |
| | TAAAGTCAGAATTTCAAGCA | | CAGAAUUA | | UUUAGGUGAU |
| | ATAA | | | | A |
| 232 | TGTCAAAACTTTATATCACCT | 640 | CUAAAGUC | 1048 | UAAAUUCUGA |
| | AAAGTCAGAATTTCAAGCAA | | AGAAUUUA | | CUUUAGGUGA |
| | TAAT | | | | U |
| 233 | GTCAAAACTTTATATCACCTA | 641 | UAAAGUCA | 1049 | UGAAAUUCUG |
| | AAGTCAGAATTTCAAGCAAT | | GAAUUUCA | | ACUUUAGGUG |
| | AATA | | | | A |
| 234 | TCAAAACTTTATATCACCTAA | 642 | AAAGUCAG | 1050 | UUGAAAUUCU |
| | AGTCAGAATTTCAAGCAATA | | AAUUUCAA | | GACUUUAGGU |
| | ATAC | | | | G |
| 235 | CAAAACTTTATATCACCTAAA | 643 | AAGUCAGA | 1051 | UUUGAAAUUC |
| | GTCAGAATTTCAAGCAATAA | | AUUUCAAA | | UGACUUUAGG |
| | TACC | | | | U |
| 236 | CCTAAAGTCAGAATTTCAAG | 644 | GCAAUAAU | 1052 | UAGCAGGUAU |
| | CAATAATACCTGCTGTTAATT | | ACCUGCUA | | UAUUGCUUGA |
| | CCCA | | | | A |
| 237 | GAATTTCAAGCAATAATACC | 645 | CUGCUGU | 1053 | UGGGAAUUAA |
| | TGCTGTTAATTCCCACATTC | | UAAUUCCC | | CAGCAGGUAU |
| | AGTCA | | A | | U |
| 238 | ATTTCAAGCAATAATACCTG | 646 | GCUGUUAA | 1054 | UGUGGGAAUU |
| | CTGTTAATTCCCACATTCAG | | UUCCCACA | | AACAGCAGGU |
| | TCAGA | | | | A |
| 239 | TTAATTCCCACATTCAGTCA | 647 | AGACUUGC | 1055 | UGUCCGGAGC |
| | GACTTGCTCCGGACCGTTAT | | UCCGGACA | | AAGUCUGACU |
| | TTTAG | | | | G |
| 240 | ATTTTAGAAATTCCTGAACTC | 648 | UCCUCAGU | 1056 | UCCACUGGAC |
| | CTCAGTCCAGTGGAGCATTA | | CCAGUGGA | | UGAGGAGUUC |
| | CTTA | | | | A |
| 241 | TTCCTGAACTCCTCAGTCCA | 649 | AGUGGAG | 1057 | UAAGUAAUGC |
| | GTGGAGCATTACTTAAAGAT | | CAUUACUU | | UCCACUGGAC |
| | ACTCA | | A | | U |
| 242 | CCTGAACTCCTCAGTCCAGT | 650 | UGGAGCAU | 1058 | UUUAAGUAAU |
| | GGAGCATTACTTAAAGATAC | | UACUUAAA | | GCUCCACUGG |
| | TCAAT | | | | A |
| 243 | AAAACTGAATTATTTAAAGAC | 651 | ACCUUUCU | 1059 | UGGAAGUCAG |
| | CTTTCTGACTTCCCTTTAATA | | GACUUCCA | | AAAGGUCUUU |
| | AAA | | | | A |
| 244 | GAATTATTTAAAGACCTTTCT | 652 | CUGACUUC | 1060 | UUUAAAGGGA |
| | GACTTCCCTTTAATAAAAAA | | CCUUUAAA | | AGUCAGAAAG |
| | GAGG | | | | G |
| 245 | AATTATTTAAAGACCTTTCTG | 653 | UGACUUCC | 1061 | UAUUAAAGGG |
| | ACTTCCCTTTAATAAAAAAGA | | CUUUAAUA | | AAGUCAGAAA |
| | GGA | | | | G |
| 246 | ATTATTTAAAGACCTTTCTGA | 654 | GACUUCCC | 1062 | UUAUUAAAGG |
| | CTTCCCTTTAATAAAAAAGA | | UUUAAUAA | | GAAGUCAGAA |
| | GGAA | | | | A |
| 247 | TTATTTAAAGACCTTTCTGAC | 655 | ACUUCCCU | 1063 | UUUAUUAAAG |
| | TTCCCTTTAATAAAAAAGAG | | UUAAUAAA | | GGAAGUCAGA |
| | GAAG | | | | A |
| 248 | TATTTAAAGACCTTTCTGACT | 656 | CUUCCCUU | 1064 | UUUUAUUAAA |
| | TCCCTTTAATAAAAAAGAGG | | UAAUAAAA | | GGGAAGUCAG |
| | AAGG | | | | A |
| 249 | GAAATTCAAGGTGTTATTGA | 657 | ACGAGAUC | 1065 | UGCAUUCGGA |
| | CGAGATCCGAATGCATTTGC | | CGAAUGCA | | UCUCGUCAAU |
| | AAGAA | | | | A |
| 250 | GTGTTATTGACGAGATCCGA | 658 | AAUGCAUU | 1066 | UUCUUGCAAA |
| | ATGCATTTGCAAGAAATACG | | UGCAAGAA | | UGCAUUCGGA |
| | AAAAA | | | | U |
| 251 | TGTTATTGACGAGATCCGAA | 659 | AUGCAUUU | 1067 | UUUCUUGCAA |
| | TGCATTTGCAAGAAATACGA | | GCAAGAAA | | AUGCAUUCGG |
| | AAAAT | | | | A |
| 252 | GTTATTGACGAGATCCGAAT | 660 | UGCAUUUG | 1068 | UUUUCUUGCA |
| | GCATTTGCAAGAAATACGAA | | CAAGAAAA | | AAUGCAUUCG |
| | AAATA | | | | G |
| 253 | TTATTGACGAGATCCGAATG | 661 | GCAUUUGC | 1069 | UAUUUCUUGC |
| | CATTTGCAAGAAATACGAAA | | AAGAAAUA | | AAAUGCAUUC |
| | AATAC | | | | G |
| 254 | TATTGACGAGATCCGAATGC | 662 | CAUUUGCA | 1070 | UUAUUUCUUG |
| | ATTTGCAAGAAATACGAAAA | | AGAAAUAA | | CAAAUGCAUU |
| | ATACT | | | | C |
| 255 | GACGAGATCCGAATGCATTT | 663 | UGCAAGAA | 1071 | UUUCGUAUUU |
| | GCAAGAAATACGAAAAATAC | | AUACGAAA | | CUUGCAAAUG |
| | TAAAA | | | | C |
| 256 | TTCTGCACAATATGTGACAG | 664 | GUAUCAGG | 1072 | UCUCCUGUCC |
| | TATCAGGACAGGAGTTTATG | | ACAGGAGA | | UGAUACUGUC |
| | ATAGA | | | | A |
| 257 | TCTGCACAATATGTGACAGT | 665 | UAUCAGGA | 1073 | UACUCCUGUC |
| | ATCAGGACAGGAGTTTATGA | | CAGGAGUA | | CUGAUACUGU |
| | TAGAA | | | | C |
| 258 | CTGCACAATATGTGACAGTA | 666 | AUCAGGAC | 1074 | UAACUCCUGU |
| | TCAGGACAGGAGTTTATGAT | | AGGAGUUA | | CCUGAUACUG |
| | AGAAA | | | | U |
| 259 | TGCACAATATGTGACAGTAT | 667 | UCAGGACA | 1075 | UAAACUCCUG |
| | CAGGACAGGAGTTTATGATA | | GGAGUUUA | | UCCUGAUACU |
| | GAAAT | | | | G |
| 260 | GCACAATATGTGACAGTATC | 668 | CAGGACAG | 1076 | UUAAACUCCU |
| | AGGACAGGAGTTTATGATAG | | GAGUUUAA | | GUCCUGAUAC |
| | AAATA | | | | U |
| 261 | CACAATATGTGACAGTATCA | 669 | AGGACAGG | 1077 | UAUAAACUCC |
| | GGACAGGAGTTTATGATAGA | | AGUUUAUA | | UGUCCUGAUA |
| | AATAA | | | | C |
| 262 | ACAATATGTGACAGTATCAG | 670 | GGACAGGA | 1078 | UCAUAAACUC |
| | GACAGGAGTTTATGATAGAA | | GUUUAUGA | | CUGUCCUGAU |
| | ATAAA | | | | A |
| 263 | TCAGGACAGGAGTTTATGAT | 671 | UAGAAAUA | 1079 | UAGUUCUUUA |
| | AGAAATAAAGAACTCTGCTG | | AAGAACUA | | UUUCUAUCAU |
| | TATCT | | | | A |
| 264 | CAGGACAGGAGTTTATGATA | 672 | AGAAAUAA | 1080 | UGAGUUCUUU |
| | GAAATAAAGAACTCTGCTGT | | AGAACUCA | | AUUUCUAUCA |
| | ATCTT | | | | U |
| 265 | AGGACAGGAGTTTATGATAG | 673 | GAAAUAAA | 1081 | UAGAGUUCUU |
| | AAATAAAGAACTCTGCTGTA | | GAACUCUA | | UAUUUCUAUC |
| | TCTTG | | | | A |
| 266 | GGACAGGAGTTTATGATAGA | 674 | AAAUAAAG | 1082 | UCAGAGUUCU |
| | AATAAAGAACTCTGCTGTAT | | AACUCUGA | | UUAUUUCUAU |
| | CTTGT | | | | C |
| 267 | GACAGGAGTTTATGATAGAA | 675 | AAUAAAGA | 1083 | UGCAGAGUUC |
| | ATAAAGAACTCTGCTGTATC | | ACUCUGCA | | UUUAUUUCUA |
| | TTGTA | | | | U |
| 268 | TAAAGAACTCTGCTGTATCT | 676 | UUGUAUAC | 1084 | UUCAGUUGGU |
| | TGTATACCAACTGATTGGGT | | CAACUGAA | | AUACAAGAUA |
| | AAAGG | | | | C |
| 269 | TACCAACTGATTGGGTAAAG | 677 | GGUUGGA | 1085 | UUUUGUGCUU |
| | GTTGGAAGCACAAAAGCTGT | | AGCACAAA | | CCAACCUUUA |
| | GAGCC | | A | | C |
| 270 | TGATTGGGTAAAGGTTGGAA | 678 | AGCACAAA | 1086 | UCACAGCUUU |
| | GCACAAAAGCTGTGAGCCG | | AGCUGUGA | | UGUGCUUCCA |
| | CTTTCA | | | | A |
| 271 | GATTGGGTAAAGGTTGGAAG | 679 | GCACAAAA | 1087 | UUCACAGCUU |
| | CACAAAAGCTGTGAGCCGCT | | GCUGUGAA | | UUGUGCUUCC |
| | TTCAC | | | | A |
| 272 | ATTGGGTAAAGGTTGGAAGC | 680 | CACAAAAG | 1088 | UCUCACAGCU |
| | ACAAAAGCTGTGAGCCGCTT | | CUGUGAGA | | UUUGUGCUUC |
| | TCACT | | | | C |
| 273 | TTGGGTAAAGGTTGGAAGCA | 681 | ACAAAAGC | 1089 | UGCUCACAGC |
| | CAAAAGCTGTGAGCCGCTTT | | UGUGAGCA | | UUUUGUGCUU |
| | CACTC | | | | C |
| 274 | TGGGTAAAGGTTGGAAGCA | 682 | CAAAAGCU | 1090 | UGGCUCACAG |
| | CAAAAGCTGTGAGCCGCTTT | | GUGAGCCA | | CUUUUGUGCU |
| | CACTCT | | | | U |
| 275 | GGGTAAAGGTTGGAAGCAC | 683 | AAAAGCUG | 1091 | UCGGCUCACA |
| | AAAAGCTGTGAGCCGCTTTC | | UGAGCCGA | | GCUUUUGUGC |
| | ACTCTC | | | | U |
| 276 | GGTAAAGGTTGGAAGCACAA | 684 | AAAGCUGU | 1092 | UGCGGCUCAC |
| | AAGCTGTGAGCCGCTTTCAC | | GAGCCGCA | | AGCUUUUGUG |
| | TCTCC | | | | C |
| 277 | GAAGCACAAAAGCTGTGAG | 685 | CCGCUUUC | 1093 | UGGAGAGUGA |
| | CCGCTTTCACTCTCCTTTTAT | | ACUCUCCA | | AAGCGGCUCA |
| | TGTAG | | | | C |
| 278 | AAGCACAAAAGCTGTGAGCC | 686 | CGCUUUCA | 1094 | UAGGAGAGUG |
| | GCTTTCACTCTCCTTTTATTG | | CUCUCCUA | | AAAGCGGCUC |
| | TAGA | | | | A |
| 279 | AGCACAAAAGCTGTGAGCC | 687 | GCUUUCAC | 1095 | UAAGGAGAGU |
| | GCTTTCACTCTCCTTTTATTG | | UCUCCUUA | | GAAAGCGGCU |
| | TAGAA | | | | C |
| 280 | GCACAAAAGCTGTGAGCCG | 688 | CUUUCACU | 1096 | UAAAGGAGAG |
| | CTTTCACTCTCCTTTTATTGT | | CUCCUUUA | | UGAAAGCGGC |
| | AGAAA | | | | U |
| 281 | CACAAAAGCTGTGAGCCGCT | 689 | UUUCACUC | 1097 | UAAAAGGAGA |
| | TTCACTCTCCTTTTATTGTAG | | UCCUUUUA | | GUGAAAGCGG |
| | AAAA | | | | C |
| 282 | ACAAAAGCTGTGAGCCGCTT | 690 | UUCACUCU | 1098 | UUAAAAGGAG |
| | TCACTCTCCTTTTATTGTAGA | | CCUUUUAA | | AGUGAAAGCG |
| | AAAT | | | | G |
| 283 | AGCTAGTCCTTGACTGCAGT | 691 | UGCUGAAU | 1099 | UUCAAGCCAU |
| | GCTGAATGGCTTGATTTTCT | | GGCUUGAA | | UCAGCACUGC |
| | AGAGA | | | | A |
| 284 | GCTAGTCCTTGACTGCAGTG | 692 | GCUGAAUG | 1100 | UAUCAAGCCA |
| | CTGAATGGCTTGATTTTCTA | | GCUUGAUA | | UUCAGCACUG |
| | GAGAA | | | | C |
| 285 | CTAGTCCTTGACTGCAGTGC | 693 | CUGAAUGG | 1101 | UAAUCAAGCC |
| | TGAATGGCTTGATTTTCTAG | | CUUGAUUA | | AUUCAGCACU |
| | AGAAA | | | | G |
| 286 | TAGTCCTTGACTGCAGTGCT | 694 | UGAAUGGC | 1102 | UAAAUCAAGC |
| | GAATGGCTTGATTTTCTAGA | | UUGAUUUA | | CAUUCAGCAC |
| | GAAAT | | | | U |
| 287 | AGTCCTTGACTGCAGTGCTG | 695 | GAAUGGCU | 1103 | UAAAAUCAAG |
| | AATGGCTTGATTTTCTAGAG | | UGAUUUUA | | CCAUUCAGCA |
| | AAATT | | | | C |
| 288 | CCTTGACTGCAGTGCTGAAT | 696 | UGGCUUG | 1104 | UUAGAAAAUC |
| | GGCTTGATTTTCTAGAGAAA | | AUUUUCUA | | AAGCCAUUCA |
| | TTCAG | | A | | G |
| 289 | CTTGACTGCAGTGCTGAATG | 697 | GGCUUGA | 1105 | UCUAGAAAAU |
| | GCTTGATTTTCTAGAGAAATT | | UUUUCUAG | | CAAGCCAUUC |
| | CAGT | | A | | A |
| 290 | TTGACTGCAGTGCTGAATGG | 698 | GCUUGAUU | 1106 | UUCUAGAAAA |
| | CTTGATTTTCTAGAGAAATTC | | UUCUAGAA | | UCAAGCCAUU |
| | AGTG | | | | C |
| 291 | TGACTGCAGTGCTGAATGGC | 699 | CUUGAUUU | 1107 | UCUCUAGAAA |
| | TTGATTTTCTAGAGAAATTCA | | UCUAGAGA | | AUCAAGCCAU |
| | GTGA | | | | U |
| 292 | GACTGCAGTGCTGAATGGCT | 700 | UUGAUUUU | 1108 | UUCUCUAGAA |
| | TGATTTTCTAGAGAAATTCA | | CUAGAGAA | | AAUCAAGCCA |
| | GTGAA | | | | U |
| 293 | ACTGCAGTGCTGAATGGCTT | 701 | UGAUUUUC | 1109 | UUUCUCUAGA |
| | GATTTTCTAGAGAAATTCAG | | UAGAGAAA | | AAAUCAAGCC |
| | TGAAC | | | | A |
| 294 | CTGCAGTGCTGAATGGCTTG | 702 | GAUUUUCU | 1110 | UUUUCUCUAG |
| | ATTTTCTAGAGAAATTCAGT | | AGAGAAAA | | AAAAUCAAGC |
| | GAACA | | | | C |
| 295 | AGTGCTGAATGGCTTGATTT | 703 | UUCUAGAG | 1111 | UUGAAUUUCU |
| | TCTAGAGAAATTCAGTGAAC | | AAAUUCAA | | CUAGAAAAUC |
| | ATTAT | | | | A |
| 296 | CTGAATGGCTTGATTTTCTA | 704 | AGAGAAAU | 1112 | UUCACUGAAU |
| | GAGAAATTCAGTGAACATTA | | UCAGUGAA | | UUCUCUAGAA |
| | TCACT | | | | A |
| 297 | ATTTTCTAGAGAAATTCAGT | 705 | UGAACAUU | 1113 | UGAGUGAUAA |
| | GAACATTATCACTCCTTGTG | | AUCACUCA | | UGUUCACUGA |
| | TAAAG | | | | A |
| 298 | TTTCTAGAGAAATTCAGTGA | 706 | AACAUUAU | 1114 | UAGGAGUGAU |
| | ACATTATCACTCCTTGTGTAA | | CACUCCUA | | AAUGUUCACU |
| | AGCA | | | | G |
| 299 | CCTTGTGTAAAGCAGTGCAT | 707 | UCACCUAG | 1115 | UACAGUUGCU |
| | CACCTAGCAACTGTTGACTG | | CAACUGUA | | AGGUGAUGCA |
| | CATTT | | | | C |
| 300 | CATCACCTAGCAACTGTTGA | 708 | ACUGCAUU | 1116 | UGGGAGAAAA |
| | CTGCATTTTCTCCCTGGCCA | | UUCUCCCA | | UGCAGUCAAC |
| | AGGTC | | | | A |
| 301 | ATTTTCTCCCTGGCCAAGGT | 709 | UCGCUAAG | 1117 | UCUCCUUGCU |
| | CGCTAAGCAAGGAGATTACT | | CAAGGAGA | | UAGCGACCUU |
| | GCAGA | | | | G |
| 302 | CCTGGCCAAGGTCGCTAAG | 710 | CAAGGAGA | 1118 | UGCAGUAAUC |
| | CAAGGAGATTACTGCAGACC | | UUACUGCA | | UCCUUGCUUA |
| | AACTGT | | | | G |
| 303 | TGTCCCAAATAATACAGATTT | 711 | UUAUCAGA | 1119 | UUGAGUCCUC |
| | ATCAGAGGACTCAGAGAGA | | GGACUCAA | | UGAUAAAUCU |
| | GTAAT | | | | G |
| 304 | AATACAGATTTATCAGAGGA | 712 | ACUCAGAG | 1120 | UUUACUCUCU |
| | CTCAGAGAGAGTAATGATAA | | AGAGUAAA | | CUGAGUCCUC |
| | TTACC | | | | U |
| 305 | GATTTATCAGAGGACTCAGA | 713 | AGAGAGUA | 1121 | UUUAUCAUUA |
| | GAGAGTAATGATAATTACCG | | AUGAUAAA | | CUCUCUCUGA |
| | GACCA | | | | G |
| 306 | ATTTATCAGAGGACTCAGAG | 714 | GAGAGUAA | 1122 | UAUUAUCAUU |
| | AGAGTAATGATAATTACCGG | | UGAUAAUA | | ACUCUCUCUG |
| | ACCAA | | | | A |
| 307 | TTTATCAGAGGACTCAGAGA | 715 | AGAGUAAU | 1123 | UAAUUAUCAU |
| | GAGTAATGATAATTACCGGA | | GAUAAUUA | | UACUCUCUCU |
| | CCAAA | | | | G |
| 308 | TTATCAGAGGACTCAGAGAG | 716 | GAGUAAUG | 1124 | UUAAUUAUCA |
| | AGTAATGATAATTACCGGAC | | AUAAUUAA | | UUACUCUCUC |
| | CAAAC | | | | U |
| 309 | CCGGACCAAACATGGGTGG | 717 | AAAGAGCU | 1125 | UAUGUAGGAG |
| | AAAGAGCTCCTACATAAAAC | | CCUACAUA | | CUCUUUCCAC |
| | AAGTTG | | | | C |
| 310 | CCAAACATGGGTGGAAAGA | 718 | GCUCCUAC | 1126 | UGUUUUAUGU |
| | GCTCCTACATAAAACAAGTT | | AUAAAACA | | AGGAGCUCUU |
| | GCATTG | | | | U |
| 311 | CAAACATGGGTGGAAAGAG | 719 | CUCCUACA | 1127 | UUGUUUUAUG |
| | CTCCTACATAAAACAAGTTG | | UAAAACAA | | UAGGAGCUCU |
| | CATTGA | | | | U |
| 312 | AACATGGGTGGAAAGAGCT | 720 | CCUACAUA | 1128 | UCUUGUUUUA |
| | CCTACATAAAACAAGTTGCA | | AAACAAGA | | UGUAGGAGCU |
| | TTGATT | | | | C |
| 313 | ACATGGGTGGAAAGAGCTC | 721 | CUACAUAA | 1129 | UACUUGUUUU |
| | CTACATAAAACAAGTTGCAT | | AACAAGUA | | AUGUAGGAGC |
| | TGATTA | | | | U |
| 314 | CATGGGTGGAAAGAGCTCC | 722 | UACAUAAA | 1130 | UAACUUGUUU |
| | TACATAAAACAAGTTGCATT | | ACAAGUUA | | UAUGUAGGAG |
| | GATTAC | | | | C |
| 315 | ATGGGTGGAAAGAGCTCCTA | 723 | ACAUAAAA | 1131 | UCAACUUGUU |
| | CATAAAACAAGTTGCATTGA | | CAAGUUGA | | UUAUGUAGGA |
| | TTACC | | | | G |
| 316 | TGGGTGGAAAGAGCTCCTA | 724 | CAUAAAAC | 1132 | UGCAACUUGU |
| | CATAAAACAAGTTGCATTGA | | AAGUUGCA | | UUUAUGUAGG |
| | TTACCA | | | | A |
| 317 | GGGTGGAAAGAGCTCCTAC | 725 | AUAAAACA | 1133 | UUGCAACUUG |
| | ATAAAACAAGTTGCATTGATT | | AGUUGCAA | | UUUUAUGUAG |
| | ACCAT | | | | G |
| 318 | GGTGGAAAGAGCTCCTACAT | 726 | UAAAACAA | 1134 | UAUGCAACUU |
| | AAAACAAGTTGCATTGATTA | | GUUGCAUA | | GUUUUAUGUA |
| | CCATC | | | | G |
| 319 | CGACAATTGGGATTGTGGAT | 727 | UGGCAUUU | 1135 | UCUUGUGAAA |
| | GGCATTTTCACAAGGATGGG | | UCACAAGA | | AUGCCAUCCA |
| | TGCTG | | | | C |
| 320 | GACAATTGGGATTGTGGATG | 728 | GGCAUUUU | 1136 | UCCUUGUGAA |
| | GCATTTTCACAAGGATGGGT | | CACAAGGA | | AAUGCCAUCC |
| | GCTGC | | | | A |
| 321 | ACAATTGGGATTGTGGATGG | 729 | GCAUUUUC | 1137 | UUCCUUGUGA |
| | CATTTTCACAAGGATGGGTG | | ACAAGGAA | | AAAUGCCAUC |
| | CTGCA | | | | C |
| 322 | CAATTGGGATTGTGGATGGC | 730 | CAUUUUCA | 1138 | UAUCCUUGUG |
| | ATTTTCACAAGGATGGGTGC | | CAAGGAUA | | AAAAUGCCAU |
| | TGCAG | | | | C |
| 323 | AATTGGGATTGTGGATGGCA | 731 | AUUUUCAC | 1139 | UCAUCCUUGU |
| | TTTTCACAAGGATGGGTGCT | | AAGGAUGA | | GAAAAUGCCA |
| | GCAGA | | | | U |
| 324 | TGGCATTTTCACAAGGATGG | 732 | GGUGCUG | 1140 | UAUUGUCUGC |
| | GTGCTGCAGACAATATATAT | | CAGACAAU | | AGCACCCAUC |
| | AAAGG | | A | | C |
| 325 | GCATTTTCACAAGGATGGGT | 733 | UGCUGCA | 1141 | UAUAUUGUCU |
| | GCTGCAGACAATATATATAA | | GACAAUAU | | GCAGCACCCA |
| | AGGAC | | A | | U |
| 326 | CATTTTCACAAGGATGGGTG | 734 | GCUGCAGA | 1142 | UUAUAUUGUC |
| | CTGCAGACAATATATATAAA | | CAAUAUAA | | UGCAGCACCC |
| | GGACA | | | | A |
| 327 | ATTTTCACAAGGATGGGTGC | 735 | CUGCAGAC | 1143 | UAUAUAUUGU |
| | TGCAGACAATATATATAAAG | | AAUAUAUA | | CUGCAGCACC |
| | GACAG | | | | C |
| 328 | TTTTCACAAGGATGGGTGCT | 736 | UGCAGACA | 1144 | UUAUAUAUUG |
| | GCAGACAATATATATAAAGG | | AUAUAUAA | | UCUGCAGCAC |
| | ACAGA | | | | C |
| 329 | TTTCACAAGGATGGGTGCTG | 737 | GCAGACAA | 1145 | UAUAUAUAUU |
| | CAGACAATATATATAAAGGA | | UAUAUAUA | | GUCUGCAGCA |
| | CAGAG | | | | C |
| 330 | TATAAAGGACAGAGTACATT | 738 | UUAUGGAA | 1146 | UUCAGUUCUU |
| | TATGGAAGAACTGACTGACA | | GAACUGAA | | CCAUAAAUGU |
| | CAGCA | | | | A |
| 331 | TTTATGGAAGAACTGACTGA | 739 | ACACAGCA | 1147 | UUUAUUUCUG |
| | CACAGCAGAAATAATCAGAA | | GAAAUAAA | | CUGUGUCAGU |
| | AAGCA | | | | C |
| 332 | AGAAATAATCAGAAAAGCAA | 740 | ACAUCACA | 1148 | UCAAGGACUG |
| | CATCACAGTCCTTGGTTATC | | GUCCUUGA | | UGAUGUUGCU |
| | TTGGA | | | | U |
| 333 | ATCAGAAAAGCAACATCACA | 741 | AGUCCUUG | 1149 | UAGAUAACCA |
| | GTCCTTGGTTATCTTGGATG | | GUUAUCUA | | AGGACUGUGA |
| | AACTA | | | | U |
| 334 | ACATCACAGTCCTTGGTTAT | 742 | UCUUGGAU | 1150 | UCUAGUUCAU |
| | CTTGGATGAACTAGGAAGAG | | GAACUAGA | | CCAAGAUAAC |
| | GGACG | | | | C |
| 335 | TCACAGTCCTTGGTTATCTT | 743 | UGGAUGAA | 1151 | UUUCCUAGUU |
| | GGATGAACTAGGAAGAGGG | | CUAGGAAA | | CAUCCAAGAU |
| | ACGAGC | | | | A |
| 336 | ACTCATGATGGAATTGCCAT | 744 | UUGCCUAU | 1152 | UGUGUAGCAU |
| | TGCCTATGCTACACTTGAGT | | GCUACACA | | AGGCAAUGGC |
| | ATTTC | | | | A |
| 337 | CCTATGCTACACTTGAGTAT | 745 | UUUCAUCA | 1153 | UACAUCUCUG |
| | TTCATCAGAGATGTGAAATC | | GAGAUGUA | | AUGAAAUACU |
| | CTTAA | | | | C |
| 338 | TATGCTACACTTGAGTATTTC | 746 | UCAUCAGA | 1154 | UUCACAUCUC |
| | ATCAGAGATGTGAAATCCTT | | GAUGUGAA | | UGAUGAAAUA |
| | AACC | | | | C |
| 339 | GTATTTCATCAGAGATGTGA | 747 | AAAUCCUU | 1155 | UCAGGGUUAA |
| | AATCCTTAACCCTGTTTGTC | | AACCCUGA | | GGAUUUCACA |
| | ACCCA | | | | U |
| 340 | TGTGAAATCCTTAACCCTGT | 748 | UUUGUCAC | 1156 | UAUAAUGGGU |
| | TTGTCACCCATTATCCGCCA | | CCAUUAUA | | GACAAACAGG |
| | GTTTG | | | | G |
| 341 | CCTTAACCCTGTTTGTCACC | 749 | CCAUUAUC | 1157 | UACUGGCGGA |
| | CATTATCCGCCAGTTTGTGA | | CGCCAGUA | | UAAUGGGUGA |
| | ACTAG | | | | C |
| 342 | GTTTGTCACCCATTATCCGC | 750 | CCAGUUUG | 1158 | UUAGUUCACA |
| | CAGTTTGTGAACTAGAAAAA | | UGAACUAA | | AACUGGCGGA |
| | AATTA | | | | U |
| 343 | GTCACCCATTATCCGCCAGT | 751 | UUUGUGAA | 1159 | UUUUCUAGUU |
| | TTGTGAACTAGAAAAAAATTA | | CUAGAAAA | | CACAAACUGG |
| | CTCA | | | | C |
| 344 | TTACCACATGGGATTCTTGG | 752 | GUCAGUGA | 1160 | UUUCAUCCUC |
| | TCAGTGAGGATGAAAGCAAA | | GGAUGAAA | | ACUGACCAAG |
| | CTGGA | | | | A |
| 345 | AGAACAAGTCCCTGATTTTG | 753 | GUCACCUU | 1161 | UGUAAAGGAA |
| | TCACCTTCCTTTACCAAATAA | | CCUUUACA | | GGUGACAAAA |
| | CTAG | | | | U |
| 346 | GAACAAGTCCCTGATTTTGT | 754 | UCACCUUC | 1162 | UGGUAAAGGA |
| | CACCTTCCTTTACCAAATAA | | CUUUACCA | | AGGUGACAAA |
| | CTAGA | | | | A |
| 347 | AAGTCCCTGATTTTGTCACC | 755 | CUUCCUUU | 1163 | UAUUUGGUAA |
| | TTCCTTTACCAAATAACTAGA | | ACCAAAUA | | AGGAAGGUGA |
| | GGAA | | | | C |
| 348 | AGTCCCTGATTTTGTCACCT | 756 | UUCCUUUA | 1164 | UUAUUUGGUA |
| | TCCTTTACCAAATAACTAGA | | CCAAAUAA | | AAGGAAGGUG |
| | GGAAT | | | | A |
| 349 | GTCCCTGATTTTGTCACCTT | 757 | UCCUUUAC | 1165 | UUUAUUUGGU |
| | CCTTTACCAAATAACTAGAG | | CAAAUAAA | | AAAGGAAGGU |
| | GAATT | | | | G |
| 350 | TCCCTGATTTTGTCACCTTC | 758 | CCUUUACC | 1166 | UGUUAUUUGG |
| | CTTTACCAAATAACTAGAGG | | AAAUAACA | | UAAAGGAAGG |
| | AATTG | | | | U |
| 351 | TTGTCACCTTCCTTTACCAAA | 759 | AAUAACUA | 1167 | UAUUCCUCUA |
| | TAACTAGAGGAATTGCAGCA | | GAGGAAUA | | GUUAUUUGGU |
| | AGGA | | | | A |
| 352 | CTTTACCAAATAACTAGAGG | 760 | GAAUUGCA | 1168 | UUCCUUGCUG |
| | AATTGCAGCAAGGAGTTATG | | GCAAGGAA | | CAAUUCCUCU |
| | GATTA | | | | A |
| 353 | CCAAATAACTAGAGGAATTG | 761 | GCAGCAAG | 1169 | UAUAACUCCU |
| | CAGCAAGGAGTTATGGATTA | | GAGUUAUA | | UGCUGCAAUU |
| | AATGT | | | | C |
| 354 | AATAACTAGAGGAATTGCAG | 762 | GCAAGGAG | 1170 | UUCCAUAACU |
| | CAAGGAGTTATGGATTAAAT | | UUAUGGAA | | CCUUGCUGCA |
| | GTGGC | | | | A |
| 355 | ATAACTAGAGGAATTGCAGC | 763 | CAAGGAGU | 1171 | UAUCCAUAAC |
| | AAGGAGTTATGGATTAAATG | | UAUGGAUA | | UCCUUGCUGC |
| | TGGCT | | | | A |
| 356 | ACTAGAGGAATTGCAGCAAG | 764 | GGAGUUAU | 1172 | UUUAAUCCAU |
| | GAGTTATGGATTAAATGTGG | | GGAUUAAA | | AACUCCUUGC |
| | CTAAA | | | | U |
| 357 | AGGAGTTATGGATTAAATGT | 765 | UGGCUAAA | 1173 | UCUGCUAGUU |
| | GGCTAAACTAGCAGATGTTC | | CUAGCAGA | | UAGCCACAUU |
| | CTGGA | | | | U |
| 358 | CCTGGAGAAATTTTGAAGAA | 766 | AAGCAGCU | 1174 | UACUUGUGAG |
| | AGCAGCTCACAAGTCAAAAG | | CACAAGUA | | CUGCUUUCUU |
| | AGCTG | | | | C |
| 359 | TAAATACGAAAAGAAAGAGA | 767 | ACUCAAGU | 1175 | UGCAAAAUAC |
| | CTCAAGTATTTTGCAAAGTTA | | AUUUUGCA | | UUGAGUCUCU |
| | TGGA | | | | U |
| 360 | AAATACGAAAAGAAAGAGAC | 768 | CUCAAGUA | 1176 | UUGCAAAAUA |
| | TCAAGTATTTTGCAAAGTTAT | | UUUUGCAA | | CUUGAGUCUC |
| | GGAC | | | | U |
| 361 | AATACGAAAAGAAAGAGACT | 769 | UCAAGUAU | 1177 | UUUGCAAAAU |
| | CAAGTATTTTGCAAAGTTAT | | UUUGCAAA | | ACUUGAGUCU |
| | GGACG | | | | C |
| 362 | ATACGAAAAGAAAGAGACTC | 770 | CAAGUAUU | 1178 | UUUUGCAAAA |
| | AAGTATTTTGCAAAGTTATG | | UUGCAAAA | | UACUUGAGUC |
| | GACGA | | | | U |
| 363 | TACGAAAAGAAAGAGACTCA | 771 | AAGUAUUU | 1179 | UCUUUGCAAA |
| | AGTATTTTGCAAAGTTATGG | | UGCAAAGA | | AUACUUGAGU |
| | ACGAT | | | | C |
| 364 | GAGACTCAAGTATTTTGCAA | 772 | AAGUUAUG | 1180 | UCAUCGUCCA |
| | AGTTATGGACGATGCATAAT | | GACGAUGA | | UAACUUUGCA |
| | GCACA | | | | A |
| 365 | AGAAGTGGACAGAGGAGTT | 773 | CAACAUGG | 1181 | UGUUUCUUCC |
| | CAACATGGAAGAAACACAGA | | AAGAAACA | | AUGUUGAACU |
| | CTTCTC | | | | C |
| 366 | GAAGTGGACAGAGGAGTTC | 774 | AACAUGGA | 1182 | UUGUUUCUUC |
| | AACATGGAAGAAACACAGAC | | AGAAACAA | | CAUGUUGAAC |
| | TTCTCT | | | | U |
| 367 | AAGTGGACAGAGGAGTTCAA | 775 | ACAUGGAA | 1183 | UGUGUUUCUU |
| | CATGGAAGAAACACAGACTT | | GAAACACA | | CCAUGUUGAA |
| | CTCTT | | | | C |
| 368 | AGTGGACAGAGGAGTTCAA | 776 | CAUGGAAG | 1184 | UUGUGUUUCU |
| | CATGGAAGAAACACAGACTT | | AAACACAA | | UCCAUGUUGA |
| | CTCTTC | | | | A |
| 369 | GTGGACAGAGGAGTTCAAC | 777 | AUGGAAGA | 1185 | UCUGUGUUUC |
| | ATGGAAGAAACACAGACTTC | | AACACAGA | | UUCCAUGUUG |
| | TCTTCT | | | | A |
| 370 | AGAGGAGTTCAACATGGAAG | 778 | GAAACACA | 1186 | UAGAAGUCUG |
| | AAACACAGACTTCTCTTCTT | | GACUUCUA | | UGUUUCUUCC |
| | CATTA | | | | A |
| 371 | GTTCAACATGGAAGAAACAC | 779 | CAGACUUC | 1187 | UAAGAAGAGA |
| | AGACTTCTCTTCTTCATTAAA | | UCUUCUUA | | AGUCUGUGUU |
| | ATGA | | | | U |
| 372 | CTTCATTAAAATGAAGACTA | 780 | ACAUUUGU | 1188 | UUUUGUUCAC |
| | CATTTGTGAACAAAAAATGG | | GAACAAAA | | AAAUGUAGUC |
| | AGAAT | | | | U |
| 373 | ATTAAAAATACCAACTGTACA | 78 | CAAAAUAA | 1189 | UUGGAGAGUU |
| | AAATAACTCTCCAGTAACAG | | CUCUCCAA | | AUUUUGUACA |
| | CCTA | | | | G |
| 374 | TTAAAAATACCAACTGTACAA | 782 | AAAAUAAC | 1190 | UCUGGAGAGU |
| | AATAACTCTCCAGTAACAGC | | UCUCCAGA | | UAUUUUGUAC |
| | CTAT | | | | A |
| 375 | TGTACAAAATAACTCTCCAG | 783 | GUAACAGC | 1191 | UAAGAUAGGC |
| | TAACAGCCTATCTTTGTGTG | | CUAUCUUA | | UGUUACUGGA |
| | ACATG | | | | G |
| 376 | AGTAACAGCCTATCTTTGTG | 784 | GUGACAUG | 1192 | UAUGCUCACA |
| | TGACATGTGAGCATAAAATT | | UGAGCAUA | | UGUCACACAA |
| | ATGAC | | | | A |
| 377 | AACAGCCTATCTTTGTGTGA | 785 | ACAUGUGA | 1193 | UUUUAUGCUC |
| | CATGTGAGCATAAAATTATG | | GCAUAAAA | | ACAUGUCACA |
| | ACCAT | | | | C |
| 378 | ATCTTTGTGTGACATGTGAG | 786 | GCAUAAAA | 1194 | UGUCAUAAUU |
| | CATAAAATTATGACCATGGT | | UUAUGACA | | UUAUGCUCAC |
| | ATATT | | | | A |
| 379 | ATGTGAGCATAAAATTATGA | 787 | ACCAUGGU | 1195 | UGGAAUAUAC |
| | CCATGGTATATTCCTATTGG | | AUAUUCCA | | CAUGGUCAUA |
| | AAACA | | | | A |
| 380 | TGTGAGCATAAAATTATGAC | 788 | CCAUGGUA | 1196 | UAGGAAUAUA |
| | CATGGTATATTCCTATTGGA | | UAUUCCUA | | CCAUGGUCAU |
| | AACAG | | | | A |
| 381 | AATTATGACCATGGTATATTC | 789 | UCCUAUUG | 1197 | UCUGUUUCCA |
| | CTATTGGAAACAGAGAGGTT | | GAAACAGA | | AUAGGAAUAU |
| | TTTC | | | | A |
| 382 | TATGACCATGGTATATTCCT | 790 | UAUUGGAA | 1198 | UUCUCUGUUU |
| | ATTGGAAACAGAGAGGTTTT | | ACAGAGAA | | CCAAUAGGAA |
| | TCTGA | | | | U |
| 383 | CCATGGTATATTCCTATTGG | 791 | GAAACAGA | 1199 | UAAACCUCUC |
| | AAACAGAGAGGTTTTTCTGA | | GAGGUUUA | | UGUUUCCAAU |
| | AGACA | | | | A |
| 384 | GTCTTTTTCAAGTTTCTGTCT | 792 | CUUCCUAA | 1200 | UAGAAAAGUU |
| | TCCTAACTTTTCTACGTATAA | | CUUUUCUA | | AGGAAGACAG |
| | ACA | | | | A |
| 385 | TTTTCAAGTTTCTGTCTTCCT | 793 | CUAACUUU | 1201 | UACGUAGAAA |
| | AACTTTTCTACGTATAAACAC | | UCUACGUA | | AGUUAGGAAG |
| | TCT | | | | A |
| 386 | TTTCAAGTTTCTGTCTTCCTA | 794 | UAACUUUU | 1202 | UUACGUAGAA |
| | ACTTTTCTACGTATAAACACT | | CUACGUAA | | AAGUUAGGAA |
| | CTT | | | | G |
| 387 | CTGTCTTCCTAACTTTTCTAC | 795 | ACGUAUAA | 1203 | UAGAGUGUUU |
| | GTATAAACACTCTTGAATAG | | ACACUCUA | | AUACGUAGAA |
| | ACTT | | | | A |
| 388 | TGTCTTCCTAACTTTTCTACG | 796 | CGUAUAAA | 1204 | UAAGAGUGUU |
| | TATAAACACTCTTGAATAGA | | CACUCUUA | | UAUACGUAGA |
| | CTTC | | | | A |
| 389 | TCTTCCTAACTTTTCTACGTA | 797 | UAUAAACA | 1205 | UUCAAGAGUG |
| | TAAACACTCTTGAATAGACTT | | CUCUUGAA | | UUUAUACGUA |
| | CCA | | | | G |
| 390 | TTTTCTACGTATAAACACTCT | 798 | CUUGAAUA | 1206 | UGGAAGUCUA |
| | TGAATAGACTTCCACTTTGT | | GACUUCCA | | UUCAAGAGUG |
| | AATT | | | | U |
| 391 | TACGTATAAACACTCTTGAAT | 799 | AUAGACUU | 1207 | UAAAGUGGAA |
| | AGACTTCCACTTTGTAATTA | | CCACUUUA | | GUCUAUUCAA |
| | GAAA | | | | G |
| 392 | ACGTATAAACACTCTTGAAT | 800 | UAGACUUC | 1208 | UCAAAGUGGA |
| | AGACTTCCACTTTGTAATTA | | CACUUUGA | | AGUCUAUUCA |
| | GAAAA | | | | A |
| 393 | TATAAACACTCTTGAATAGA | 801 | ACUUCCAC | 1209 | UUUACAAAGU |
| | CTTCCACTTTGTAATTAGAAA | | UUUGUAAA | | GGAAGUCUAU |
| | ATTT | | | | U |
| 394 | AAACACTCTTGAATAGACTT | 802 | UCCACUUU | 1210 | UUAAUUACAA |
| | CCACTTTGTAATTAGAAAATT | | GUAAUUAA | | AGUGGAAGUC |
| | TTAT | | | | U |
| 395 | AACACTCTTGAATAGACTTC | 803 | CCACUUUG | 1211 | UCUAAUUACA |
| | CACTTTGTAATTAGAAAATTT | | UAAUUAGA | | AAGUGGAAGU |
| | TATG | | | | C |
| 396 | AATTTTATGGACAGTAAGTC | 804 | CCAGUAAA | 1212 | UUUAAGGCUU |
| | CAGTAAAGCCTTAAGTGGCA | | GCCUUAAA | | UACUGGACUU |
| | GAATA | | | | A |
| 397 | AGTAAGTCCAGTAAAGCCTT | 805 | UAAGUGGC | 1213 | UAUAUUCUGC |
| | AAGTGGCAGAATATAATTCC | | AGAAUAUA | | CACUUAAGGC |
| | CAAGC | | | | U |
| 398 | TCCAGTAAAGCCTTAAGTGG | 806 | GCAGAAUA | 1214 | UGGAAUUAUA |
| | CAGAATATAATTCCCAAGCT | | UAAUUCCA | | UUCUGCCACU |
| | TTTGG | | | | U |
| 399 | TGGCAACTGGGTGAATCTG | 807 | GCAGGAAU | 1215 | UUGGAUAGAU |
| | GCAGGAATCTATCCATTGAA | | CUAUCCAA | | UCCUGCCAGA |
| | CTAAAA | | | | U |
| 400 | CTGGGTGAATCTGGCAGGA | 808 | AUCUAUCC | 1216 | UGUUCAAUGG |
| | ATCTATCCATTGAACTAAAAT | | AUUGAACA | | AUAGAUUCCU |
| | AATTT | | | | G |
| 401 | AAAATAATTTTATTATGCAAC | 809 | ACCAGUUU | 1217 | UGGUGGAUAA |
| | CAGTTTATCCACCAAGAACA | | AUCCACCA | | ACUGGUUGCA |
| | TAAG | | | | U |
| 402 | ATTTTATTATGCAACCAGTTT | 810 | UUAUCCAC | 1218 | UGUUCUUGGU |
| | ATCCACCAAGAACATAAGAA | | CAAGAACA | | GGAUAAACUG |
| | TTTT | | | | G |
| 403 | TATGCAACCAGTTTATCCAC | 811 | CCAAGAAC | 1219 | UUUCUUAUGU |
| | CAAGAACATAAGAATTTTTTA | | AUAAGAAA | | UCUUGGUGGA |
| | TAAG | | | | U |
| 404 | AAGCTTTTAAAAACTAGAGC | 812 | CACAGAAG | 1220 | UCUUAUUCCU |
| | ACAGAAGGAATAAGGTCATG | | GAAUAAGA | | UCUGUGCUCU |
| | AAATT | | | | A |
| 405 | TTTTAAAAACTAGAGCACAG | 813 | GAAGGAAU | 1221 | UUGACCUUAU |
| | AAGGAATAAGGTCATGAAAT | | AAGGUCAA | | UCCUUCUGUG |
| | TTAAA | | | | C |
| 406 | TTTAAAAACTAGAGCACAGA | 814 | AAGGAAUA | 1222 | UAUGACCUUA |
| | AGGAATAAGGTCATGAAATT | | AGGUCAUA | | UUCCUUCUGU |
| | TAAAA | | | | G |
| 407 | AAGGTTAAATATTGTCATAG | 815 | GGAUUAAG | 1223 | UUAAACUGCU |
| | GATTAAGCAGTTTAAAGATT | | CAGUUUAA | | UAAUCCUAUG |
| | GTTGG | | | | A |
| 408 | TATTGTCATAGGATTAAGCA | 816 | AGUUUAAA | 1224 | UAACAAUCUU |
| | GTTTAAAGATTGTTGGATGA | | GAUUGUUA | | UAAACUGCUU |
| | AATTA | | | | A |

In some embodiments, the siRNA molecules of the disclosure have complementarity sufficient to hybridize to a region within an MSH3 mRNA transcript that is within the open reading frame of the MSH3 mRNA transcript. In some embodiments, the siRNA molecules of the disclosure have complementarity sufficient to hybridize to a region within an MSH3 mRNA transcript that is within the 3-untranslated region.

In some embodiments, the siRNA molecules of the disclosure have complementarity sufficient to hybridize to a region within exon 1 of an MSH3 mRNA transcript. In some embodiments, the siRNA molecules of the disclosure have complementarity sufficient to hybridize to a region within exon 2 of an MSH3 mRNA transcript. In some embodiments, the siRNA molecules of the disclosure have complementarity sufficient to hybridize to a region within exon 3 of an MSH3 mRNA transcript. In some embodiments, the siRNA molecules of the disclosure have complementarity sufficient to hybridize to a region within exon 4 of an MSH3 mRNA transcript. In some embodiments, the siRNA molecules of the disclosure have complementarity sufficient to hybridize to a region within exon 5 of an MSH3 mRNA transcript. In some embodiments, the siRNA molecules of the disclosure have complementarity sufficient to hybridize to a region within exon 6 of an MSH3 mRNA transcript. In some embodiments, the siRNA molecules of the disclosure have complementarity sufficient to hybridize to a region within exon 7 of an MSH3 mRNA transcript. In some embodiments, the siRNA molecules of the disclosure have complementarity sufficient to hybridize to a region within exon 8 of an MSH3 mRNA transcript. In some embodiments, the siRNA molecules of the disclosure have complementarity sufficient to hybridize to a region within exon 9 of an MSH3 mRNA transcript. In some embodiments, the siRNA molecules of the disclosure have complementarity sufficient to hybridize to a region within exon 10 of an MSH3 mRNA transcript. In some embodiments, the siRNA molecules of the disclosure have complementarity sufficient to hybridize to a region within exon 11 of an MSH3 mRNA transcript. In some embodiments, the siRNA molecules of the disclosure have complementarity sufficient to hybridize to a region within exon 12 of an MSH3 mRNA transcript. In some embodiments, the siRNA molecules of the disclosure have complementarity sufficient to hybridize to a region within exon 13 of an MSH3 mRNA transcript. In some embodiments, the siRNA molecules of the disclosure have complementarity sufficient to hybridize to a region within exon 14 of an MSH3 mRNA transcript. In some embodiments, the siRNA molecules of the disclosure have complementarity sufficient to hybridize to a region within exon 15 of an MSH3 mRNA transcript. In some embodiments, the siRNA molecules of the disclosure have complementarity sufficient to hybridize to a region within exon 16 of an MSH3 mRNA transcript. In some embodiments, the siRNA molecules of the disclosure have complementarity sufficient to hybridize to a region within exon 17 of an MSH3 mRNA transcript. In some embodiments, the siRNA molecules of the disclosure have complementarity sufficient to hybridize to a region within exon 18 of an MSH3 mRNA transcript. In some embodiments, the siRNA molecules of the disclosure have complementarity sufficient to hybridize to a region within exon 19 of an MSH3 mRNA transcript. In some embodiments, the siRNA molecules of the disclosure have complementarity sufficient to hybridize to a region within exon 20 of an MSH3 mRNA transcript. In some embodiments, the siRNA molecules of the disclosure have complementarity sufficient to hybridize to a region within exon 21 of an MSH3 mRNA transcript. In some embodiments, the siRNA molecules of the disclosure have complementarity sufficient to hybridize to a region within exon 22 of an MSH3 mRNA transcript. In some embodiments, the siRNA molecules of the disclosure have complementarity sufficient to hybridize to a region within exon 23 of an MSH3 mRNA transcript. In some embodiments, the siRNA molecules of the disclosure have complementarity sufficient to hybridize to a region within exon 24 of an MSH3 mRNA transcript.

Table 1B, below, discloses which position of the gene in which each target region is contained (e.g., open reading frame or 3′-untranslated region) and the exon in which each target region is contained.

TABLE 1B
Location of mRNA sequences for gene-
specific MSH3-targeting siRNA

| Gene Region SEQ ID NO: | Gene Position | Exon |
|---|---|---|
| 1 | ORF | 1 |
| 2 | ORF | 1 |
| 3 | ORF | 1 |
| 4 | ORF | 1 |
| 5 | ORF | 2 |
| 6 | ORF | 2 |
| 7 | ORF | 2 |
| 8 | ORF | 2 |
| 9 | ORF | 2 |
| 10 | ORF | 2 |
| 11 | ORF | 2 |
| 12 | ORF | 2 |
| 13 | ORF | 2 |
| 14 | ORF | 2 |
| 15 | ORF | 2 |
| 16 | ORF | 2-3 |
| 17 | ORF | 2-3 |
| 18 | ORF | 2-3 |
| 19 | ORF | 2-3 |
| 20 | ORF | 2-3 |
| 21 | ORF | 3 |
| 22 | ORF | 3 |
| 23 | ORF | 3 |
| 24 | ORF | 3 |
| 25 | ORF | 3 |
| 26 | ORF | 3 |
| 27 | ORF | 3 |
| 28 | ORF | 3 |
| 29 | ORF | 3 |
| 30 | ORF | 3 |
| 31 | ORF | 3 |
| 32 | ORF | 3 |
| 33 | ORF | 3 |
| 34 | ORF | 3 |
| 35 | ORF | 3 |
| 36 | ORF | 3 |
| 37 | ORF | 3 |
| 38 | ORF | 3 |
| 39 | ORF | 3 |
| 40 | ORF | 3 |
| 41 | ORF | 3 |
| 42 | ORF | 3 |
| 43 | ORF | 3 |
| 44 | ORF | 3 |
| 45 | ORF | 3 |
| 46 | ORF | 3 |
| 47 | ORF | 3 |
| 48 | ORF | 4 |
| 49 | ORF | 4 |
| 50 | ORF | 4 |
| 51 | ORF | 4 |
| 52 | ORF | 4 |
| 53 | ORF | 4 |
| 54 | ORF | 4 |
| 55 | ORF | 4 |
| 56 | ORF | 4 |
| 57 | ORF | 4 |
| 58 | ORF | 4 |
| 59 | ORF | 4 |
| 60 | ORF | 4 |
| 61 | ORF | 4 |
| 62 | ORF | 4 |
| 63 | ORF | 4 |
| 64 | ORF | 4 |
| 65 | ORF | 4 |
| 66 | ORF | 4 |
| 67 | ORF | 4 |
| 68 | ORF | 4 |
| 69 | ORF | 4 |
| 70 | ORF | 4 |
| 71 | ORF | 4 |
| 72 | ORF | 4 |
| 73 | ORF | 4 |
| 74 | ORF | 4 |
| 75 | ORF | 4 |
| 76 | ORF | 4 |
| 77 | ORF | 4 |
| 78 | ORF | 4 |
| 79 | ORF | 4 |
| 80 | ORF | 4 |
| 81 | ORF | 4 |
| 82 | ORF | 4 |
| 83 | ORF | 4 |
| 84 | ORF | 4 |
| 85 | ORF | 5 |
| 86 | ORF | 5 |
| 87 | ORF | 5 |
| 88 | ORF | 5 |
| 89 | ORF | 5 |
| 90 | ORF | 5 |
| 91 | ORF | 5 |
| 92 | ORF | 5 |
| 93 | ORF | 5 |
| 94 | ORF | 5 |
| 95 | ORF | 5 |
| 96 | ORF | 5 |
| 97 | ORF | 5 |
| 98 | ORF | 5 |
| 99 | ORF | 5 |
| 100 | ORF | 5 |
| 101 | ORF | 5-6 |
| 102 | ORF | 5-6 |
| 103 | ORF | 5-6 |
| 104 | ORF | 6 |
| 105 | ORF | 6 |
| 106 | ORF | 6 |
| 107 | ORF | 6 |
| 108 | ORF | 6 |
| 109 | ORF | 6 |
| 110 | ORF | 6 |
| 111 | ORF | 6 |
| 112 | ORF | 6 |
| 113 | ORF | 6 |
| 114 | ORF | 6 |
| 115 | ORF | 6 |
| 116 | ORF | 6 |
| 117 | ORF | 6 |
| 118 | ORF | 6 |
| 119 | ORF | 6 |
| 120 | ORF | 6 |
| 121 | ORF | 7 |
| 122 | ORF | 7 |
| 123 | ORF | 7 |
| 124 | ORF | 7 |
| 125 | ORF | 7 |
| 126 | ORF | 7 |
| 127 | ORF | 7 |
| 128 | ORF | 7 |
| 129 | ORF | 7 |
| 130 | ORF | 7 |
| 131 | ORF | 7 |
| 132 | ORF | 7 |
| 133 | ORF | 7 |
| 134 | ORF | 8 |
| 135 | ORF | 8 |
| 136 | ORF | 8 |
| 137 | ORF | 8 |
| 138 | ORF | 8 |
| 139 | ORF | 8 |
| 140 | ORF | 9 |
| 141 | ORF | 9 |
| 142 | ORF | 9 |
| 143 | ORF | 9 |
| 144 | ORF | 9 |
| 145 | ORF | 9 |
| 146 | ORF | 9 |
| 147 | ORF | 9 |
| 148 | ORF | 9 |
| 149 | ORF | 9 |
| 150 | ORF | 9 |
| 151 | ORF | 9 |
| 152 | ORF | 9 |
| 153 | ORF | 9 |
| 154 | ORF | 9 |
| 155 | ORF | 9-10 |
| 156 | ORF | 9-10 |
| 157 | ORF | 9-10 |
| 158 | ORF | 9-10 |
| 159 | ORF | 9-10 |
| 160 | ORF | 9-10 |
| 161 | ORF | 9-10 |
| 162 | ORF | 9-10 |
| 163 | ORF | 10 |
| 164 | ORF | 10 |
| 165 | ORF | 10 |
| 166 | ORF | 10 |
| 167 | ORF | 10 |
| 168 | ORF | 10 |
| 169 | ORF | 10 |
| 170 | ORF | 10 |
| 171 | ORF | 10 |
| 172 | ORF | 10 |
| 173 | ORF | 10 |
| 174 | ORF | 10 |
| 175 | ORF | 10 |
| 176 | ORF | 10 |
| 177 | ORF | 10 |
| 178 | ORF | 10 |
| 179 | ORF | 10 |
| 180 | ORF | 10 |
| 181 | ORF | 10 |
| 182 | ORF | 10 |
| 183 | ORF | 10-11 |
| 184 | ORF | 10-11 |
| 185 | ORF | 10-11 |
| 186 | ORF | 10-11 |
| 187 | ORF | 10-11 |
| 188 | ORF | 11 |
| 189 | ORF | 11 |
| 190 | ORF | 11 |
| 191 | ORF | 11 |
| 192 | ORF | 11 |
| 193 | ORF | 11-12 |
| 194 | ORF | 11-12 |
| 195 | ORF | 11-12 |
| 196 | ORF | 11-12 |
| 197 | ORF | 11-12 |
| 198 | ORF | 11-12 |
| 199 | ORF | 11-12 |
| 200 | ORF | 11-12 |
| 201 | ORF | 12 |
| 202 | ORF | 12 |
| 203 | ORF | 12 |
| 204 | ORF | 12 |
| 205 | ORF | 12 |
| 206 | ORF | 13 |
| 207 | ORF | 13 |
| 208 | ORF | 13 |
| 209 | ORF | 13 |
| 210 | ORF | 13 |
| 211 | ORF | 13 |
| 212 | ORF | 13 |
| 213 | ORF | 13 |
| 214 | ORF | 13 |
| 215 | ORF | 13 |
| 216 | ORF | 13 |
| 217 | ORF | 13 |
| 218 | ORF | 13 |
| 219 | ORF | 13 |
| 220 | ORF | 13 |
| 221 | ORF | 13 |
| 222 | ORF | 13 |
| 223 | ORF | 13-14 |
| 224 | ORF | 14 |
| 225 | ORF | 14 |
| 226 | ORF | 14 |
| 227 | ORF | 14 |
| 228 | ORF | 14 |
| 229 | ORF | 14 |
| 230 | ORF | 14 |
| 231 | ORF | 14 |
| 232 | ORF | 14 |
| 233 | ORF | 14 |
| 234 | ORF | 14 |
| 235 | ORF | 14 |
| 236 | ORF | 14 |
| 237 | ORF | 14 |
| 238 | ORF | 14 |
| 239 | ORF | 14 |
| 240 | ORF | 14 |
| 241 | ORF | 14 |
| 242 | ORF | 14 |
| 243 | ORF | 15 |
| 244 | ORF | 15 |
| 245 | ORF | 15 |
| 246 | ORF | 15 |
| 247 | ORF | 15 |
| 248 | ORF | 15 |
| 249 | ORF | 15 |
| 250 | ORF | 15 |
| 251 | ORF | 15 |
| 252 | ORF | 15 |
| 253 | ORF | 15 |
| 254 | ORF | 15 |
| 255 | ORF | 15 |
| 256 | ORF | 15-16 |
| 257 | ORF | 15-16 |
| 258 | ORF | 15-16 |
| 259 | ORF | 15-16 |
| 260 | ORF | 15-16 |
| 261 | ORF | 15-16 |
| 262 | ORF | 15-16 |
| 263 | ORF | 16 |
| 264 | ORF | 16 |
| 265 | ORF | 16 |
| 266 | ORF | 16 |
| 267 | ORF | 16 |
| 268 | ORF | 16 |
| 269 | ORF | 16-17 |
| 270 | ORF | 16-17 |
| 271 | ORF | 16-17 |
| 272 | ORF | 16-17 |
| 273 | ORF | 16-17 |
| 274 | ORF | 16-17 |
| 275 | ORF | 16-17 |
| 276 | ORF | 16-17 |
| 277 | ORF | 17 |
| 278 | ORF | 17 |
| 279 | ORF | 17 |
| 280 | ORF | 17 |
| 281 | ORF | 17 |
| 282 | ORF | 17 |
| 283 | ORF | 17 |
| 284 | ORF | 17 |
| 285 | ORF | 17 |
| 286 | ORF | 17 |
| 287 | ORF | 17 |
| 288 | ORF | 17 |
| 289 | ORF | 17 |
| 290 | ORF | 17-18 |
| 291 | ORF | 17-18 |
| 292 | ORF | 17-18 |
| 293 | ORF | 17-18 |
| 294 | ORF | 17-18 |
| 295 | ORF | 17-18 |
| 296 | ORF | 17-18 |
| 297 | ORF | 18 |
| 298 | ORF | 18 |
| 299 | ORF | 18 |
| 300 | ORF | 18 |
| 301 | ORF | 18 |
| 302 | ORF | 18 |
| 303 | ORF | 19-20 |
| 304 | ORF | 19-20 |
| 305 | ORF | 20 |
| 306 | ORF | 20 |
| 307 | ORF | 20 |
| 308 | ORF | 20 |
| 309 | ORF | 20 |
| 310 | ORF | 20 |
| 311 | ORF | 20 |
| 312 | ORF | 20 |
| 313 | ORF | 20 |
| 314 | ORF | 20 |
| 315 | ORF | 20 |
| 316 | ORF | 20 |
| 317 | ORF | 20 |
| 318 | ORF | 20 |
| 319 | ORF | 20-21 |
| 320 | ORF | 20-21 |
| 321 | ORF | 20-21 |
| 322 | ORF | 20-21 |
| 323 | ORF | 20-21 |
| 324 | ORF | 20-21 |
| 325 | ORF | 21 |
| 326 | ORF | 21 |
| 327 | ORF | 21 |
| 328 | ORF | 21 |
| 329 | ORF | 21 |
| 330 | ORF | 21 |
| 331 | ORF | 21 |
| 332 | ORF | 21 |
| 333 | ORF | 21 |
| 334 | ORF | 21 |
| 335 | ORF | 21 |
| 336 | ORF | 21 |
| 337 | ORF | 21-22 |
| 338 | ORF | 21-22 |
| 339 | ORF | 21-22 |
| 340 | ORF | 22 |
| 341 | ORF | 22 |
| 342 | ORF | 22 |
| 343 | ORF | 22 |
| 344 | ORF | 22 |
| 345 | ORF | 23 |
| 346 | ORF | 23 |
| 347 | ORF | 23 |
| 348 | ORF | 23 |
| 349 | ORF | 23 |
| 350 | ORF | 23 |
| 351 | ORF | 23 |
| 352 | ORF | 23 |
| 353 | ORF | 23 |
| 354 | ORF | 23 |
| 355 | ORF | 23 |
| 356 | ORF | 23 |
| 357 | ORF | 23 |
| 358 | ORF | 23 |
| 359 | ORF | 24 |
| 360 | ORF | 24 |
| 361 | ORF | 24 |
| 362 | ORF | 24 |
| 363 | ORF | 24 |
| 364 | ORF | 24 |
| 365 | ORF | 24 |
| 366 | ORF | 24 |
| 367 | ORF | 24 |
| 368 | ORF | 24 |
| 369 | ORF | 24 |
| 370 | ORF | 24 |
| 371 | ORF | 24 |
| 372 | ORF_3UTR | 24 |
| 373 | 3UTR | 24 |
| 374 | 3UTR | 24 |
| 375 | 3UTR | 24 |
| 376 | 3UTR | 24 |
| 377 | 3UTR | 24 |
| 378 | 3UTR | 24 |
| 379 | 3UTR | 24 |
| 380 | 3UTR | 24 |
| 381 | 3UTR | 24 |
| 382 | 3UTR | 24 |
| 383 | 3UTR | 24 |
| 384 | 3UTR | 24 |
| 385 | 3UTR | 24 |
| 386 | 3UTR | 24 |
| 387 | 3UTR | 24 |
| 388 | 3UTR | 24 |
| 389 | 3UTR | 24 |
| 390 | 3UTR | 24 |
| 391 | 3UTR | 24 |
| 392 | 3UTR | 24 |
| 393 | 3UTR | 24 |
| 394 | 3UTR | 24 |
| 395 | 3UTR | 24 |
| 396 | 3UTR | 24 |
| 397 | 3UTR | 24 |
| 398 | 3UTR | 24 |
| 399 | 3UTR | 24 |
| 400 | 3UTR | 24 |
| 401 | 3UTR | 24 |
| 402 | 3UTR | 24 |
| 403 | 3UTR | 24 |
| 404 | 3UTR | 24 |
| 405 | 3UTR | 24 |
| 406 | 3UTR | 24 |
| 407 | 3UTR | 24 |
| 408 | 3UTR | 24 |

siRNA Structure

The siRNA molecules of the disclosure may be in the form of a single-stranded (ss) or double-stranded (ds) oligonucleotide structure. In some embodiments, the siRNA molecules may be di-branched, tri-branched, or tetra-branched molecules. Furthermore, the siRNA molecules of the disclosure may contain one or more phosphodiester internucleoside linkages and/or an analog thereof, such as a phosphorothioate internucleoside linkage. The siRNA molecules of the disclosure may further contain chemically modified nucleosides having 2′ sugar modifications.

The simplest siRNAs consist of a ribonucleic acid, including a ss- or ds-structure, formed by a first strand (i.e., antisense strand), and in the case of a ds-siRNA, a second strand (i.e., sense strand). The first strand includes a stretch of contiguous nucleotides that is at least partially complementary to a target nucleic acid. The second strand also includes a stretch of contiguous nucleotides where the second stretch is at least partially identical to a target nucleic acid. The first strand and said second strand may be hybridized to each other to form a double-stranded structure. The hybridization typically occurs by Watson Crick base pairing.

Depending on the sequence of the first and second strand, the hybridization or base pairing is not necessarily complete or perfect, which means that the first and second strand are not 100% base-paired due to mismatches. One or more mismatches may also be present within the duplex without necessarily impacting the siRNA RNAi activity.

The first strand contains a stretch of contiguous nucleotides which is essentially complementary to a target nucleic acid. Typically, the target nucleic acid sequence is, in accordance with the mode of action of interfering ribonucleic acids, a ss-RNA, preferably an mRNA. Such hybridization occurs most likely through Watson Crick base pairing but is not necessarily limited thereto. The extent to which the first strand has a complementary stretch of contiguous nucleotides to a target nucleic acid sequence may be between 80% and 100%, e.g., 80%, 85%, 90%, 95%, or 100% complementary.

The siRNA molecules described herein may employ modifications to the nucleobase, phosphate backbone, ribose core, 5′- and 3′-ends, and branching, wherein multiple strands of siRNA may be covalently linked.

### Lengths of Small Interfering RNA Molecules

It is within the scope of the disclosure that any length, known and previously unknown in the art, may be employed for the current invention. As described herein, potential lengths for an antisense strand of the siRNA molecules of the present disclosure is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the antisense strand is 20 nucleotides. In some embodiments, the antisense strand is 21 nucleotides. In some embodiments, the antisense strand is 22 nucleotides. In some embodiments, the antisense strand is 23 nucleotides. In some embodiments, the antisense strand is 24 nucleotides. In some embodiments, the antisense strand is 25 nucleotides. In some embodiments, the antisense strand is 26 nucleotides. In some embodiments, the antisense strand is 27 nucleotides. In some embodiments, the antisense strand is 28 nucleotides. In some embodiments, the antisense strand is 29 nucleotides. In some embodiments, the antisense strand is 30 nucleotides.

In some embodiments, the sense strand of the siRNA molecules of the present disclosure is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 23 nucleotides (e.g., 14 nucleotides, 15 nucleotides, 16 nucleotides, 17 nucleotides, 18 nucleotides, 19 nucleotides, 20 nucleotides, 21 nucleotides, 22 nucleotides, or 23 nucleotides). In some embodiments, the sense strand is 15 nucleotides. In some embodiments, the sense strand is 16 nucleotides. In some embodiments, the sense strand is 17 nucleotides. In some embodiments, the sense strand is 18 nucleotides. In some embodiments, the sense strand is 19 nucleotides. In some embodiments, the sense strand is 20 nucleotides. In some embodiments, the sense strand is 21 nucleotides. In some embodiments, the sense strand is 22 nucleotides. In some embodiments, the sense strand is 23 nucleotides. In some embodiments, the sense strand is 24 nucleotides. In some embodiments, the sense strand is 25 nucleotides. In some embodiments, the sense strand is 26 nucleotides. In some embodiments, the sense strand is 27 nucleotides. In some embodiments, the sense strand is 28 nucleotides. In some embodiments, the sense strand is 29 nucleotides. In some embodiments, the sense strand is 30 nucleotides.

### 2′ Sugar Modifications

The present disclosure may include ss- and ds-siRNA molecule compositions including at least one (e.g., at least 1, 2, 3, 4, 5, 6, 7, 8, 9, 10, 11, or more) nucleosides having 2′ sugar modifications. Possible 2′-modifications include all possible orientations of OH; F; O—, S—, or N-alkyl; O—, S—, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. In some embodiments, the modification includes a 2′-O-methyl (2′-O-Me) modification. Other potential sugar substituent groups include: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, a group for improving the pharmacokinetic properties of an oligonucleotide, or a group for improving the pharmacodynamic properties of an oligonucleotide, and other substituents having similar properties. In some embodiments, the modification includes 2′-methoxyethoxy (2′-O—CH₂CH₂OCH₃, also known as 2′-O-(2-methoxyethyl) or 2′-MOE). In some embodiments, the modification includes 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other potential sugar substituent groups include, e.g., aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the siRNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

### Nucleobase Modifications

The siRNA molecules of the disclosure may also include nucleosides or other surrogate or mimetic monomeric subunits that include a nucleobase (often referred to in the art simply as “base” or “heterocyclic base moiety”). The nucleobase is another moiety that has been extensively modified or substituted and such modified and or substituted nucleobases are amenable to the present disclosure. As used herein, “unmodified” or “natural” nucleobases include the purine bases adenine (A) and guanine (G), and the pyrimidine bases thymine (T), cytosine (C) and uracil (U). Modified nucleobases also referred herein as heterocyclic base moieties include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in Kroschwitz, J. I., ed. The Concise Encyclopedia of Polymer Science and Engineering, New York, John Wiley & Sons, 1990, pp. 858-859; those disclosed by Englisch et al., Angewandte Chemie, International Edition 30:613, 1991; and those disclosed by Sanghvi, Y. S., Chapter 16, Antisense Research and Applications, CRC Press, Gait, M. J. ed., 1993, pp. 289-302. The siRNA molecules of the present disclosure may also include polycyclic heterocyclic compounds in place of one or more heterocyclic base moieties. A number of tricyclic heterocyclic compounds have been previously reported. These compounds are routinely used in antisense applications to increase the binding properties of the modified strand to a target strand.

Representative cytosine analogs that make three hydrogen bonds with a guanosine in a second strand include 1,3-diazaphenoxazine-2-one (Kurchavov et al., Nucleosides and Nucleotides, 16:1837-46, 1997), 1,3-diazaphenothiazine-2-one (Lin et al. Am. Chem. Soc., 117:3873-4, 1995), and 6,7,8,9-tetrafluoro-1,3-diazaphenoxazine-2-one (Wang et al., Tetrahedron Lett., 39:8385-8, 1998). Incorporated into oligonucleotides, these base modifications were shown to hybridize with complementary guanine and the latter was also shown to hybridize with adenine and to enhance helical thermal stability by extended stacking interactions (also see U.S. Ser. No. 10/155,920 and U.S. Ser. No. 10/013,295, both of which are herein incorporated by reference in their entirety). Further helix-stabilizing properties have been observed when a cytosine analog/substitute has an aminoethoxy moiety attached to the rigid 1,3-diazaphenoxazine-2-one scaffold (Lin et al., Am. Chem. Soc., 120:8531-2, 1998).

### Internucleoside Linkage Modifications

Another variable in the design of the present disclosure is the internucleoside linkage making up the phosphate backbone of the siRNA molecule. Although the natural RNA phosphate backbone may be employed here, derivatives thereof may be used which enhance desirable characteristics of the siRNA molecule. Although not limiting, of particular importance in the present disclosure is protecting parts, or the whole, of the siRNA molecule from hydrolysis. One example of a modification that decreases the rate of hydrolysis is phosphorothioates. Any portion or the whole of the backbone may contain phosphate substitutions (e.g., phosphorothioates). For instance, the internucleoside linkages may be between 0 and 100% phosphorothioate, e.g., between 0 and 100%, 10 and 100%, 20 and 100%, 30 and 100%, 40 and 100%, 50 and 100%, 60 and 100%, 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphorothioate linkages. Similarly, the internucleoside linkages may be between 0 and 100% phosphodiester linkages, e.g., between 0 and 100%, 10 and 100%, 20 and 100%, 30 and 100%, 40 and 100%, 50 and 100%, 60 and 100% 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphodiester linkages.

Specific examples of some potential siRNA molecules useful in this invention include oligonucleotides containing modified e.g., non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides. A preferred phosphorus containing modified internucleoside linkage is the phosphorothioate internucleoside linkage. In some embodiments, the modified oligonucleotide backbones containing a phosphorus atom therein include, for example, phosphorothioates, phosphorodithioates, phosphotriesters, aminoalkylphosphotriesters, methyl and other alkyl phosphonates including 3-alkylene phosphonates, 5′-alkylene phosphonates, phosphinates, phosphoramidates including 3-amino phosphoramidate and aminoalkylphosphoramidates, thionophosphoramidates, thionoalkylphosphonates, thionoalkylphosphotriesters, selenophosphates, and boranophosphates having normal 3-5′ linkages, 2′-5′ linked analogs of these, and those having inverted polarity wherein one or more internucleotide linkages is a 3′ to 3′, 5′ to 5′ or 2′ to 2′ linkage. Exemplary U.S. patents describing the preparation of phosphorus-containing linkages include but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

In some embodiments, the modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. Non-limiting examples of U.S. patents that teach the preparation of non-phosphorus backbones include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, the entire contents of each of which are hereby incorporated herein by reference.

Patterns of Modifications of siRNA Molecules

The following section provides a set of exemplary scaffolds into which the siRNA molecules of the disclosure may be incorporated.

In some embodiments of the disclosure, the siRNA may contain an antisense strand including a region represented by Formula I, wherein Formula I is, in the 5′-to-3′ direction

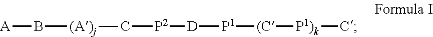

wherein A is represented by the formula C—P¹-D-P¹; each A′ is represented by the formula C—P²-D-P²; B is represented by the formula C—P²-D-P²-D-P²-D-P²; each C is a 2′-O-methyl (2′-O-Me) ribonucleoside; each C′, independently, is a 2′-O-Me ribonucleoside or a 2′-fluoro (2′-F) ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments, the antisense strand includes a structure represented by Formula A1, wherein Formula A1 is, in the 5′-to-3′ direction:

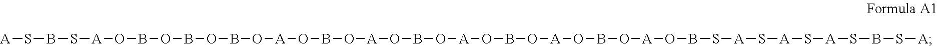

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula II, wherein Formula II is, in the 5′-to-3′ direction:

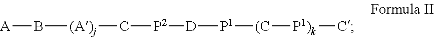

wherein A is represented by the formula C—P¹-D-P¹; each A′ is represented by the formula C—P²-D-P²; B is represented by the formula C—P²-D-P²-D-P²-D-P²; each C is a 2′-O-methyl (2′-O-Me) ribonucleoside; each C′, independently, is a 2′-O-Me ribonucleoside or a 2′-fluoro (2′-F) ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments of the disclosure, the antisense strand includes a structure represented by Formula A2, wherein Formula A2 is, in the 5′-to-3′ direction:

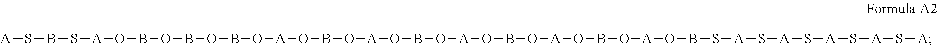

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula III, wherein Formula III is, in the 5′-to-3′ direction:

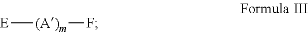

wherein E is represented by the formula (C—P¹)₂; F is represented by the formula (C—P²)₃-D-P¹—C—P¹—C, (C—P²)₃-D-P²—C—P²—C, (C—P²)₃-D-P¹—C—P¹-D, or (C—P²)₃-D-P²—C—P²-D; A′, C, D, P¹, and P² are as defined in Formula I; and m is an integer from 1 to 7 (e.g., 1, 2, 3, 4, 5, 6, or 7). In some embodiments, m is 4. The sense strand is complementary (e.g., fully or partially complementary) to the antisense strand.

In some embodiments of the disclosure, the sense strand includes a structure represented by Formula S1, wherein Formula S1 is, in the 5′-to-3′ direction:

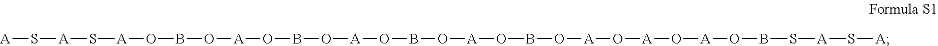

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S2, wherein Formula S2 is, in the 5′-to-3′ direction:

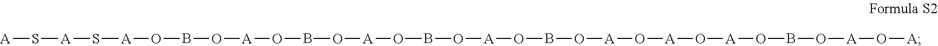

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S3, wherein Formula S3 is, in the 5′-to-3′ direction:

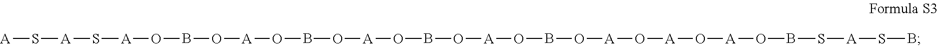

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S4, wherein Formula S4 is, in the 5′-to-3′ direction:

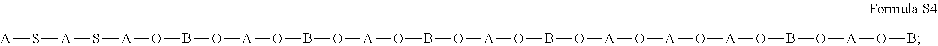

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula IV, wherein Formula IV is, in the 5′-to-3′ direction:

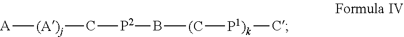

wherein A is represented by the formula C—P¹-D-P¹; each A′ is represented by the formula C—P²-D-P²; B is represented by the formula D-P¹—C—P¹-D-P¹; each C is a 2′-O-Me ribonucleoside; each C′, independently, is a 2′-O-Me ribonucleoside or a 2′-F ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to7 (e.g., 1, 2, 3, 4, 5, 6, or 7). In some embodiments, j is 6. In some embodiments, k is 4. In some embodiments, j is 6 and k is 4. The antisense strand is complementary (e.g., fully or partially complementary) to a target nucleic acid.

In some embodiments of the disclosure, the antisense strand includes a structure represented by Formula A3, wherein Formula A3 is, in the 5′-to-3′ direction:

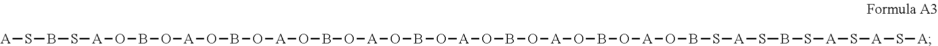

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA of the disclosure may have a sense strand represented by Formula V, wherein Formula V is, in the 5′-to-3′ direction:

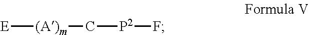

wherein E is represented by the formula (C—P¹)₂; F is represented by the formula D-P¹—C—P¹—C, D-P²—C—P²—C, D-P¹—C—P¹-D, or D-P²—C—P²-D; A′, C, D, P¹, and P² are as defined in Formula IV; and m is an integer from 1 to 7 (e.g., 1, 2, 3, 4, 5, 6, or 7). In some embodiments, m is 5. The sense strand is complementary (e.g., fully or partially complementary) to the antisense strand.

In some embodiments of the disclosure, the sense strand includes a structure represented by Formula S5, wherein Formula S5 is, in the 5′-to-3′ direction:

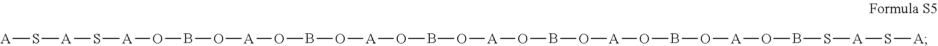

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S6, wherein Formula S6 is, in the 5′-to-3′ direction:

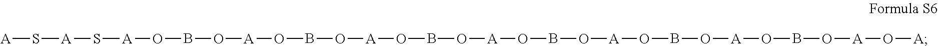

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S7, wherein Formula S7 is, in the 5′-to-3′ direction:

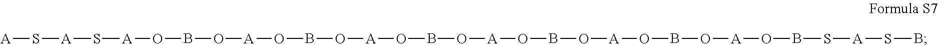

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S8, wherein Formula S8 is, in the 5′-to-3′ direction:

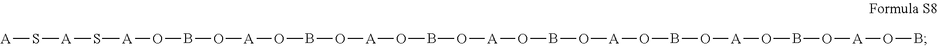

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula VI, wherein Formula VI is, in the 5′-to-3′ direction:

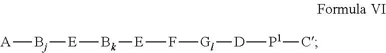

wherein A is represented by the formula C—P¹-D-P¹; each B is represented by the formula C—P²; each C is a 2′-O-Me ribonucleoside; each C′, independently, is a 2′-O-Me ribonucleoside or a 2′-F ribonucleoside; each D is a 2′-F ribonucleoside; each E is represented by the formula D-P²—C—P²; F is represented by the formula D-P¹—C—P¹; each G is represented by the formula C—P¹; each P¹ is a phosphorothioate internucleoside linkage; each P² is a phosphodiester internucleoside linkage; j is an integer from 1 to 7 (e.g., 1, 2, 3, 4, 5, 6, or 7); k is an integer from 1 to 7 (e.g., 1, 2, 3, 4, 5, 6, or 7); and l is an integer from 1 to 7 (e.g., 1, 2, 3, 4, 5, 6, or 7). In some embodiments, j is 3. In some embodiments, k is 6. In some embodiments, I is 2. In some embodiments, j is 3, k is 6, and l is 2. The antisense strand is complementary (e.g., fully or partially complementary) to a target nucleic acid.

In some embodiments of the disclosure, the antisense strand includes a structure represented by Formula A4, wherein Formula A4 is, in the 5′-to-3′ direction:

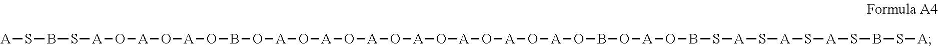

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain a sense strand including a region represented by Formula VII, wherein Formula VII is, in the 5′-to-3′ direction:

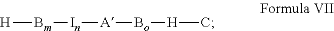

wherein A′ is represented by the formula C—P²-D-P²; each H is represented by the formula (C—P¹)₂; each I is represented by the formula (D-P²); B, C, D, P¹, and P² are as defined in Formula VI; m is an integer from 1 to 7 (e.g., 1, 2, 3, 4, 5, 6, or 7); n is an integer from 1 to 7 (e.g., 1, 2, 3, 4, 5, 6, or 7); and o is an integer from 1 to 7 (e.g., 1, 2, 3, 4, 5, 6, or 7). In some embodiments, m is 3. In some embodiments, n is 3. In some embodiments, o is 3. In some embodiments, m is 3, n is 3, and o is 3. The sense strand is complementary (e.g., fully or partially complementary) to the antisense strand.

In some embodiments of the disclosure, the sense strand includes a structure represented by Formula S9, wherein Formula S9 is, in the 5′-to-3′ direction:

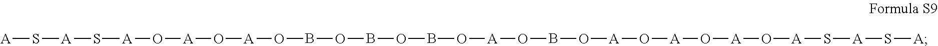

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region that is represented by Formula VIII:

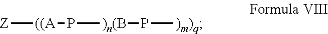

wherein Z is a 5′ phosphorus stabilizing moiety; each A is a 2′-O-methyl (2′-O-Me) ribonucleoside; each B is a 2′-fluoro-ribonucleoside; each P is, independently, an internucleoside linkage selected from a phosphodiester linkage and a phosphorothioate linkage; n is an integer from 1 to 5 (e.g., 1, 2, 3, 4, or 5); m is an integer from 1 to 5 (e.g., 1, 2, 3, 4, or 5); and q is an integer between 1 and 30 (1, 2, 3, 4, 5, 6, 7, 8, 9, 10, 11, 12, 13, 14, 15, 16, 17, 18, 19, 20, 21, 22, 23, 24, 25, 26, 27, 28, 29, or 30).

Methods of siRNA Synthesis

The siRNA molecules of the disclosure can be synthesized by standard methods known in the art as further discussed below, e.g., by use of an automated DNA synthesizer, such as are commercially available from, for example, Biosearch, Applied Biosystems, Inc.

The siRNA agent can be prepared using solution-phase or solid-phase organic synthesis or both. Organic synthesis offers the advantage that the oligonucleotide including unnatural or modified nucleotides can be easily prepared. siRNA molecules of the disclosure can be prepared using solution-phase or solid-phase organic synthesis or both.

Further, it is contemplated that for any siRNA agent disclosed herein, further optimization could be achieved by systematically either adding or removing linked nucleosides to generate longer or shorter sequences. Further still, such optimized sequences can be adjusted by, e.g., the introduction of modified nucleosides, and/or modified internucleoside linkages as described herein or as known in the art, including alternative nucleosides, alternative sugar moieties, and/or alternative internucleoside linkages as known in the art and/or discussed herein to further optimize the molecule (e.g., increasing serum stability or circulating half-life, increasing thermal stability, enhancing transmembrane delivery, and/or targeting to a particular location or cell type).

### Divalent Cations

The siRNA molecules of the disclosure may contain one or more phosphodiester internucleoside linkages and/or an analog thereof, such as a phosphorothioate internucleoside linkage, in which oxyanion moieties are electrostatically neutralized by ionic bonding to a divalent metal cation, such as Ba²⁺, Be²⁺, Ca²⁺, Cu²⁺, Mg²⁺, Mn²⁺, Ni²⁺, or Zn²⁺.

The siRNA molecules of the disclosure may include a plurality of cationic binding sites (e.g., electron-dense sites) that are saturated by one or more divalent cations (e.g., Ba²⁺, Be²⁺, Ca²⁺, Cu²⁺, Mg²⁺, Mn²⁺, Ni²⁺, or Zn²⁺, or a combination thereof). Because of their positive charge, divalent cations are typically reactive with negatively charges atoms (e.g., oxyanion from a phosphate group or phosphorothioate group carrying a unit or partial negative charge).

The one or more divalent cations may have an ionic radius, when measured in the form of a crystal lattice, of about 30 picometers to about 150 picometers (e.g., from about 30 picometers to about 140 picometers, from about 40 picometers to about 130 picometers, from about 50 picometers to about 120 picometers, from about 60 picometers to about 110 picometers, from about 60 picometers to about 100 picometers, or from about 60 picometers to about 90 picometers). The calculated crystal radii of the divalent cations disclosed by R. D. Shannon, Acta Crystallographica A. 32:751-767, 1976, are herein incorporated by reference.

The degree of saturation of a siRNA molecule's cationic binding sites by the one or more divalent cations may range from about 10% to about 100% (e.g., from about 20% to about 100%, from about 30% to about 100%, from about 40% to about 100%, from about 50% to about 100%, from about 60% to about 100%, from about 70% to about 100%, from about 80% to about 100%, or from about 90% to about 100%).

In some embodiments, the antisense strand of the siRNA molecule may have a length of from 10 to 30 nucleotides and may be ionically bound to a total of from 10 to 30 divalent cations. For example, the molar ratio of antisense strand nucleotides to divalent cations in the siRNA molecule could range from 1:3 to 3:1 (e.g., 1:3, 1.1:3, 1.2:3, 1.3:3, 1.4:3, 1.5:3, 1.6:3, 1.7:3, 1.8:3, 1.9:3, 2:3, 2.1:3, 2.2:3, 2.3:3, 2.4:3, 2.5:3, 2.6:3, 2.7:3, 2.8:3, 2.9:3, 1:1, 3:2.9, 3:2.8, 3:2.7, 3:2.6, 3:2.5, 3:2.4, 3:2.3, 3:2.2, 3:2.1, 3:2, 3:1.9, 3:1.8, 3:1.7, 3:1.6, 3:1.5, 3:1.4, 3:1.3, 3:1.2, 3:1.1, or 3:1).

In some embodiments, the sense strand of the siRNA molecule may have a length of from 10 to 30 nucleotides and may be ionically bound to a total of from 10 to 30 divalent cations. For example, the molar ratio of sense strand nucleotides to divalent cations in the siRNA molecule could range from 1:3 to 3:1 (e.g., 1:3, 1.1:3, 1.2:3, 1.3:3, 1.4:3, 1.5:3, 1.6:3, 1.7:3, 1.8:3, 1.9:3, 2:3, 2.1:3, 2.2:3, 2.3:3, 2.4:3, 2.5:3, 2.6:3, 2.7:3, 2.8:3, 2.9:3, 1:1, 3:2.9, 3:2.8, 3:2.7, 3:2.6, 3:2.5, 3:2.4, 3:2.3, 3:2.2, 3:2.1, 3:2, 3:1.9, 3:1.8, 3:1.7, 3:1.6, 3:1.5, 3:1.4, 3:1.3, 3:1.2, 3:1.1, or 3:1).

The siRNA molecules of the disclosure may be combined with one or more divalent cations in a specific molar ratio. The specific molar ratio of siRNA molecule to divalent cation may be relevant to the toxicity benefit achieved by the divalent cation. For example, the molar ratio of siRNA molecule to divalent cation may range from 1:10 to 1:50 (e.g., 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40. 1:41, 1:42, 1:43. 1:44, 1:45, 1:46, 1:47, 1:48, 1:49, or 1:50). In some embodiments, the molar ratio of siRNA molecule to divalent cation may range from 1:18 to 1:38 (e.g., 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, or 1:38). In some embodiments, the molar ratio of siRNA molecule to divalent cation may range from 1:20 to 1:25 (e.g., 1:20, 1:21, 1:22, 1:23, 1:24, or 1:25). In some embodiments, the molar ratio of siRNA molecule to divalent cation may be 1:20. In some embodiments, the molar ratio of siRNA molecule to divalent cation may be 1:25.

The siRNA molecules of the disclosure may be combined with one or more divalent cations in which the divalent cation is present in a specific concentration or range of concentrations. The concentration of the divalent cation may be relevant to the toxicity benefit achieved by the divalent cation. For example, the concentration of the divalent cation may be from 20 mM to 150 mM (e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122,123, 124, 125, 126, 127, 128, 129, 130, 131, 132, 133, 134, 135, 136, 137, 138, 139, 140, 141, 142, 143, 144, 145, 146, 147, 148, 149, or 150 mM). In some embodiments, the concentration of the divalent cation is from 20 mM to 100 mM (e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM). In some embodiments, the concentration of the divalent cation is from 35 mM to 75 mM (e.g., 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 mM). In some embodiments, the concentration of the divalent cation may be from 40 mM to 70 mM (e.g., 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 mM).

The siRNA molecule may include one or more atoms having a negative charge and the divalent cation may include a positive charge. In some embodiments, the siRNA molecule and divalent cation are present in an amount so that there is a specific ratio of negative to positive charge present within the composition. Methods of determining the negative to positive charge ratio are known in the art, for example, in Furst et al., Electrophoresis., 37:2685-2691, 2016, the disclosure of which is hereby incorporated by reference. In some embodiments, the ratio of negative charge to positive charge is from 0.75 to 7.5 (e.g., 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5). In some embodiments, the ratio of negative charge to positive charge is from 1.0 to 2.0 (e.g., from 1.0 to 1.9, from 1.0 to 1.8, from 1.0 to 1.7, from 1.0 to 1.6, from 1.0 to 1.5, from 1.0 to 1.4, from 1.0 to 1.3, from 1.0 to 1.2, from 1.0 to 1.1, from 1.1 to 2.0, from 1.2 to 2.0, from 1.3 to 2.0, from 1.4 to 2.0, from 1.5 to 2.0, from 1.6 to 2.0, from 1.7 to 2.0, from 1.8 to 2.0, or from 1.9 to 2.0). In some embodiments, the ratio of negative charge to positive charge is from 0.75 to 6.5 (e.g., from 0.75 to 5.5, from 0.75 to 4.5, from 0.75 to 3.5, from 0.75 to 2.5, from 0.75 to 1.5, or from 0.75 to 1). In some embodiments, the ratio of negative charge to positive charge is from 1 to 7.5 (e.g., from 1.5 to 7.5, from 2.5 to 7.5, from 3.5 to 7.5, from 4.5 to 7.5, from 5.5 to 7.5, or from 6.5 to 7.5).

### 5′ Phosphorus Stabilizing Moieties

To further protect the siRNA molecules of this disclosure from degradation, a 5′-phosphorus stabilizing moiety may be employed. A 5′-phosphorus stabilizing moiety replaces the 5′-phosphate to prevent hydrolysis of the phosphate. Hydrolysis of the 5′-phosphate prevents binding to RISC, a necessary step in gene silencing. Any replacement for phosphate that does not impede binding to RISC is contemplated in this disclosure. In some embodiments, the replacement for the 5′-phosphate is also stable to in vivo hydrolysis. Each strand of a siRNA molecule may independently and optionally employ any suitable 5′-phosphorus stabilizing moiety.

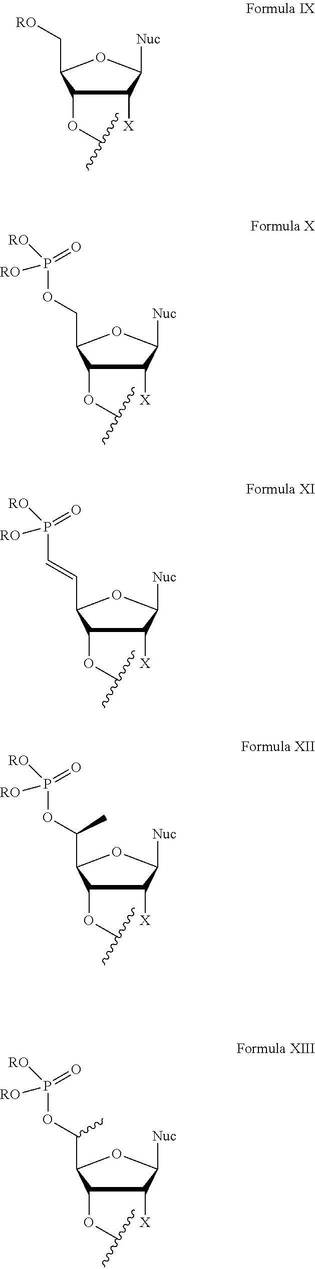

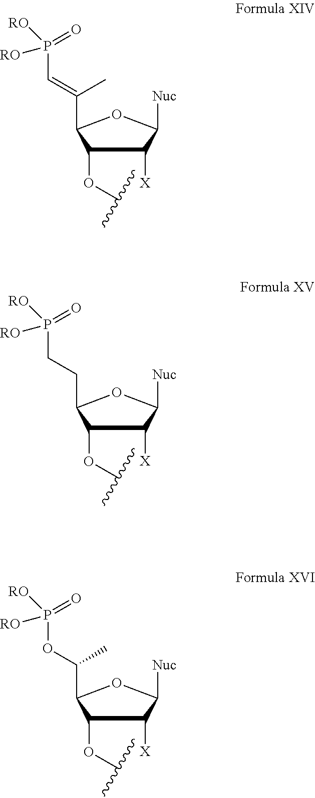

Some exemplary endcaps are demonstrated in Formulas IX-XVI. Nuc in Formulas IX-XVI represents a nucleobase or nucleobase derivative or replacement as described herein. X in formula IX-XVI represents a 2′-modification as described herein. Some embodiments employ hydroxy as in Formula IX, phosphate as in Formula X, vinylphosphonates as in Formula XI and XIV, 5′-methyl-substitued phosphates as in Formula XII, XIII, and XVI, methylenephosphonates as in Formula XV, or vinyl 5′-vinylphsophonate as a 5′-phosphorus stabilizing moiety as demonstrated in Formula XI.

### Hydrophobic Moieties

The present disclosure further provides siRNA molecules having one or more hydrophobic moieties attached thereto. The hydrophobic moiety may be covalently attached to the 5′ end or the 3′ end of the siRNA molecules of the disclosure. Non-limiting examples of hydrophobic moieties suitable for use with the siRNA molecules of the disclosure may include cholesterol, vitamin D, tocopherol, phosphatidylcholine (PC), docosahexaenoic acid, docosanoic acid, PC-docosanoic acid, eicosapentaenoic acid, Iithocholic acid or any combination of the aforementioned hydrophobic moieties with PC.

siRNA Branching

The siRNA molecules of the disclosure may be branched. For example, the siRNA molecules of the disclosure may have one of several branching patterns, as described herein.

According to the present disclosure, the siRNA molecules disclosed herein may be branched siRNA molecules. The siRNA molecule may not be branched, or may be di-branched, tri-branched, or tetra-branched, connected through a linker. Each main branch may be further branched to allow for 2, 3, 4, 5, 6, 7, or 8 separate RNA single- or double-strands. The branch points on the linker may stem from the same atom, or separate atoms along the linker. Some exemplary embodiments are listed in Table 2.

TABLE 2
Branched siRNA structures

| Di-branched | Tri-branched | Tetra-branched |
|---|---|---|
| RNA—L—RNA Formula XVII | 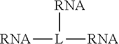 Formula XX | 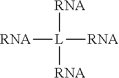 Formula XXIV |
| 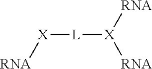 Formula XVIII | 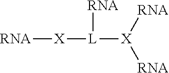 Formula XXI | 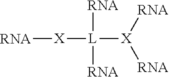 Formula XXV |
| 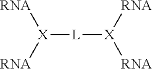 Formula XIX | 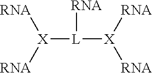 Formula XXII | 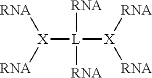 Formula XXVI |
| | 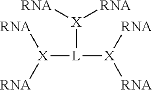 Formula XXIII | 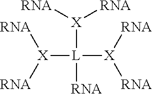 Formula XXVII |
| | | 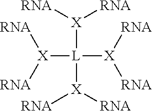 Formula XXVIII |

In some embodiments, the siRNA molecule is a branched siRNA molecule. In some embodiments, the branched siRNA molecule is di-branched, tri-branched, or tetra-branched. In some embodiments, the di-branched siRNA molecule is represented by any one of Formulas XVII-XIX, wherein each RNA, independently, is an siRNA molecule, L is a linker, and each X, independently, represents a branch point moiety (e.g., phosphoroamidite, tosylated solketal, 1,3-diaminopropanol, pentaerythritol, or any one of the branch point moieties described in U.S. Pat. No. 10,478,503).

In some embodiments, the tri-branched siRNA molecule represented by any one of Formulas XX-XXIII, wherein each RNA, independently, is an siRNA molecule, L is a linker, and each X, independently, represents a branch point moiety.

In some embodiments, the tetra-branched siRNA molecule represented by any one of Formulas XXIV-XXVIII, wherein each RNA, independently, is an siRNA molecule, L is a linker, and each X, independently, represents a branch point moiety.

### Linkers

Multiple strands of siRNA described herein may be covalently attached by way of a linker. The effect of this branching improves, inter alia, cell permeability allowing better access into cells (e.g., neurons or glial cells) in the CNS. Any linking moiety may be employed which is not incompatible with the siRNAs of the present invention. Linkers include ethylene glycol chains of 2 to 10 subunits (e.g., 2, 3, 4, 5, 6, 7, 8, 9, or 10 subunits), alkyl chains, carbohydrate chains, block copolymers, peptides, RNA, DNA, and others. In some embodiments, any carbon or oxygen atom of the linker is optionally replaced with a nitrogen atom, bears a hydroxyl substituent, or bears an oxo substituent. In some embodiments, the linker is a poly-ethylene glycol (PEG) linker. The PEG linkers suitable for use with the disclosed compositions and methods include linear or non-linear PEG linkers. Examples of non-linear PEG linkers include branched PEGs, linear forked PEGs, or branched forked PEGs.

PEG linkers of various weights may be used with the disclosed compositions and methods. For example, the PEG linker may have a weight that is between 5 and 500 Daltons. In some embodiments, a PEG linker having a weight that is between 500 and 1,000 Dalton may be used. In some embodiments, a PEG linker having a weight that is between 1,000 and 10,000 Dalton may be used. In some embodiments, a PEG linker having a weight that is between 200 and 20,000 Dalton may be used. In some embodiments, the linker is covalently attached to a sense strand of the siRNA. In some embodiments, the linker is covalently attached to an antisense strand of the siRNA. In some embodiments, the PEG linker is a triethylene glycol (TrEG) linker. In some embodiments, the PEG linker is a tetraethylene glycol (TEG) linker.

In some embodiments, the linker is an alkyl chain linker. In some embodiments, the linker is a peptide linker. In some embodiments, the linker is an RNA linker. In some embodiments, the linker is a DNA linker.

Linkers may covalently link 2, 3, 4, or 5 unique siRNA strands. The linker may covalently bind to any part of the siRNA oligomer. In some embodiments, the linker attaches to the 3′ end of nucleosides of each siRNA strand. In some embodiments, the linker attaches to the 5′ end of nucleosides of each siRNA strand. In some embodiments, the linker attaches to a nucleoside of an siRNA strand (e.g., sense or antisense strand) by way of a covalent bond-forming moiety. In some embodiments, the covalent-bond-forming moiety is selected from the group consisting of an alkyl, ester, amide, carbonate, carbamate, triazole, urea, formacetal, phosphonate, phosphate, and phosphate derivative (e.g., phosphorothioate, phosphoramidate, etc.).

In some embodiments, the linker has a structure of Formula L1:

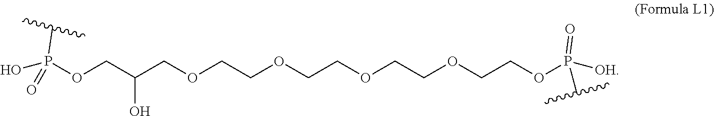 [SMILES: *P(=O)(O)OCCOCCOCCOCCOCC(O)COP(*)(=O)O]

In some embodiments, the linker has a structure of Formula L2:

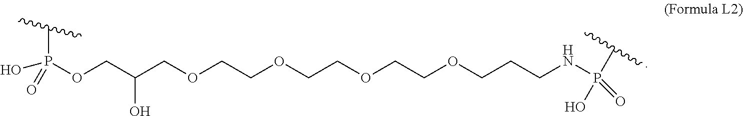

In some embodiments, the linker has a structure of Formula L3:

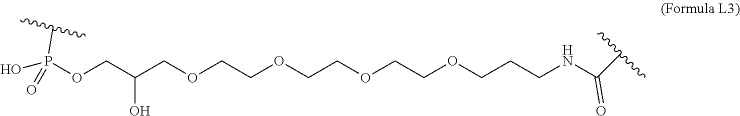 [SMILES: *C(=O)NCCCOCCOCCOCCOCC(O)COP(*)(=O)O]

In some embodiments, the linker has a structure of Formula L4:

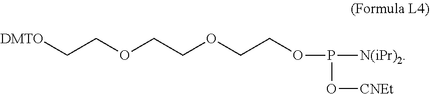

In some embodiments, the linker has a structure of Formula L5:

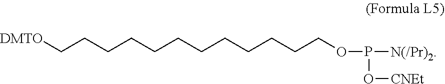

In some embodiments, the linker has a structure of Formula L6:

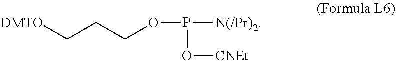

In some embodiments, the linker has a structure of Formula L7, as is shown below:

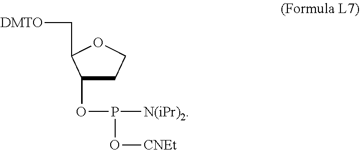

In some embodiments, the linker has a structure of Formula L8:

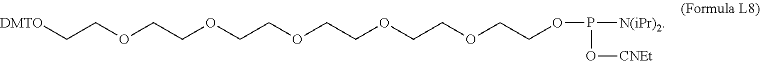

In some embodiments, the linker has a structure of Formula L9:

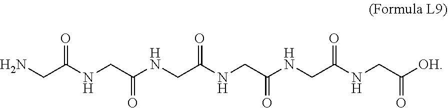 [SMILES: NCC(=O)NCC(=O)NCC(=O)NCC(=O)NCC(=O)NCC(=O)O]

In some embodiments, the selection of a linker for use with one or more of the branched siRNA molecules disclosed herein may be based on the hydrophobicity of the linker, such that, e.g., desirable hydrophobicity is achieved for the one or more branched siRNA molecules of the disclosure. For example, a linker containing an alkyl chain may be used to increase the hydrophobicity of the branched siRNA molecule as compared to a branched siRNA molecule having a less hydrophobic linker or a hydrophilic linker.

The siRNA agents disclosed herein may be synthesized and/or modified by methods well established in the art, such as those described in Beaucage, S. L. et al. (edrs.), Current Protocols in Nucleic Acid Chemistry, John Wiley & Sons, Inc., New York, N.Y., 2000, which is hereby incorporated herein by reference.

### Methods of Treatment

The MSH3-targeting siRNA molecules of the disclosure may be delivered to a subject, thereby treating a microsatellite repeat expansion disorder and/or mitigating phenotypes associated with the disorder. For example, the siRNA molecules may be delivered to a subject, thereby treating Huntington's disease and/or mitigating Huntington's disease associated phenotypes (e.g., movement disorders such as chorea and dystonia, cognitive impairments, and psychiatric disorders such as depression and anxiety). Alternatively, the MSH3-targeting siRNA molecules of the disclosure may be delivered to a subject, thereby treating myotonic dystrophy type 1 and/or mitigating myotonic dystrophy type 1-associated phenotypes (e.g., muscle wasting and weakness). As a further alternative, the MSH-targeting siRNA molecules of the disclosure may be delivered to a subject, thereby treating and mitigating the effects of spinocerebellar ataxias or any one of the many syndromes caused by expansions of DNA microsatellite repeats. Exemplary microsatellite repeat expansion disorders are described herein and discussed in Rodriguez et al., Neurobiology of Disease, 130:104515, 2019, the disclosure of which is incorporated herein by reference. Furthermore, the siRNA molecules of the disclosure may also be delivered to a subject having a variant of the MSH3 gene for which siRNA-mediated gene silencing of the MSH3 variant gene reduces the expression level of MSH3 transcript, thereby treating Huntington's Disease, myotonic dystrophies, spinocerebellar ataxias, or another microsatellite repeat expansion disorder.

The disclosure provides methods of treating a subject by way of MSH3 gene silencing with one or more of the siRNA molecules described herein. The gene silencing may be performed in a subject to silence wild type MSH3 transcripts, mutant MSH3 transcripts, splice isoforms of MSH3 transcripts, and/or overexpressed MSH3 transcripts thereof, relative to a healthy subject. The method may include delivering to the CNS or affected tissues of the subject (e.g., a human) the siRNA molecules of the disclosure or a pharmaceutical composition containing the same by any appropriate route of administration (e.g., intracerebroventricular, intrathecal injection, intrastriatal injection, intra-cisterna magna injection by catheterization, intraparenchymal injection, intravenous injection, subcutaneous injection, or intramuscular injection). The active compound can be administered in any suitable dose. The actual dosage amount of a composition of the present disclosure administered to a patient can be determined by physical and physiological factors such as body weight, severity of condition, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject. Administration may occur any suitable number of times per day, and for as long as necessary. Subjects may be adult or pediatric humans, with or without comorbid diseases.

### Selection of Subjects

Subjects that may be treated with the siRNA molecules disclosed herein are subjects in need of treatment of, for example, a microsatellite repeat expansion disorder, such as Huntington's Disease, and/or any other medical risk(s) associated with DNA microsatellite repeat expansions or a gain of function mutation in the MSH3 gene. Subjects that may be treated with the siRNA molecules disclosed herein may include, for example, humans, monkeys, rats, mice, pigs, and other mammals containing at least one orthologous copy of the MSH3 gene. Subjects may be adult or pediatric humans, with or without comorbid diseases.

### Pharmaceutical Compositions

The siRNA molecules in the present disclosure may be formulated into a pharmaceutical composition for administration to a subject in a biologically compatible form suitable for administration in vivo. Accordingly, the present disclosure provides a pharmaceutical composition containing a siRNA molecule of the disclosure in admixture with a suitable diluent, carrier, or excipient. The siRNA molecules may be administered, for example, directly into the CNS or affected tissues of the subject (e.g., by way of intracerebroventricular, intrastriatally, intrathecal injection, intra-cisterna magna injection by catheterization, intraparenchymal injection, intravenous injection, subcutaneous injection, or intramuscular injection).

Conventional procedures and ingredients for the selection and preparation of suitable formulations are described, for example, in Remington, J. P. The Science and Practice of Pharmacy, Easton, PA. Mack Publishers, 2012, 22^{nd} ed. and in The United States Pharmacopeial Convention, The National Formulary, United States Pharmacopeial, 2015, USP 38 NF 33).

Under ordinary conditions of storage and use, a pharmaceutical composition may contain a preservative, e.g., to prevent the growth of microorganisms. Pharmaceutical compositions may include sterile aqueous solutions, dispersions, or powders, e.g., for the extemporaneous preparation of sterile solutions or dispersions. In all cases the form may be sterilized using techniques known in the art and may be fluidized to the extent that may be easily administered to a subject in need of treatment.

A pharmaceutical composition may be administered to a subject, e.g., a human subject, alone or in combination with pharmaceutically acceptable carriers, as noted herein, the proportion of which may be determined by the solubility and/or chemical nature of the compound, chosen route of administration, and standard pharmaceutical practice.

### Dosing Regimens

A physician having ordinary skill in the art can readily determine an effective amount of the siRNA molecule for administration to a mammalian subject (e.g., a human) in need thereof. For example, a physician could start prescribing doses of one the siRNA molecules of the disclosure at levels lower than that required to achieve the desired therapeutic effect and gradually increase the dosage until the desired effect is achieved. Alternatively, a physician may begin a treatment regimen by administering one of the siRNA molecules of the disclosure at a high dose and subsequently administer progressively lower doses until reaching a minimal dosage at which a therapeutic effect is achieved (e.g., a reduction in expression of a target gene sequence). In general, a suitable daily dose of one of the siRNA molecules of the disclosure will be an amount of the siRNA molecule which is the lowest dose effective to produce a therapeutic effect. The ss- or ds-siRNA molecules of the disclosure may be administered by injection, e.g., intrathecally, intracerebroventricularly, by intra-cisterna magna injection by catheterization, intraparenchymally, intravenously, subcutaneously, or intramuscularly. A daily dose of a therapeutic composition of the siRNA molecules of the disclosure may be administered as a single dose or as two, three, four, five, six or more doses administered separately at appropriate intervals throughout the day, week, month, or year, optionally, in unit dosage forms. While it is possible for the siRNA molecules of the disclosure to be administered alone, it may also be administered as a pharmaceutical formulation in combination with excipients, carriers, and optionally, additional therapeutic agents.

### Routes of Administration

The method of the disclosure contemplates any route of administration tolerated by the therapeutic composition. Some embodiments of the method include injection intrathecally, intracerebroventricularly, intrastriatally, intraparenchymally, or by intra-cisterna magna injection by catheterization.

Intrathecal injection is the direct injection into the spinal column or subarachnoid space. By injecting directly into the CSF of the spinal column the siRNA molecules of the disclosure have direct access to cells (e.g., neurons and glial cells) in the spinal column and a route to access the cells in the brain by bypassing the blood brain barrier.

Intracerebroventricular (ICV) injection is a method to directly inject into the CSF of the cerebral ventricles. Similar to intrathecal injection, ICV is a method of injection which bypasses the blood brain barrier. Using ICV allows the advantage of access to the cells of the brain and spinal column without the danger of the therapeutic being degraded in the blood.

Intrastriatal injection is the direct injection into the striatum, or corpus striatum. The striatum is an area in the subcortical basal ganglia in the brain. Injecting into the striatum bypasses the blood brain barrier and the pharmacokinetic challenges of injection into the blood stream and allows for direct access to the cells of the brain.

Intraparenchymal administration is the direct injection into the parenchyma (e.g., the brain parenchyma). Injection into the brain parenchyma allows for injection directly into brain regions affected by a disease or disorder while bypassing the blood brain barrier.

Intra-cisterna magna injection by catheterization is the direct injection into the cisterna magna. The cisterna magna is the area of the brain located between the cerebellum and the dorsal surface of the medulla oblongata. Injecting into the cisterna magna results in more direct delivery to the cells of the cerebellum, brainstem, and spinal cord.

In some embodiments of the methods described herein, the therapeutic composition may be delivered to the subject by way of systemic administration, e.g., intravenously, intramuscularly, or subcutaneously.

Intravenous (IV) injection is a method to directly inject into the bloodstream of a subject. The IV administration may be in the form of a bolus dose or by way of continuous infusion, or any other method tolerated by the therapeutic composition.

Intramuscular (IM) injection is injection into a muscle of a subject, such as the deltoid muscle or gluteal muscle. IM may allow for rapid absorption of the therapeutic composition.

Subcutaneous injection is injection into subcutaneous tissue. Absorption of compositions delivered subcutaneously may be slower than IV or IM injection, which may be beneficial for compositions requiring continuous absorption.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. MSH3 Knockdown

### Objective

This Example describes the results of a series of experiments undertaken to investigate the ability of siRNA molecules complementary to specific regions within a human MSH3 mRNA transcript to effectuate reduced expression of the MSH3 gene.

### Materials and Methods

MeWo cells (metastatic malignant melanoma cells, ATCC cat #HTB-65) were seeded at 30,000 cells per well in a 96-well tissue culture plate in Modified Eagle's Medium alpha (MEM-alpha) with 6% fetal bovine serum without antibiotics or antimycotics. An equal volume of siRNA at either 4 M or 1 M in serum-free Opti-MEM was added to the cells, resulting in a final assay condition of 3% fetal bovine serum and either 2 M or 0.5 M siRNA. Non-siRNA treated cells (untreated) exposed to the same media composition were used as controls. Cells were grown in a standard cell culture incubator at 37° C. and 5% CO₂. At 72 hours after siRNA treatment, the TaqMan Fast Advanced Cells-to-CT Kit (Thermo) was used to lyse all cells, digest the genomic DNA, perform reverse-transcription on the resulting RNA, and perform quantitative polymerase chain reaction (qPCR) on the resulting cDNA. Exon spanning MSH3-specific primer/probes (Thermo) were multiplexed with primer/probes for a housekeeping gene (ATP5B, Thermo), and all reactions were run on a Quantstudio 7 Flex (Thermo). MSH3 mRNA expression was calculated relative to untreated control cells using the delta-delta-Ct method. Biological duplicates and technical (qPCR) duplicates were run for each condition, and all results are expressed as the mean of all technical and biological duplicates, and as residual mRNA expression as a percent of untreated control cells.

### Results

Cells were treated with an siRNA molecule of the disclosure having an antisense strand and a sense strand as shown in Tables 3 and 4, below. The knockdown efficiency of the siRNA molecule was measured as the residual mRNA expression as a percent of untreated at 2 μM and 0.5 μM. The knockdown results are reported in Table 3 (2 μM) and Table 4 (0.5 μM).

TABLE 3
MSH3 knockdown with siRNA molecules
of the disclosure at 2 μM

| Sense | Antisense | Residual mRNA expression |
|---|---|---|
| SEQ ID NO: | SEQ ID NO: | (% of untreated) at 2 μM |
| 409 | 817 | 79.71 |
| 410 | 818 | 71.48 |
| 411 | 819 | 57.49 |
| 412 | 820 | 98.66 |
| 413 | 821 | 37.54 |
| 414 | 822 | 67.50 |
| 415 | 823 | 53.76 |
| 416 | 824 | 65.76 |
| 417 | 825 | 57.11 |
| 418 | 826 | 62.49 |
| 419 | 827 | 103.15 |
| 420 | 828 | 105.86 |
| 421 | 829 | 52.04 |
| 422 | 830 | 75.11 |
| 423 | 831 | 94.35 |
| 424 | 832 | 78.95 |
| 425 | 833 | 46.45 |
| 426 | 834 | 46.61 |
| 427 | 835 | 50.45 |
| 428 | 836 | 59.60 |
| 429 | 837 | 52.48 |
| 430 | 838 | 60.31 |
| 431 | 839 | 69.64 |
| 432 | 840 | 90.76 |
| 433 | 841 | 56.73 |
| 434 | 842 | 80.17 |
| 435 | 843 | 54.32 |
| 436 | 844 | 58.78 |
| 437 | 845 | 57.20 |
| 438 | 846 | 91.48 |
| 439 | 847 | 99.44 |
| 440 | 848 | 84.66 |
| 441 | 849 | 44.81 |
| 442 | 850 | 67.08 |
| 443 | 851 | 58.85 |
| 444 | 852 | 86.94 |
| 445 | 853 | 112.90 |
| 446 | 854 | 82.09 |
| 447 | 855 | 97.11 |
| 448 | 856 | 72.82 |
| 449 | 857 | 49.91 |
| 450 | 858 | 43.33 |
| 451 | 859 | 51.85 |
| 452 | 860 | 42.73 |
| 453 | 861 | 54.19 |
| 454 | 862 | 59.81 |
| 455 | 863 | 67.51 |
| 456 | 864 | 82.02 |
| 457 | 865 | 70.78 |
| 458 | 866 | 76.71 |
| 459 | 867 | 65.61 |
| 460 | 868 | 61.29 |
| 461 | 869 | 55.52 |
| 462 | 870 | 76.27 |
| 463 | 871 | 70.81 |
| 464 | 872 | 50.19 |
| 465 | 873 | 74.56 |
| 466 | 874 | 82.97 |
| 467 | 875 | 69.95 |
| 468 | 876 | 65.28 |
| 469 | 877 | 81.10 |
| 470 | 878 | 49.85 |
| 471 | 879 | 72.75 |
| 472 | 880 | 74.21 |
| 473 | 881 | 85.74 |
| 474 | 882 | 73.75 |
| 475 | 883 | 81.86 |
| 476 | 884 | 75.36 |
| 477 | 885 | 73.92 |
| 478 | 886 | 50.32 |
| 479 | 887 | 84.02 |
| 480 | 888 | 78.30 |
| 481 | 889 | 59.83 |
| 482 | 890 | 64.15 |
| 483 | 891 | 77.19 |
| 484 | 892 | 53.36 |
| 485 | 893 | 50.10 |
| 486 | 894 | 73.70 |
| 487 | 895 | 60.81 |
| 488 | 896 | 56.12 |
| 489 | 897 | 65.98 |
| 490 | 898 | 53.98 |
| 491 | 899 | 53.69 |
| 492 | 900 | 51.98 |
| 493 | 901 | 100.25 |
| 494 | 902 | 78.42 |
| 495 | 903 | 58.87 |
| 496 | 904 | 48.63 |
| 497 | 905 | 52.86 |
| 498 | 906 | 64.19 |
| 499 | 907 | 67.25 |
| 500 | 908 | 81.16 |
| 501 | 909 | 54.58 |
| 502 | 910 | 67.87 |
| 503 | 911 | 91.69 |
| 504 | 912 | 47.65 |
| 505 | 913 | 66.15 |
| 506 | 914 | 85.73 |
| 507 | 915 | 68.10 |
| 508 | 916 | 91.63 |
| 509 | 917 | 91.68 |
| 510 | 918 | 52.11 |
| 511 | 919 | 53.71 |
| 512 | 920 | 63.69 |
| 513 | 921 | 51.55 |
| 514 | 922 | 58.13 |
| 515 | 923 | 60.12 |
| 516 | 924 | 53.69 |
| 517 | 925 | 54.26 |
| 518 | 926 | 69.21 |
| 519 | 927 | 62.27 |
| 520 | 928 | 68.71 |
| 521 | 929 | 52.53 |
| 522 | 930 | 63.53 |
| 523 | 931 | 79.42 |
| 524 | 932 | 73.47 |
| 525 | 933 | 58.95 |
| 526 | 934 | 81.77 |
| 527 | 935 | 78.49 |
| 528 | 936 | 98.91 |
| 529 | 937 | 81.68 |
| 530 | 938 | 68.35 |
| 531 | 939 | 58.51 |
| 532 | 940 | 85.78 |
| 533 | 941 | 52.76 |
| 534 | 942 | 46.68 |
| 535 | 943 | 54.05 |
| 536 | 944 | 71.73 |
| 537 | 945 | 44.89 |
| 538 | 946 | 43.26 |
| 539 | 947 | 55.17 |
| 540 | 948 | 55.40 |
| 541 | 949 | 68.45 |
| 542 | 950 | 92.75 |
| 543 | 951 | 100.48 |
| 544 | 952 | 107.19 |
| 545 | 953 | 82.62 |
| 546 | 954 | 87.97 |
| 547 | 955 | 96.21 |
| 548 | 956 | 89.30 |
| 549 | 957 | 73.09 |
| 550 | 958 | 82.96 |
| 551 | 959 | 83.71 |
| 552 | 960 | 68.42 |
| 553 | 961 | 66.12 |
| 554 | 962 | 90.78 |
| 555 | 963 | 87.65 |
| 556 | 964 | 68.41 |
| 557 | 965 | 66.48 |
| 558 | 966 | 54.95 |
| 559 | 967 | 90.47 |
| 560 | 968 | 49.85 |
| 561 | 969 | 51.03 |
| 562 | 970 | 56.88 |
| 563 | 971 | 61.04 |
| 564 | 972 | 36.30 |
| 565 | 973 | 60.75 |
| 566 | 974 | 35.83 |
| 567 | 975 | 69.60 |
| 568 | 976 | 55.74 |
| 569 | 977 | 39.69 |
| 570 | 978 | 67.71 |
| 571 | 979 | 76.56 |
| 572 | 980 | 53.45 |
| 573 | 981 | 86.90 |
| 574 | 982 | 47.04 |
| 575 | 983 | 61.96 |
| 576 | 984 | 47.35 |
| 577 | 985 | 46.14 |
| 578 | 986 | 61.29 |
| 579 | 987 | 69.36 |
| 580 | 988 | 73.60 |
| 581 | 989 | 65.91 |
| 582 | 990 | 65.69 |
| 583 | 991 | 70.98 |
| 584 | 992 | 71.46 |
| 585 | 993 | 39.19 |
| 586 | 994 | 49.35 |
| 587 | 995 | 67.84 |
| 588 | 996 | 55.19 |
| 589 | 997 | 58.84 |
| 590 | 998 | 68.46 |
| 591 | 999 | 47.97 |
| 592 | 1000 | 56.13 |
| 593 | 1001 | 56.84 |
| 594 | 1002 | 59.13 |
| 595 | 1003 | 71.73 |
| 596 | 1004 | 63.92 |
| 597 | 1005 | 83.57 |
| 598 | 1006 | 52.75 |
| 599 | 1007 | 58.26 |
| 600 | 1008 | 75.40 |
| 601 | 1009 | 57.99 |
| 602 | 1010 | 49.36 |
| 603 | 1011 | 91.99 |
| 604 | 1012 | 56.14 |
| 605 | 1013 | 51.87 |
| 606 | 1014 | 65.15 |
| 607 | 1015 | 73.40 |
| 608 | 1016 | 65.72 |
| 609 | 1017 | 110.38 |
| 610 | 1018 | 103.03 |
| 611 | 1019 | 106.90 |
| 612 | 1020 | 107.16 |
| 613 | 1021 | 99.09 |
| 614 | 1022 | 83.60 |
| 615 | 1023 | 70.01 |
| 616 | 1024 | 52.20 |
| 617 | 1025 | 46.48 |
| 618 | 1026 | 70.49 |
| 619 | 1027 | 64.25 |
| 620 | 1028 | 62.01 |
| 621 | 1029 | 55.97 |
| 622 | 1030 | 83.59 |
| 623 | 1031 | 62.21 |
| 624 | 1032 | 53.05 |
| 625 | 1033 | 49.31 |
| 626 | 1034 | 64.53 |
| 627 | 1035 | 104.06 |
| 628 | 1036 | 90.15 |
| 629 | 1037 | 89.89 |
| 630 | 1038 | 103.08 |
| 631 | 1039 | 97.36 |
| 632 | 1040 | 81.76 |
| 633 | 1041 | 96.45 |
| 634 | 1042 | 75.79 |
| 635 | 1043 | 61.02 |
| 636 | 1044 | 99.23 |
| 637 | 1045 | 68.90 |
| 638 | 1046 | 96.66 |
| 639 | 1047 | 42.03 |
| 640 | 1048 | 54.92 |
| 641 | 1049 | 53.71 |
| 642 | 1050 | 55.07 |
| 643 | 1051 | 79.82 |
| 644 | 1052 | 75.00 |
| 645 | 1053 | 78.96 |
| 646 | 1054 | 63.34 |
| 647 | 1055 | 108.68 |
| 648 | 1056 | 83.29 |
| 649 | 1057 | 68.90 |
| 650 | 1058 | 89.59 |
| 651 | 1059 | 85.62 |
| 652 | 1060 | 90.10 |
| 653 | 1061 | 94.65 |
| 654 | 1062 | 104.42 |
| 655 | 1063 | 87.09 |
| 656 | 1064 | 103.27 |
| 657 | 1065 | 58.93 |
| 658 | 1066 | 75.45 |
| 659 | 1067 | 76.61 |
| 660 | 1068 | 54.92 |
| 661 | 1069 | 63.94 |
| 662 | 1070 | 74.26 |
| 663 | 1071 | 64.29 |
| 664 | 1072 | 73.77 |
| 665 | 1073 | 99.95 |
| 666 | 1074 | 94.59 |
| 667 | 1075 | 54.39 |
| 668 | 1076 | 94.02 |
| 669 | 1077 | 89.46 |
| 670 | 1078 | 72.55 |
| 671 | 1079 | 66.46 |
| 672 | 1080 | 81.74 |
| 673 | 1081 | 59.50 |
| 674 | 1082 | 80.86 |
| 675 | 1083 | 101.55 |
| 676 | 1084 | 81.57 |
| 677 | 1085 | 94.93 |
| 678 | 1086 | 89.57 |
| 679 | 1087 | 54.96 |
| 680 | 1088 | 93.29 |
| 681 | 1089 | 58.71 |
| 682 | 1090 | 70.33 |
| 683 | 1091 | 63.98 |
| 684 | 1092 | 84.47 |
| 685 | 1093 | 82.48 |
| 686 | 1094 | 89.31 |
| 687 | 1095 | 60.46 |
| 688 | 1096 | 59.77 |
| 689 | 1097 | 63.76 |
| 690 | 1098 | 79.95 |
| 691 | 1099 | 82.48 |
| 692 | 1100 | 51.20 |
| 693 | 1101 | 53.67 |
| 694 | 1102 | 69.30 |
| 695 | 1103 | 86.24 |
| 696 | 1104 | 71.71 |
| 697 | 1105 | 66.21 |
| 698 | 1106 | 57.48 |
| 699 | 1107 | 45.06 |
| 700 | 1108 | 68.92 |
| 701 | 1109 | 51.70 |
| 702 | 1110 | 45.48 |
| 703 | 1111 | 66.77 |
| 704 | 1112 | 76.64 |
| 705 | 1113 | 90.16 |
| 706 | 1114 | 91.76 |
| 707 | 1115 | 67.83 |
| 708 | 1116 | 107.98 |
| 709 | 1117 | 126.83 |
| 710 | 1118 | 92.48 |
| 711 | 1119 | 107.20 |
| 712 | 1120 | 74.13 |
| 713 | 1121 | 63.09 |
| 714 | 1122 | 66.13 |
| 715 | 1123 | 54.88 |
| 716 | 1124 | 52.78 |
| 717 | 1125 | 90.02 |
| 718 | 1126 | 78.54 |
| 719 | 1127 | 114.14 |
| 720 | 1128 | 80.97 |
| 721 | 1129 | 79.12 |
| 722 | 1130 | 94.49 |
| 723 | 1131 | 49.19 |
| 724 | 1132 | 70.61 |
| 725 | 1133 | 46.40 |
| 726 | 1134 | 44.40 |
| 727 | 1135 | 79.07 |
| 728 | 1136 | 96.00 |
| 729 | 1137 | 60.78 |
| 730 | 1138 | 72.12 |
| 731 | 1139 | 68.20 |
| 732 | 1140 | 66.94 |
| 733 | 1141 | 85.64 |
| 734 | 1142 | 78.77 |
| 735 | 1143 | 69.33 |
| 736 | 1144 | 111.13 |
| 737 | 1145 | 66.06 |
| 738 | 1146 | 62.89 |
| 739 | 1147 | 79.78 |
| 740 | 1148 | 71.67 |
| 741 | 1149 | 70.72 |
| 742 | 1150 | 62.73 |
| 743 | 1151 | 79.43 |
| 744 | 1152 | 107.08 |
| 745 | 1153 | 64.96 |
| 746 | 1154 | 63.22 |
| 747 | 1155 | 66.68 |
| 748 | 1156 | 88.00 |
| 749 | 1157 | 82.10 |
| 750 | 1158 | 72.87 |
| 751 | 1159 | 63.09 |
| 752 | 1160 | 105.79 |
| 753 | 1161 | 98.46 |
| 754 | 1162 | 65.79 |
| 755 | 1163 | 97.10 |
| 756 | 1164 | 99.02 |
| 757 | 1165 | 70.49 |
| 758 | 1166 | 63.81 |
| 759 | 1167 | 61.81 |
| 760 | 1168 | 79.67 |
| 761 | 1169 | 57.12 |
| 762 | 1170 | 68.75 |
| 763 | 1171 | 58.57 |
| 764 | 1172 | 58.15 |
| 765 | 1173 | 83.25 |
| 766 | 1174 | 94.88 |
| 767 | 1175 | 57.79 |
| 768 | 1176 | 57.47 |
| 769 | 1177 | 55.73 |
| 770 | 1178 | 42.24 |
| 771 | 1179 | 71.62 |
| 772 | 1180 | 102.18 |
| 773 | 1181 | 62.32 |
| 774 | 1182 | 84.18 |
| 775 | 1183 | 59.04 |
| 776 | 1184 | 59.38 |
| 777 | 1185 | 103.57 |
| 778 | 1186 | 71.30 |
| 779 | 1187 | 70.66 |
| 780 | 1188 | 71.49 |
| 781 | 1189 | 92.59 |
| 782 | 1190 | 101.31 |
| 783 | 1191 | 83.54 |
| 784 | 1192 | 75.05 |
| 785 | 1193 | 73.80 |
| 786 | 1194 | 67.84 |
| 787 | 1195 | 83.47 |
| 788 | 1196 | 70.13 |
| 789 | 1197 | 64.43 |
| 790 | 1198 | 69.35 |
| 791 | 1199 | 69.76 |
| 792 | 1200 | 82.56 |
| 793 | 1201 | 70.80 |
| 794 | 1202 | 68.86 |
| 795 | 1203 | 72.63 |
| 796 | 1204 | 73.36 |
| 797 | 1205 | 73.87 |
| 798 | 1206 | 82.09 |
| 799 | 1207 | 74.84 |
| 800 | 1208 | 77.65 |
| 801 | 1209 | 68.04 |
| 802 | 1210 | 79.74 |
| 803 | 1211 | 71.54 |
| 804 | 1212 | 98.64 |
| 805 | 1213 | 76.58 |
| 806 | 1214 | 71.30 |
| 807 | 1215 | 77.80 |
| 808 | 1216 | 91.63 |
| 809 | 1217 | 108.13 |
| 810 | 1218 | 101.54 |
| 811 | 1219 | 81.90 |
| 812 | 1220 | 86.96 |
| 813 | 1221 | 77.45 |
| 814 | 1222 | 81.28 |
| 815 | 1223 | 73.28 |
| 816 | 1224 | 70.08 |

TABLE 4
MSH3 knockdown with siRNA molecules
of the disclosure at 0.5 μM

| Sense | Antisense | Residual mRNA expression |
|---|---|---|
| SEQ ID NO: | SEQ ID NO: | (% of untreated) at 0.5 μM |
| 409 | 817 | 106.60 |
| 410 | 818 | 91.38 |
| 411 | 819 | 81.97 |
| 412 | 820 | 111.01 |
| 413 | 821 | 57.28 |
| 414 | 822 | 95.80 |
| 415 | 823 | 89.32 |
| 416 | 824 | 92.49 |
| 417 | 825 | 88.58 |
| 418 | 826 | 88.15 |
| 419 | 827 | 111.33 |
| 420 | 828 | 114.43 |
| 421 | 829 | 78.55 |
| 422 | 830 | 83.20 |
| 423 | 831 | 104.02 |
| 424 | 832 | 99.33 |
| 425 | 833 | 71.85 |
| 426 | 834 | 54.84 |
| 427 | 835 | 68.66 |
| 428 | 836 | 76.46 |
| 429 | 837 | 87.86 |
| 430 | 838 | 75.89 |
| 431 | 839 | 97.10 |
| 432 | 840 | 113.52 |
| 433 | 841 | 76.31 |
| 434 | 842 | 79.59 |
| 435 | 843 | 88.62 |
| 436 | 844 | 70.46 |
| 437 | 845 | 91.44 |
| 438 | 846 | 109.11 |
| 439 | 847 | 92.58 |
| 440 | 848 | 80.69 |
| 441 | 849 | 76.47 |
| 442 | 850 | 78.63 |
| 443 | 851 | 90.36 |
| 444 | 852 | 99.71 |
| 445 | 853 | 110.44 |
| 446 | 854 | 100.23 |
| 447 | 855 | 91.27 |
| 448 | 856 | 97.87 |
| 449 | 857 | 93.03 |
| 450 | 858 | 76.70 |
| 451 | 859 | 75.71 |
| 452 | 860 | 62.06 |
| 453 | 861 | 83.82 |
| 454 | 862 | 100.20 |
| 455 | 863 | 88.36 |
| 456 | 864 | 99.25 |
| 457 | 865 | 96.99 |
| 458 | 866 | 98.53 |
| 459 | 867 | 94.89 |
| 460 | 868 | 91.31 |
| 461 | 869 | 87.34 |
| 462 | 870 | 68.39 |
| 463 | 871 | 79.70 |
| 464 | 872 | 77.37 |
| 465 | 873 | 79.48 |
| 466 | 874 | 99.77 |
| 467 | 875 | 101.18 |
| 468 | 876 | 81.61 |
| 469 | 877 | 79.70 |
| 470 | 878 | 71.42 |
| 471 | 879 | 104.14 |
| 472 | 880 | 82.01 |
| 473 | 881 | 89.26 |
| 474 | 882 | 100.12 |
| 475 | 883 | 113.04 |
| 476 | 884 | 103.38 |
| 477 | 885 | 78.43 |
| 478 | 886 | 76.80 |
| 479 | 887 | 75.48 |
| 480 | 888 | 96.02 |
| 481 | 889 | 87.69 |
| 482 | 890 | 79.88 |
| 483 | 891 | 94.05 |
| 484 | 892 | 79.37 |
| 485 | 893 | 87.45 |
| 486 | 894 | 85.13 |
| 487 | 895 | 71.05 |
| 488 | 896 | 89.13 |
| 489 | 897 | 85.22 |
| 490 | 898 | 76.98 |
| 491 | 899 | 75.90 |
| 492 | 900 | 68.93 |
| 493 | 901 | 100.54 |
| 494 | 902 | 86.36 |
| 495 | 903 | 84.43 |
| 496 | 904 | 58.35 |
| 497 | 905 | 61.41 |
| 498 | 906 | 78.73 |
| 499 | 907 | 77.32 |
| 500 | 908 | 81.41 |
| 501 | 909 | 65.25 |
| 502 | 910 | 86.56 |
| 503 | 911 | 93.27 |
| 504 | 912 | 75.33 |
| 505 | 913 | 83.07 |
| 506 | 914 | 86.66 |
| 507 | 915 | 75.71 |
| 508 | 916 | 99.40 |
| 509 | 917 | 94.00 |
| 510 | 918 | 69.43 |
| 511 | 919 | 73.17 |
| 512 | 920 | 79.61 |
| 513 | 921 | 73.21 |
| 514 | 922 | 67.67 |
| 515 | 923 | 70.66 |
| 516 | 924 | 74.20 |
| 517 | 925 | 79.86 |
| 518 | 926 | 84.20 |
| 519 | 927 | 80.05 |
| 520 | 928 | 83.95 |
| 521 | 929 | 64.03 |
| 522 | 930 | 81.25 |
| 523 | 931 | 91.81 |
| 524 | 932 | 80.77 |
| 525 | 933 | 80.91 |
| 526 | 934 | 86.91 |
| 527 | 935 | 98.00 |
| 528 | 936 | 100.59 |
| 529 | 937 | 95.16 |
| 530 | 938 | 93.38 |
| 531 | 939 | 83.26 |
| 532 | 940 | 93.89 |
| 533 | 941 | 79.06 |
| 534 | 942 | 65.76 |
| 535 | 943 | 73.94 |
| 536 | 944 | 70.60 |
| 537 | 945 | 69.82 |
| 538 | 946 | 75.67 |
| 539 | 947 | 74.41 |
| 540 | 948 | 84.16 |
| 541 | 949 | 74.98 |
| 542 | 950 | 99.60 |
| 543 | 951 | 105.74 |
| 544 | 952 | 108.40 |
| 545 | 953 | 99.15 |
| 546 | 954 | 97.13 |
| 547 | 955 | 103.19 |
| 548 | 956 | 94.19 |
| 549 | 957 | 74.11 |
| 550 | 958 | 95.53 |
| 551 | 959 | 91.29 |
| 552 | 960 | 87.63 |
| 553 | 961 | 83.68 |
| 554 | 962 | 88.08 |
| 555 | 963 | 91.77 |
| 556 | 964 | 95.50 |
| 557 | 965 | 82.47 |
| 558 | 966 | 60.85 |
| 559 | 967 | 107.81 |
| 560 | 968 | 103.82 |
| 561 | 969 | 83.38 |
| 562 | 970 | 82.95 |
| 563 | 971 | 106.95 |
| 564 | 972 | 80.11 |
| 565 | 973 | 86.68 |
| 566 | 974 | 54.48 |
| 567 | 975 | 89.37 |
| 568 | 976 | 72.72 |
| 569 | 977 | 59.06 |
| 570 | 978 | 83.77 |
| 571 | 979 | 83.27 |
| 572 | 980 | 69.77 |
| 573 | 981 | 127.93 |
| 574 | 982 | 70.21 |
| 575 | 983 | 113.37 |
| 576 | 984 | 91.72 |
| 577 | 985 | 89.88 |
| 578 | 986 | 95.96 |
| 579 | 987 | 90.25 |
| 580 | 988 | 96.81 |
| 581 | 989 | 83.64 |
| 582 | 990 | 108.91 |
| 583 | 991 | 88.20 |
| 584 | 992 | 77.01 |
| 585 | 993 | 72.20 |
| 586 | 994 | 91.58 |
| 587 | 995 | 138.65 |
| 588 | 996 | 78.65 |
| 589 | 997 | 83.41 |
| 590 | 998 | 89.23 |
| 591 | 999 | 72.63 |
| 592 | 1000 | 89.82 |
| 593 | 1001 | 71.98 |
| 594 | 1002 | 73.32 |
| 595 | 1003 | 89.67 |
| 596 | 1004 | 85.73 |
| 597 | 1005 | 95.90 |
| 598 | 1006 | 70.61 |
| 599 | 1007 | 79.40 |
| 600 | 1008 | 93.18 |
| 601 | 1009 | 80.30 |
| 602 | 1010 | 75.47 |
| 603 | 1011 | 99.56 |
| 604 | 1012 | 80.18 |
| 605 | 1013 | 63.43 |
| 606 | 1014 | 53.78 |
| 607 | 1015 | 80.72 |
| 608 | 1016 | 87.67 |
| 609 | 1017 | 104.80 |
| 610 | 1018 | 101.59 |
| 611 | 1019 | 107.72 |
| 612 | 1020 | 98.99 |
| 613 | 1021 | 110.95 |
| 614 | 1022 | 83.32 |
| 615 | 1023 | 86.80 |
| 616 | 1024 | 71.75 |
| 617 | 1025 | 73.68 |
| 618 | 1026 | 77.14 |
| 619 | 1027 | 91.42 |
| 620 | 1028 | 98.34 |
| 621 | 1029 | 80.63 |
| 622 | 1030 | 105.56 |
| 623 | 1031 | 67.47 |
| 624 | 1032 | 65.65 |
| 625 | 1033 | 52.97 |
| 626 | 1034 | 86.79 |
| 627 | 1035 | 99.31 |
| 628 | 1036 | 96.45 |
| 629 | 1037 | 90.74 |
| 630 | 1038 | 106.32 |
| 631 | 1039 | 111.00 |
| 632 | 1040 | 76.82 |
| 633 | 1041 | 110.51 |
| 634 | 1042 | 96.40 |
| 635 | 1043 | 90.74 |
| 636 | 1044 | 106.79 |
| 637 | 1045 | 79.07 |
| 638 | 1046 | 109.88 |
| 639 | 1047 | 73.85 |
| 640 | 1048 | 97.72 |
| 641 | 1049 | 74.53 |
| 642 | 1050 | 73.58 |
| 643 | 1051 | 100.37 |
| 644 | 1052 | 97.16 |
| 645 | 1053 | 88.22 |
| 646 | 1054 | 67.62 |
| 647 | 1055 | 107.80 |
| 648 | 1056 | 100.38 |
| 649 | 1057 | 83.87 |
| 650 | 1058 | 91.96 |
| 651 | 1059 | 103.82 |
| 652 | 1060 | 93.18 |
| 653 | 1061 | 99.64 |
| 654 | 1062 | 108.08 |
| 655 | 1063 | 91.19 |
| 656 | 1064 | 94.15 |
| 657 | 1065 | 85.19 |
| 658 | 1066 | 92.38 |
| 659 | 1067 | 97.61 |
| 660 | 1068 | 71.59 |
| 661 | 1069 | 72.06 |
| 662 | 1070 | 89.32 |
| 663 | 1071 | 70.54 |
| 664 | 1072 | 87.27 |
| 665 | 1073 | 101.59 |
| 666 | 1074 | 83.85 |
| 667 | 1075 | 61.54 |
| 668 | 1076 | 98.88 |
| 669 | 1077 | 122.82 |
| 670 | 1078 | 89.96 |
| 671 | 1079 | 93.69 |
| 672 | 1080 | 104.02 |
| 673 | 1081 | 70.44 |
| 674 | 1082 | 95.60 |
| 675 | 1083 | 101.74 |
| 676 | 1084 | 100.23 |
| 677 | 1085 | 104.86 |
| 678 | 1086 | 94.93 |
| 679 | 1087 | 71.97 |
| 680 | 1088 | 88.84 |
| 681 | 1089 | 86.82 |
| 682 | 1090 | 90.50 |
| 683 | 1091 | 76.45 |
| 684 | 1092 | 88.29 |
| 685 | 1093 | 88.49 |
| 686 | 1094 | 101.01 |
| 687 | 1095 | 71.63 |
| 688 | 1096 | 71.79 |
| 689 | 1097 | 75.00 |
| 690 | 1098 | 100.67 |
| 691 | 1099 | 87.34 |
| 692 | 1100 | 59.12 |
| 693 | 1101 | 76.82 |
| 694 | 1102 | 91.01 |
| 695 | 1103 | 105.39 |
| 696 | 1104 | 76.67 |
| 697 | 1105 | 115.83 |
| 698 | 1106 | 65.57 |
| 699 | 1107 | 67.04 |
| 700 | 1108 | 87.74 |
| 701 | 1109 | 70.12 |
| 702 | 1110 | 57.74 |
| 703 | 1111 | 95.21 |
| 704 | 1112 | 92.85 |
| 705 | 1113 | 94.98 |
| 706 | 1114 | 92.90 |
| 707 | 1115 | 87.04 |
| 708 | 1116 | 107.86 |
| 709 | 1117 | 107.45 |
| 710 | 1118 | 103.78 |
| 711 | 1119 | 108.86 |
| 712 | 1120 | 90.01 |
| 713 | 1121 | 54.44 |
| 714 | 1122 | 84.26 |
| 715 | 1123 | 53.33 |
| 716 | 1124 | 85.37 |
| 717 | 1125 | 92.38 |
| 718 | 1126 | 95.92 |
| 719 | 1127 | 94.69 |
| 720 | 1128 | 92.61 |
| 721 | 1129 | 111.25 |
| 722 | 1130 | 120.34 |
| 723 | 1131 | 69.15 |
| 724 | 1132 | 77.09 |
| 725 | 1133 | 62.79 |
| 726 | 1134 | 61.40 |
| 727 | 1135 | 75.81 |
| 728 | 1136 | 88.14 |
| 729 | 1137 | 94.56 |
| 730 | 1138 | 80.12 |
| 731 | 1139 | 122.12 |
| 732 | 1140 | 100.63 |
| 733 | 1141 | 137.86 |
| 734 | 1142 | 84.96 |
| 735 | 1143 | 82.41 |
| 736 | 1144 | 102.07 |
| 737 | 1145 | 79.28 |
| 738 | 1146 | 88.70 |
| 739 | 1147 | 94.56 |
| 740 | 1148 | 84.26 |
| 741 | 1149 | 75.88 |
| 742 | 1150 | 80.08 |
| 743 | 1151 | 100.19 |
| 744 | 1152 | 104.19 |
| 745 | 1153 | 80.12 |
| 746 | 1154 | 90.86 |
| 747 | 1155 | 78.24 |
| 748 | 1156 | 100.07 |
| 749 | 1157 | 99.73 |
| 750 | 1158 | 92.14 |
| 751 | 1159 | 74.79 |
| 752 | 1160 | 101.44 |
| 753 | 1161 | 95.43 |
| 754 | 1162 | 72.49 |
| 755 | 1163 | 98.63 |
| 756 | 1164 | 97.39 |
| 757 | 1165 | 92.31 |
| 758 | 1166 | 66.14 |
| 759 | 1167 | 81.59 |
| 760 | 1168 | 101.57 |
| 761 | 1169 | 76.73 |
| 762 | 1170 | 80.29 |
| 763 | 1171 | 79.24 |
| 764 | 1172 | 82.98 |
| 765 | 1173 | 100.86 |
| 766 | 1174 | 103.00 |
| 767 | 1175 | 83.89 |
| 768 | 1176 | 84.24 |
| 769 | 1177 | 69.27 |
| 770 | 1178 | 66.23 |
| 771 | 1179 | 98.28 |
| 772 | 1180 | 104.11 |
| 773 | 1181 | 78.20 |
| 774 | 1182 | 85.88 |
| 775 | 1183 | 72.31 |
| 776 | 1184 | 121.83 |
| 777 | 1185 | 119.11 |
| 778 | 1186 | 87.85 |
| 779 | 1187 | 88.59 |
| 780 | 1188 | 90.80 |
| 781 | 1189 | 106.77 |
| 782 | 1190 | 104.98 |
| 783 | 1191 | 91.77 |
| 784 | 1192 | 89.42 |
| 785 | 1193 | 83.93 |
| 786 | 1194 | 80.78 |
| 787 | 1195 | 92.86 |
| 788 | 1196 | 72.89 |
| 789 | 1197 | 88.08 |
| 790 | 1198 | 75.66 |
| 791 | 1199 | 80.93 |
| 792 | 1200 | 81.20 |
| 793 | 1201 | 91.38 |
| 794 | 1202 | 80.11 |
| 795 | 1203 | 77.43 |
| 796 | 1204 | 81.19 |
| 797 | 1205 | 86.25 |
| 798 | 1206 | 90.22 |
| 799 | 1207 | 91.62 |
| 800 | 1208 | 94.68 |
| 801 | 1209 | 76.04 |
| 802 | 1210 | 90.74 |
| 803 | 1211 | 81.58 |
| 804 | 1212 | 100.77 |
| 805 | 1213 | 84.17 |
| 806 | 1214 | 88.63 |
| 807 | 1215 | 95.86 |
| 808 | 1216 | 95.96 |
| 809 | 1217 | 95.17 |
| 810 | 1218 | 89.06 |
| 811 | 1219 | 73.14 |
| 812 | 1220 | 81.30 |
| 813 | 1221 | 81.14 |
| 814 | 1222 | 84.53 |
| 815 | 1223 | 77.04 |
| 816 | 1224 | 83.46 |

### Example 2. IC50 Determination of MSH3-Targeting siRNA Molecules

### Objective

This Example describes the results of a series of experiments undertaken to determine the IC50 of siRNA molecules complementary to specific regions within a human MSH3 mRNA.

### Materials and Methods

MeWo cells (metastatic malignant melanoma cells, ATCC cat #HTB-65) were actively transfected at concentrations of 1 fM to 100 nM using 0.05% RNAiMax as the transfection reagent. Non-siRNA treated cells (untreated) exposed to the same media composition were used as controls. Cells were grown in a standard cell culture incubator at 37° C. and 5% CO₂. At 72 hours after siRNA treatment, the TaqMan Fast Advanced Cells-to-CT Kit (Thermo) was used to lyse all cells, digest the genomic DNA, perform reverse-transcription on the resulting RNA, and perform quantitative polymerase chain reaction (qPCR) on the resulting cDNA. Exon spanning MSH3-specific primer/probes (Thermo) were multiplexed with primer/probes for a housekeeping gene (ATP5B, Thermo), and all reactions were run on a Quantstudio 7 Flex (Thermo). MSH3 mRNA expression was calculated relative to untreated control cells using the delta-delta-Ct method. Biological duplicates and technical (qPCR) duplicates were run for each condition, and all results are expressed as the mean of all technical and biological duplicates, and as residual mRNA expression as a percent of untreated control cells.

### Results

Cells were treated with an siRNA molecule of the disclosure having an antisense strand and a sense strand as shown in Table 5, below. The IC50 was calculated and the knockdown results are reported in Table 5.

TABLE 5
IC50 of MSH3-Targeting siRNA Molecules

| Sense SEQ ID NO: | Antisense SEQ ID NO: | IC50 (pM) |
|---|---|---|
| 413 | 821 | 55.45 |
| 425 | 833 | 120.10 |
| 426 | 834 | 58.99 |
| 427 | 835 | 87.66 |
| 428 | 836 | 165.80 |
| 441 | 849 | 5.50 |
| 450 | 858 | 71.32 |
| 452 | 860 | 79.12 |
| 510 | 918 | 78.53 |
| 511 | 919 | 116.90 |
| 513 | 921 | 63.72 |
| 514 | 922 | 38.53 |
| 516 | 924 | 32.86 |
| 517 | 925 | 24.82 |
| 521 | 929 | 34.45 |
| 534 | 942 | 4.20 |
| 537 | 945 | 28.78 |
| 538 | 946 | 66.17 |
| 566 | 974 | 28.35 |
| 569 | 977 | 20.68 |
| 574 | 982 | 1.78 |
| 585 | 993 | 22.67 |
| 591 | 999 | 7.11 |
| 601 | 1009 | 111.20 |
| 602 | 1010 | 10.57 |
| 604 | 1012 | 7.17 |
| 605 | 1013 | 1.13 |
| 610 | 1018 | 2150.00 |
| 617 | 1025 | 4.88 |
| 625 | 1033 | 8.69 |
| 639 | 1047 | 45.54 |
| 679 | 1087 | 198.60 |
| 681 | 1089 | 148.50 |
| 699 | 1107 | 40.16 |
| 702 | 1110 | 104.30 |
| 713 | 1121 | 44.80 |
| 715 | 1123 | 14.33 |
| 725 | 1133 | 24.90 |
| 726 | 1134 | 109.40 |
| 770 | 1178 | 22.15 |

### Example 3. In Vivo Inhibition of MSH3 Gene Expression Using Di-siRNA Sequence Variants in Mouse

### In Vivo Administration of Di-siRNA

12 siRNA sequences targeting MSH3 were synthesized as di-siRNAs having the structure of Formula XVII. Test articles are shown in Table 6, below.

TABLE 6
MSH3-targeting siRNA molecules used for in vivo experiments

| di-siRNA ID | Sense SEQ ID NO: | Antisense SEQ ID NO: |
|---|---|---|
| A | 426 | 834 |
| B | 427 | 835 |
| C | 428 | 836 |
| D | 510 | 918 |
| E | 513 | 921 |
| F | 514 | 922 |
| G | 516 | 924 |
| H | 517 | 925 |
| I | 521 | 929 |
| J | 591 | 999 |
| K | 602 | 1010 |
| L | 604 | 1012 |

Di-siRNAs were formulated in PBS. siRNAs at the 20 nmol dose were formulated as a divalent cation salt. In vivo testing was performed in FVB/NJ female mice with intracerebroventricular (ICV) dosing, in groups of 8 animals. On day 1, stereotactic injection was performed, wherein bilateral ICV injections of test article or PBS control (5 μL per side, 10 μL total) were performed at 0.5-2 μL/min after needle placement at the following coordinates from bregma: −0.45 mm or −0.25 mm anteriorposterior, +/−1 mm mediolateral and −2.5 mm dorsoventral. Dose levels of 0.5 nmol, 1 nmol, 2.5 nmol, 5 nmol, and/or 20 nmol were utilized. At 28 days after injection, animals were perfused with cold 1×PBS and brains were collected and sliced. Tissue punches of fixed diameter and thickness were collected from different brain regions (motor cortex, hippocampus and striatum) and snap frozen on dry ice.

### Protein Expression Analysis

To evaluate MSH3 protein expression levels, mouse brain tissue punches were homogenized in Cell Lysis Buffer with protease/phosphatase inhibitor cocktail (Cell Signaling Technologies) using a TissueLyser II. Tissue homogenate was centrifuged for 5 minutes at 1000 rpm to remove bubbles. Total protein content was normalized to using BCA (Pierce) to 0.125 mg/mL in 16 μL.

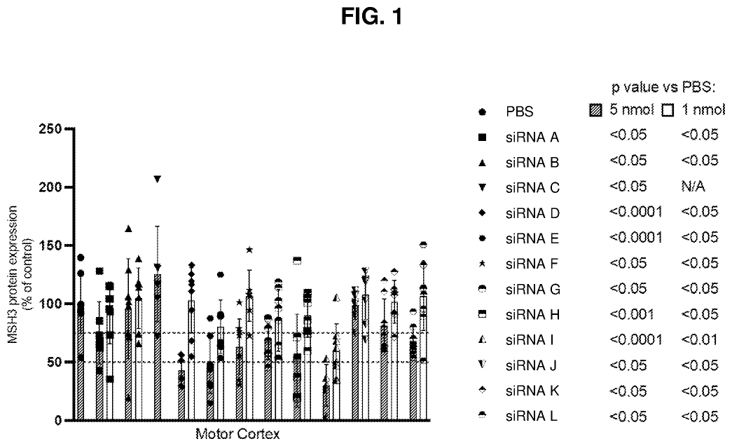

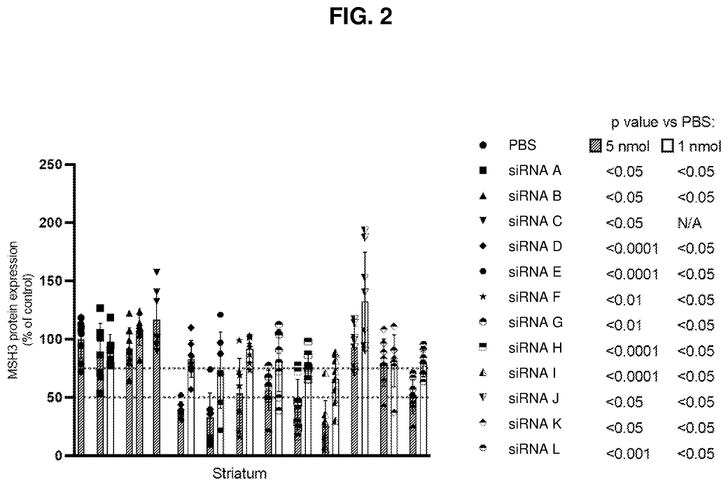

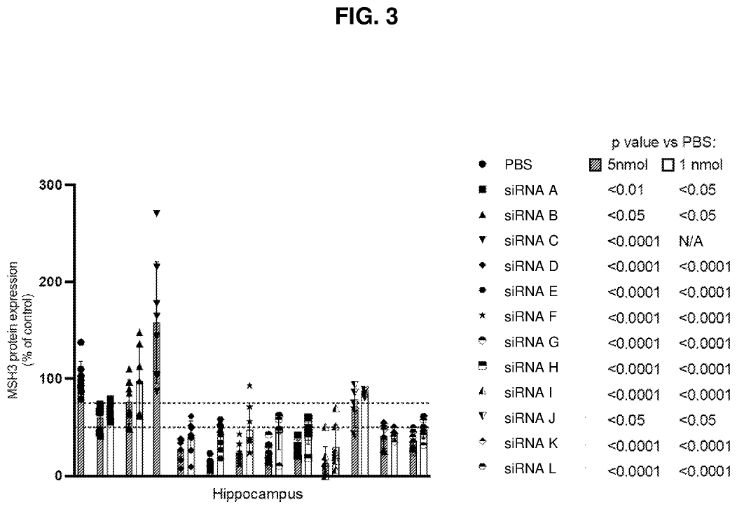

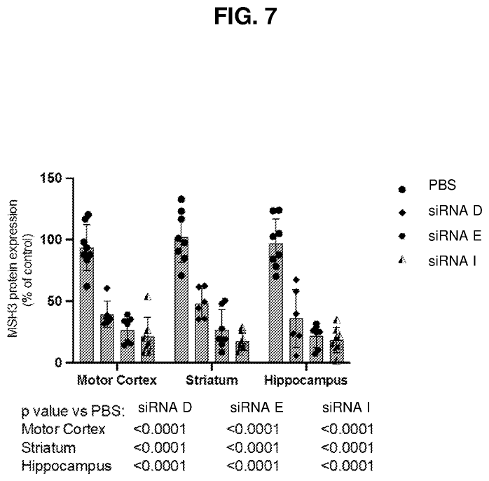

For MSH3 protein expression analysis, Western blot was performed using the Jess (Protein Simple) system and reagents as recommended by the manufacturer. The 12-230 kD separation cartridge was utilized, with Fluorescent Master Mix 1 and the anti-mouse detection module (all from Protein Simple). Samples were probed for mouse total MSH3 expression (MABE324 Antibody, EMD Millipore, 1:100 dilution) and were normalized against the multiplexed housekeeping protein reference mouse actin (MAB8929, R&D Systems, 1:20,000 dilution). Comparative analysis was performed for relative MSH3 quantitation against samples taken from PBS treated mice. Results are shown in FIGS. 1, 2, 3, 7, and 9.

### RNA Expression Analysis

To evaluate MSH3 mRNA expression levels, total RNA was extracted from mouse brain tissue punches using phenol:chloroform extraction. This method was performed by first disrupting tissue samples in TRIzol reagent (Invitrogen) using a TissueLyser II (Qiagen) and adding chloroform to the homogenized samples at a ratio of 5:1 TRIzol:chloroform. Tubes were then shaken vigorously and spun at 12,000×g for 15 mins and the resulting upper aqueous phase containing total RNA was carefully removed and added to a clean tube. An equal volume of 70% ethanol was added to each sample and mixed gently. The sample was further purified using Qiagen RNeasy column purification according to standard kit protocol. Samples were eluted in 40 μL RNase-free water. Following elution, RNA was analyzed on a TapeStation 4200 Bioanalyzer (Agilent) to assess concentration and quality. All samples were normalized for total RNA. cDNA synthesis was performed in a 20 μL reaction volume using the Applied Biosystems High-Capacity cDNA Reverse Transcription kit. Following RT-PCR, nuclease-free water was added to cDNA for a final sample volume of 50 μL.

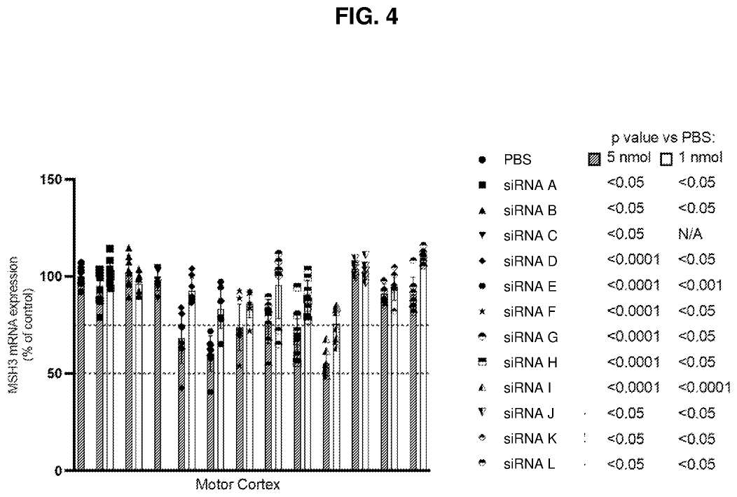

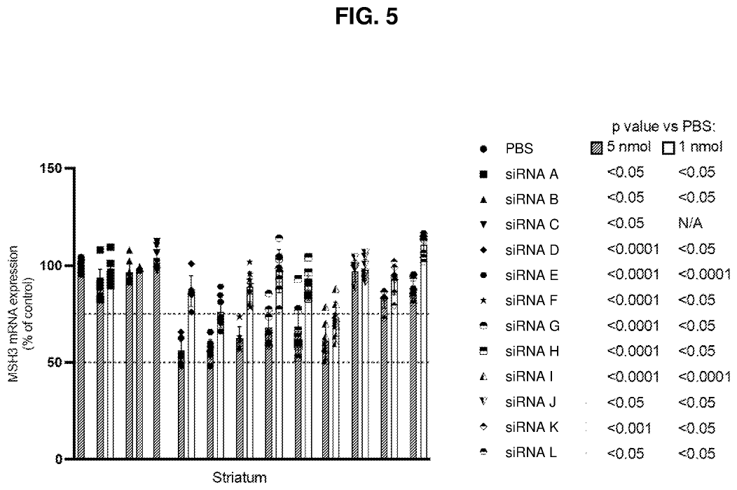

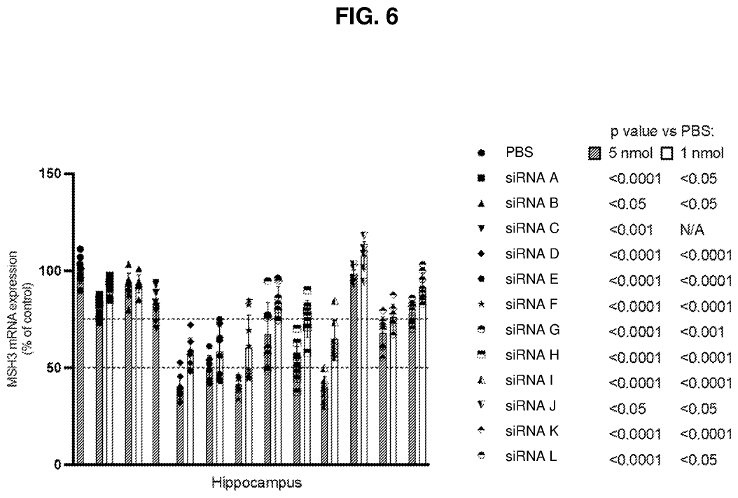

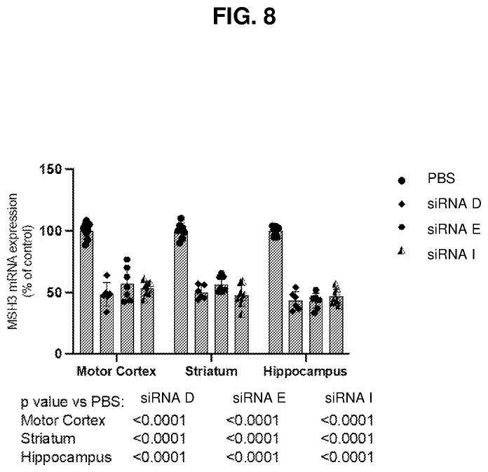

For Htt gene expression analysis, qPCR was performed using TaqMan reagents on a QuantStudio7 Real-Time PCR instrument (Life Technologies). Samples were probed for mouse total MSH3 expression (assay ID Mm00487756_ml) and were normalized against the multiplexed housekeeping gene reference mouse TBP (Assay ID Mm01277042_ml). Comparative analysis was performed for relative MSH3 quantitation (DeltaDeltaCt) against samples taken from PBS treated mice. Results are shown in FIGS. 4, 5, 6, and 8.

### Statistical Assessment

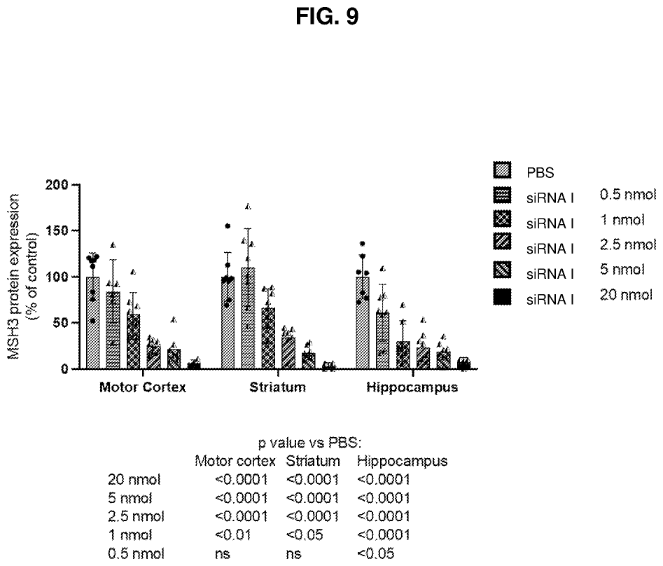

Statistical significance between PBS control and each treatment group was determined using two-way ANOVA and Dunnett's multiple comparisons test, except for FIG. 9, which utilized Sidak's multiple comparisons test.

### Example 4. Generating MSH3-Targeting siRNA Molecules

The siRNA molecules of the disclosure can be synthesized by standard methods known in the art as further discussed below, e.g., by use of an automated DNA synthesizer, such as are commercially available from, for example, Biosearch, Applied Biosystems, Inc.

The siRNA agent can be prepared using solution-phase or solid-phase organic synthesis or both. Organic synthesis offers the advantage that the oligonucleotide including unnatural or modified nucleotides can be easily prepared. Specific examples of siRNA molecules, with the nucleotide sequence of the sense and antisense strand, as well as the MutS Homolog 3 (MSH3) mRNA target sequence, are shown in Table 1A, above. It is appreciated that one of skill in the art could anneal the antisense (AS) strand to the corresponding sense (S) strand to yield a ds-siRNA molecule. Alternatively, one of skill in the art could derive a ss-siRNA molecule using antisense strand only.

### Example 5. Optimizing MSH3-Targeting siRNA Molecules

It is contemplated that for any small interfering RNA (siRNA) agent disclosed herein, modifications to the siRNA may further optimize the molecule's efficacy or biophysical properties (e.g., increasing serum stability or circulating half-life, increasing thermal stability, enhancing transmembrane delivery, and/or targeting to a particular location or cell type). Such optimization could be achieved by systematically either adding or removing linked nucleosides to generate longer or shorter sequences. Further siRNA optimization could include the incorporation of, for example, one or more alternative nucleosides, alternative 2′ sugar moieties, and/or alternative internucleoside linkages. Further still, such optimized siRNA molecules may include the introduction of hydrophobic and/or stabilizing moieties at the 5′ and/or 3′ ends.

siRNA Optimization with Alternative Nucleosides

Optimization of the siRNA molecules of the disclosure may include one or more of the following nucleoside modifications: 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, and/or 3-deazaguanine and 3-deazaadenine. The siRNA molecules may also include nucleobases in which the purine or pyrimidine base is replaced with other heterocycles, for example 7-deaza-adenine, 7-deazaguanosine, 2-aminopyridine, and/or 2-pyridone. Further optimization of the siRNA molecules of the disclosure may include nucleobases disclosed in U.S. Pat. No. 3,687,808; Kroschwitz, J. I., ed. The Concise Encyclopedia of Polymer Science and Engineering, New York, John Wiley & Sons, 1990, pp. 858-859; Englisch et al., Angewandte Chemie, International Edition 30:613, 1991; and Sanghvi, Y. S., Chapter 16, Antisense Research and Applications, CRC Press, Gait, M. J. ed., 1993, pp. 289-302.

siRNA Optimization with Alternative Sugar Modifications

Optimization of the siRNA molecules of the disclosure may include one or more of the following 2′ sugar modifications: 2′-O-methyl (2′-O-Me), 2′-methoxyethoxy (2′-O—CH₂CH₂OCH₃, also known as 2′-O-(2-methoxyethyl) or 2′-MOE), 2′-dimethylaminooxyethoxy, i.e., a O(CH₂)₂ON(CH₃)₂ group, also known as 2′-DMAOE, and/or 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other possible 2′-modifications that can optimize the siRNA molecules of the disclosure include all possible orientations of OH; F; O—, S—, or N-alkyl; O—, S—, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Other potential sugar substituent groups include, e.g., aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the siRNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

siRNA Optimization with Alternative Internucleoside Linkages

Optimization of the siRNA molecules of the disclosure may include one or more of the following internucleoside modifications: phosphorothioates, phosphorodithioates, phosphotriesters, aminoalkylphosphotriesters, methyl and other alkyl phosphonates including 3-alkylene phosphonates, 5′-alkylene phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3-5′ linkages, 2′-5′ linked analogs of these, and those having inverted polarity wherein one or more internucleotide linkages is a 3′ to 3′, 5′ to 5′ or 2′ to 2′ linkage.

siRNA Optimization with Hydrophobic Moieties

Optimization of the siRNA molecules of the disclosure may include hydrophobic moieties covalently attached to the 5′ end or the 3′ end. Non-limiting examples of hydrophobic moieties suitable for use with the siRNA molecules of the disclosure may include cholesterol, vitamin D, tocopherol, phosphatidylcholine (PC), docosahexaenoic acid, docosanoic acid, PC-docosanoic acid, eicosapentaenoic acid, lithocholic acid or any combination of the aforementioned hydrophobic moieties with PC.

siRNA Optimization with Stabilizing Moieties

Optimization of the siRNA molecules of the disclosure may include a 5′-phosphorous stabilizing moiety that protects the siRNA molecules from degradation. A 5′-phosphorus stabilizing moiety replaces the 5′-phosphate to prevent hydrolysis of the phosphate. Hydrolysis of the 5′-phosphate prevents binding to RISC, a necessary step in gene silencing. Any replacement for phosphate that does not impede binding to RISC is contemplated in this disclosure. In some embodiments, the replacement for the 5′-phosphate is also stable to in vivo hydrolysis. Each siRNA strand may independently and optionally employ any suitable 5′-phosphorus stabilizing moiety. Non-limiting examples of 5′ stabilizing moieties suitable for use with the siRNA molecules of the disclosure may include those demonstrated by Formulas IX-XVI above.

siRNA Optimization with Branched siRNA

Optimization of the siRNA molecules of the disclosure may include the incorporation of branching patterns, such as, for example, di-branched, tri-branched, or tetra-branched siRNAs connected by way of a linker. Each main branch may be further branched to allow for 2, 3, 4, 5, 6, 7, or 8 separate RNA single- or double-strands. The branch points on the linker may stem from the same atom, or separate atoms along the linker. Some exemplary embodiments are listed in Table 2, above.

The siRNA composition of the disclosure may be optimized to be in the form of: di-branched siRNA molecules, as represented by any one of Formulas XVII-XIX; tri-branched siRNA molecules, as represented by any one of Formulas XX-XXIII; and/or tetra-branched siRNA molecules, as represented by any one of Formulas XXIV-XXVIII, wherein each RNA, independently, is an siRNA molecule, L is a linker, and each X, independently, represents a branch point moiety (e.g., phosphoroamidite, tosylated solketal, 1,3-diaminopropanol, pentaerythritol, or any one of the branch point moieties described in U.S. Pat. No. 10,478,503).

### Example 6. Preparation and Administrating MSH3-Targeting siRNA Molecules

The siRNA molecules in the present disclosure may be formulated into a pharmaceutical composition for administration to a subject in a biologically compatible form suitable for administration in vivo. For example, the siRNA molecules of the disclosure may be administered in a suitable diluent, carrier, or excipient, and may further contain a preservative, e.g., to prevent the growth of microorganisms. Conventional procedures and ingredients for the selection and preparation of suitable formulations are described, for example, in Remington, J. P. The Science and Practice of Pharmacy, Easton, PA. Mack Publishers, 2012, 22^{nd} ed. and in The United States Pharmacopeial Convention, The National Formulary, United States Pharmacopeial, 2015, USP 38 NF 33).

The method of the disclosure contemplates any route of administration to the subject that is tolerated by the siRNA compositions of the disclosure. Non-limiting examples of siRNA injections into the CNS include intrathecally, intracerebroventricularly, intrastriatally, intraparenchymally, or intra-cisterna magna injection by catheterization. Examples of systemic administration include intravenous, intramuscular, and subcutaneous injection. A physician having ordinary skill in the art can readily determine an effective route of administration.

### Example 7. Methods for the Treatment of Microsatellite Repeat Expansion Diseases Using MSH3-Targeting siRNA Molecules

A subject in need of treatment of a microsatellite repeat expansion disease, such as Huntington's Disease, is treated with a dosage of the siRNA molecule or siRNA composition of the disclosure, formulated as a salt, at frequency determined by a practitioner. A physician having ordinary skill in the art can readily determine an effective amount of the siRNA molecule for administration to a mammalian subject (e.g., a human) in need thereof. For example, a physician could start prescribing doses of one of the siRNA molecules of the disclosure at levels lower than that required to achieve the desired therapeutic effect and gradually increase the dosage until the desired effect is achieved. Alternatively, a physician may begin a treatment regimen by administering one of the siRNA molecules of the disclosure at a high dose and subsequently administer progressively lower doses until a minimum dose that produces a therapeutic effect (e.g., a reduction in expression of MSH3 mRNA or suitable biomarker) is achieved. In general, a suitable daily dose of one of one of the siRNA molecules of the disclosure will be an amount which is the lowest dose effective to produce a therapeutic effect. The ss- or ds-siRNA molecules of the disclosure may be administered by injection, e.g., intrathecally, intracerebroventricularly, intrastriatally, intraparenchymally, intravenously, intramuscularly, subcutaneously, or by intra-cisterna magna injection via catheterization. A daily dose of a therapeutic composition of one of the siRNA molecules of the disclosure may be administered as a single dose or as two, three, four, five, six or more doses administered separately at appropriate intervals throughout the day, week, month, or year, optionally, in unit dosage forms. While it is possible for any of the siRNA molecules of the disclosure to be administered alone, it may also be administered as a pharmaceutical formulation in combination with excipients, carriers, and optionally, additional therapeutic agents. Dosage and frequency are determined based on the subject's height, weight, age, sex, and other disorders.

The siRNA molecule(s) of the disclosure is selected by the practitioner for compatibility with the subject. Single- or double-stranded siRNA molecules (e.g., non-branched siRNA, di-branched siRNA, tri-branched siRNA, tetra-branched siRNA) are available for selection. The siRNA molecule chosen has an antisense strand and may have a sense strand with a sequence and RNA modifications (e.g., natural and non-natural internucleoside linkages, modified sugars, 5′-phosphorus stabilizing moieties, hydrophobic moieties, and/or branching structures) best suited to the patient.

The siRNA molecule is delivered by the route best suited the patient (e.g., intrathecally, intracerebroventricularly, intrastriatally, intraparenchymally, intravenously, intramuscularly, subcutaneously, or by intra-cisterna magna injection via catheterization) and condition at a rate tolerable to the patient until the subject has reached a maximum tolerated dose, or until symptoms are ameliorated satisfactorily.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A small interfering RNA (siRNA) molecule comprising an antisense strand and sense strand having complementarity to the antisense strand, wherein the antisense strand is from 10 to 30 nucleotides in length and has complementarity sufficient to hybridize to a region within a MutS Homolog 3 (MSH3) mRNA transcript having the nucleic acid sequence of any one of SEQ ID NOs: 1-408.

2. The siRNA molecule of claim 1, wherein the antisense strand has at least 70% complementarity to a region of 21 contiguous nucleobases within the MSH3 mRNA transcript having the nucleic acid sequence of any one of SEQ ID NOs: 1-408.

3. The siRNA molecule of claim 2, wherein the antisense strand has at least 75% complementarity to a region of 21 contiguous nucleobases within the MSH3 mRNA transcript having the nucleic acid sequence of any one of SEQ ID NOs: 1-408, optionally wherein the antisense strand has at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to the region within the MSH3 mRNA transcript having the nucleic acid sequence of any one of SEQ ID Nos: 1-408.

4. The siRNA molecule of any one of claims 1-3, wherein the antisense strand comprises at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 contiguous nucleotides that are fully complementary to a contiguous polynucleotide segment of equal length within the region of the MSH3 RNA transcript having the nucleic acid sequence of any one of SEQ ID NOs: 1-408.

5. The siRNA molecule of claim 4, wherein the antisense strand comprises from 10 to 30 contiguous nucleotides that are fully complementary to a contiguous polynucleotide segment of equal length within the region of the MSH3 RNA transcript having the nucleic acid sequence of any one of SEQ ID NOs: 1-408.

6. The siRNA molecule of claim 5, wherein the antisense strand comprises from 12 to 30 contiguous nucleotides that are fully complementary to a contiguous polynucleotide segment of equal length within the region of the MSH3 RNA transcript having the nucleic acid sequence of any one of SEQ ID NOs: 1-408.

7. The siRNA molecule of claim 6, wherein the antisense strand comprises from 15 to 30 contiguous nucleotides that are fully complementary to a contiguous polynucleotide segment of equal length within the region of the MSH3 RNA transcript having the nucleic acid sequence of any one of SEQ ID NOs: 1-408.

8. The siRNA molecule of claim 7, wherein the antisense strand comprises from 18 to 30 contiguous nucleotides that are fully complementary to a contiguous polynucleotide segment of equal length within the region of the MSH3 RNA transcript having the nucleic acid sequence of any one of SEQ ID NOs: 1-408.

9. The siRNA molecule of claim 8, wherein the antisense strand comprises from 18 to 25 contiguous nucleotides that are fully complementary to a contiguous polynucleotide segment of equal length within the region of the MSH3 RNA transcript having the nucleic acid sequence of any one of SEQ ID NOs: 1-408.

10. The siRNA molecule of any one of claims 1-9, wherein the antisense strand comprises from 18 to 21 contiguous nucleotides that are fully complementary to a contiguous polynucleotide segment of equal length within the region of the MSH3 RNA transcript having the nucleic acid sequence of any one of SEQ ID NOs: 1-408.

11. The siRNA molecule of claim 10, wherein the antisense strand comprises 21 contiguous nucleotides that are fully complementary to a contiguous polynucleotide segment of equal length within the region of the MSH3 RNA transcript having the nucleic acid sequence of any one of SEQ ID NOs: 1-408.

12. The siRNA molecule of any one of claims 1-11, wherein the antisense strand comprises 9 or fewer nucleotide mismatches relative to a region of 21 contiguous nucleobases of the MSH3 RNA transcript having the nucleic acid sequence of any one of SEQ ID NOs: 1-408, optionally wherein the antisense strand comprises 8 or fewer, 7 or fewer, 6 or fewer, 5 or fewer, 4 or fewer, 3 or fewer, 2 or fewer, or only 1 mismatch relative to the region of the MSH3 RNA transcript having the nucleic acid sequence of any one of SEQ ID NOs: 1-408.

13. The siRNA molecule of any one of claims 1-12, wherein the region of the MSH3 RNA transcript has the nucleic acid sequence of any one of SEQ ID NOs: 1-24, 40-84, 100, 118-139, 173-181, 193-205, 220-222, 240-242, 249-308, 319-330, 336-377, 384-386, and 398-408.

14. The siRNA molecule of any one of claims 1-12, wherein the region of the MSH3 RNA transcript has the nucleic acid sequence of any one of SEQ ID NOs: 5, 17-20, 33, 42, 44, 102, 103, 105, 106, 108, 109, 113, 126, 129, 130, 158, 161, 166, 177, 183, 193, 194, 196, 197, 202, 209, 217, 231, 271, 273, 291, 294, 305, 307, 317, 318, and 362, optionally wherein the region of the MSH3 RNA transcript has the nucleic acid sequence of any one of SEQ ID NOs: 18-20, 102, 105, 106, 108, 109, 113, 183, 194, and 196.

15. The siRNA molecule of claim 14, wherein the region of the MSH3 RNA transcript has the nucleic acid sequence of any one of SEQ ID NOs: 5, 17-20, 42, 44, 126, 129, 130, 158, 177, 183, 193, 194, 196, 197, 202, 209, 231, 271, 273, 291, 294, 305, 307, 317, 318, and 362, optionally wherein the region of the MSH3 RNA transcript has the nucleic acid sequence of any one of SEQ ID NOs: 18-20, 183, 194, and 196.

16. The siRNA molecule of any one of claims 1-15, wherein the antisense strand has a nucleic acid sequence that is at least 85% identical to the nucleic acid sequence of any one of SEQ ID NOs: 817-1224.

17. The siRNA molecule of claim 16, wherein the antisense strand has a nucleic acid sequence that is at least 90% identical to the nucleic acid sequence of any one of SEQ ID NOs: 817-1224.

18. The siRNA molecule of claim 17, wherein the antisense strand has a nucleic acid sequence that is at least 95% identical to the nucleic acid sequence of SEQ ID NOs: 817-1224, optionally wherein the antisense strand has a nucleic acid sequence that is at least 96%, 97%, 98%, or 99% identical to the nucleic acid sequence of any one of SEQ ID NOs: 817-1224.

19. The siRNA molecule of claim 18, wherein the antisense strand has the nucleic acid sequence of any one of SEQ ID NOs: 817-1224.

20. The siRNA molecules of any one of claims 16-19, wherein the nucleic acid sequence is any one of SEQ ID NOs: 817-840, 856-900, 916, 934-955, 989-997, 1009-1021, 1036-1038, 1056-1058, 1065-1124,1135-1146, 1152-1193, 1200-1202, and 1214-1224.

21. The siRNA molecule of any one of claims 16-19, wherein the nucleic acid sequence is any one of SEQ ID NOs: 821, 833-836, 849, 858, 860, 918, 919, 921, 922, 924, 925, 929, 942, 945, 946, 974, 977, 982, 993, 999, 1009, 1010, 1012, 1013, 1018, 1025, 1033, 1047, 1087, 1089, 1107, 1110, 1121, 1123, 1133, and 1134, optionally wherein the nucleic acid sequence is any one of SEQ ID NOs: 834-836, 918, 921, 922, 924, 925, 999, 1010, and 1012.

22. The siRNA molecule of claim 21, wherein the nucleic acid sequence is any one of SEQ ID NOs:
821, 833-836, 858, 860, 942, 945, 946, 974, 993, 999, 1009, 1010, 1012, 1013, 1018, 1025, 1047, 1087, 1089, 1107, 1110, 1121, 1123, 1133, and 1134, optionally wherein the nucleic acid sequence is any one of SEQ ID NOs: 834-836, 999, 1010, and 1012.

23. The siRNA molecule of any one of claims 1-22, wherein the sense strand has a nucleic acid sequence that is at least 85% identical to the nucleic acid sequence of any one of SEQ ID NOs: 409-816.

24. The siRNA molecule of claim 23, wherein the sense strand has a nucleic acid sequence that is at least 90% identical to the nucleic acid sequence of any one of SEQ ID NOs: 409-816.

25. The siRNA molecule of claim 24, wherein the sense strand has a nucleic acid sequence that is at least 95% identical to the nucleic acid sequence of SEQ ID NOs: 409-816, optionally wherein the sense strand has a nucleic acid sequence that is at least 96%, 97%, 98%, or 99% identical to the nucleic acid sequence of any one of SEQ ID NOs: 409-816.

26. The siRNA molecule of claim 25, wherein the sense strand has the nucleic acid sequence of any one of SEQ ID NOs: 409-816.

27. The siRNA molecule of any one of claims 23-26, wherein the nucleic acid sequence is any one of SEQ ID NOs: 409-432, 448-492, 508, 526-547, 581-589, 601-613, 628-630, 648-650, 657-716, 727-738, 744-785, 792-794, and 806-816.

28. The siRNA molecule of any one of claims 23-26, wherein the nucleic acid sequence is any one of SEQ ID NOs: 413, 425-428, 441, 450, 452, 510, 511, 513, 514, 516, 517, 521, 534, 537, 538, 566, 569, 574, 585, 591, 601, 602, 604, 605, 610, 617, 625, 639, 679, 681, 699, 702, 713, 715, 725, 726, and 770, optionally wherein the nucleic acid sequence is any one of SEQ ID NOs: 426-428, 513, 516, 514, 516, 517, 521, 591, 602, and 604.

29. The siRNA molecule of claim 28, wherein the nucleic acid sequence is any one of SEQ ID NOs: 413, 425-428, 450, 452, 534, 537, 538, 566, 585, 591, 601, 602, 604, 605, 610, 617, 639, 679, 681, 699, 702, 713, 715, 725, 726, and 770, optionally wherein the nucleic acid sequence is any one of SEQ ID NOs: 426-428, 591, 602, and 604.

30. The siRNA molecule of any one of claims 1-29, wherein the antisense strand comprises a structure represented by Formula I, wherein Formula I is, in the 5′-to-3′ direction: 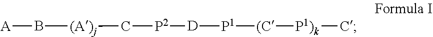
wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

31. The siRNA molecule of claim 30, wherein the antisense strand comprises a structure represented by Formula A1, wherein Formula A1 is, in the 5′-to-3′ direction: 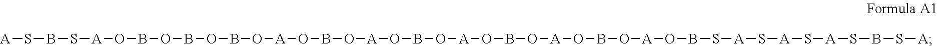
wherein A represents a 2′-O-Me ribonucleoside, B represents a 2′-F ribonucleoside, O represents a phosphodiester internucleoside linkage, and S represents a phosphorothioate internucleoside linkage.

32. The siRNA molecule of any one of claims 1-29, wherein the antisense strand comprises a structure represented by Formula II, wherein Formula II is, in the 5′-to-3′ direction: 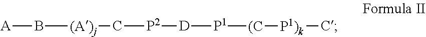
wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

33. The siRNA molecule of claim 32, wherein the antisense strand comprises a structure represented by Formula A2, wherein Formula A2 is, in the 5′-to-3′ direction: 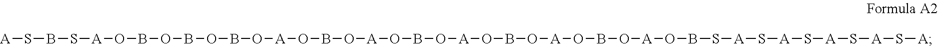
wherein A represents a 2′-O-Me ribonucleoside, B represents a 2′-F ribonucleoside, O represents a phosphodiester internucleoside linkage, and S represents a phosphorothioate internucleoside linkage.

34. The siRNA molecule of any one of claims 1-33, wherein the sense strand comprises a structure represented by Formula III, wherein Formula III is, in the 5′-to-3′ direction: 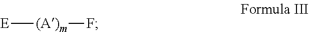
wherein E is represented by the formula (C—P¹)₂;
F is represented by the formula (C—P²)₃-D-P¹—C—P¹—C, (C—P²)₃-D-P²—C—P²—C, (C—P²)₃-D-P¹—C—P¹-D, or (C—P²)₃-D-P²—C—P²-D;
A′, C, D, P¹, and P² are as defined in Formula II; and
m is an integer from 1 to 7.

35. The siRNA molecule of claim 34, wherein the sense strand comprises a structure represented by Formula S1, wherein Formula S1 is, in the 5′-to-3′ direction: 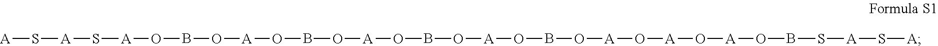
wherein A represents a 2′-O-Me ribonucleoside, B represents a 2′-F ribonucleoside, O represents a phosphodiester internucleoside linkage, and S represents a phosphorothioate internucleoside linkage.

36. The siRNA molecule of claim 34, wherein the sense strand comprises a structure represented by Formula S2, wherein Formula S2 is, in the 5′-to-3′ direction: 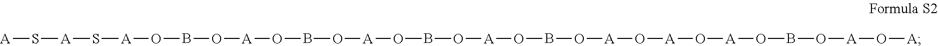
wherein A represents a 2′-O-Me ribonucleoside, B represents a 2′-F ribonucleoside, O represents a phosphodiester internucleoside linkage, and S represents a phosphorothioate internucleoside linkage.

37. The siRNA molecule of claim 34, wherein the sense strand comprises a structure represented by Formula S3, wherein Formula S3 is, in the 5′-to-3′ direction: 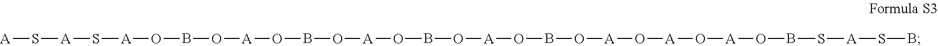
wherein A represents a 2′-O-Me ribonucleoside, B represents a 2′-F ribonucleoside, O represents a phosphodiester internucleoside linkage, and S represents a phosphorothioate internucleoside linkage.

38. The siRNA molecule of claim 34, wherein the sense strand comprises a structure represented by Formula S4, wherein Formula S4 is, in the 5′-to-3′ direction: 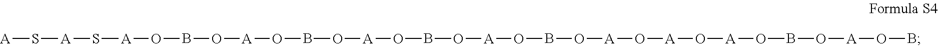
wherein A represents a 2′-O-Me ribonucleoside, B represents a 2′-F ribonucleoside, O represents a phosphodiester internucleoside linkage, and S represents a phosphorothioate internucleoside linkage.

39. The siRNA molecule of any one of claims 1-32 and 34-38, wherein the antisense strand comprises a structure represented by Formula IV, wherein Formula IV is, in the 5′-to-3′ direction: 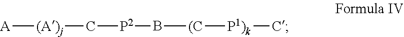
wherein A is represented by the formula C—P¹-D-P¹;
each A′ is represented by the formula C—P²-D-P²;
B is represented by the formula D-P¹—C—P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

40. The siRNA molecule of claim 39, wherein the antisense strand comprises a structure represented by Formula A3, wherein Formula A3 is, in the 5′-to-3′ direction: 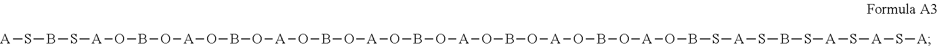
wherein A represents a 2′-O-Me ribonucleoside, B represents a 2′-F ribonucleoside, O represents a phosphodiester internucleoside linkage, and S represents a phosphorothioate internucleoside linkage.

41. The siRNA molecule of any one of claims 1-33, 39, and 40, wherein the sense strand comprises a structure represented by Formula V, wherein Formula V is, in the 5′-to-3′ direction: 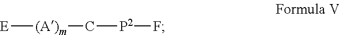
wherein E is represented by the formula (C—P¹)₂;
F is represented by the formula D-P¹—C—P¹—C, D-P²—C—P²—C, D-P¹—C—P¹-D, or D-P²—C—P²-D;
A′, C, D, P¹ and P² are as defined in Formula IV; and
m is an integer from 1 to 7.

42. The siRNA molecule of claim 41, wherein the sense strand comprises a structure represented by Formula S5, wherein Formula S5 is, in the 5′-to-3′ direction: 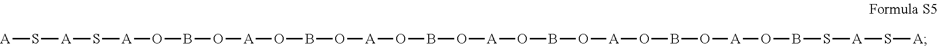
wherein A represents a 2′-O-Me ribonucleoside, B represents a 2′-F ribonucleoside, O represents a phosphodiester internucleoside linkage, and S represents a phosphorothioate internucleoside linkage.

43. The siRNA molecule of claim 41, wherein the sense strand comprises a structure represented by Formula S6, wherein Formula S6 is, in the 5′-to-3′ direction: 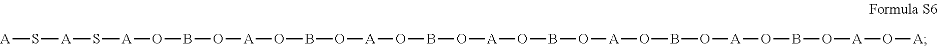
wherein A represents a 2′-O-Me ribonucleoside, B represents a 2′-F ribonucleoside, O represents a phosphodiester internucleoside linkage, and S represents a phosphorothioate internucleoside linkage.

44. The siRNA molecule of claim 41, wherein the sense strand comprises a structure represented by Formula S7, wherein Formula S7 is, in the 5′-to-3′ direction: 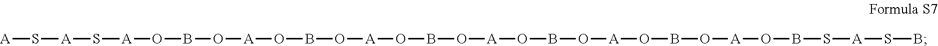
wherein A represents a 2′-O-Me ribonucleoside, B represents a 2′-F ribonucleoside, O represents a phosphodiester internucleoside linkage, and S represents a phosphorothioate internucleoside linkage.

45. The siRNA molecule of claim 41, wherein the sense strand comprises a structure represented by Formula S8, wherein Formula S8 is, in the 5′-to-3′ direction: 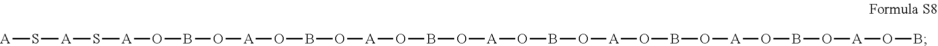
wherein A represents a 2′-O-Me ribonucleoside, B represents a 2′-F ribonucleoside, O represents a phosphodiester internucleoside linkage, and S represents a phosphorothioate internucleoside linkage.

46. The siRNA molecule of any one of claims 1-32, 34-38 and 41-45, wherein the antisense strand comprises a structure represented by Formula VI, wherein Formula VI is, in the 5′-to-3′ direction: 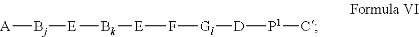
wherein A is represented by the formula C—P¹-D-P¹;
each B is represented by the formula C—P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²—C—P²;
F is represented by the formula D-P¹—C—P¹;
each G is represented by the formula C—P¹;
each P¹ is a phosphorothioate internucleoside linkage;
each P² is a phosphodiester internucleoside linkage;
j is an integer from 1 to 7;
k is an integer from 1 to 7; and
I is an integer from 1 to 7.

47. The siRNA molecule of claim 46, wherein the antisense strand comprises a structure represented by Formula A4, wherein Formula A4 is, in the 5′-to-3′ direction: 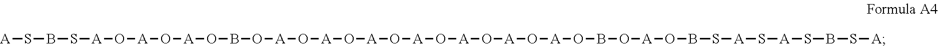
wherein A represents a 2′-O-Me ribonucleoside, B represents a 2′-F ribonucleoside, O represents a phosphodiester internucleoside linkage, and S represents a phosphorothioate internucleoside linkage.

48. The siRNA molecule of any one of claims 1-33, 39, 40, 46, and 47, wherein the sense strand comprises a structure represented by Formula VII, wherein Formula VII is, in the 5′-to-3′ direction: 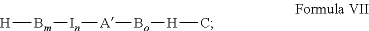
wherein A′ is represented by the formula C—P²-D-P²;
each H is represented by the formula (C—P¹)₂;
each I is represented by the formula (D-P²);
B, C, D, P¹ and P² are as defined in Formula VI;
m is an integer from 1 to 7;
n is an integer from 1 to 7; and
o is an integer from 1 to 7.

49. The siRNA molecule of claim 48, wherein the sense strand comprises a structure represented by Formula S9, wherein Formula S9 is, in the 5′-to-3′ direction: 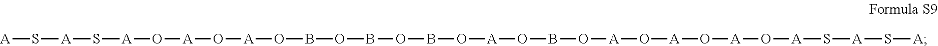
wherein A represents a 2′-O-Me ribonucleoside, B represents a 2′-F ribonucleoside, O represents a phosphodiester internucleoside linkage, and S represents a phosphorothioate internucleoside linkage.

50. The siRNA molecule of any one of claims 1-49, wherein the antisense strand further comprises a 5′ phosphorus stabilizing moiety at the 5′ end of the antisense strand.

51. The siRNA molecule of any one of claims 1-50, wherein the sense strand further comprises a 5′ phosphorus stabilizing moiety at the 5′ end of the sense strand.

52. The siRNA molecule of claim 50 or 51, wherein each 5′ phosphorus stabilizing moiety is, independently, represented by any one of Formulas IX-XVI: 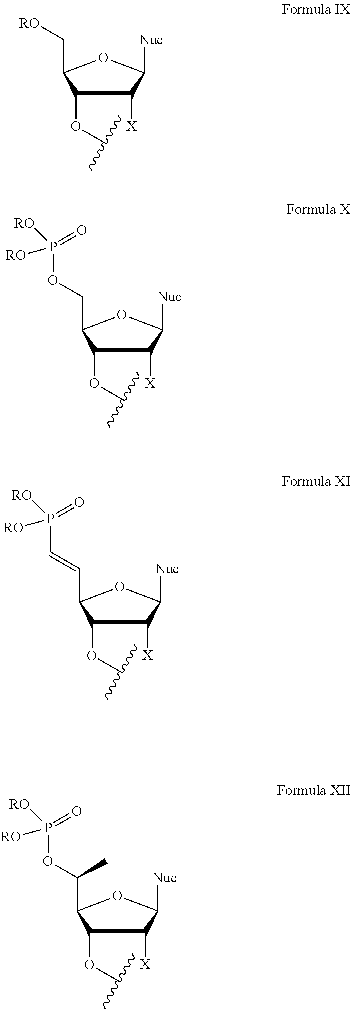 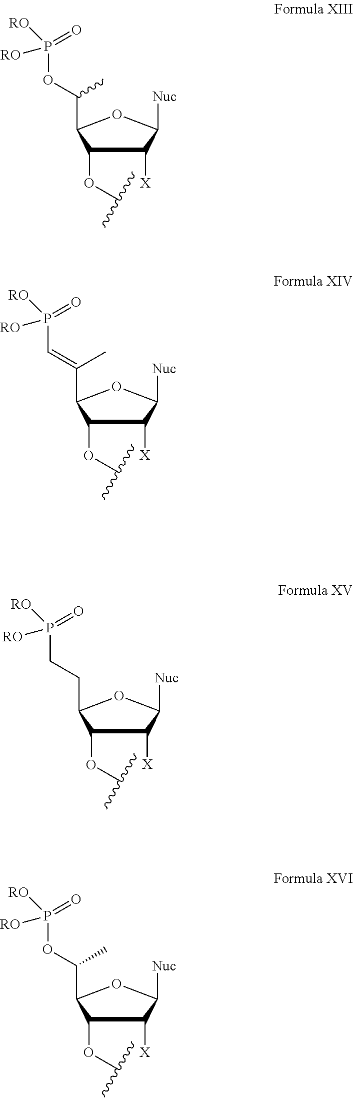
wherein Nuc represents a nucleobase selected from the group consisting of adenine, uracil, guanine, thymine, and cytosine, and R represents an optionally substituted alkyl, optionally substituted alkenyl, optionally substituted alkynyl, phenyl, benzyl, hydroxy, or hydrogen.

53. The siRNA molecule of claim 52, wherein the nucleobase is an adenine, uracil, guanine, thymine, or cytosine.

54. The siRNA molecule of any one of claims 50-53, wherein the 5′ phosphorus stabilizing moiety is (E)-vinylphosphonate represented by Formula XI.

55. The siRNA molecule of any one of claims 1-54, wherein the siRNA molecule further comprises a hydrophobic moiety at the 5′ or the 3′ end of the siRNA molecule.

56. The siRNA molecule of claim 55, wherein the hydrophobic moiety is selected from a group consisting of cholesterol, vitamin D, or tocopherol.

57. The siRNA molecule of any one of claims 1-56, wherein the length of the sense strand is between 12 and 30 nucleotides.

58. The siRNA molecule of any one of claims 1-57, wherein the siRNA molecule is a branched siRNA molecule.

59. The siRNA molecule of claim 58, wherein the branched siRNA molecule is di-branched, tri-branched, or tetra-branched.

60. The siRNA molecule of claim 59, wherein the siRNA molecule is a di-branched siRNA molecule, optionally wherein the di-branched siRNA molecule is represented by any one of Formulas XVII-XIX: 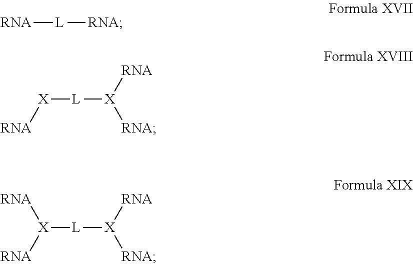
wherein each RNA is, independently, an siRNA molecule, L is a linker, and each X, independently, represents a branch point moiety.

61. The siRNA molecule of claim 59, wherein the siRNA molecule is a tri-branched siRNA molecule, optionally wherein the tri-branched siRNA molecule is represented by any one of Formulas XX-XXIII: 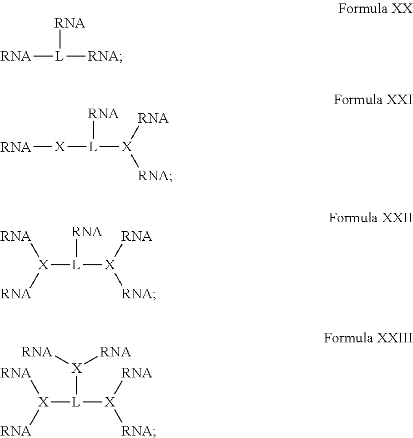
wherein each RNA is, independently, an siRNA molecule, L is a linker, and each X, independently, represents a branch point moiety.

62. The siRNA molecule of claim 59, wherein the siRNA molecule is a tetra-branched siRNA molecule, optionally wherein the tetra-branched siRNA molecule is represented by any one of Formulas XXIV-XXVIII: 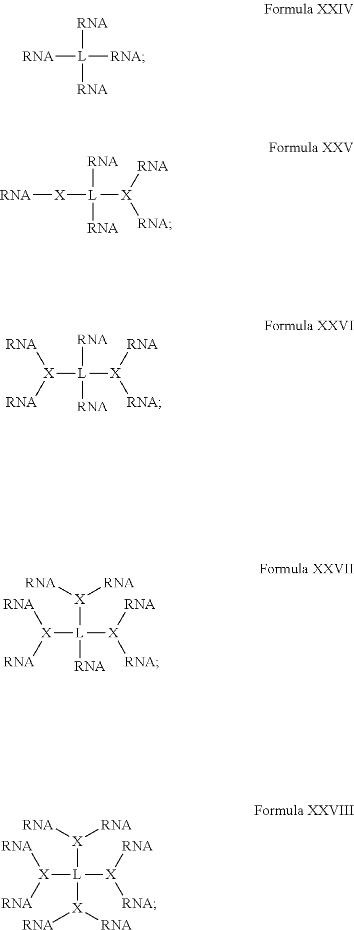
wherein each RNA is, independently, an siRNA molecule, L is a linker, and each X, independently, represents a branch point moiety.

63. The siRNA molecule of any one of claims 60-62, wherein the linker is selected from a group consisting of one or more contiguous subunits of an ethylene glycol, alkyl, carbohydrate, block copolymer, peptide, RNA, and DNA.

64. The siRNA molecule of claim 63, wherein the one or more contiguous subunits is 2 to 20 contiguous subunits.

65. A pharmaceutical composition comprising the siRNA molecule of any one of claims 1-64 and a pharmaceutically acceptable excipient, carrier, or diluent.

66. A method of delivering an siRNA molecule to a subject diagnosed as having a microsatellite repeat expansion disorder, the method comprising administering a therapeutically effective amount of the siRNA molecule of any one of claims 1-64 or the pharmaceutical composition of claim 65 to the subject.

67. A method of treating a microsatellite repeat expansion disorder in a subject in need thereof, the method comprising administering a therapeutically effective amount of the siRNA molecule of any one of claims 1-64 or the pharmaceutical composition of claim 65 to the subject.

68. The method of claim 67, wherein the microsatellite repeat expansion disorder is Huntington's disease.

69. The method of claim 67, wherein the microsatellite repeat expansion disorder is a spinocerebellar ataxia.

70. The method of claim 67, wherein the microsatellite repeat expansion disorder is a Fragile X syndrome.

71. The method of claim 67, wherein the microsatellite repeat expansion disorder is a myotonic dystrophy.

72. A method of reducing MSH3 expression in a subject in need thereof, the method comprising administering a therapeutically effective amount of the siRNA molecule of any one of claims 1-64 or the pharmaceutical composition of claim 65 to the subject.

73. The method of any one of claims 66-72, wherein the siRNA molecule or the pharmaceutical composition is administered to the subject by way of intracerebroventricular, intrastriatal, intraparenchymal, or intrathecal injection.

74. The method of any one of claims 66-73, wherein the siRNA molecule or the pharmaceutical composition is administered to the subject by way of intravenous, intramuscular, or subcutaneous injection.

75. The method of any one of claims 66-74, wherein the subject is a human.

76. A kit comprising the siRNA molecule of any one of claims 1-64, or the pharmaceutical composition of claim 65, and a package insert, wherein the package insert instructs a user of the kit to perform the method of any one of claims 66-75.


SEQUENCE LISTING (1224 sequences)

SEQ ID NO: 1 - RNA, 45 nt, Homo sapiens
  source 1..45: mol_type = mRNA
gcgaggcaagcggttttgagccgattcttccagtctacgggaagc

SEQ ID NO: 2 - RNA, 45 nt, Homo sapiens
  source 1..45: mol_type = mRNA
ccgattcttccagtctacgggaagcctgaaatccacctcctcctc

SEQ ID NO: 3 - RNA, 45 nt, Homo sapiens
  source 1..45: mol_type = mRNA
cttccagtctacgggaagcctgaaatccacctcctcctccacagg

SEQ ID NO: 4 - RNA, 45 nt, Homo sapiens
  source 1..45: mol_type = mRNA
agtctacgggaagcctgaaatccacctcctcctccacaggtgcag

SEQ ID NO: 5 - RNA, 45 nt, Homo sapiens
  source 1..45: mol_type = mRNA
gccgcacatagctacagaaattgacagaagaaagaagagaccatt

SEQ ID NO: 6 - RNA, 45 nt, Homo sapiens
  source 1..45: mol_type = mRNA
ccgcacatagctacagaaattgacagaagaaagaagagaccattg

SEQ ID NO: 7 - RNA, 45 nt, Homo sapiens
  source 1..45: mol_type = mRNA
cgcacatagctacagaaattgacagaagaaagaagagaccattgg

SEQ ID NO: 8 - RNA, 45 nt, Homo sapiens
  source 1..45: mol_type = mRNA
gcacatagctacagaaattgacagaagaaagaagagaccattgga

SEQ ID NO: 9 - RNA, 45 nt, Homo sapiens
  source 1..45: mol_type = mRNA
cacatagctacagaaattgacagaagaaagaagagaccattggaa

SEQ ID NO: 10 - RNA, 45 nt, Homo sapiens
  source 1..45: mol_type = mRNA
atagctacagaaattgacagaagaaagaagagaccattggaaaat

SEQ ID NO: 11 - RNA, 45 nt, Homo sapiens
  source 1..45: mol_type = mRNA
aagaaagaagagaccattggaaaatgatgggcctgttaaaaagaa

SEQ ID NO: 12 - RNA, 45 nt, Homo sapiens
  source 1..45: mol_type = mRNA
agaaagaagagaccattggaaaatgatgggcctgttaaaaagaaa

SEQ ID NO: 13 - RNA, 45 nt, Homo sapiens
  source 1..45: mol_type = mRNA
gaaagaagagaccattggaaaatgatgggcctgttaaaaagaaag

SEQ ID NO: 14 - RNA, 45 nt, Homo sapiens
  source 1..45: mol_type = mRNA
aaagaagagaccattggaaaatgatgggcctgttaaaaagaaagt

SEQ ID NO: 15 - RNA, 45 nt, Homo sapiens
  source 1..45: mol_type = mRNA
aagaagagaccattggaaaatgatgggcctgttaaaaagaaagta

SEQ ID NO: 16 - RNA, 45 nt, Homo sapiens
  source 1..45: mol_type = mRNA
gatctgggaatgtctggcaactctgagccaaagaaatgtctgagg

SEQ ID NO: 17 - RNA, 45 nt, Homo sapiens
  source 1..45: mol_type = mRNA
gaatgtctggcaactctgagccaaagaaatgtctgaggaccagga

SEQ ID NO: 18 - RNA, 45 nt, Homo sapiens
  source 1..45: mol_type = mRNA
aatgtctggcaactctgagccaaagaaatgtctgaggaccaggaa

SEQ ID NO: 19 - RNA, 45 nt, Homo sapiens
  source 1..45: mol_type = mRNA
atgtctggcaactctgagccaaagaaatgtctgaggaccaggaat

SEQ ID NO: 20 - RNA, 45 nt, Homo sapiens
  source 1..45: mol_type = mRNA
tgtctggcaactctgagccaaagaaatgtctgaggaccaggaatg

SEQ ID NO: 21 - RNA, 45 nt, Homo sapiens
  source 1..45: mol_type = mRNA
gtctggcaactctgagccaaagaaatgtctgaggaccaggaatgt

SEQ ID NO: 22 - RNA, 45 nt, Homo sapiens
  source 1..45: mol_type = mRNA
tctggcaactctgagccaaagaaatgtctgaggaccaggaatgtt

SEQ ID NO: 23 - RNA, 45 nt, Homo sapiens
  source 1..45: mol_type = mRNA
ctggcaactctgagccaaagaaatgtctgaggaccaggaatgttt

SEQ ID NO: 24 - RNA, 45 nt, Homo sapiens
  source 1..45: mol_type = mRNA
ttgaaagaattctgctgcgattctgcccttcctcaaagtagagtc

SEQ ID NO: 25 - RNA, 45 nt, Homo sapiens
  source 1..45: mol_type = mRNA
tccagacagaatctctgcaggagagatttgcagttctgccaaaat

SEQ ID NO: 26 - RNA, 45 nt, Homo sapiens
  source 1..45: mol_type = mRNA
ccagacagaatctctgcaggagagatttgcagttctgccaaaatg

SEQ ID NO: 27 - RNA, 45 nt, Homo sapiens
  source 1..45: mol_type = mRNA
cagacagaatctctgcaggagagatttgcagttctgccaaaatgt

SEQ ID NO: 28 - RNA, 45 nt, Homo sapiens
  source 1..45: mol_type = mRNA
agacagaatctctgcaggagagatttgcagttctgccaaaatgta

SEQ ID NO: 29 - RNA, 45 nt, Homo sapiens
  source 1..45: mol_type = mRNA
gacagaatctctgcaggagagatttgcagttctgccaaaatgtac

SEQ ID NO: 30 - RNA, 45 nt, Homo sapiens
  source 1..45: mol_type = mRNA
gcaggagagatttgcagttctgccaaaatgtactgattttgatga

SEQ ID NO: 31 - RNA, 45 nt, Homo sapiens
  source 1..45: mol_type = mRNA
caggagagatttgcagttctgccaaaatgtactgattttgatgat

SEQ ID NO: 32 - RNA, 45 nt, Homo sapiens
  source 1..45: mol_type = mRNA
aggagagatttgcagttctgccaaaatgtactgattttgatgata

SEQ ID NO: 33 - RNA, 45 nt, Homo sapiens
  source 1..45: mol_type = mRNA
ggagagatttgcagttctgccaaaatgtactgattttgatgatat

SEQ ID NO: 34 - RNA, 45 nt, Homo sapiens
  source 1..45: mol_type = mRNA
gagagatttgcagttctgccaaaatgtactgattttgatgatatc

SEQ ID NO: 35 - RNA, 45 nt, Homo sapiens
  source 1..45: mol_type = mRNA
agagatttgcagttctgccaaaatgtactgattttgatgatatca

SEQ ID NO: 36 - RNA, 45 nt, Homo sapiens
  source 1..45: mol_type = mRNA
gagatttgcagttctgccaaaatgtactgattttgatgatatcag

SEQ ID NO: 37 - RNA, 45 nt, Homo sapiens
  source 1..45: mol_type = mRNA
gattttgatgatatcagtcttctacacgcaaagaatgcagtttct

SEQ ID NO: 38 - RNA, 45 nt, Homo sapiens
  source 1..45: mol_type = mRNA
attttgatgatatcagtcttctacacgcaaagaatgcagtttctt

SEQ ID NO: 39 - RNA, 45 nt, Homo sapiens
  source 1..45: mol_type = mRNA
atgatatcagtcttctacacgcaaagaatgcagtttcttctgaag

SEQ ID NO: 40 - RNA, 45 nt, Homo sapiens
  source 1..45: mol_type = mRNA
tgatatcagtcttctacacgcaaagaatgcagtttcttctgaaga

SEQ ID NO: 41 - RNA, 45 nt, Homo sapiens
  source 1..45: mol_type = mRNA
gatatcagtcttctacacgcaaagaatgcagtttcttctgaagat

SEQ ID NO: 42 - RNA, 45 nt, Homo sapiens
  source 1..45: mol_type = mRNA
atatcagtcttctacacgcaaagaatgcagtttcttctgaagatt

SEQ ID NO: 43 - RNA, 45 nt, Homo sapiens
  source 1..45: mol_type = mRNA
tatcagtcttctacacgcaaagaatgcagtttcttctgaagattc

SEQ ID NO: 44 - RNA, 45 nt, Homo sapiens
  source 1..45: mol_type = mRNA
atcagtcttctacacgcaaagaatgcagtttcttctgaagattcg

SEQ ID NO: 45 - RNA, 45 nt, Homo sapiens
  source 1..45: mol_type = mRNA
tcagtcttctacacgcaaagaatgcagtttcttctgaagattcga

SEQ ID NO: 46 - RNA, 45 nt, Homo sapiens
  source 1..45: mol_type = mRNA
atgcagtttcttctgaagattcgaaacgtcaaattaatcaaaagg

SEQ ID NO: 47 - RNA, 45 nt, Homo sapiens
  source 1..45: mol_type = mRNA
tttcttctgaagattcgaaacgtcaaattaatcaaaaggacacaa

SEQ ID NO: 48 - RNA, 45 nt, Homo sapiens
  source 1..45: mol_type = mRNA
aaggacacaacactttttgatctcagtcagtttggatcatcaaat

SEQ ID NO: 49 - RNA, 45 nt, Homo sapiens
  source 1..45: mol_type = mRNA
caacactttttgatctcagtcagtttggatcatcaaatacaagtc

SEQ ID NO: 50 - RNA, 45 nt, Homo sapiens
  source 1..45: mol_type = mRNA
acactttttgatctcagtcagtttggatcatcaaatacaagtcat

SEQ ID NO: 51 - RNA, 45 nt, Homo sapiens
  source 1..45: mol_type = mRNA
tttttgatctcagtcagtttggatcatcaaatacaagtcatgaaa

SEQ ID NO: 52 - RNA, 45 nt, Homo sapiens
  source 1..45: mol_type = mRNA
tgatctcagtcagtttggatcatcaaatacaagtcatgaaaattt

SEQ ID NO: 53 - RNA, 45 nt, Homo sapiens
  source 1..45: mol_type = mRNA
atacaagtcatgaaaatttacagaaaactgcttccaaatcagcta

SEQ ID NO: 54 - RNA, 45 nt, Homo sapiens
  source 1..45: mol_type = mRNA
agtcatgaaaatttacagaaaactgcttccaaatcagctaacaaa

SEQ ID NO: 55 - RNA, 45 nt, Homo sapiens
  source 1..45: mol_type = mRNA
gtcatgaaaatttacagaaaactgcttccaaatcagctaacaaac

SEQ ID NO: 56 - RNA, 45 nt, Homo sapiens
  source 1..45: mol_type = mRNA
tcatgaaaatttacagaaaactgcttccaaatcagctaacaaacg

SEQ ID NO: 57 - RNA, 45 nt, Homo sapiens
  source 1..45: mol_type = mRNA
catgaaaatttacagaaaactgcttccaaatcagctaacaaacgg

SEQ ID NO: 58 - RNA, 45 nt, Homo sapiens
  source 1..45: mol_type = mRNA
atgaaaatttacagaaaactgcttccaaatcagctaacaaacggt

SEQ ID NO: 59 - RNA, 45 nt, Homo sapiens
  source 1..45: mol_type = mRNA
tttacagaaaactgcttccaaatcagctaacaaacggtccaaaag

SEQ ID NO: 60 - RNA, 45 nt, Homo sapiens
  source 1..45: mol_type = mRNA
ttacagaaaactgcttccaaatcagctaacaaacggtccaaaagc

SEQ ID NO: 61 - RNA, 45 nt, Homo sapiens
  source 1..45: mol_type = mRNA
tacagaaaactgcttccaaatcagctaacaaacggtccaaaagca

SEQ ID NO: 62 - RNA, 45 nt, Homo sapiens
  source 1..45: mol_type = mRNA
acagaaaactgcttccaaatcagctaacaaacggtccaaaagcat

SEQ ID NO: 63 - RNA, 45 nt, Homo sapiens
  source 1..45: mol_type = mRNA
cagaaaactgcttccaaatcagctaacaaacggtccaaaagcatc

SEQ ID NO: 64 - RNA, 45 nt, Homo sapiens
  source 1..45: mol_type = mRNA
agaaaactgcttccaaatcagctaacaaacggtccaaaagcatct

SEQ ID NO: 65 - RNA, 45 nt, Homo sapiens
  source 1..45: mol_type = mRNA
gaaaactgcttccaaatcagctaacaaacggtccaaaagcatcta

SEQ ID NO: 66 - RNA, 45 nt, Homo sapiens
  source 1..45: mol_type = mRNA
tgcttccaaatcagctaacaaacggtccaaaagcatctatacgcc

SEQ ID NO: 67 - RNA, 45 nt, Homo sapiens
  source 1..45: mol_type = mRNA
cttccaaatcagctaacaaacggtccaaaagcatctatacgccgc

SEQ ID NO: 68 - RNA, 45 nt, Homo sapiens
  source 1..45: mol_type = mRNA
aatcagctaacaaacggtccaaaagcatctatacgccgctagaat

SEQ ID NO: 69 - RNA, 45 nt, Homo sapiens
  source 1..45: mol_type = mRNA
atcagctaacaaacggtccaaaagcatctatacgccgctagaatt

SEQ ID NO: 70 - RNA, 45 nt, Homo sapiens
  source 1..45: mol_type = mRNA
tcagctaacaaacggtccaaaagcatctatacgccgctagaatta

SEQ ID NO: 71 - RNA, 45 nt, Homo sapiens
  source 1..45: mol_type = mRNA
cagctaacaaacggtccaaaagcatctatacgccgctagaattac

SEQ ID NO: 72 - RNA, 45 nt, Homo sapiens
  source 1..45: mol_type = mRNA
atagaaatgaagcagcagcacaaagatgcagttttgtgtgtggaa

SEQ ID NO: 73 - RNA, 45 nt, Homo sapiens
  source 1..45: mol_type = mRNA
tagaaatgaagcagcagcacaaagatgcagttttgtgtgtggaat

SEQ ID NO: 74 - RNA, 45 nt, Homo sapiens
  source 1..45: mol_type = mRNA
caaagatgcagttttgtgtgtggaatgtggatataagtatagatt

SEQ ID NO: 75 - RNA, 45 nt, Homo sapiens
  source 1..45: mol_type = mRNA
aaagatgcagttttgtgtgtggaatgtggatataagtatagattc

SEQ ID NO: 76 - RNA, 45 nt, Homo sapiens
  source 1..45: mol_type = mRNA
aagatgcagttttgtgtgtggaatgtggatataagtatagattct

SEQ ID NO: 77 - RNA, 45 nt, Homo sapiens
  source 1..45: mol_type = mRNA
agatgcagttttgtgtgtggaatgtggatataagtatagattctt

SEQ ID NO: 78 - RNA, 45 nt, Homo sapiens
  source 1..45: mol_type = mRNA
gatgcagttttgtgtgtggaatgtggatataagtatagattcttt

SEQ ID NO: 79 - RNA, 45 nt, Homo sapiens
  source 1..45: mol_type = mRNA
atgcagttttgtgtgtggaatgtggatataagtatagattctttg

SEQ ID NO: 80 - RNA, 45 nt, Homo sapiens
  source 1..45: mol_type = mRNA
agttttgtgtgtggaatgtggatataagtatagattctttgggga

SEQ ID NO: 81 - RNA, 45 nt, Homo sapiens
  source 1..45: mol_type = mRNA
gttttgtgtgtggaatgtggatataagtatagattctttggggaa

SEQ ID NO: 82 - RNA, 45 nt, Homo sapiens
  source 1..45: mol_type = mRNA
ttttgtgtgtggaatgtggatataagtatagattctttggggaag

SEQ ID NO: 83 - RNA, 45 nt, Homo sapiens
  source 1..45: mol_type = mRNA
tttgtgtgtggaatgtggatataagtatagattctttggggaaga

SEQ ID NO: 84 - RNA, 45 nt, Homo sapiens
  source 1..45: mol_type = mRNA
ttgtgtgtggaatgtggatataagtatagattctttggggaagat

SEQ ID NO: 85 - RNA, 45 nt, Homo sapiens
  source 1..45: mol_type = mRNA
ccgagagctcaatatttattgccatttagatcacaactttatgac

SEQ ID NO: 86 - RNA, 45 nt, Homo sapiens
  source 1..45: mol_type = mRNA
agagctcaatatttattgccatttagatcacaactttatgacagc

SEQ ID NO: 87 - RNA, 45 nt, Homo sapiens
  source 1..45: mol_type = mRNA
gagctcaatatttattgccatttagatcacaactttatgacagca

SEQ ID NO: 88 - RNA, 45 nt, Homo sapiens
  source 1..45: mol_type = mRNA
agctcaatatttattgccatttagatcacaactttatgacagcaa

SEQ ID NO: 89 - RNA, 45 nt, Homo sapiens
  source 1..45: mol_type = mRNA
gctcaatatttattgccatttagatcacaactttatgacagcaag

SEQ ID NO: 90 - RNA, 45 nt, Homo sapiens
  source 1..45: mol_type = mRNA
ctcaatatttattgccatttagatcacaactttatgacagcaagt

SEQ ID NO: 91 - RNA, 45 nt, Homo sapiens
  source 1..45: mol_type = mRNA
tcaatatttattgccatttagatcacaactttatgacagcaagta

SEQ ID NO: 92 - RNA, 45 nt, Homo sapiens
  source 1..45: mol_type = mRNA
caatatttattgccatttagatcacaactttatgacagcaagtat

SEQ ID NO: 93 - RNA, 45 nt, Homo sapiens
  source 1..45: mol_type = mRNA
atttattgccatttagatcacaactttatgacagcaagtatacct

SEQ ID NO: 94 - RNA, 45 nt, Homo sapiens
  source 1..45: mol_type = mRNA
tttattgccatttagatcacaactttatgacagcaagtataccta

SEQ ID NO: 95 - RNA, 45 nt, Homo sapiens
  source 1..45: mol_type = mRNA
ttattgccatttagatcacaactttatgacagcaagtatacctac

SEQ ID NO: 96 - RNA, 45 nt, Homo sapiens
  source 1..45: mol_type = mRNA
tattgccatttagatcacaactttatgacagcaagtatacctact

SEQ ID NO: 97 - RNA, 45 nt, Homo sapiens
  source 1..45: mol_type = mRNA
attgccatttagatcacaactttatgacagcaagtatacctactc

SEQ ID NO: 98 - RNA, 45 nt, Homo sapiens
  source 1..45: mol_type = mRNA
ttgccatttagatcacaactttatgacagcaagtatacctactca

SEQ ID NO: 99 - RNA, 45 nt, Homo sapiens
  source 1..45: mol_type = mRNA
tgccatttagatcacaactttatgacagcaagtatacctactcac

SEQ ID NO: 100 - RNA, 45 nt, Homo sapiens
  source 1..45: mol_type = mRNA
tttatgacagcaagtatacctactcacagactgtttgttcatgta

SEQ ID NO: 101 - RNA, 45 nt, Homo sapiens
  source 1..45: mol_type = mRNA
gtggcaaaaggatataaggtgggagttgtgaagcaaactgaaact

SEQ ID NO: 102 - RNA, 45 nt, Homo sapiens
  source 1..45: mol_type = mRNA
tggcaaaaggatataaggtgggagttgtgaagcaaactgaaactg

SEQ ID NO: 103 - RNA, 45 nt, Homo sapiens
  source 1..45: mol_type = mRNA
ggcaaaaggatataaggtgggagttgtgaagcaaactgaaactgc

SEQ ID NO: 104 - RNA, 45 nt, Homo sapiens
  source 1..45: mol_type = mRNA
caaaaggatataaggtgggagttgtgaagcaaactgaaactgcag

SEQ ID NO: 105 - RNA, 45 nt, Homo sapiens
  source 1..45: mol_type = mRNA
aaaaggatataaggtgggagttgtgaagcaaactgaaactgcagc

SEQ ID NO: 106 - RNA, 45 nt, Homo sapiens
  source 1..45: mol_type = mRNA
aaaggatataaggtgggagttgtgaagcaaactgaaactgcagca

SEQ ID NO: 107 - RNA, 45 nt, Homo sapiens
  source 1..45: mol_type = mRNA
aaggatataaggtgggagttgtgaagcaaactgaaactgcagcat

SEQ ID NO: 108 - RNA, 45 nt, Homo sapiens
  source 1..45: mol_type = mRNA
aggatataaggtgggagttgtgaagcaaactgaaactgcagcatt

SEQ ID NO: 109 - RNA, 45 nt, Homo sapiens
  source 1..45: mol_type = mRNA
ggatataaggtgggagttgtgaagcaaactgaaactgcagcatta

SEQ ID NO: 110 - RNA, 45 nt, Homo sapiens
  source 1..45: mol_type = mRNA
gatataaggtgggagttgtgaagcaaactgaaactgcagcattaa

SEQ ID NO: 111 - RNA, 45 nt, Homo sapiens
  source 1..45: mol_type = mRNA
atataaggtgggagttgtgaagcaaactgaaactgcagcattaaa

SEQ ID NO: 112 - RNA, 45 nt, Homo sapiens
  source 1..45: mol_type = mRNA
tataaggtgggagttgtgaagcaaactgaaactgcagcattaaag

SEQ ID NO: 113 - RNA, 45 nt, Homo sapiens
  source 1..45: mol_type = mRNA
ataaggtgggagttgtgaagcaaactgaaactgcagcattaaagg

SEQ ID NO: 114 - RNA, 45 nt, Homo sapiens
  source 1..45: mol_type = mRNA
taaggtgggagttgtgaagcaaactgaaactgcagcattaaaggc

SEQ ID NO: 115 - RNA, 45 nt, Homo sapiens
  source 1..45: mol_type = mRNA
aaggtgggagttgtgaagcaaactgaaactgcagcattaaaggcc

SEQ ID NO: 116 - RNA, 45 nt, Homo sapiens
  source 1..45: mol_type = mRNA
aggtgggagttgtgaagcaaactgaaactgcagcattaaaggcca

SEQ ID NO: 117 - RNA, 45 nt, Homo sapiens
  source 1..45: mol_type = mRNA
cagcattaaaggccattggagacaacagaagttcactcttttccc

SEQ ID NO: 118 - RNA, 45 nt, Homo sapiens
  source 1..45: mol_type = mRNA
gaagttcactcttttcccggaaattgactgccctttatacaaaat

SEQ ID NO: 119 - RNA, 45 nt, Homo sapiens
  source 1..45: mol_type = mRNA
ttcactcttttcccggaaattgactgccctttatacaaaatctac

SEQ ID NO: 120 - RNA, 45 nt, Homo sapiens
  source 1..45: mol_type = mRNA
tcttttcccggaaattgactgccctttatacaaaatctacactta

SEQ ID NO: 121 - RNA, 45 nt, Homo sapiens
  source 1..45: mol_type = mRNA
gtgaatcccctaatcaagctggatgatgctgtaaatgttgatgag

SEQ ID NO: 122 - RNA, 45 nt, Homo sapiens
  source 1..45: mol_type = mRNA
ctggatgatgctgtaaatgttgatgagataatgactgatacttct

SEQ ID NO: 123 - RNA, 45 nt, Homo sapiens
  source 1..45: mol_type = mRNA
tgctgtaaatgttgatgagataatgactgatacttctaccagcta

SEQ ID NO: 124 - RNA, 45 nt, Homo sapiens
  source 1..45: mol_type = mRNA
ttctaccagctatcttctgtgcatctctgaaaataaggaaaatgt

SEQ ID NO: 125 - RNA, 45 nt, Homo sapiens
  source 1..45: mol_type = mRNA
tctaccagctatcttctgtgcatctctgaaaataaggaaaatgtt

SEQ ID NO: 126 - RNA, 45 nt, Homo sapiens
  source 1..45: mol_type = mRNA
ctaccagctatcttctgtgcatctctgaaaataaggaaaatgtta

SEQ ID NO: 127 - RNA, 45 nt, Homo sapiens
  source 1..45: mol_type = mRNA
taccagctatcttctgtgcatctctgaaaataaggaaaatgttag

SEQ ID NO: 128 - RNA, 45 nt, Homo sapiens
  source 1..45: mol_type = mRNA
accagctatcttctgtgcatctctgaaaataaggaaaatgttagg

SEQ ID NO: 129 - RNA, 45 nt, Homo sapiens
  source 1..45: mol_type = mRNA
ccagctatcttctgtgcatctctgaaaataaggaaaatgttaggg

SEQ ID NO: 130 - RNA, 45 nt, Homo sapiens
  source 1..45: mol_type = mRNA
cagctatcttctgtgcatctctgaaaataaggaaaatgttaggga

SEQ ID NO: 131 - RNA, 45 nt, Homo sapiens
  source 1..45: mol_type = mRNA
agctatcttctgtgcatctctgaaaataaggaaaatgttagggac

SEQ ID NO: 132 - RNA, 45 nt, Homo sapiens
  source 1..45: mol_type = mRNA
ctatcttctgtgcatctctgaaaataaggaaaatgttagggacaa

SEQ ID NO: 133 - RNA, 45 nt, Homo sapiens
  source 1..45: mol_type = mRNA
tatcttctgtgcatctctgaaaataaggaaaatgttagggacaaa

SEQ ID NO: 134 - RNA, 45 nt, Homo sapiens
  source 1..45: mol_type = mRNA
ctcgttcagagctagaaacccggatgtcaagcctgcagccagtag

SEQ ID NO: 135 - RNA, 45 nt, Homo sapiens
  source 1..45: mol_type = mRNA
tcagagctagaaacccggatgtcaagcctgcagccagtagagctg

SEQ ID NO: 136 - RNA, 45 nt, Homo sapiens
  source 1..45: mol_type = mRNA
ctagaaacccggatgtcaagcctgcagccagtagagctgctgctt

SEQ ID NO: 137 - RNA, 45 nt, Homo sapiens
  source 1..45: mol_type = mRNA
gtagagctgctgcttccttcggccttgtccgagcaaacagaggcg

SEQ ID NO: 138 - RNA, 45 nt, Homo sapiens
  source 1..45: mol_type = mRNA
ttcggccttgtccgagcaaacagaggcgctcatccacagagccac

SEQ ID NO: 139 - RNA, 45 nt, Homo sapiens
  source 1..45: mol_type = mRNA
agcaaacagaggcgctcatccacagagccacatctgttagtgtgc

SEQ ID NO: 140 - RNA, 45 nt, Homo sapiens
  source 1..45: mol_type = mRNA
gatggataacatttattttgaatacagccatgctttccaggcagt

SEQ ID NO: 141 - RNA, 45 nt, Homo sapiens
  source 1..45: mol_type = mRNA
atggataacatttattttgaatacagccatgctttccaggcagtt

SEQ ID NO: 142 - RNA, 45 nt, Homo sapiens
  source 1..45: mol_type = mRNA
tggataacatttattttgaatacagccatgctttccaggcagtta

SEQ ID NO: 143 - RNA, 45 nt, Homo sapiens
  source 1..45: mol_type = mRNA
ggataacatttattttgaatacagccatgctttccaggcagttac

SEQ ID NO: 144 - RNA, 45 nt, Homo sapiens
  source 1..45: mol_type = mRNA
gataacatttattttgaatacagccatgctttccaggcagttaca

SEQ ID NO: 145 - RNA, 45 nt, Homo sapiens
  source 1..45: mol_type = mRNA
ataacatttattttgaatacagccatgctttccaggcagttacag

SEQ ID NO: 146 - RNA, 45 nt, Homo sapiens
  source 1..45: mol_type = mRNA
taacatttattttgaatacagccatgctttccaggcagttacaga

SEQ ID NO: 147 - RNA, 45 nt, Homo sapiens
  source 1..45: mol_type = mRNA
tgaatacagccatgctttccaggcagttacagagttttatgcaaa

SEQ ID NO: 148 - RNA, 45 nt, Homo sapiens
  source 1..45: mol_type = mRNA
gaatacagccatgctttccaggcagttacagagttttatgcaaaa

SEQ ID NO: 149 - RNA, 45 nt, Homo sapiens
  source 1..45: mol_type = mRNA
aatacagccatgctttccaggcagttacagagttttatgcaaaag

SEQ ID NO: 150 - RNA, 45 nt, Homo sapiens
  source 1..45: mol_type = mRNA
atacagccatgctttccaggcagttacagagttttatgcaaaaga

SEQ ID NO: 151 - RNA, 45 nt, Homo sapiens
  source 1..45: mol_type = mRNA
tacagccatgctttccaggcagttacagagttttatgcaaaagat

SEQ ID NO: 152 - RNA, 45 nt, Homo sapiens
  source 1..45: mol_type = mRNA
acagccatgctttccaggcagttacagagttttatgcaaaagata

SEQ ID NO: 153 - RNA, 45 nt, Homo sapiens
  source 1..45: mol_type = mRNA
cagccatgctttccaggcagttacagagttttatgcaaaagatac

SEQ ID NO: 154 - RNA, 45 nt, Homo sapiens
  source 1..45: mol_type = mRNA
ggcagttacagagttttatgcaaaagatacagttgacatcaaagg

SEQ ID NO: 155 - RNA, 45 nt, Homo sapiens
  source 1..45: mol_type = mRNA
agttttatgcaaaagatacagttgacatcaaaggttctcaaatta

SEQ ID NO: 156 - RNA, 45 nt, Homo sapiens
  source 1..45: mol_type = mRNA
gttttatgcaaaagatacagttgacatcaaaggttctcaaattat

SEQ ID NO: 157 - RNA, 45 nt, Homo sapiens
  source 1..45: mol_type = mRNA
ttttatgcaaaagatacagttgacatcaaaggttctcaaattatt

SEQ ID NO: 158 - RNA, 45 nt, Homo sapiens
  source 1..45: mol_type = mRNA
tttatgcaaaagatacagttgacatcaaaggttctcaaattattt

SEQ ID NO: 159 - RNA, 45 nt, Homo sapiens
  source 1..45: mol_type = mRNA
ttatgcaaaagatacagttgacatcaaaggttctcaaattatttc

SEQ ID NO: 160 - RNA, 45 nt, Homo sapiens
  source 1..45: mol_type = mRNA
tatgcaaaagatacagttgacatcaaaggttctcaaattatttct

SEQ ID NO: 161 - RNA, 45 nt, Homo sapiens
  source 1..45: mol_type = mRNA
atgcaaaagatacagttgacatcaaaggttctcaaattatttctg

SEQ ID NO: 162 - RNA, 45 nt, Homo sapiens
  source 1..45: mol_type = mRNA
tgcaaaagatacagttgacatcaaaggttctcaaattatttctgg

SEQ ID NO: 163 - RNA, 45 nt, Homo sapiens
  source 1..45: mol_type = mRNA
aaaggttctcaaattatttctggcattgttaacttagagaagcct

SEQ ID NO: 164 - RNA, 45 nt, Homo sapiens
  source 1..45: mol_type = mRNA
aaggttctcaaattatttctggcattgttaacttagagaagcctg

SEQ ID NO: 165 - RNA, 45 nt, Homo sapiens
  source 1..45: mol_type = mRNA
aggttctcaaattatttctggcattgttaacttagagaagcctgt

SEQ ID NO: 166 - RNA, 45 nt, Homo sapiens
  source 1..45: mol_type = mRNA
ggttctcaaattatttctggcattgttaacttagagaagcctgtg

SEQ ID NO: 167 - RNA, 45 nt, Homo sapiens
  source 1..45: mol_type = mRNA
gttctcaaattatttctggcattgttaacttagagaagcctgtga

SEQ ID NO: 168 - RNA, 45 nt, Homo sapiens
  source 1..45: mol_type = mRNA
ttctcaaattatttctggcattgttaacttagagaagcctgtgat

SEQ ID NO: 169 - RNA, 45 nt, Homo sapiens
  source 1..45: mol_type = mRNA
tctcaaattatttctggcattgttaacttagagaagcctgtgatt

SEQ ID NO: 170 - RNA, 45 nt, Homo sapiens
  source 1..45: mol_type = mRNA
atttctggcattgttaacttagagaagcctgtgatttgctctttg

SEQ ID NO: 171 - RNA, 45 nt, Homo sapiens
  source 1..45: mol_type = mRNA
ttagagaagcctgtgatttgctctttggctgccatcataaaatac

SEQ ID NO: 172 - RNA, 45 nt, Homo sapiens
  source 1..45: mol_type = mRNA
gaagcctgtgatttgctctttggctgccatcataaaatacctcaa

SEQ ID NO: 173 - RNA, 45 nt, Homo sapiens
  source 1..45: mol_type = mRNA
gtgatttgctctttggctgccatcataaaatacctcaaagaattc

SEQ ID NO: 174 - RNA, 45 nt, Homo sapiens
  source 1..45: mol_type = mRNA
gatttgctctttggctgccatcataaaatacctcaaagaattcaa

SEQ ID NO: 175 - RNA, 45 nt, Homo sapiens
  source 1..45: mol_type = mRNA
atttgctctttggctgccatcataaaatacctcaaagaattcaac

SEQ ID NO: 176 - RNA, 45 nt, Homo sapiens
  source 1..45: mol_type = mRNA
tttgctctttggctgccatcataaaatacctcaaagaattcaact

SEQ ID NO: 177 - RNA, 45 nt, Homo sapiens
  source 1..45: mol_type = mRNA
ttgctctttggctgccatcataaaatacctcaaagaattcaactt

SEQ ID NO: 178 - RNA, 45 nt, Homo sapiens
  source 1..45: mol_type = mRNA
tgctctttggctgccatcataaaatacctcaaagaattcaacttg

SEQ ID NO: 179 - RNA, 45 nt, Homo sapiens
  source 1..45: mol_type = mRNA
gctctttggctgccatcataaaatacctcaaagaattcaacttgg

SEQ ID NO: 180 - RNA, 45 nt, Homo sapiens
  source 1..45: mol_type = mRNA
ctctttggctgccatcataaaatacctcaaagaattcaacttgga

SEQ ID NO: 181 - RNA, 45 nt, Homo sapiens
  source 1..45: mol_type = mRNA
catcataaaatacctcaaagaattcaacttggaaaagatgctctc

SEQ ID NO: 182 - RNA, 45 nt, Homo sapiens
  source 1..45: mol_type = mRNA
atacctcaaagaattcaacttggaaaagatgctctccaaacctga

SEQ ID NO: 183 - RNA, 45 nt, Homo sapiens
  source 1..45: mol_type = mRNA
agatgctctccaaacctgagaattttaaacagctatcaagtaaaa

SEQ ID NO: 184 - RNA, 45 nt, Homo sapiens
  source 1..45: mol_type = mRNA
gatgctctccaaacctgagaattttaaacagctatcaagtaaaat

SEQ ID NO: 185 - RNA, 45 nt, Homo sapiens
  source 1..45: mol_type = mRNA
atgctctccaaacctgagaattttaaacagctatcaagtaaaatg

SEQ ID NO: 186 - RNA, 45 nt, Homo sapiens
  source 1..45: mol_type = mRNA
tgctctccaaacctgagaattttaaacagctatcaagtaaaatgg

SEQ ID NO: 187 - RNA, 45 nt, Homo sapiens
  source 1..45: mol_type = mRNA
gctctccaaacctgagaattttaaacagctatcaagtaaaatgga

SEQ ID NO: 188 - RNA, 45 nt, Homo sapiens
  source 1..45: mol_type = mRNA
tttatgacaattaatggaacaacattaaggaatctggaaatccta

SEQ ID NO: 189 - RNA, 45 nt, Homo sapiens
  source 1..45: mol_type = mRNA
ttatgacaattaatggaacaacattaaggaatctggaaatcctac

SEQ ID NO: 190 - RNA, 45 nt, Homo sapiens
  source 1..45: mol_type = mRNA
tatgacaattaatggaacaacattaaggaatctggaaatcctaca

SEQ ID NO: 191 - RNA, 45 nt, Homo sapiens
  source 1..45: mol_type = mRNA
atgacaattaatggaacaacattaaggaatctggaaatcctacag

SEQ ID NO: 192 - RNA, 45 nt, Homo sapiens
  source 1..45: mol_type = mRNA
tgacaattaatggaacaacattaaggaatctggaaatcctacaga

SEQ ID NO: 193 - RNA, 45 nt, Homo sapiens
  source 1..45: mol_type = mRNA
aggaatctggaaatcctacagaatcagactgatatgaaaaccaaa

SEQ ID NO: 194 - RNA, 45 nt, Homo sapiens
  source 1..45: mol_type = mRNA
ggaatctggaaatcctacagaatcagactgatatgaaaaccaaag

SEQ ID NO: 195 - RNA, 45 nt, Homo sapiens
  source 1..45: mol_type = mRNA
gaatctggaaatcctacagaatcagactgatatgaaaaccaaagg

SEQ ID NO: 196 - RNA, 45 nt, Homo sapiens
  source 1..45: mol_type = mRNA
aatctggaaatcctacagaatcagactgatatgaaaaccaaagga

SEQ ID NO: 197 - RNA, 45 nt, Homo sapiens
  source 1..45: mol_type = mRNA
atctggaaatcctacagaatcagactgatatgaaaaccaaaggaa

SEQ ID NO: 198 - RNA, 45 nt, Homo sapiens
  source 1..45: mol_type = mRNA
tctggaaatcctacagaatcagactgatatgaaaaccaaaggaag

SEQ ID NO: 199 - RNA, 45 nt, Homo sapiens
  source 1..45: mol_type = mRNA
tggaaatcctacagaatcagactgatatgaaaaccaaaggaagtt

SEQ ID NO: 200 - RNA, 45 nt, Homo sapiens
  source 1..45: mol_type = mRNA
aaatcctacagaatcagactgatatgaaaaccaaaggaagtttgc

SEQ ID NO: 201 - RNA, 45 nt, Homo sapiens
  source 1..45: mol_type = mRNA
caaaggaagtttgctgtgggttttagaccacactaaaacttcatt

SEQ ID NO: 202 - RNA, 45 nt, Homo sapiens
  source 1..45: mol_type = mRNA
aaaggaagtttgctgtgggttttagaccacactaaaacttcattt

SEQ ID NO: 203 - RNA, 45 nt, Homo sapiens
  source 1..45: mol_type = mRNA
aaggaagtttgctgtgggttttagaccacactaaaacttcatttg

SEQ ID NO: 204 - RNA, 45 nt, Homo sapiens
  source 1..45: mol_type = mRNA
accacactaaaacttcatttgggagacggaagttaaagaagtggg

SEQ ID NO: 205 - RNA, 45 nt, Homo sapiens
  source 1..45: mol_type = mRNA
ttaaagaagtgggtgacccagccactccttaaattaagggaaata

SEQ ID NO: 206 - RNA, 45 nt, Homo sapiens
  source 1..45: mol_type = mRNA
gaaataaatgcccggcttgatgctgtatcggaagttctccattca

SEQ ID NO: 207 - RNA, 45 nt, Homo sapiens
  source 1..45: mol_type = mRNA
aaataaatgcccggcttgatgctgtatcggaagttctccattcag

SEQ ID NO: 208 - RNA, 45 nt, Homo sapiens
  source 1..45: mol_type = mRNA
aataaatgcccggcttgatgctgtatcggaagttctccattcaga

SEQ ID NO: 209 - RNA, 45 nt, Homo sapiens
  source 1..45: mol_type = mRNA
ataaatgcccggcttgatgctgtatcggaagttctccattcagaa

SEQ ID NO: 210 - RNA, 45 nt, Homo sapiens
  source 1..45: mol_type = mRNA
taaatgcccggcttgatgctgtatcggaagttctccattcagaat

SEQ ID NO: 211 - RNA, 45 nt, Homo sapiens
  source 1..45: mol_type = mRNA
aaatgcccggcttgatgctgtatcggaagttctccattcagaatc

SEQ ID NO: 212 - RNA, 45 nt, Homo sapiens
  source 1..45: mol_type = mRNA
aatgcccggcttgatgctgtatcggaagttctccattcagaatct

SEQ ID NO: 213 - RNA, 45 nt, Homo sapiens
  source 1..45: mol_type = mRNA
cattcagaatctagtgtgtttggtcagatagaaaatcatctacgt

SEQ ID NO: 214 - RNA, 45 nt, Homo sapiens
  source 1..45: mol_type = mRNA
ttcagaatctagtgtgtttggtcagatagaaaatcatctacgtaa

SEQ ID NO: 215 - RNA, 45 nt, Homo sapiens
  source 1..45: mol_type = mRNA
tcagaatctagtgtgtttggtcagatagaaaatcatctacgtaaa

SEQ ID NO: 216 - RNA, 45 nt, Homo sapiens
  source 1..45: mol_type = mRNA
cagaatctagtgtgtttggtcagatagaaaatcatctacgtaaat

SEQ ID NO: 217 - RNA, 45 nt, Homo sapiens
  source 1..45: mol_type = mRNA
agaatctagtgtgtttggtcagatagaaaatcatctacgtaaatt

SEQ ID NO: 218 - RNA, 45 nt, Homo sapiens
  source 1..45: mol_type = mRNA
gaatctagtgtgtttggtcagatagaaaatcatctacgtaaattg

SEQ ID NO: 219 - RNA, 45 nt, Homo sapiens
  source 1..45: mol_type = mRNA
gaaaatcatctacgtaaattgcccgacatagagaggggactctgt

SEQ ID NO: 220 - RNA, 45 nt, Homo sapiens
  source 1..45: mol_type = mRNA
gacatagagaggggactctgtagcatttatcacaaaaaatgttct

SEQ ID NO: 221 - RNA, 45 nt, Homo sapiens
  source 1..45: mol_type = mRNA
acatagagaggggactctgtagcatttatcacaaaaaatgttcta

SEQ ID NO: 222 - RNA, 45 nt, Homo sapiens
  source 1..45: mol_type = mRNA
catagagaggggactctgtagcatttatcacaaaaaatgttctac

SEQ ID NO: 223 - RNA, 45 nt, Homo sapiens
  source 1..45: mol_type = mRNA
atttatcacaaaaaatgttctacccaagagttcttcttgattgtc

SEQ ID NO: 224 - RNA, 45 nt, Homo sapiens
  source 1..45: mol_type = mRNA
ttatcacaaaaaatgttctacccaagagttcttcttgattgtcaa

SEQ ID NO: 225 - RNA, 45 nt, Homo sapiens
  source 1..45: mol_type = mRNA
tatcacaaaaaatgttctacccaagagttcttcttgattgtcaaa

SEQ ID NO: 226 - RNA, 45 nt, Homo sapiens
  source 1..45: mol_type = mRNA
atcacaaaaaatgttctacccaagagttcttcttgattgtcaaaa

SEQ ID NO: 227 - RNA, 45 nt, Homo sapiens
  source 1..45: mol_type = mRNA
tcacaaaaaatgttctacccaagagttcttcttgattgtcaaaac

SEQ ID NO: 228 - RNA, 45 nt, Homo sapiens
  source 1..45: mol_type = mRNA
cacaaaaaatgttctacccaagagttcttcttgattgtcaaaact

SEQ ID NO: 229 - RNA, 45 nt, Homo sapiens
  source 1..45: mol_type = mRNA
acaaaaaatgttctacccaagagttcttcttgattgtcaaaactt

SEQ ID NO: 230 - RNA, 45 nt, Homo sapiens
  source 1..45: mol_type = mRNA
ttctacccaagagttcttcttgattgtcaaaactttatatcacct

SEQ ID NO: 231 - RNA, 45 nt, Homo sapiens
  source 1..45: mol_type = mRNA
ttgtcaaaactttatatcacctaaagtcagaatttcaagcaataa

SEQ ID NO: 232 - RNA, 45 nt, Homo sapiens
  source 1..45: mol_type = mRNA
tgtcaaaactttatatcacctaaagtcagaatttcaagcaataat

SEQ ID NO: 233 - RNA, 45 nt, Homo sapiens
  source 1..45: mol_type = mRNA
gtcaaaactttatatcacctaaagtcagaatttcaagcaataata

SEQ ID NO: 234 - RNA, 45 nt, Homo sapiens
  source 1..45: mol_type = mRNA
tcaaaactttatatcacctaaagtcagaatttcaagcaataatac

SEQ ID NO: 235 - RNA, 45 nt, Homo sapiens
  source 1..45: mol_type = mRNA
caaaactttatatcacctaaagtcagaatttcaagcaataatacc

SEQ ID NO: 236 - RNA, 45 nt, Homo sapiens
  source 1..45: mol_type = mRNA
cctaaagtcagaatttcaagcaataatacctgctgttaattccca

SEQ ID NO: 237 - RNA, 45 nt, Homo sapiens
  source 1..45: mol_type = mRNA
gaatttcaagcaataatacctgctgttaattcccacattcagtca

SEQ ID NO: 238 - RNA, 45 nt, Homo sapiens
  source 1..45: mol_type = mRNA
atttcaagcaataatacctgctgttaattcccacattcagtcaga

SEQ ID NO: 239 - RNA, 45 nt, Homo sapiens
  source 1..45: mol_type = mRNA
ttaattcccacattcagtcagacttgctccggaccgttattttag

SEQ ID NO: 240 - RNA, 45 nt, Homo sapiens
  source 1..45: mol_type = mRNA
attttagaaattcctgaactcctcagtccagtggagcattactta

SEQ ID NO: 241 - RNA, 45 nt, Homo sapiens
  source 1..45: mol_type = mRNA
ttcctgaactcctcagtccagtggagcattacttaaagatactca

SEQ ID NO: 242 - RNA, 45 nt, Homo sapiens
  source 1..45: mol_type = mRNA
cctgaactcctcagtccagtggagcattacttaaagatactcaat

SEQ ID NO: 243 - RNA, 45 nt, Homo sapiens
  source 1..45: mol_type = mRNA
aaaactgaattatttaaagacctttctgacttccctttaataaaa

SEQ ID NO: 244 - RNA, 45 nt, Homo sapiens
  source 1..45: mol_type = mRNA
gaattatttaaagacctttctgacttccctttaataaaaaagagg

SEQ ID NO: 245 - RNA, 45 nt, Homo sapiens
  source 1..45: mol_type = mRNA
aattatttaaagacctttctgacttccctttaataaaaaagagga

SEQ ID NO: 246 - RNA, 45 nt, Homo sapiens
  source 1..45: mol_type = mRNA
attatttaaagacctttctgacttccctttaataaaaaagaggaa

SEQ ID NO: 247 - RNA, 45 nt, Homo sapiens
  source 1..45: mol_type = mRNA
ttatttaaagacctttctgacttccctttaataaaaaagaggaag

SEQ ID NO: 248 - RNA, 45 nt, Homo sapiens
  source 1..45: mol_type = mRNA
tatttaaagacctttctgacttccctttaataaaaaagaggaagg

SEQ ID NO: 249 - RNA, 45 nt, Homo sapiens
  source 1..45: mol_type = mRNA
gaaattcaaggtgttattgacgagatccgaatgcatttgcaagaa

SEQ ID NO: 250 - RNA, 45 nt, Homo sapiens
  source 1..45: mol_type = mRNA
gtgttattgacgagatccgaatgcatttgcaagaaatacgaaaaa

SEQ ID NO: 251 - RNA, 45 nt, Homo sapiens
  source 1..45: mol_type = mRNA
tgttattgacgagatccgaatgcatttgcaagaaatacgaaaaat

SEQ ID NO: 252 - RNA, 45 nt, Homo sapiens
  source 1..45: mol_type = mRNA
gttattgacgagatccgaatgcatttgcaagaaatacgaaaaata

SEQ ID NO: 253 - RNA, 45 nt, Homo sapiens
  source 1..45: mol_type = mRNA
ttattgacgagatccgaatgcatttgcaagaaatacgaaaaatac

SEQ ID NO: 254 - RNA, 45 nt, Homo sapiens
  source 1..45: mol_type = mRNA
tattgacgagatccgaatgcatttgcaagaaatacgaaaaatact

SEQ ID NO: 255 - RNA, 45 nt, Homo sapiens
  source 1..45: mol_type = mRNA
gacgagatccgaatgcatttgcaagaaatacgaaaaatactaaaa

SEQ ID NO: 256 - RNA, 45 nt, Homo sapiens
  source 1..45: mol_type = mRNA
ttctgcacaatatgtgacagtatcaggacaggagtttatgataga

SEQ ID NO: 257 - RNA, 45 nt, Homo sapiens
  source 1..45: mol_type = mRNA
tctgcacaatatgtgacagtatcaggacaggagtttatgatagaa

SEQ ID NO: 258 - RNA, 45 nt, Homo sapiens
  source 1..45: mol_type = mRNA
ctgcacaatatgtgacagtatcaggacaggagtttatgatagaaa

SEQ ID NO: 259 - RNA, 45 nt, Homo sapiens
  source 1..45: mol_type = mRNA
tgcacaatatgtgacagtatcaggacaggagtttatgatagaaat

SEQ ID NO: 260 - RNA, 45 nt, Homo sapiens
  source 1..45: mol_type = mRNA
gcacaatatgtgacagtatcaggacaggagtttatgatagaaata

SEQ ID NO: 261 - RNA, 45 nt, Homo sapiens
  source 1..45: mol_type = mRNA
cacaatatgtgacagtatcaggacaggagtttatgatagaaataa

SEQ ID NO: 262 - RNA, 45 nt, Homo sapiens
  source 1..45: mol_type = mRNA
acaatatgtgacagtatcaggacaggagtttatgatagaaataaa

SEQ ID NO: 263 - RNA, 45 nt, Homo sapiens
  source 1..45: mol_type = mRNA
tcaggacaggagtttatgatagaaataaagaactctgctgtatct

SEQ ID NO: 264 - RNA, 45 nt, Homo sapiens
  source 1..45: mol_type = mRNA
caggacaggagtttatgatagaaataaagaactctgctgtatctt

SEQ ID NO: 265 - RNA, 45 nt, Homo sapiens
  source 1..45: mol_type = mRNA
aggacaggagtttatgatagaaataaagaactctgctgtatcttg

SEQ ID NO: 266 - RNA, 45 nt, Homo sapiens
  source 1..45: mol_type = mRNA
ggacaggagtttatgatagaaataaagaactctgctgtatcttgt

SEQ ID NO: 267 - RNA, 45 nt, Homo sapiens
  source 1..45: mol_type = mRNA
gacaggagtttatgatagaaataaagaactctgctgtatcttgta

SEQ ID NO: 268 - RNA, 45 nt, Homo sapiens
  source 1..45: mol_type = mRNA
taaagaactctgctgtatcttgtataccaactgattgggtaaagg

SEQ ID NO: 269 - RNA, 45 nt, Homo sapiens
  source 1..45: mol_type = mRNA
taccaactgattgggtaaaggttggaagcacaaaagctgtgagcc

SEQ ID NO: 270 - RNA, 45 nt, Homo sapiens
  source 1..45: mol_type = mRNA
tgattgggtaaaggttggaagcacaaaagctgtgagccgctttca

SEQ ID NO: 271 - RNA, 45 nt, Homo sapiens
  source 1..45: mol_type = mRNA
gattgggtaaaggttggaagcacaaaagctgtgagccgctttcac

SEQ ID NO: 272 - RNA, 45 nt, Homo sapiens
  source 1..45: mol_type = mRNA
attgggtaaaggttggaagcacaaaagctgtgagccgctttcact

SEQ ID NO: 273 - RNA, 45 nt, Homo sapiens
  source 1..45: mol_type = mRNA
ttgggtaaaggttggaagcacaaaagctgtgagccgctttcactc

SEQ ID NO: 274 - RNA, 45 nt, Homo sapiens
  source 1..45: mol_type = mRNA
tgggtaaaggttggaagcacaaaagctgtgagccgctttcactct

SEQ ID NO: 275 - RNA, 45 nt, Homo sapiens
  source 1..45: mol_type = mRNA
gggtaaaggttggaagcacaaaagctgtgagccgctttcactctc

SEQ ID NO: 276 - RNA, 45 nt, Homo sapiens
  source 1..45: mol_type = mRNA
ggtaaaggttggaagcacaaaagctgtgagccgctttcactctcc

SEQ ID NO: 277 - RNA, 45 nt, Homo sapiens
  source 1..45: mol_type = mRNA
gaagcacaaaagctgtgagccgctttcactctccttttattgtag

SEQ ID NO: 278 - RNA, 45 nt, Homo sapiens
  source 1..45: mol_type = mRNA
aagcacaaaagctgtgagccgctttcactctccttttattgtaga

SEQ ID NO: 279 - RNA, 45 nt, Homo sapiens
  source 1..45: mol_type = mRNA
agcacaaaagctgtgagccgctttcactctccttttattgtagaa

SEQ ID NO: 280 - RNA, 45 nt, Homo sapiens
  source 1..45: mol_type = mRNA
gcacaaaagctgtgagccgctttcactctccttttattgtagaaa

SEQ ID NO: 281 - RNA, 45 nt, Homo sapiens
  source 1..45: mol_type = mRNA
cacaaaagctgtgagccgctttcactctccttttattgtagaaaa

SEQ ID NO: 282 - RNA, 45 nt, Homo sapiens
  source 1..45: mol_type = mRNA
acaaaagctgtgagccgctttcactctccttttattgtagaaaat

SEQ ID NO: 283 - RNA, 45 nt, Homo sapiens
  source 1..45: mol_type = mRNA
agctagtccttgactgcagtgctgaatggcttgattttctagaga

SEQ ID NO: 284 - RNA, 45 nt, Homo sapiens
  source 1..45: mol_type = mRNA
gctagtccttgactgcagtgctgaatggcttgattttctagagaa

SEQ ID NO: 285 - RNA, 45 nt, Homo sapiens
  source 1..45: mol_type = mRNA
ctagtccttgactgcagtgctgaatggcttgattttctagagaaa

SEQ ID NO: 286 - RNA, 45 nt, Homo sapiens
  source 1..45: mol_type = mRNA
tagtccttgactgcagtgctgaatggcttgattttctagagaaat

SEQ ID NO: 287 - RNA, 45 nt, Homo sapiens
  source 1..45: mol_type = mRNA
agtccttgactgcagtgctgaatggcttgattttctagagaaatt

SEQ ID NO: 288 - RNA, 45 nt, Homo sapiens
  source 1..45: mol_type = mRNA
ccttgactgcagtgctgaatggcttgattttctagagaaattcag

SEQ ID NO: 289 - RNA, 45 nt, Homo sapiens
  source 1..45: mol_type = mRNA
cttgactgcagtgctgaatggcttgattttctagagaaattcagt

SEQ ID NO: 290 - RNA, 45 nt, Homo sapiens
  source 1..45: mol_type = mRNA
ttgactgcagtgctgaatggcttgattttctagagaaattcagtg

SEQ ID NO: 291 - RNA, 45 nt, Homo sapiens
  source 1..45: mol_type = mRNA
tgactgcagtgctgaatggcttgattttctagagaaattcagtga

SEQ ID NO: 292 - RNA, 45 nt, Homo sapiens
  source 1..45: mol_type = mRNA
gactgcagtgctgaatggcttgattttctagagaaattcagtgaa

SEQ ID NO: 293 - RNA, 45 nt, Homo sapiens
  source 1..45: mol_type = mRNA
actgcagtgctgaatggcttgattttctagagaaattcagtgaac

SEQ ID NO: 294 - RNA, 45 nt, Homo sapiens
  source 1..45: mol_type = mRNA
ctgcagtgctgaatggcttgattttctagagaaattcagtgaaca

SEQ ID NO: 295 - RNA, 45 nt, Homo sapiens
  source 1..45: mol_type = mRNA
agtgctgaatggcttgattttctagagaaattcagtgaacattat

SEQ ID NO: 296 - RNA, 45 nt, Homo sapiens
  source 1..45: mol_type = mRNA
ctgaatggcttgattttctagagaaattcagtgaacattatcact

SEQ ID NO: 297 - RNA, 45 nt, Homo sapiens
  source 1..45: mol_type = mRNA
attttctagagaaattcagtgaacattatcactccttgtgtaaag

SEQ ID NO: 298 - RNA, 45 nt, Homo sapiens
  source 1..45: mol_type = mRNA
tttctagagaaattcagtgaacattatcactccttgtgtaaagca

SEQ ID NO: 299 - RNA, 45 nt, Homo sapiens
  source 1..45: mol_type = mRNA
ccttgtgtaaagcagtgcatcacctagcaactgttgactgcattt

SEQ ID NO: 300 - RNA, 45 nt, Homo sapiens
  source 1..45: mol_type = mRNA
catcacctagcaactgttgactgcattttctccctggccaaggtc

SEQ ID NO: 301 - RNA, 45 nt, Homo sapiens
  source 1..45: mol_type = mRNA
attttctccctggccaaggtcgctaagcaaggagattactgcaga

SEQ ID NO: 302 - RNA, 45 nt, Homo sapiens
  source 1..45: mol_type = mRNA
cctggccaaggtcgctaagcaaggagattactgcagaccaactgt

SEQ ID NO: 303 - RNA, 45 nt, Homo sapiens
  source 1..45: mol_type = mRNA
tgtcccaaataatacagatttatcagaggactcagagagagtaat

SEQ ID NO: 304 - RNA, 45 nt, Homo sapiens
  source 1..45: mol_type = mRNA
aatacagatttatcagaggactcagagagagtaatgataattacc

SEQ ID NO: 305 - RNA, 45 nt, Homo sapiens
  source 1..45: mol_type = mRNA
gatttatcagaggactcagagagagtaatgataattaccggacca

SEQ ID NO: 306 - RNA, 45 nt, Homo sapiens
  source 1..45: mol_type = mRNA
atttatcagaggactcagagagagtaatgataattaccggaccaa

SEQ ID NO: 307 - RNA, 45 nt, Homo sapiens
  source 1..45: mol_type = mRNA
tttatcagaggactcagagagagtaatgataattaccggaccaaa

SEQ ID NO: 308 - RNA, 45 nt, Homo sapiens
  source 1..45: mol_type = mRNA
ttatcagaggactcagagagagtaatgataattaccggaccaaac

SEQ ID NO: 309 - RNA, 45 nt, Homo sapiens
  source 1..45: mol_type = mRNA
ccggaccaaacatgggtggaaagagctcctacataaaacaagttg

SEQ ID NO: 310 - RNA, 45 nt, Homo sapiens
  source 1..45: mol_type = mRNA
ccaaacatgggtggaaagagctcctacataaaacaagttgcattg

SEQ ID NO: 311 - RNA, 45 nt, Homo sapiens
  source 1..45: mol_type = mRNA
caaacatgggtggaaagagctcctacataaaacaagttgcattga

SEQ ID NO: 312 - RNA, 45 nt, Homo sapiens
  source 1..45: mol_type = mRNA
aacatgggtggaaagagctcctacataaaacaagttgcattgatt

SEQ ID NO: 313 - RNA, 45 nt, Homo sapiens
  source 1..45: mol_type = mRNA
acatgggtggaaagagctcctacataaaacaagttgcattgatta

SEQ ID NO: 314 - RNA, 45 nt, Homo sapiens
  source 1..45: mol_type = mRNA
catgggtggaaagagctcctacataaaacaagttgcattgattac

SEQ ID NO: 315 - RNA, 45 nt, Homo sapiens
  source 1..45: mol_type = mRNA
atgggtggaaagagctcctacataaaacaagttgcattgattacc

SEQ ID NO: 316 - RNA, 45 nt, Homo sapiens
  source 1..45: mol_type = mRNA
tgggtggaaagagctcctacataaaacaagttgcattgattacca

SEQ ID NO: 317 - RNA, 45 nt, Homo sapiens
  source 1..45: mol_type = mRNA
gggtggaaagagctcctacataaaacaagttgcattgattaccat

SEQ ID NO: 318 - RNA, 45 nt, Homo sapiens
  source 1..45: mol_type = mRNA
ggtggaaagagctcctacataaaacaagttgcattgattaccatc

SEQ ID NO: 319 - RNA, 45 nt, Homo sapiens
  source 1..45: mol_type = mRNA
cgacaattgggattgtggatggcattttcacaaggatgggtgctg

SEQ ID NO: 320 - RNA, 45 nt, Homo sapiens
  source 1..45: mol_type = mRNA
gacaattgggattgtggatggcattttcacaaggatgggtgctgc

SEQ ID NO: 321 - RNA, 45 nt, Homo sapiens
  source 1..45: mol_type = mRNA
acaattgggattgtggatggcattttcacaaggatgggtgctgca

SEQ ID NO: 322 - RNA, 45 nt, Homo sapiens
  source 1..45: mol_type = mRNA
caattgggattgtggatggcattttcacaaggatgggtgctgcag

SEQ ID NO: 323 - RNA, 45 nt, Homo sapiens
  source 1..45: mol_type = mRNA
aattgggattgtggatggcattttcacaaggatgggtgctgcaga

SEQ ID NO: 324 - RNA, 45 nt, Homo sapiens
  source 1..45: mol_type = mRNA
tggcattttcacaaggatgggtgctgcagacaatatatataaagg

SEQ ID NO: 325 - RNA, 45 nt, Homo sapiens
  source 1..45: mol_type = mRNA
gcattttcacaaggatgggtgctgcagacaatatatataaaggac

SEQ ID NO: 326 - RNA, 45 nt, Homo sapiens
  source 1..45: mol_type = mRNA
cattttcacaaggatgggtgctgcagacaatatatataaaggaca

SEQ ID NO: 327 - RNA, 45 nt, Homo sapiens
  source 1..45: mol_type = mRNA
attttcacaaggatgggtgctgcagacaatatatataaaggacag

SEQ ID NO: 328 - RNA, 45 nt, Homo sapiens
  source 1..45: mol_type = mRNA
ttttcacaaggatgggtgctgcagacaatatatataaaggacaga

SEQ ID NO: 329 - RNA, 45 nt, Homo sapiens
  source 1..45: mol_type = mRNA
tttcacaaggatgggtgctgcagacaatatatataaaggacagag

SEQ ID NO: 330 - RNA, 45 nt, Homo sapiens
  source 1..45: mol_type = mRNA
tataaaggacagagtacatttatggaagaactgactgacacagca

SEQ ID NO: 331 - RNA, 45 nt, Homo sapiens
  source 1..45: mol_type = mRNA
tttatggaagaactgactgacacagcagaaataatcagaaaagca

SEQ ID NO: 332 - RNA, 45 nt, Homo sapiens
  source 1..45: mol_type = mRNA
agaaataatcagaaaagcaacatcacagtccttggttatcttgga

SEQ ID NO: 333 - RNA, 45 nt, Homo sapiens
  source 1..45: mol_type = mRNA
atcagaaaagcaacatcacagtccttggttatcttggatgaacta

SEQ ID NO: 334 - RNA, 45 nt, Homo sapiens
  source 1..45: mol_type = mRNA
acatcacagtccttggttatcttggatgaactaggaagagggacg

SEQ ID NO: 335 - RNA, 45 nt, Homo sapiens
  source 1..45: mol_type = mRNA
tcacagtccttggttatcttggatgaactaggaagagggacgagc

SEQ ID NO: 336 - RNA, 45 nt, Homo sapiens
  source 1..45: mol_type = mRNA
actcatgatggaattgccattgcctatgctacacttgagtatttc

SEQ ID NO: 337 - RNA, 45 nt, Homo sapiens
  source 1..45: mol_type = mRNA
cctatgctacacttgagtatttcatcagagatgtgaaatccttaa

SEQ ID NO: 338 - RNA, 45 nt, Homo sapiens
  source 1..45: mol_type = mRNA
tatgctacacttgagtatttcatcagagatgtgaaatccttaacc

SEQ ID NO: 339 - RNA, 45 nt, Homo sapiens
  source 1..45: mol_type = mRNA
gtatttcatcagagatgtgaaatccttaaccctgtttgtcaccca

SEQ ID NO: 340 - RNA, 45 nt, Homo sapiens
  source 1..45: mol_type = mRNA
tgtgaaatccttaaccctgtttgtcacccattatccgccagtttg

SEQ ID NO: 341 - RNA, 45 nt, Homo sapiens
  source 1..45: mol_type = mRNA
ccttaaccctgtttgtcacccattatccgccagtttgtgaactag

SEQ ID NO: 342 - RNA, 45 nt, Homo sapiens
  source 1..45: mol_type = mRNA
gtttgtcacccattatccgccagtttgtgaactagaaaaaaatta

SEQ ID NO: 343 - RNA, 45 nt, Homo sapiens
  source 1..45: mol_type = mRNA
gtcacccattatccgccagtttgtgaactagaaaaaaattactca

SEQ ID NO: 344 - RNA, 45 nt, Homo sapiens
  source 1..45: mol_type = mRNA
ttaccacatgggattcttggtcagtgaggatgaaagcaaactgga

SEQ ID NO: 345 - RNA, 45 nt, Homo sapiens
  source 1..45: mol_type = mRNA
agaacaagtccctgattttgtcaccttcctttaccaaataactag

SEQ ID NO: 346 - RNA, 45 nt, Homo sapiens
  source 1..45: mol_type = mRNA
gaacaagtccctgattttgtcaccttcctttaccaaataactaga

SEQ ID NO: 347 - RNA, 45 nt, Homo sapiens
  source 1..45: mol_type = mRNA
aagtccctgattttgtcaccttcctttaccaaataactagaggaa

SEQ ID NO: 348 - RNA, 45 nt, Homo sapiens
  source 1..45: mol_type = mRNA
agtccctgattttgtcaccttcctttaccaaataactagaggaat

SEQ ID NO: 349 - RNA, 45 nt, Homo sapiens
  source 1..45: mol_type = mRNA
gtccctgattttgtcaccttcctttaccaaataactagaggaatt

SEQ ID NO: 350 - RNA, 45 nt, Homo sapiens
  source 1..45: mol_type = mRNA
tccctgattttgtcaccttcctttaccaaataactagaggaattg

SEQ ID NO: 351 - RNA, 45 nt, Homo sapiens
  source 1..45: mol_type = mRNA
ttgtcaccttcctttaccaaataactagaggaattgcagcaagga

SEQ ID NO: 352 - RNA, 45 nt, Homo sapiens
  source 1..45: mol_type = mRNA
ctttaccaaataactagaggaattgcagcaaggagttatggatta

SEQ ID NO: 353 - RNA, 45 nt, Homo sapiens
  source 1..45: mol_type = mRNA
ccaaataactagaggaattgcagcaaggagttatggattaaatgt

SEQ ID NO: 354 - RNA, 45 nt, Homo sapiens
  source 1..45: mol_type = mRNA
aataactagaggaattgcagcaaggagttatggattaaatgtggc

SEQ ID NO: 355 - RNA, 45 nt, Homo sapiens
  source 1..45: mol_type = mRNA
ataactagaggaattgcagcaaggagttatggattaaatgtggct

SEQ ID NO: 356 - RNA, 45 nt, Homo sapiens
  source 1..45: mol_type = mRNA
actagaggaattgcagcaaggagttatggattaaatgtggctaaa

SEQ ID NO: 357 - RNA, 45 nt, Homo sapiens
  source 1..45: mol_type = mRNA
aggagttatggattaaatgtggctaaactagcagatgttcctgga

SEQ ID NO: 358 - RNA, 45 nt, Homo sapiens
  source 1..45: mol_type = mRNA
cctggagaaattttgaagaaagcagctcacaagtcaaaagagctg

SEQ ID NO: 359 - RNA, 45 nt, Homo sapiens
  source 1..45: mol_type = mRNA
taaatacgaaaagaaagagactcaagtattttgcaaagttatgga

SEQ ID NO: 360 - RNA, 45 nt, Homo sapiens
  source 1..45: mol_type = mRNA
aaatacgaaaagaaagagactcaagtattttgcaaagttatggac

SEQ ID NO: 361 - RNA, 45 nt, Homo sapiens
  source 1..45: mol_type = mRNA
aatacgaaaagaaagagactcaagtattttgcaaagttatggacg

SEQ ID NO: 362 - RNA, 45 nt, Homo sapiens
  source 1..45: mol_type = mRNA
atacgaaaagaaagagactcaagtattttgcaaagttatggacga

SEQ ID NO: 363 - RNA, 45 nt, Homo sapiens
  source 1..45: mol_type = mRNA
tacgaaaagaaagagactcaagtattttgcaaagttatggacgat

SEQ ID NO: 364 - RNA, 45 nt, Homo sapiens
  source 1..45: mol_type = mRNA
gagactcaagtattttgcaaagttatggacgatgcataatgcaca

SEQ ID NO: 365 - RNA, 45 nt, Homo sapiens
  source 1..45: mol_type = mRNA
agaagtggacagaggagttcaacatggaagaaacacagacttctc

SEQ ID NO: 366 - RNA, 45 nt, Homo sapiens
  source 1..45: mol_type = mRNA
gaagtggacagaggagttcaacatggaagaaacacagacttctct

SEQ ID NO: 367 - RNA, 45 nt, Homo sapiens
  source 1..45: mol_type = mRNA
aagtggacagaggagttcaacatggaagaaacacagacttctctt

SEQ ID NO: 368 - RNA, 45 nt, Homo sapiens
  source 1..45: mol_type = mRNA
agtggacagaggagttcaacatggaagaaacacagacttctcttc

SEQ ID NO: 369 - RNA, 45 nt, Homo sapiens
  source 1..45: mol_type = mRNA
gtggacagaggagttcaacatggaagaaacacagacttctcttct

SEQ ID NO: 370 - RNA, 45 nt, Homo sapiens
  source 1..45: mol_type = mRNA
agaggagttcaacatggaagaaacacagacttctcttcttcatta

SEQ ID NO: 371 - RNA, 45 nt, Homo sapiens
  source 1..45: mol_type = mRNA
gttcaacatggaagaaacacagacttctcttcttcattaaaatga

SEQ ID NO: 372 - RNA, 45 nt, Homo sapiens
  source 1..45: mol_type = mRNA
cttcattaaaatgaagactacatttgtgaacaaaaaatggagaat

SEQ ID NO: 373 - RNA, 45 nt, Homo sapiens
  source 1..45: mol_type = mRNA
attaaaaataccaactgtacaaaataactctccagtaacagccta

SEQ ID NO: 374 - RNA, 45 nt, Homo sapiens
  source 1..45: mol_type = mRNA
ttaaaaataccaactgtacaaaataactctccagtaacagcctat

SEQ ID NO: 375 - RNA, 45 nt, Homo sapiens
  source 1..45: mol_type = mRNA
tgtacaaaataactctccagtaacagcctatctttgtgtgacatg

SEQ ID NO: 376 - RNA, 45 nt, Homo sapiens
  source 1..45: mol_type = mRNA
agtaacagcctatctttgtgtgacatgtgagcataaaattatgac

SEQ ID NO: 377 - RNA, 45 nt, Homo sapiens
  source 1..45: mol_type = mRNA
aacagcctatctttgtgtgacatgtgagcataaaattatgaccat

SEQ ID NO: 378 - RNA, 45 nt, Homo sapiens
  source 1..45: mol_type = mRNA
atctttgtgtgacatgtgagcataaaattatgaccatggtatatt

SEQ ID NO: 379 - RNA, 45 nt, Homo sapiens
  source 1..45: mol_type = mRNA
atgtgagcataaaattatgaccatggtatattcctattggaaaca

SEQ ID NO: 380 - RNA, 45 nt, Homo sapiens
  source 1..45: mol_type = mRNA
tgtgagcataaaattatgaccatggtatattcctattggaaacag

SEQ ID NO: 381 - RNA, 45 nt, Homo sapiens
  source 1..45: mol_type = mRNA
aattatgaccatggtatattcctattggaaacagagaggtttttc

SEQ ID NO: 382 - RNA, 45 nt, Homo sapiens
  source 1..45: mol_type = mRNA
tatgaccatggtatattcctattggaaacagagaggtttttctga

SEQ ID NO: 383 - RNA, 45 nt, Homo sapiens
  source 1..45: mol_type = mRNA
ccatggtatattcctattggaaacagagaggtttttctgaagaca

SEQ ID NO: 384 - RNA, 45 nt, Homo sapiens
  source 1..45: mol_type = mRNA
gtctttttcaagtttctgtcttcctaacttttctacgtataaaca

SEQ ID NO: 385 - RNA, 45 nt, Homo sapiens
  source 1..45: mol_type = mRNA
ttttcaagtttctgtcttcctaacttttctacgtataaacactct

SEQ ID NO: 386 - RNA, 45 nt, Homo sapiens
  source 1..45: mol_type = mRNA
tttcaagtttctgtcttcctaacttttctacgtataaacactctt

SEQ ID NO: 387 - RNA, 45 nt, Homo sapiens
  source 1..45: mol_type = mRNA
ctgtcttcctaacttttctacgtataaacactcttgaatagactt

SEQ ID NO: 388 - RNA, 45 nt, Homo sapiens
  source 1..45: mol_type = mRNA
tgtcttcctaacttttctacgtataaacactcttgaatagacttc

SEQ ID NO: 389 - RNA, 45 nt, Homo sapiens
  source 1..45: mol_type = mRNA
tcttcctaacttttctacgtataaacactcttgaatagacttcca

SEQ ID NO: 390 - RNA, 45 nt, Homo sapiens
  source 1..45: mol_type = mRNA
ttttctacgtataaacactcttgaatagacttccactttgtaatt

SEQ ID NO: 391 - RNA, 45 nt, Homo sapiens
  source 1..45: mol_type = mRNA
tacgtataaacactcttgaatagacttccactttgtaattagaaa

SEQ ID NO: 392 - RNA, 45 nt, Homo sapiens
  source 1..45: mol_type = mRNA
acgtataaacactcttgaatagacttccactttgtaattagaaaa

SEQ ID NO: 393 - RNA, 45 nt, Homo sapiens
  source 1..45: mol_type = mRNA
tataaacactcttgaatagacttccactttgtaattagaaaattt

SEQ ID NO: 394 - RNA, 45 nt, Homo sapiens
  source 1..45: mol_type = mRNA
aaacactcttgaatagacttccactttgtaattagaaaattttat

SEQ ID NO: 395 - RNA, 45 nt, Homo sapiens
  source 1..45: mol_type = mRNA
aacactcttgaatagacttccactttgtaattagaaaattttatg

SEQ ID NO: 396 - RNA, 45 nt, Homo sapiens
  source 1..45: mol_type = mRNA
aattttatggacagtaagtccagtaaagccttaagtggcagaata

SEQ ID NO: 397 - RNA, 45 nt, Homo sapiens
  source 1..45: mol_type = mRNA
agtaagtccagtaaagccttaagtggcagaatataattcccaagc

SEQ ID NO: 398 - RNA, 45 nt, Homo sapiens
  source 1..45: mol_type = mRNA
tccagtaaagccttaagtggcagaatataattcccaagcttttgg

SEQ ID NO: 399 - RNA, 45 nt, Homo sapiens
  source 1..45: mol_type = mRNA
tggcaactgggtgaatctggcaggaatctatccattgaactaaaa

SEQ ID NO: 400 - RNA, 45 nt, Homo sapiens
  source 1..45: mol_type = mRNA
ctgggtgaatctggcaggaatctatccattgaactaaaataattt

SEQ ID NO: 401 - RNA, 45 nt, Homo sapiens
  source 1..45: mol_type = mRNA
aaaataattttattatgcaaccagtttatccaccaagaacataag

SEQ ID NO: 402 - RNA, 45 nt, Homo sapiens
  source 1..45: mol_type = mRNA
attttattatgcaaccagtttatccaccaagaacataagaatttt

SEQ ID NO: 403 - RNA, 45 nt, Homo sapiens
  source 1..45: mol_type = mRNA
tatgcaaccagtttatccaccaagaacataagaattttttataag

SEQ ID NO: 404 - RNA, 45 nt, Homo sapiens
  source 1..45: mol_type = mRNA
aagcttttaaaaactagagcacagaaggaataaggtcatgaaatt

SEQ ID NO: 405 - RNA, 45 nt, Homo sapiens
  source 1..45: mol_type = mRNA
ttttaaaaactagagcacagaaggaataaggtcatgaaatttaaa

SEQ ID NO: 406 - RNA, 45 nt, Homo sapiens
  source 1..45: mol_type = mRNA
tttaaaaactagagcacagaaggaataaggtcatgaaatttaaaa

SEQ ID NO: 407 - RNA, 45 nt, Homo sapiens
  source 1..45: mol_type = mRNA
aaggttaaatattgtcataggattaagcagtttaaagattgttgg

SEQ ID NO: 408 - RNA, 45 nt, Homo sapiens
  source 1..45: mol_type = mRNA
tattgtcataggattaagcagtttaaagattgttggatgaaatta

SEQ ID NO: 409 - RNA, 16 nt, synthetic construct
  source 1..16: mol_type = other RNA
gccgattcttccagta

SEQ ID NO: 410 - RNA, 16 nt, synthetic construct
  source 1..16: mol_type = other RNA
ggaagcctgaaatcca

SEQ ID NO: 411 - RNA, 16 nt, synthetic construct
  source 1..16: mol_type = other RNA
ctgaaatccacctcca

SEQ ID NO: 412 - RNA, 16 nt, synthetic construct
  source 1..16: mol_type = other RNA
atccacctcctcctca

SEQ ID NO: 413 - RNA, 16 nt, synthetic construct
  source 1..16: mol_type = other RNA
attgacagaagaaaga

SEQ ID NO: 414 - RNA, 16 nt, synthetic construct
  source 1..16: mol_type = other RNA
ttgacagaagaaagaa

SEQ ID NO: 415 - RNA, 16 nt, synthetic construct
  source 1..16: mol_type = other RNA
tgacagaagaaagaaa

SEQ ID NO: 416 - RNA, 16 nt, synthetic construct
  source 1..16: mol_type = other RNA
gacagaagaaagaaga

SEQ ID NO: 417 - RNA, 16 nt, synthetic construct
  source 1..16: mol_type = other RNA
acagaagaaagaagaa

SEQ ID NO: 418 - RNA, 16 nt, synthetic construct
  source 1..16: mol_type = other RNA
gaagaaagaagagaca

SEQ ID NO: 419 - RNA, 16 nt, synthetic construct
  source 1..16: mol_type = other RNA
gaaaatgatgggccta

SEQ ID NO: 420 - RNA, 16 nt, synthetic construct
  source 1..16: mol_type = other RNA
aaaatgatgggcctga

SEQ ID NO: 421 - RNA, 16 nt, synthetic construct
  source 1..16: mol_type = other RNA
aaatgatgggcctgta

SEQ ID NO: 422 - RNA, 16 nt, synthetic construct
  source 1..16: mol_type = other RNA
aatgatgggcctgtta

SEQ ID NO: 423 - RNA, 16 nt, synthetic construct
  source 1..16: mol_type = other RNA
atgatgggcctgttaa

SEQ ID NO: 424 - RNA, 16 nt, synthetic construct
  source 1..16: mol_type = other RNA
actctgagccaaagaa

SEQ ID NO: 425 - RNA, 16 nt, synthetic construct
  source 1..16: mol_type = other RNA
gccaaagaaatgtcta

SEQ ID NO: 426 - RNA, 16 nt, synthetic construct
  source 1..16: mol_type = other RNA
ccaaagaaatgtctga

SEQ ID NO: 427 - RNA, 16 nt, synthetic construct
  source 1..16: mol_type = other RNA
caaagaaatgtctgaa

SEQ ID NO: 428 - RNA, 16 nt, synthetic construct
  source 1..16: mol_type = other RNA
aaagaaatgtctgaga

SEQ ID NO: 429 - RNA, 16 nt, synthetic construct
  source 1..16: mol_type = other RNA
aagaaatgtctgagga

SEQ ID NO: 430 - RNA, 16 nt, synthetic construct
  source 1..16: mol_type = other RNA
agaaatgtctgaggaa

SEQ ID NO: 431 - RNA, 16 nt, synthetic construct
  source 1..16: mol_type = other RNA
gaaatgtctgaggaca

SEQ ID NO: 432 - RNA, 16 nt, synthetic construct
  source 1..16: mol_type = other RNA
attctgcccttcctca

SEQ ID NO: 433 - RNA, 16 nt, synthetic construct
  source 1..16: mol_type = other RNA
ggagagatttgcagta

SEQ ID NO: 434 - RNA, 16 nt, synthetic construct
  source 1..16: mol_type = other RNA
gagagatttgcagtta

SEQ ID NO: 435 - RNA, 16 nt, synthetic construct
  source 1..16: mol_type = other RNA
agagatttgcagttca

SEQ ID NO: 436 - RNA, 16 nt, synthetic construct
  source 1..16: mol_type = other RNA
gagatttgcagttcta

SEQ ID NO: 437 - RNA, 16 nt, synthetic construct
  source 1..16: mol_type = other RNA
agatttgcagttctga

SEQ ID NO: 438 - RNA, 16 nt, synthetic construct
  source 1..16: mol_type = other RNA
ctgccaaaatgtacta

SEQ ID NO: 439 - RNA, 16 nt, synthetic construct
  source 1..16: mol_type = other RNA
tgccaaaatgtactga

SEQ ID NO: 440 - RNA, 16 nt, synthetic construct
  source 1..16: mol_type = other RNA
gccaaaatgtactgaa

SEQ ID NO: 441 - RNA, 16 nt, synthetic construct
  source 1..16: mol_type = other RNA
ccaaaatgtactgata

SEQ ID NO: 442 - RNA, 16 nt, synthetic construct
  source 1..16: mol_type = other RNA
caaaatgtactgatta

SEQ ID NO: 443 - RNA, 16 nt, synthetic construct
  source 1..16: mol_type = other RNA
aaaatgtactgattta

SEQ ID NO: 444 - RNA, 16 nt, synthetic construct
  source 1..16: mol_type = other RNA
aaatgtactgatttta

SEQ ID NO: 445 - RNA, 16 nt, synthetic construct
  source 1..16: mol_type = other RNA
ttctacacgcaaagaa

SEQ ID NO: 446 - RNA, 16 nt, synthetic construct
  source 1..16: mol_type = other RNA
tctacacgcaaagaaa

SEQ ID NO: 447 - RNA, 16 nt, synthetic construct
  source 1..16: mol_type = other RNA
cgcaaagaatgcagta

SEQ ID NO: 448 - RNA, 16 nt, synthetic construct
  source 1..16: mol_type = other RNA
gcaaagaatgcagtta

SEQ ID NO: 449 - RNA, 16 nt, synthetic construct
  source 1..16: mol_type = other RNA
caaagaatgcagttta

SEQ ID NO: 450 - RNA, 16 nt, synthetic construct
  source 1..16: mol_type = other RNA
aaagaatgcagtttca

SEQ ID NO: 451 - RNA, 16 nt, synthetic construct
  source 1..16: mol_type = other RNA
aagaatgcagtttcta

SEQ ID NO: 452 - RNA, 16 nt, synthetic construct
  source 1..16: mol_type = other RNA
agaatgcagtttctta

SEQ ID NO: 453 - RNA, 16 nt, synthetic construct
  source 1..16: mol_type = other RNA
gaatgcagtttcttca

SEQ ID NO: 454 - RNA, 16 nt, synthetic construct
  source 1..16: mol_type = other RNA
ttcgaaacgtcaaata

SEQ ID NO: 455 - RNA, 16 nt, synthetic construct
  source 1..16: mol_type = other RNA
acgtcaaattaatcaa

SEQ ID NO: 456 - RNA, 16 nt, synthetic construct
  source 1..16: mol_type = other RNA
atctcagtcagtttga

SEQ ID NO: 457 - RNA, 16 nt, synthetic construct
  source 1..16: mol_type = other RNA
tcagtttggatcatca

SEQ ID NO: 458 - RNA, 16 nt, synthetic construct
  source 1..16: mol_type = other RNA
agtttggatcatcaaa

SEQ ID NO: 459 - RNA, 16 nt, synthetic construct
  source 1..16: mol_type = other RNA
tggatcatcaaataca

SEQ ID NO: 460 - RNA, 16 nt, synthetic construct
  source 1..16: mol_type = other RNA
tcatcaaatacaagta

SEQ ID NO: 461 - RNA, 16 nt, synthetic construct
  source 1..16: mol_type = other RNA
acagaaaactgcttca

SEQ ID NO: 462 - RNA, 16 nt, synthetic construct
  source 1..16: mol_type = other RNA
aaactgcttccaaata

SEQ ID NO: 463 - RNA, 16 nt, synthetic construct
  source 1..16: mol_type = other RNA
aactgcttccaaatca

SEQ ID NO: 464 - RNA, 16 nt, synthetic construct
  source 1..16: mol_type = other RNA
actgcttccaaatcaa

SEQ ID NO: 465 - RNA, 16 nt, synthetic construct
  source 1..16: mol_type = other RNA
ctgcttccaaatcaga

SEQ ID NO: 466 - RNA, 16 nt, synthetic construct
  source 1..16: mol_type = other RNA
tgcttccaaatcagca

SEQ ID NO: 467 - RNA, 16 nt, synthetic construct
  source 1..16: mol_type = other RNA
aaatcagctaacaaaa

SEQ ID NO: 468 - RNA, 16 nt, synthetic construct
  source 1..16: mol_type = other RNA
aatcagctaacaaaca

SEQ ID NO: 469 - RNA, 16 nt, synthetic construct
  source 1..16: mol_type = other RNA
atcagctaacaaacga

SEQ ID NO: 470 - RNA, 16 nt, synthetic construct
  source 1..16: mol_type = other RNA
tcagctaacaaacgga

SEQ ID NO: 471 - RNA, 16 nt, synthetic construct
  source 1..16: mol_type = other RNA
cagctaacaaacggta

SEQ ID NO: 472 - RNA, 16 nt, synthetic construct
  source 1..16: mol_type = other RNA
agctaacaaacggtca

SEQ ID NO: 473 - RNA, 16 nt, synthetic construct
  source 1..16: mol_type = other RNA
gctaacaaacggtcca

SEQ ID NO: 474 - RNA, 16 nt, synthetic construct
  source 1..16: mol_type = other RNA
aaacggtccaaaagca

SEQ ID NO: 475 - RNA, 16 nt, synthetic construct
  source 1..16: mol_type = other RNA
acggtccaaaagcata

SEQ ID NO: 476 - RNA, 16 nt, synthetic construct
  source 1..16: mol_type = other RNA
caaaagcatctataca

SEQ ID NO: 477 - RNA, 16 nt, synthetic construct
  source 1..16: mol_type = other RNA
aaaagcatctatacga

SEQ ID NO: 478 - RNA, 16 nt, synthetic construct
  source 1..16: mol_type = other RNA
aaagcatctatacgca

SEQ ID NO: 479 - RNA, 16 nt, synthetic construct
  source 1..16: mol_type = other RNA
aagcatctatacgcca

SEQ ID NO: 480 - RNA, 16 nt, synthetic construct
  source 1..16: mol_type = other RNA
acaaagatgcagttta

SEQ ID NO: 481 - RNA, 16 nt, synthetic construct
  source 1..16: mol_type = other RNA
caaagatgcagtttta

SEQ ID NO: 482 - RNA, 16 nt, synthetic construct
  source 1..16: mol_type = other RNA
gtggaatgtggatata

SEQ ID NO: 483 - RNA, 16 nt, synthetic construct
  source 1..16: mol_type = other RNA
tggaatgtggatataa

SEQ ID NO: 484 - RNA, 16 nt, synthetic construct
  source 1..16: mol_type = other RNA
ggaatgtggatataaa

SEQ ID NO: 485 - RNA, 16 nt, synthetic construct
  source 1..16: mol_type = other RNA
gaatgtggatataaga

SEQ ID NO: 486 - RNA, 16 nt, synthetic construct
  source 1..16: mol_type = other RNA
aatgtggatataagta

SEQ ID NO: 487 - RNA, 16 nt, synthetic construct
  source 1..16: mol_type = other RNA
atgtggatataagtaa

SEQ ID NO: 488 - RNA, 16 nt, synthetic construct
  source 1..16: mol_type = other RNA
ggatataagtatagaa

SEQ ID NO: 489 - RNA, 16 nt, synthetic construct
  source 1..16: mol_type = other RNA
gatataagtatagata

SEQ ID NO: 490 - RNA, 16 nt, synthetic construct
  source 1..16: mol_type = other RNA
atataagtatagatta

SEQ ID NO: 491 - RNA, 16 nt, synthetic construct
  source 1..16: mol_type = other RNA
tataagtatagattca

SEQ ID NO: 492 - RNA, 16 nt, synthetic construct
  source 1..16: mol_type = other RNA
ataagtatagattcta

SEQ ID NO: 493 - RNA, 16 nt, synthetic construct
  source 1..16: mol_type = other RNA
tgccatttagatcaca

SEQ ID NO: 494 - RNA, 16 nt, synthetic construct
  source 1..16: mol_type = other RNA
catttagatcacaaca

SEQ ID NO: 495 - RNA, 16 nt, synthetic construct
  source 1..16: mol_type = other RNA
atttagatcacaacta

SEQ ID NO: 496 - RNA, 16 nt, synthetic construct
  source 1..16: mol_type = other RNA
tttagatcacaactta

SEQ ID NO: 497 - RNA, 16 nt, synthetic construct
  source 1..16: mol_type = other RNA
ttagatcacaacttta

SEQ ID NO: 498 - RNA, 16 nt, synthetic construct
  source 1..16: mol_type = other RNA
tagatcacaactttaa

SEQ ID NO: 499 - RNA, 16 nt, synthetic construct
  source 1..16: mol_type = other RNA
agatcacaactttata

SEQ ID NO: 500 - RNA, 16 nt, synthetic construct
  source 1..16: mol_type = other RNA
gatcacaactttatga

SEQ ID NO: 501 - RNA, 16 nt, synthetic construct
  source 1..16: mol_type = other RNA
acaactttatgacaga

SEQ ID NO: 502 - RNA, 16 nt, synthetic construct
  source 1..16: mol_type = other RNA
caactttatgacagca

SEQ ID NO: 503 - RNA, 16 nt, synthetic construct
  source 1..16: mol_type = other RNA
aactttatgacagcaa

SEQ ID NO: 504 - RNA, 16 nt, synthetic construct
  source 1..16: mol_type = other RNA
actttatgacagcaaa

SEQ ID NO: 505 - RNA, 16 nt, synthetic construct
  source 1..16: mol_type = other RNA
ctttatgacagcaaga

SEQ ID NO: 506 - RNA, 16 nt, synthetic construct
  source 1..16: mol_type = other RNA
tttatgacagcaagta

SEQ ID NO: 507 - RNA, 16 nt, synthetic construct
  source 1..16: mol_type = other RNA
ttatgacagcaagtaa

SEQ ID NO: 508 - RNA, 16 nt, synthetic construct
  source 1..16: mol_type = other RNA
ctactcacagactgta

SEQ ID NO: 509 - RNA, 16 nt, synthetic construct
  source 1..16: mol_type = other RNA
tgggagttgtgaagca

SEQ ID NO: 510 - RNA, 16 nt, synthetic construct
  source 1..16: mol_type = other RNA
gggagttgtgaagcaa

SEQ ID NO: 511 - RNA, 16 nt, synthetic construct
  source 1..16: mol_type = other RNA
ggagttgtgaagcaaa

SEQ ID NO: 512 - RNA, 16 nt, synthetic construct
  source 1..16: mol_type = other RNA
agttgtgaagcaaaca

SEQ ID NO: 513 - RNA, 16 nt, synthetic construct
  source 1..16: mol_type = other RNA
gttgtgaagcaaacta

SEQ ID NO: 514 - RNA, 16 nt, synthetic construct
  source 1..16: mol_type = other RNA
ttgtgaagcaaactga

SEQ ID NO: 515 - RNA, 16 nt, synthetic construct
  source 1..16: mol_type = other RNA
tgtgaagcaaactgaa

SEQ ID NO: 516 - RNA, 16 nt, synthetic construct
  source 1..16: mol_type = other RNA
gtgaagcaaactgaaa

SEQ ID NO: 517 - RNA, 16 nt, synthetic construct
  source 1..16: mol_type = other RNA
tgaagcaaactgaaaa

SEQ ID NO: 518 - RNA, 16 nt, synthetic construct
  source 1..16: mol_type = other RNA
gaagcaaactgaaaca

SEQ ID NO: 519 - RNA, 16 nt, synthetic construct
  source 1..16: mol_type = other RNA
aagcaaactgaaacta

SEQ ID NO: 520 - RNA, 16 nt, synthetic construct
  source 1..16: mol_type = other RNA
agcaaactgaaactga

SEQ ID NO: 521 - RNA, 16 nt, synthetic construct
  source 1..16: mol_type = other RNA
gcaaactgaaactgca

SEQ ID NO: 522 - RNA, 16 nt, synthetic construct
  source 1..16: mol_type = other RNA
caaactgaaactgcaa

SEQ ID NO: 523 - RNA, 16 nt, synthetic construct
  source 1..16: mol_type = other RNA
aaactgaaactgcaga

SEQ ID NO: 524 - RNA, 16 nt, synthetic construct
  source 1..16: mol_type = other RNA
aactgaaactgcagca

SEQ ID NO: 525 - RNA, 16 nt, synthetic construct
  source 1..16: mol_type = other RNA
agacaacagaagttca

SEQ ID NO: 526 - RNA, 16 nt, synthetic construct
  source 1..16: mol_type = other RNA
gaaattgactgcccta

SEQ ID NO: 527 - RNA, 16 nt, synthetic construct
  source 1..16: mol_type = other RNA
ttgactgccctttata

SEQ ID NO: 528 - RNA, 16 nt, synthetic construct
  source 1..16: mol_type = other RNA
tgccctttatacaaaa

SEQ ID NO: 529 - RNA, 16 nt, synthetic construct
  source 1..16: mol_type = other RNA
tggatgatgctgtaaa

SEQ ID NO: 530 - RNA, 16 nt, synthetic construct
  source 1..16: mol_type = other RNA
ttgatgagataatgaa

SEQ ID NO: 531 - RNA, 16 nt, synthetic construct
  source 1..16: mol_type = other RNA
ataatgactgatacta

SEQ ID NO: 532 - RNA, 16 nt, synthetic construct
  source 1..16: mol_type = other RNA
tgcatctctgaaaata

SEQ ID NO: 533 - RNA, 16 nt, synthetic construct
  source 1..16: mol_type = other RNA
gcatctctgaaaataa

SEQ ID NO: 534 - RNA, 16 nt, synthetic construct
  source 1..16: mol_type = other RNA
catctctgaaaataaa

SEQ ID NO: 535 - RNA, 16 nt, synthetic construct
  source 1..16: mol_type = other RNA
atctctgaaaataaga

SEQ ID NO: 536 - RNA, 16 nt, synthetic construct
  source 1..16: mol_type = other RNA
tctctgaaaataagga

SEQ ID NO: 537 - RNA, 16 nt, synthetic construct
  source 1..16: mol_type = other RNA
ctctgaaaataaggaa

SEQ ID NO: 538 - RNA, 16 nt, synthetic construct
  source 1..16: mol_type = other RNA
tctgaaaataaggaaa

SEQ ID NO: 539 - RNA, 16 nt, synthetic construct
  source 1..16: mol_type = other RNA
ctgaaaataaggaaaa

SEQ ID NO: 540 - RNA, 16 nt, synthetic construct
  source 1..16: mol_type = other RNA
gaaaataaggaaaata

SEQ ID NO: 541 - RNA, 16 nt, synthetic construct
  source 1..16: mol_type = other RNA
aaaataaggaaaatga

SEQ ID NO: 542 - RNA, 16 nt, synthetic construct
  source 1..16: mol_type = other RNA
ccggatgtcaagccta

SEQ ID NO: 543 - RNA, 16 nt, synthetic construct
  source 1..16: mol_type = other RNA
tgtcaagcctgcagca

SEQ ID NO: 544 - RNA, 16 nt, synthetic construct
  source 1..16: mol_type = other RNA
gcctgcagccagtaga

SEQ ID NO: 545 - RNA, 16 nt, synthetic construct
  source 1..16: mol_type = other RNA
cggccttgtccgagca

SEQ ID NO: 546 - RNA, 16 nt, synthetic construct
  source 1..16: mol_type = other RNA
acagaggcgctcatca

SEQ ID NO: 547 - RNA, 16 nt, synthetic construct
  source 1..16: mol_type = other RNA
ccacagagccacatca

SEQ ID NO: 548 - RNA, 16 nt, synthetic construct
  source 1..16: mol_type = other RNA
gaatacagccatgcta

SEQ ID NO: 549 - RNA, 16 nt, synthetic construct
  source 1..16: mol_type = other RNA
aatacagccatgctta

SEQ ID NO: 550 - RNA, 16 nt, synthetic construct
  source 1..16: mol_type = other RNA
atacagccatgcttta

SEQ ID NO: 551 - RNA, 16 nt, synthetic construct
  source 1..16: mol_type = other RNA
tacagccatgctttca

SEQ ID NO: 552 - RNA, 16 nt, synthetic construct
  source 1..16: mol_type = other RNA
acagccatgctttcca

SEQ ID NO: 553 - RNA, 16 nt, synthetic construct
  source 1..16: mol_type = other RNA
cagccatgctttccaa

SEQ ID NO: 554 - RNA, 16 nt, synthetic construct
  source 1..16: mol_type = other RNA
agccatgctttccaga

SEQ ID NO: 555 - RNA, 16 nt, synthetic construct
  source 1..16: mol_type = other RNA
caggcagttacagaga

SEQ ID NO: 556 - RNA, 16 nt, synthetic construct
  source 1..16: mol_type = other RNA
aggcagttacagagta

SEQ ID NO: 557 - RNA, 16 nt, synthetic construct
  source 1..16: mol_type = other RNA
ggcagttacagagtta

SEQ ID NO: 558 - RNA, 16 nt, synthetic construct
  source 1..16: mol_type = other RNA
gcagttacagagttta

SEQ ID NO: 559 - RNA, 16 nt, synthetic construct
  source 1..16: mol_type = other RNA
cagttacagagtttta

SEQ ID NO: 560 - RNA, 16 nt, synthetic construct
  source 1..16: mol_type = other RNA
agttacagagttttaa

SEQ ID NO: 561 - RNA, 16 nt, synthetic construct
  source 1..16: mol_type = other RNA
gttacagagttttata

SEQ ID NO: 562 - RNA, 16 nt, synthetic construct
  source 1..16: mol_type = other RNA
gcaaaagatacagtta

SEQ ID NO: 563 - RNA, 16 nt, synthetic construct
  source 1..16: mol_type = other RNA
agttgacatcaaagga

SEQ ID NO: 564 - RNA, 16 nt, synthetic construct
  source 1..16: mol_type = other RNA
gttgacatcaaaggta

SEQ ID NO: 565 - RNA, 16 nt, synthetic construct
  source 1..16: mol_type = other RNA
ttgacatcaaaggtta

SEQ ID NO: 566 - RNA, 16 nt, synthetic construct
  source 1..16: mol_type = other RNA
tgacatcaaaggttca

SEQ ID NO: 567 - RNA, 16 nt, synthetic construct
  source 1..16: mol_type = other RNA
gacatcaaaggttcta

SEQ ID NO: 568 - RNA, 16 nt, synthetic construct
  source 1..16: mol_type = other RNA
acatcaaaggttctca

SEQ ID NO: 569 - RNA, 16 nt, synthetic construct
  source 1..16: mol_type = other RNA
catcaaaggttctcaa

SEQ ID NO: 570 - RNA, 16 nt, synthetic construct
  source 1..16: mol_type = other RNA
atcaaaggttctcaaa

SEQ ID NO: 571 - RNA, 16 nt, synthetic construct
  source 1..16: mol_type = other RNA
ctggcattgttaacta

SEQ ID NO: 572 - RNA, 16 nt, synthetic construct
  source 1..16: mol_type = other RNA
tggcattgttaactta

SEQ ID NO: 573 - RNA, 16 nt, synthetic construct
  source 1..16: mol_type = other RNA
ggcattgttaacttaa

SEQ ID NO: 574 - RNA, 16 nt, synthetic construct
  source 1..16: mol_type = other RNA
gcattgttaacttaga

SEQ ID NO: 575 - RNA, 16 nt, synthetic construct
  source 1..16: mol_type = other RNA
cattgttaacttagaa

SEQ ID NO: 576 - RNA, 16 nt, synthetic construct
  source 1..16: mol_type = other RNA
attgttaacttagaga

SEQ ID NO: 577 - RNA, 16 nt, synthetic construct
  source 1..16: mol_type = other RNA
ttgttaacttagagaa

SEQ ID NO: 578 - RNA, 16 nt, synthetic construct
  source 1..16: mol_type = other RNA
tagagaagcctgtgaa

SEQ ID NO: 579 - RNA, 16 nt, synthetic construct
  source 1..16: mol_type = other RNA
gctctttggctgccaa

SEQ ID NO: 580 - RNA, 16 nt, synthetic construct
  source 1..16: mol_type = other RNA
ttggctgccatcataa

SEQ ID NO: 581 - RNA, 16 nt, synthetic construct
  source 1..16: mol_type = other RNA
ccatcataaaatacca

SEQ ID NO: 582 - RNA, 16 nt, synthetic construct
  source 1..16: mol_type = other RNA
atcataaaatacctca

SEQ ID NO: 583 - RNA, 16 nt, synthetic construct
  source 1..16: mol_type = other RNA
tcataaaatacctcaa

SEQ ID NO: 584 - RNA, 16 nt, synthetic construct
  source 1..16: mol_type = other RNA
cataaaatacctcaaa

SEQ ID NO: 585 - RNA, 16 nt, synthetic construct
  source 1..16: mol_type = other RNA
ataaaatacctcaaaa

SEQ ID NO: 586 - RNA, 16 nt, synthetic construct
  source 1..16: mol_type = other RNA
taaaatacctcaaaga

SEQ ID NO: 587 - RNA, 16 nt, synthetic construct
  source 1..16: mol_type = other RNA
aaaatacctcaaagaa

SEQ ID NO: 588 - RNA, 16 nt, synthetic construct
  source 1..16: mol_type = other RNA
aaatacctcaaagaaa

SEQ ID NO: 589 - RNA, 16 nt, synthetic construct
  source 1..16: mol_type = other RNA
gaattcaacttggaaa

SEQ ID NO: 590 - RNA, 16 nt, synthetic construct
  source 1..16: mol_type = other RNA
ttggaaaagatgctca

SEQ ID NO: 591 - RNA, 16 nt, synthetic construct
  source 1..16: mol_type = other RNA
gaattttaaacagcta

SEQ ID NO: 592 - RNA, 16 nt, synthetic construct
  source 1..16: mol_type = other RNA
aattttaaacagctaa

SEQ ID NO: 593 - RNA, 16 nt, synthetic construct
  source 1..16: mol_type = other RNA
attttaaacagctata

SEQ ID NO: 594 - RNA, 16 nt, synthetic construct
  source 1..16: mol_type = other RNA
ttttaaacagctatca

SEQ ID NO: 595 - RNA, 16 nt, synthetic construct
  source 1..16: mol_type = other RNA
tttaaacagctatcaa

SEQ ID NO: 596 - RNA, 16 nt, synthetic construct
  source 1..16: mol_type = other RNA
caacattaaggaatca

SEQ ID NO: 597 - RNA, 16 nt, synthetic construct
  source 1..16: mol_type = other RNA
aacattaaggaatcta

SEQ ID NO: 598 - RNA, 16 nt, synthetic construct
  source 1..16: mol_type = other RNA
acattaaggaatctga

SEQ ID NO: 599 - RNA, 16 nt, synthetic construct
  source 1..16: mol_type = other RNA
cattaaggaatctgga

SEQ ID NO: 600 - RNA, 16 nt, synthetic construct
  source 1..16: mol_type = other RNA
attaaggaatctggaa

SEQ ID NO: 601 - RNA, 16 nt, synthetic construct
  source 1..16: mol_type = other RNA
agaatcagactgataa

SEQ ID NO: 602 - RNA, 16 nt, synthetic construct
  source 1..16: mol_type = other RNA
gaatcagactgatata

SEQ ID NO: 603 - RNA, 16 nt, synthetic construct
  source 1..16: mol_type = other RNA
aatcagactgatatga

SEQ ID NO: 604 - RNA, 16 nt, synthetic construct
  source 1..16: mol_type = other RNA
atcagactgatatgaa

SEQ ID NO: 605 - RNA, 16 nt, synthetic construct
  source 1..16: mol_type = other RNA
tcagactgatatgaaa

SEQ ID NO: 606 - RNA, 16 nt, synthetic construct
  source 1..16: mol_type = other RNA
cagactgatatgaaaa

SEQ ID NO: 607 - RNA, 16 nt, synthetic construct
  source 1..16: mol_type = other RNA
gactgatatgaaaaca

SEQ ID NO: 608 - RNA, 16 nt, synthetic construct
  source 1..16: mol_type = other RNA
tgatatgaaaaccaaa

SEQ ID NO: 609 - RNA, 16 nt, synthetic construct
  source 1..16: mol_type = other RNA
gttttagaccacacta

SEQ ID NO: 610 - RNA, 16 nt, synthetic construct
  source 1..16: mol_type = other RNA
ttttagaccacactaa

SEQ ID NO: 611 - RNA, 16 nt, synthetic construct
  source 1..16: mol_type = other RNA
tttagaccacactaaa

SEQ ID NO: 612 - RNA, 16 nt, synthetic construct
  source 1..16: mol_type = other RNA
tgggagacggaagtta

SEQ ID NO: 613 - RNA, 16 nt, synthetic construct
  source 1..16: mol_type = other RNA
agccactccttaaata

SEQ ID NO: 614 - RNA, 16 nt, synthetic construct
  source 1..16: mol_type = other RNA
atgctgtatcggaaga

SEQ ID NO: 615 - RNA, 16 nt, synthetic construct
  source 1..16: mol_type = other RNA
tgctgtatcggaagta

SEQ ID NO: 616 - RNA, 16 nt, synthetic construct
  source 1..16: mol_type = other RNA
gctgtatcggaagtta

SEQ ID NO: 617 - RNA, 16 nt, synthetic construct
  source 1..16: mol_type = other RNA
ctgtatcggaagttca

SEQ ID NO: 618 - RNA, 16 nt, synthetic construct
  source 1..16: mol_type = other RNA
tgtatcggaagttcta

SEQ ID NO: 619 - RNA, 16 nt, synthetic construct
  source 1..16: mol_type = other RNA
gtatcggaagttctca

SEQ ID NO: 620 - RNA, 16 nt, synthetic construct
  source 1..16: mol_type = other RNA
tatcggaagttctcca

SEQ ID NO: 621 - RNA, 16 nt, synthetic construct
  source 1..16: mol_type = other RNA
ttggtcagatagaaaa

SEQ ID NO: 622 - RNA, 16 nt, synthetic construct
  source 1..16: mol_type = other RNA
ggtcagatagaaaata

SEQ ID NO: 623 - RNA, 16 nt, synthetic construct
  source 1..16: mol_type = other RNA
gtcagatagaaaatca

SEQ ID NO: 624 - RNA, 16 nt, synthetic construct
  source 1..16: mol_type = other RNA
tcagatagaaaatcaa

SEQ ID NO: 625 - RNA, 16 nt, synthetic construct
  source 1..16: mol_type = other RNA
cagatagaaaatcata

SEQ ID NO: 626 - RNA, 16 nt, synthetic construct
  source 1..16: mol_type = other RNA
agatagaaaatcatca

SEQ ID NO: 627 - RNA, 16 nt, synthetic construct
  source 1..16: mol_type = other RNA
tgcccgacatagagaa

SEQ ID NO: 628 - RNA, 16 nt, synthetic construct
  source 1..16: mol_type = other RNA
gtagcatttatcacaa

SEQ ID NO: 629 - RNA, 16 nt, synthetic construct
  source 1..16: mol_type = other RNA
tagcatttatcacaaa

SEQ ID NO: 630 - RNA, 16 nt, synthetic construct
  source 1..16: mol_type = other RNA
agcatttatcacaaaa

SEQ ID NO: 631 - RNA, 16 nt, synthetic construct
  source 1..16: mol_type = other RNA
ctacccaagagttcta

SEQ ID NO: 632 - RNA, 16 nt, synthetic construct
  source 1..16: mol_type = other RNA
acccaagagttcttca

SEQ ID NO: 633 - RNA, 16 nt, synthetic construct
  source 1..16: mol_type = other RNA
cccaagagttcttcta

SEQ ID NO: 634 - RNA, 16 nt, synthetic construct
  source 1..16: mol_type = other RNA
ccaagagttcttctta

SEQ ID NO: 635 - RNA, 16 nt, synthetic construct
  source 1..16: mol_type = other RNA
caagagttcttcttga

SEQ ID NO: 636 - RNA, 16 nt, synthetic construct
  source 1..16: mol_type = other RNA
aagagttcttcttgaa

SEQ ID NO: 637 - RNA, 16 nt, synthetic construct
  source 1..16: mol_type = other RNA
agagttcttcttgata

SEQ ID NO: 638 - RNA, 16 nt, synthetic construct
  source 1..16: mol_type = other RNA
ttgattgtcaaaacta

SEQ ID NO: 639 - RNA, 16 nt, synthetic construct
  source 1..16: mol_type = other RNA
cctaaagtcagaatta

SEQ ID NO: 640 - RNA, 16 nt, synthetic construct
  source 1..16: mol_type = other RNA
ctaaagtcagaattta

SEQ ID NO: 641 - RNA, 16 nt, synthetic construct
  source 1..16: mol_type = other RNA
taaagtcagaatttca

SEQ ID NO: 642 - RNA, 16 nt, synthetic construct
  source 1..16: mol_type = other RNA
aaagtcagaatttcaa

SEQ ID NO: 643 - RNA, 16 nt, synthetic construct
  source 1..16: mol_type = other RNA
aagtcagaatttcaaa

SEQ ID NO: 644 - RNA, 16 nt, synthetic construct
  source 1..16: mol_type = other RNA
gcaataatacctgcta

SEQ ID NO: 645 - RNA, 16 nt, synthetic construct
  source 1..16: mol_type = other RNA
ctgctgttaattccca

SEQ ID NO: 646 - RNA, 16 nt, synthetic construct
  source 1..16: mol_type = other RNA
gctgttaattcccaca

SEQ ID NO: 647 - RNA, 16 nt, synthetic construct
  source 1..16: mol_type = other RNA
agacttgctccggaca

SEQ ID NO: 648 - RNA, 16 nt, synthetic construct
  source 1..16: mol_type = other RNA
tcctcagtccagtgga

SEQ ID NO: 649 - RNA, 16 nt, synthetic construct
  source 1..16: mol_type = other RNA
agtggagcattactta

SEQ ID NO: 650 - RNA, 16 nt, synthetic construct
  source 1..16: mol_type = other RNA
tggagcattacttaaa

SEQ ID NO: 651 - RNA, 16 nt, synthetic construct
  source 1..16: mol_type = other RNA
acctttctgacttcca

SEQ ID NO: 652 - RNA, 16 nt, synthetic construct
  source 1..16: mol_type = other RNA
ctgacttccctttaaa

SEQ ID NO: 653 - RNA, 16 nt, synthetic construct
  source 1..16: mol_type = other RNA
tgacttccctttaata

SEQ ID NO: 654 - RNA, 16 nt, synthetic construct
  source 1..16: mol_type = other RNA
gacttccctttaataa

SEQ ID NO: 655 - RNA, 16 nt, synthetic construct
  source 1..16: mol_type = other RNA
acttccctttaataaa

SEQ ID NO: 656 - RNA, 16 nt, synthetic construct
  source 1..16: mol_type = other RNA
cttccctttaataaaa

SEQ ID NO: 657 - RNA, 16 nt, synthetic construct
  source 1..16: mol_type = other RNA
acgagatccgaatgca

SEQ ID NO: 658 - RNA, 16 nt, synthetic construct
  source 1..16: mol_type = other RNA
aatgcatttgcaagaa

SEQ ID NO: 659 - RNA, 16 nt, synthetic construct
  source 1..16: mol_type = other RNA
atgcatttgcaagaaa

SEQ ID NO: 660 - RNA, 16 nt, synthetic construct
  source 1..16: mol_type = other RNA
tgcatttgcaagaaaa

SEQ ID NO: 661 - RNA, 16 nt, synthetic construct
  source 1..16: mol_type = other RNA
gcatttgcaagaaata

SEQ ID NO: 662 - RNA, 16 nt, synthetic construct
  source 1..16: mol_type = other RNA
catttgcaagaaataa

SEQ ID NO: 663 - RNA, 16 nt, synthetic construct
  source 1..16: mol_type = other RNA
tgcaagaaatacgaaa

SEQ ID NO: 664 - RNA, 16 nt, synthetic construct
  source 1..16: mol_type = other RNA
gtatcaggacaggaga

SEQ ID NO: 665 - RNA, 16 nt, synthetic construct
  source 1..16: mol_type = other RNA
tatcaggacaggagta

SEQ ID NO: 666 - RNA, 16 nt, synthetic construct
  source 1..16: mol_type = other RNA
atcaggacaggagtta

SEQ ID NO: 667 - RNA, 16 nt, synthetic construct
  source 1..16: mol_type = other RNA
tcaggacaggagttta

SEQ ID NO: 668 - RNA, 16 nt, synthetic construct
  source 1..16: mol_type = other RNA
caggacaggagtttaa

SEQ ID NO: 669 - RNA, 16 nt, synthetic construct
  source 1..16: mol_type = other RNA
aggacaggagtttata

SEQ ID NO: 670 - RNA, 16 nt, synthetic construct
  source 1..16: mol_type = other RNA
ggacaggagtttatga

SEQ ID NO: 671 - RNA, 16 nt, synthetic construct
  source 1..16: mol_type = other RNA
tagaaataaagaacta

SEQ ID NO: 672 - RNA, 16 nt, synthetic construct
  source 1..16: mol_type = other RNA
agaaataaagaactca

SEQ ID NO: 673 - RNA, 16 nt, synthetic construct
  source 1..16: mol_type = other RNA
gaaataaagaactcta

SEQ ID NO: 674 - RNA, 16 nt, synthetic construct
  source 1..16: mol_type = other RNA
aaataaagaactctga

SEQ ID NO: 675 - RNA, 16 nt, synthetic construct
  source 1..16: mol_type = other RNA
aataaagaactctgca

SEQ ID NO: 676 - RNA, 16 nt, synthetic construct
  source 1..16: mol_type = other RNA
ttgtataccaactgaa

SEQ ID NO: 677 - RNA, 16 nt, synthetic construct
  source 1..16: mol_type = other RNA
ggttggaagcacaaaa

SEQ ID NO: 678 - RNA, 16 nt, synthetic construct
  source 1..16: mol_type = other RNA
agcacaaaagctgtga

SEQ ID NO: 679 - RNA, 16 nt, synthetic construct
  source 1..16: mol_type = other RNA
gcacaaaagctgtgaa

SEQ ID NO: 680 - RNA, 16 nt, synthetic construct
  source 1..16: mol_type = other RNA
cacaaaagctgtgaga

SEQ ID NO: 681 - RNA, 16 nt, synthetic construct
  source 1..16: mol_type = other RNA
acaaaagctgtgagca

SEQ ID NO: 682 - RNA, 16 nt, synthetic construct
  source 1..16: mol_type = other RNA
caaaagctgtgagcca

SEQ ID NO: 683 - RNA, 16 nt, synthetic construct
  source 1..16: mol_type = other RNA
aaaagctgtgagccga

SEQ ID NO: 684 - RNA, 16 nt, synthetic construct
  source 1..16: mol_type = other RNA
aaagctgtgagccgca

SEQ ID NO: 685 - RNA, 16 nt, synthetic construct
  source 1..16: mol_type = other RNA
ccgctttcactctcca

SEQ ID NO: 686 - RNA, 16 nt, synthetic construct
  source 1..16: mol_type = other RNA
cgctttcactctccta

SEQ ID NO: 687 - RNA, 16 nt, synthetic construct
  source 1..16: mol_type = other RNA
gctttcactctcctta

SEQ ID NO: 688 - RNA, 16 nt, synthetic construct
  source 1..16: mol_type = other RNA
ctttcactctccttta

SEQ ID NO: 689 - RNA, 16 nt, synthetic construct
  source 1..16: mol_type = other RNA
tttcactctcctttta

SEQ ID NO: 690 - RNA, 16 nt, synthetic construct
  source 1..16: mol_type = other RNA
ttcactctccttttaa

SEQ ID NO: 691 - RNA, 16 nt, synthetic construct
  source 1..16: mol_type = other RNA
tgctgaatggcttgaa

SEQ ID NO: 692 - RNA, 16 nt, synthetic construct
  source 1..16: mol_type = other RNA
gctgaatggcttgata

SEQ ID NO: 693 - RNA, 16 nt, synthetic construct
  source 1..16: mol_type = other RNA
ctgaatggcttgatta

SEQ ID NO: 694 - RNA, 16 nt, synthetic construct
  source 1..16: mol_type = other RNA
tgaatggcttgattta

SEQ ID NO: 695 - RNA, 16 nt, synthetic construct
  source 1..16: mol_type = other RNA
gaatggcttgatttta

SEQ ID NO: 696 - RNA, 16 nt, synthetic construct
  source 1..16: mol_type = other RNA
tggcttgattttctaa

SEQ ID NO: 697 - RNA, 16 nt, synthetic construct
  source 1..16: mol_type = other RNA
ggcttgattttctaga

SEQ ID NO: 698 - RNA, 16 nt, synthetic construct
  source 1..16: mol_type = other RNA
gcttgattttctagaa

SEQ ID NO: 699 - RNA, 16 nt, synthetic construct
  source 1..16: mol_type = other RNA
cttgattttctagaga

SEQ ID NO: 700 - RNA, 16 nt, synthetic construct
  source 1..16: mol_type = other RNA
ttgattttctagagaa

SEQ ID NO: 701 - RNA, 16 nt, synthetic construct
  source 1..16: mol_type = other RNA
tgattttctagagaaa

SEQ ID NO: 702 - RNA, 16 nt, synthetic construct
  source 1..16: mol_type = other RNA
gattttctagagaaaa

SEQ ID NO: 703 - RNA, 16 nt, synthetic construct
  source 1..16: mol_type = other RNA
ttctagagaaattcaa

SEQ ID NO: 704 - RNA, 16 nt, synthetic construct
  source 1..16: mol_type = other RNA
agagaaattcagtgaa

SEQ ID NO: 705 - RNA, 16 nt, synthetic construct
  source 1..16: mol_type = other RNA
tgaacattatcactca

SEQ ID NO: 706 - RNA, 16 nt, synthetic construct
  source 1..16: mol_type = other RNA
aacattatcactccta

SEQ ID NO: 707 - RNA, 16 nt, synthetic construct
  source 1..16: mol_type = other RNA
tcacctagcaactgta

SEQ ID NO: 708 - RNA, 16 nt, synthetic construct
  source 1..16: mol_type = other RNA
actgcattttctccca

SEQ ID NO: 709 - RNA, 16 nt, synthetic construct
  source 1..16: mol_type = other RNA
tcgctaagcaaggaga

SEQ ID NO: 710 - RNA, 16 nt, synthetic construct
  source 1..16: mol_type = other RNA
caaggagattactgca

SEQ ID NO: 711 - RNA, 16 nt, synthetic construct
  source 1..16: mol_type = other RNA
ttatcagaggactcaa

SEQ ID NO: 712 - RNA, 16 nt, synthetic construct
  source 1..16: mol_type = other RNA
actcagagagagtaaa

SEQ ID NO: 713 - RNA, 16 nt, synthetic construct
  source 1..16: mol_type = other RNA
agagagtaatgataaa

SEQ ID NO: 714 - RNA, 16 nt, synthetic construct
  source 1..16: mol_type = other RNA
gagagtaatgataata

SEQ ID NO: 715 - RNA, 16 nt, synthetic construct
  source 1..16: mol_type = other RNA
agagtaatgataatta

SEQ ID NO: 716 - RNA, 16 nt, synthetic construct
  source 1..16: mol_type = other RNA
gagtaatgataattaa

SEQ ID NO: 717 - RNA, 16 nt, synthetic construct
  source 1..16: mol_type = other RNA
aaagagctcctacata

SEQ ID NO: 718 - RNA, 16 nt, synthetic construct
  source 1..16: mol_type = other RNA
gctcctacataaaaca

SEQ ID NO: 719 - RNA, 16 nt, synthetic construct
  source 1..16: mol_type = other RNA
ctcctacataaaacaa

SEQ ID NO: 720 - RNA, 16 nt, synthetic construct
  source 1..16: mol_type = other RNA
cctacataaaacaaga

SEQ ID NO: 721 - RNA, 16 nt, synthetic construct
  source 1..16: mol_type = other RNA
ctacataaaacaagta

SEQ ID NO: 722 - RNA, 16 nt, synthetic construct
  source 1..16: mol_type = other RNA
tacataaaacaagtta

SEQ ID NO: 723 - RNA, 16 nt, synthetic construct
  source 1..16: mol_type = other RNA
acataaaacaagttga

SEQ ID NO: 724 - RNA, 16 nt, synthetic construct
  source 1..16: mol_type = other RNA
cataaaacaagttgca

SEQ ID NO: 725 - RNA, 16 nt, synthetic construct
  source 1..16: mol_type = other RNA
ataaaacaagttgcaa

SEQ ID NO: 726 - RNA, 16 nt, synthetic construct
  source 1..16: mol_type = other RNA
taaaacaagttgcata

SEQ ID NO: 727 - RNA, 16 nt, synthetic construct
  source 1..16: mol_type = other RNA
tggcattttcacaaga

SEQ ID NO: 728 - RNA, 16 nt, synthetic construct
  source 1..16: mol_type = other RNA
ggcattttcacaagga

SEQ ID NO: 729 - RNA, 16 nt, synthetic construct
  source 1..16: mol_type = other RNA
gcattttcacaaggaa

SEQ ID NO: 730 - RNA, 16 nt, synthetic construct
  source 1..16: mol_type = other RNA
cattttcacaaggata

SEQ ID NO: 731 - RNA, 16 nt, synthetic construct
  source 1..16: mol_type = other RNA
attttcacaaggatga

SEQ ID NO: 732 - RNA, 16 nt, synthetic construct
  source 1..16: mol_type = other RNA
ggtgctgcagacaata

SEQ ID NO: 733 - RNA, 16 nt, synthetic construct
  source 1..16: mol_type = other RNA
tgctgcagacaatata

SEQ ID NO: 734 - RNA, 16 nt, synthetic construct
  source 1..16: mol_type = other RNA
gctgcagacaatataa

SEQ ID NO: 735 - RNA, 16 nt, synthetic construct
  source 1..16: mol_type = other RNA
ctgcagacaatatata

SEQ ID NO: 736 - RNA, 16 nt, synthetic construct
  source 1..16: mol_type = other RNA
tgcagacaatatataa

SEQ ID NO: 737 - RNA, 16 nt, synthetic construct
  source 1..16: mol_type = other RNA
gcagacaatatatata

SEQ ID NO: 738 - RNA, 16 nt, synthetic construct
  source 1..16: mol_type = other RNA
ttatggaagaactgaa

SEQ ID NO: 739 - RNA, 16 nt, synthetic construct
  source 1..16: mol_type = other RNA
acacagcagaaataaa

SEQ ID NO: 740 - RNA, 16 nt, synthetic construct
  source 1..16: mol_type = other RNA
acatcacagtccttga

SEQ ID NO: 741 - RNA, 16 nt, synthetic construct
  source 1..16: mol_type = other RNA
agtccttggttatcta

SEQ ID NO: 742 - RNA, 16 nt, synthetic construct
  source 1..16: mol_type = other RNA
tcttggatgaactaga

SEQ ID NO: 743 - RNA, 16 nt, synthetic construct
  source 1..16: mol_type = other RNA
tggatgaactaggaaa

SEQ ID NO: 744 - RNA, 16 nt, synthetic construct
  source 1..16: mol_type = other RNA
ttgcctatgctacaca

SEQ ID NO: 745 - RNA, 16 nt, synthetic construct
  source 1..16: mol_type = other RNA
tttcatcagagatgta

SEQ ID NO: 746 - RNA, 16 nt, synthetic construct
  source 1..16: mol_type = other RNA
tcatcagagatgtgaa

SEQ ID NO: 747 - RNA, 16 nt, synthetic construct
  source 1..16: mol_type = other RNA
aaatccttaaccctga

SEQ ID NO: 748 - RNA, 16 nt, synthetic construct
  source 1..16: mol_type = other RNA
tttgtcacccattata

SEQ ID NO: 749 - RNA, 16 nt, synthetic construct
  source 1..16: mol_type = other RNA
ccattatccgccagta

SEQ ID NO: 750 - RNA, 16 nt, synthetic construct
  source 1..16: mol_type = other RNA
ccagtttgtgaactaa

SEQ ID NO: 751 - RNA, 16 nt, synthetic construct
  source 1..16: mol_type = other RNA
tttgtgaactagaaaa

SEQ ID NO: 752 - RNA, 16 nt, synthetic construct
  source 1..16: mol_type = other RNA
gtcagtgaggatgaaa

SEQ ID NO: 753 - RNA, 16 nt, synthetic construct
  source 1..16: mol_type = other RNA
gtcaccttcctttaca

SEQ ID NO: 754 - RNA, 16 nt, synthetic construct
  source 1..16: mol_type = other RNA
tcaccttcctttacca

SEQ ID NO: 755 - RNA, 16 nt, synthetic construct
  source 1..16: mol_type = other RNA
cttcctttaccaaata

SEQ ID NO: 756 - RNA, 16 nt, synthetic construct
  source 1..16: mol_type = other RNA
ttcctttaccaaataa

SEQ ID NO: 757 - RNA, 16 nt, synthetic construct
  source 1..16: mol_type = other RNA
tcctttaccaaataaa

SEQ ID NO: 758 - RNA, 16 nt, synthetic construct
  source 1..16: mol_type = other RNA
cctttaccaaataaca

SEQ ID NO: 759 - RNA, 16 nt, synthetic construct
  source 1..16: mol_type = other RNA
aataactagaggaata

SEQ ID NO: 760 - RNA, 16 nt, synthetic construct
  source 1..16: mol_type = other RNA
gaattgcagcaaggaa

SEQ ID NO: 761 - RNA, 16 nt, synthetic construct
  source 1..16: mol_type = other RNA
gcagcaaggagttata

SEQ ID NO: 762 - RNA, 16 nt, synthetic construct
  source 1..16: mol_type = other RNA
gcaaggagttatggaa

SEQ ID NO: 763 - RNA, 16 nt, synthetic construct
  source 1..16: mol_type = other RNA
caaggagttatggata

SEQ ID NO: 764 - RNA, 16 nt, synthetic construct
  source 1..16: mol_type = other RNA
ggagttatggattaaa

SEQ ID NO: 765 - RNA, 16 nt, synthetic construct
  source 1..16: mol_type = other RNA
tggctaaactagcaga

SEQ ID NO: 766 - RNA, 16 nt, synthetic construct
  source 1..16: mol_type = other RNA
aagcagctcacaagta

SEQ ID NO: 767 - RNA, 16 nt, synthetic construct
  source 1..16: mol_type = other RNA
actcaagtattttgca

SEQ ID NO: 768 - RNA, 16 nt, synthetic construct
  source 1..16: mol_type = other RNA
ctcaagtattttgcaa

SEQ ID NO: 769 - RNA, 16 nt, synthetic construct
  source 1..16: mol_type = other RNA
tcaagtattttgcaaa

SEQ ID NO: 770 - RNA, 16 nt, synthetic construct
  source 1..16: mol_type = other RNA
caagtattttgcaaaa

SEQ ID NO: 771 - RNA, 16 nt, synthetic construct
  source 1..16: mol_type = other RNA
aagtattttgcaaaga

SEQ ID NO: 772 - RNA, 16 nt, synthetic construct
  source 1..16: mol_type = other RNA
aagttatggacgatga

SEQ ID NO: 773 - RNA, 16 nt, synthetic construct
  source 1..16: mol_type = other RNA
caacatggaagaaaca

SEQ ID NO: 774 - RNA, 16 nt, synthetic construct
  source 1..16: mol_type = other RNA
aacatggaagaaacaa

SEQ ID NO: 775 - RNA, 16 nt, synthetic construct
  source 1..16: mol_type = other RNA
acatggaagaaacaca

SEQ ID NO: 776 - RNA, 16 nt, synthetic construct
  source 1..16: mol_type = other RNA
catggaagaaacacaa

SEQ ID NO: 777 - RNA, 16 nt, synthetic construct
  source 1..16: mol_type = other RNA
atggaagaaacacaga

SEQ ID NO: 778 - RNA, 16 nt, synthetic construct
  source 1..16: mol_type = other RNA
gaaacacagacttcta

SEQ ID NO: 779 - RNA, 16 nt, synthetic construct
  source 1..16: mol_type = other RNA
cagacttctcttctta

SEQ ID NO: 780 - RNA, 16 nt, synthetic construct
  source 1..16: mol_type = other RNA
acatttgtgaacaaaa

SEQ ID NO: 781 - RNA, 16 nt, synthetic construct
  source 1..16: mol_type = other RNA
caaaataactctccaa

SEQ ID NO: 782 - RNA, 16 nt, synthetic construct
  source 1..16: mol_type = other RNA
aaaataactctccaga

SEQ ID NO: 783 - RNA, 16 nt, synthetic construct
  source 1..16: mol_type = other RNA
gtaacagcctatctta

SEQ ID NO: 784 - RNA, 16 nt, synthetic construct
  source 1..16: mol_type = other RNA
gtgacatgtgagcata

SEQ ID NO: 785 - RNA, 16 nt, synthetic construct
  source 1..16: mol_type = other RNA
acatgtgagcataaaa

SEQ ID NO: 786 - RNA, 16 nt, synthetic construct
  source 1..16: mol_type = other RNA
gcataaaattatgaca

SEQ ID NO: 787 - RNA, 16 nt, synthetic construct
  source 1..16: mol_type = other RNA
accatggtatattcca

SEQ ID NO: 788 - RNA, 16 nt, synthetic construct
  source 1..16: mol_type = other RNA
ccatggtatattccta

SEQ ID NO: 789 - RNA, 16 nt, synthetic construct
  source 1..16: mol_type = other RNA
tcctattggaaacaga

SEQ ID NO: 790 - RNA, 16 nt, synthetic construct
  source 1..16: mol_type = other RNA
tattggaaacagagaa

SEQ ID NO: 791 - RNA, 16 nt, synthetic construct
  source 1..16: mol_type = other RNA
gaaacagagaggttta

SEQ ID NO: 792 - RNA, 16 nt, synthetic construct
  source 1..16: mol_type = other RNA
cttcctaacttttcta

SEQ ID NO: 793 - RNA, 16 nt, synthetic construct
  source 1..16: mol_type = other RNA
ctaacttttctacgta

SEQ ID NO: 794 - RNA, 16 nt, synthetic construct
  source 1..16: mol_type = other RNA
taacttttctacgtaa

SEQ ID NO: 795 - RNA, 16 nt, synthetic construct
  source 1..16: mol_type = other RNA
acgtataaacactcta

SEQ ID NO: 796 - RNA, 16 nt, synthetic construct
  source 1..16: mol_type = other RNA
cgtataaacactctta

SEQ ID NO: 797 - RNA, 16 nt, synthetic construct
  source 1..16: mol_type = other RNA
tataaacactcttgaa

SEQ ID NO: 798 - RNA, 16 nt, synthetic construct
  source 1..16: mol_type = other RNA
cttgaatagacttcca

SEQ ID NO: 799 - RNA, 16 nt, synthetic construct
  source 1..16: mol_type = other RNA
atagacttccacttta

SEQ ID NO: 800 - RNA, 16 nt, synthetic construct
  source 1..16: mol_type = other RNA
tagacttccactttga

SEQ ID NO: 801 - RNA, 16 nt, synthetic construct
  source 1..16: mol_type = other RNA
acttccactttgtaaa

SEQ ID NO: 802 - RNA, 16 nt, synthetic construct
  source 1..16: mol_type = other RNA
tccactttgtaattaa

SEQ ID NO: 803 - RNA, 16 nt, synthetic construct
  source 1..16: mol_type = other RNA
ccactttgtaattaga

SEQ ID NO: 804 - RNA, 16 nt, synthetic construct
  source 1..16: mol_type = other RNA
ccagtaaagccttaaa

SEQ ID NO: 805 - RNA, 16 nt, synthetic construct
  source 1..16: mol_type = other RNA
taagtggcagaatata

SEQ ID NO: 806 - RNA, 16 nt, synthetic construct
  source 1..16: mol_type = other RNA
gcagaatataattcca

SEQ ID NO: 807 - RNA, 16 nt, synthetic construct
  source 1..16: mol_type = other RNA
gcaggaatctatccaa

SEQ ID NO: 808 - RNA, 16 nt, synthetic construct
  source 1..16: mol_type = other RNA
atctatccattgaaca

SEQ ID NO: 809 - RNA, 16 nt, synthetic construct
  source 1..16: mol_type = other RNA
accagtttatccacca

SEQ ID NO: 810 - RNA, 16 nt, synthetic construct
  source 1..16: mol_type = other RNA
ttatccaccaagaaca

SEQ ID NO: 811 - RNA, 16 nt, synthetic construct
  source 1..16: mol_type = other RNA
ccaagaacataagaaa

SEQ ID NO: 812 - RNA, 16 nt, synthetic construct
  source 1..16: mol_type = other RNA
cacagaaggaataaga

SEQ ID NO: 813 - RNA, 16 nt, synthetic construct
  source 1..16: mol_type = other RNA
gaaggaataaggtcaa

SEQ ID NO: 814 - RNA, 16 nt, synthetic construct
  source 1..16: mol_type = other RNA
aaggaataaggtcata

SEQ ID NO: 815 - RNA, 16 nt, synthetic construct
  source 1..16: mol_type = other RNA
ggattaagcagtttaa

SEQ ID NO: 816 - RNA, 16 nt, synthetic construct
  source 1..16: mol_type = other RNA
agtttaaagattgtta

SEQ ID NO: 817 - RNA, 21 nt, synthetic construct
  source 1..21: mol_type = other RNA
tactggaagaatcggctcaaa

SEQ ID NO: 818 - RNA, 21 nt, synthetic construct
  source 1..21: mol_type = other RNA
tggatttcaggcttcccgtag

SEQ ID NO: 819 - RNA, 21 nt, synthetic construct
  source 1..21: mol_type = other RNA
tggaggtggatttcaggcttc

SEQ ID NO: 820 - RNA, 21 nt, synthetic construct
  source 1..21: mol_type = other RNA
tgaggaggaggtggatttcag

SEQ ID NO: 821 - RNA, 21 nt, synthetic construct
  source 1..21: mol_type = other RNA
tctttcttctgtcaatttctg

SEQ ID NO: 822 - RNA, 21 nt, synthetic construct
  source 1..21: mol_type = other RNA
ttctttcttctgtcaatttct

SEQ ID NO: 823 - RNA, 21 nt, synthetic construct
  source 1..21: mol_type = other RNA
tttctttcttctgtcaatttc

SEQ ID NO: 824 - RNA, 21 nt, synthetic construct
  source 1..21: mol_type = other RNA
tcttctttcttctgtcaattt

SEQ ID NO: 825 - RNA, 21 nt, synthetic construct
  source 1..21: mol_type = other RNA
ttcttctttcttctgtcaatt

SEQ ID NO: 826 - RNA, 21 nt, synthetic construct
  source 1..21: mol_type = other RNA
tgtctcttctttcttctgtca

SEQ ID NO: 827 - RNA, 21 nt, synthetic construct
  source 1..21: mol_type = other RNA
taggcccatcattttccaatg

SEQ ID NO: 828 - RNA, 21 nt, synthetic construct
  source 1..21: mol_type = other RNA
tcaggcccatcattttccaat

SEQ ID NO: 829 - RNA, 21 nt, synthetic construct
  source 1..21: mol_type = other RNA
tacaggcccatcattttccaa

SEQ ID NO: 830 - RNA, 21 nt, synthetic construct
  source 1..21: mol_type = other RNA
taacaggcccatcattttcca

SEQ ID NO: 831 - RNA, 21 nt, synthetic construct
  source 1..21: mol_type = other RNA
ttaacaggcccatcattttcc

SEQ ID NO: 832 - RNA, 21 nt, synthetic construct
  source 1..21: mol_type = other RNA
ttctttggctcagagttgcca

SEQ ID NO: 833 - RNA, 21 nt, synthetic construct
  source 1..21: mol_type = other RNA
tagacatttctttggctcaga

SEQ ID NO: 834 - RNA, 21 nt, synthetic construct
  source 1..21: mol_type = other RNA
tcagacatttctttggctcag

SEQ ID NO: 835 - RNA, 21 nt, synthetic construct
  source 1..21: mol_type = other RNA
ttcagacatttctttggctca

SEQ ID NO: 836 - RNA, 21 nt, synthetic construct
  source 1..21: mol_type = other RNA
tctcagacatttctttggctc

SEQ ID NO: 837 - RNA, 21 nt, synthetic construct
  source 1..21: mol_type = other RNA
tcctcagacatttctttggct

SEQ ID NO: 838 - RNA, 21 nt, synthetic construct
  source 1..21: mol_type = other RNA
ttcctcagacatttctttggc

SEQ ID NO: 839 - RNA, 21 nt, synthetic construct
  source 1..21: mol_type = other RNA
tgtcctcagacatttctttgg

SEQ ID NO: 840 - RNA, 21 nt, synthetic construct
  source 1..21: mol_type = other RNA
tgaggaagggcagaatcgcag

SEQ ID NO: 841 - RNA, 21 nt, synthetic construct
  source 1..21: mol_type = other RNA
tactgcaaatctctcctgcag

SEQ ID NO: 842 - RNA, 21 nt, synthetic construct
  source 1..21: mol_type = other RNA
taactgcaaatctctcctgca

SEQ ID NO: 843 - RNA, 21 nt, synthetic construct
  source 1..21: mol_type = other RNA
tgaactgcaaatctctcctgc

SEQ ID NO: 844 - RNA, 21 nt, synthetic construct
  source 1..21: mol_type = other RNA
tagaactgcaaatctctcctg

SEQ ID NO: 845 - RNA, 21 nt, synthetic construct
  source 1..21: mol_type = other RNA
tcagaactgcaaatctctcct

SEQ ID NO: 846 - RNA, 21 nt, synthetic construct
  source 1..21: mol_type = other RNA
tagtacattttggcagaactg

SEQ ID NO: 847 - RNA, 21 nt, synthetic construct
  source 1..21: mol_type = other RNA
tcagtacattttggcagaact

SEQ ID NO: 848 - RNA, 21 nt, synthetic construct
  source 1..21: mol_type = other RNA
ttcagtacattttggcagaac

SEQ ID NO: 849 - RNA, 21 nt, synthetic construct
  source 1..21: mol_type = other RNA
tatcagtacattttggcagaa

SEQ ID NO: 850 - RNA, 21 nt, synthetic construct
  source 1..21: mol_type = other RNA
taatcagtacattttggcaga

SEQ ID NO: 851 - RNA, 21 nt, synthetic construct
  source 1..21: mol_type = other RNA
taaatcagtacattttggcag

SEQ ID NO: 852 - RNA, 21 nt, synthetic construct
  source 1..21: mol_type = other RNA
taaaatcagtacattttggca

SEQ ID NO: 853 - RNA, 21 nt, synthetic construct
  source 1..21: mol_type = other RNA
ttctttgcgtgtagaagactg

SEQ ID NO: 854 - RNA, 21 nt, synthetic construct
  source 1..21: mol_type = other RNA
tttctttgcgtgtagaagact

SEQ ID NO: 855 - RNA, 21 nt, synthetic construct
  source 1..21: mol_type = other RNA
tactgcattctttgcgtgtag

SEQ ID NO: 856 - RNA, 21 nt, synthetic construct
  source 1..21: mol_type = other RNA
taactgcattctttgcgtgta

SEQ ID NO: 857 - RNA, 21 nt, synthetic construct
  source 1..21: mol_type = other RNA
taaactgcattctttgcgtgt

SEQ ID NO: 858 - RNA, 21 nt, synthetic construct
  source 1..21: mol_type = other RNA
tgaaactgcattctttgcgtg

SEQ ID NO: 859 - RNA, 21 nt, synthetic construct
  source 1..21: mol_type = other RNA
tagaaactgcattctttgcgt

SEQ ID NO: 860 - RNA, 21 nt, synthetic construct
  source 1..21: mol_type = other RNA
taagaaactgcattctttgcg

SEQ ID NO: 861 - RNA, 21 nt, synthetic construct
  source 1..21: mol_type = other RNA
tgaagaaactgcattctttgc

SEQ ID NO: 862 - RNA, 21 nt, synthetic construct
  source 1..21: mol_type = other RNA
tatttgacgtttcgaatcttc

SEQ ID NO: 863 - RNA, 21 nt, synthetic construct
  source 1..21: mol_type = other RNA
ttgattaatttgacgtttcga

SEQ ID NO: 864 - RNA, 21 nt, synthetic construct
  source 1..21: mol_type = other RNA
tcaaactgactgagatcaaaa

SEQ ID NO: 865 - RNA, 21 nt, synthetic construct
  source 1..21: mol_type = other RNA
tgatgatccaaactgactgag

SEQ ID NO: 866 - RNA, 21 nt, synthetic construct
  source 1..21: mol_type = other RNA
tttgatgatccaaactgactg

SEQ ID NO: 867 - RNA, 21 nt, synthetic construct
  source 1..21: mol_type = other RNA
tgtatttgatgatccaaactg

SEQ ID NO: 868 - RNA, 21 nt, synthetic construct
  source 1..21: mol_type = other RNA
tacttgtatttgatgatccaa

SEQ ID NO: 869 - RNA, 21 nt, synthetic construct
  source 1..21: mol_type = other RNA
tgaagcagttttctgtaaatt

SEQ ID NO: 870 - RNA, 21 nt, synthetic construct
  source 1..21: mol_type = other RNA
tatttggaagcagttttctgt

SEQ ID NO: 871 - RNA, 21 nt, synthetic construct
  source 1..21: mol_type = other RNA
tgatttggaagcagttttctg

SEQ ID NO: 872 - RNA, 21 nt, synthetic construct
  source 1..21: mol_type = other RNA
ttgatttggaagcagttttct

SEQ ID NO: 873 - RNA, 21 nt, synthetic construct
  source 1..21: mol_type = other RNA
tctgatttggaagcagttttc

SEQ ID NO: 874 - RNA, 21 nt, synthetic construct
  source 1..21: mol_type = other RNA
tgctgatttggaagcagtttt

SEQ ID NO: 875 - RNA, 21 nt, synthetic construct
  source 1..21: mol_type = other RNA
ttttgttagctgatttggaag

SEQ ID NO: 876 - RNA, 21 nt, synthetic construct
  source 1..21: mol_type = other RNA
tgtttgttagctgatttggaa

SEQ ID NO: 877 - RNA, 21 nt, synthetic construct
  source 1..21: mol_type = other RNA
tcgtttgttagctgatttgga

SEQ ID NO: 878 - RNA, 21 nt, synthetic construct
  source 1..21: mol_type = other RNA
tccgtttgttagctgatttgg

SEQ ID NO: 879 - RNA, 21 nt, synthetic construct
  source 1..21: mol_type = other RNA
taccgtttgttagctgatttg

SEQ ID NO: 880 - RNA, 21 nt, synthetic construct
  source 1..21: mol_type = other RNA
tgaccgtttgttagctgattt

SEQ ID NO: 881 - RNA, 21 nt, synthetic construct
  source 1..21: mol_type = other RNA
tggaccgtttgttagctgatt

SEQ ID NO: 882 - RNA, 21 nt, synthetic construct
  source 1..21: mol_type = other RNA
tgcttttggaccgtttgttag

SEQ ID NO: 883 - RNA, 21 nt, synthetic construct
  source 1..21: mol_type = other RNA
tatgcttttggaccgtttgtt

SEQ ID NO: 884 - RNA, 21 nt, synthetic construct
  source 1..21: mol_type = other RNA
tgtatagatgcttttggaccg

SEQ ID NO: 885 - RNA, 21 nt, synthetic construct
  source 1..21: mol_type = other RNA
tcgtatagatgcttttggacc

SEQ ID NO: 886 - RNA, 21 nt, synthetic construct
  source 1..21: mol_type = other RNA
tgcgtatagatgcttttggac

SEQ ID NO: 887 - RNA, 21 nt, synthetic construct
  source 1..21: mol_type = other RNA
tggcgtatagatgcttttgga

SEQ ID NO: 888 - RNA, 21 nt, synthetic construct
  source 1..21: mol_type = other RNA
taaactgcatctttgtgctgc

SEQ ID NO: 889 - RNA, 21 nt, synthetic construct
  source 1..21: mol_type = other RNA
taaaactgcatctttgtgctg

SEQ ID NO: 890 - RNA, 21 nt, synthetic construct
  source 1..21: mol_type = other RNA
tatatccacattccacacaca

SEQ ID NO: 891 - RNA, 21 nt, synthetic construct
  source 1..21: mol_type = other RNA
ttatatccacattccacacac

SEQ ID NO: 892 - RNA, 21 nt, synthetic construct
  source 1..21: mol_type = other RNA
tttatatccacattccacaca

SEQ ID NO: 893 - RNA, 21 nt, synthetic construct
  source 1..21: mol_type = other RNA
tcttatatccacattccacac

SEQ ID NO: 894 - RNA, 21 nt, synthetic construct
  source 1..21: mol_type = other RNA
tacttatatccacattccaca

SEQ ID NO: 895 - RNA, 21 nt, synthetic construct
  source 1..21: mol_type = other RNA
ttacttatatccacattccac

SEQ ID NO: 896 - RNA, 21 nt, synthetic construct
  source 1..21: mol_type = other RNA
ttctatacttatatccacatt

SEQ ID NO: 897 - RNA, 21 nt, synthetic construct
  source 1..21: mol_type = other RNA
tatctatacttatatccacat

SEQ ID NO: 898 - RNA, 21 nt, synthetic construct
  source 1..21: mol_type = other RNA
taatctatacttatatccaca

SEQ ID NO: 899 - RNA, 21 nt, synthetic construct
  source 1..21: mol_type = other RNA
tgaatctatacttatatccac

SEQ ID NO: 900 - RNA, 21 nt, synthetic construct
  source 1..21: mol_type = other RNA
tagaatctatacttatatcca

SEQ ID NO: 901 - RNA, 21 nt, synthetic construct
  source 1..21: mol_type = other RNA
tgtgatctaaatggcaataaa

SEQ ID NO: 902 - RNA, 21 nt, synthetic construct
  source 1..21: mol_type = other RNA
tgttgtgatctaaatggcaat

SEQ ID NO: 903 - RNA, 21 nt, synthetic construct
  source 1..21: mol_type = other RNA
tagttgtgatctaaatggcaa

SEQ ID NO: 904 - RNA, 21 nt, synthetic construct
  source 1..21: mol_type = other RNA
taagttgtgatctaaatggca

SEQ ID NO: 905 - RNA, 21 nt, synthetic construct
  source 1..21: mol_type = other RNA
taaagttgtgatctaaatggc

SEQ ID NO: 906 - RNA, 21 nt, synthetic construct
  source 1..21: mol_type = other RNA
ttaaagttgtgatctaaatgg

SEQ ID NO: 907 - RNA, 21 nt, synthetic construct
  source 1..21: mol_type = other RNA
tataaagttgtgatctaaatg

SEQ ID NO: 908 - RNA, 21 nt, synthetic construct
  source 1..21: mol_type = other RNA
tcataaagttgtgatctaaat

SEQ ID NO: 909 - RNA, 21 nt, synthetic construct
  source 1..21: mol_type = other RNA
tctgtcataaagttgtgatct

SEQ ID NO: 910 - RNA, 21 nt, synthetic construct
  source 1..21: mol_type = other RNA
tgctgtcataaagttgtgatc

SEQ ID NO: 911 - RNA, 21 nt, synthetic construct
  source 1..21: mol_type = other RNA
ttgctgtcataaagttgtgat

SEQ ID NO: 912 - RNA, 21 nt, synthetic construct
  source 1..21: mol_type = other RNA
tttgctgtcataaagttgtga

SEQ ID NO: 913 - RNA, 21 nt, synthetic construct
  source 1..21: mol_type = other RNA
tcttgctgtcataaagttgtg

SEQ ID NO: 914 - RNA, 21 nt, synthetic construct
  source 1..21: mol_type = other RNA
tacttgctgtcataaagttgt

SEQ ID NO: 915 - RNA, 21 nt, synthetic construct
  source 1..21: mol_type = other RNA
ttacttgctgtcataaagttg

SEQ ID NO: 916 - RNA, 21 nt, synthetic construct
  source 1..21: mol_type = other RNA
tacagtctgtgagtaggtata

SEQ ID NO: 917 - RNA, 21 nt, synthetic construct
  source 1..21: mol_type = other RNA
tgcttcacaactcccacctta

SEQ ID NO: 918 - RNA, 21 nt, synthetic construct
  source 1..21: mol_type = other RNA
ttgcttcacaactcccacctt

SEQ ID NO: 919 - RNA, 21 nt, synthetic construct
  source 1..21: mol_type = other RNA
tttgcttcacaactcccacct

SEQ ID NO: 920 - RNA, 21 nt, synthetic construct
  source 1..21: mol_type = other RNA
tgtttgcttcacaactcccac

SEQ ID NO: 921 - RNA, 21 nt, synthetic construct
  source 1..21: mol_type = other RNA
tagtttgcttcacaactccca

SEQ ID NO: 922 - RNA, 21 nt, synthetic construct
  source 1..21: mol_type = other RNA
tcagtttgcttcacaactccc

SEQ ID NO: 923 - RNA, 21 nt, synthetic construct
  source 1..21: mol_type = other RNA
ttcagtttgcttcacaactcc

SEQ ID NO: 924 - RNA, 21 nt, synthetic construct
  source 1..21: mol_type = other RNA
tttcagtttgcttcacaactc

SEQ ID NO: 925 - RNA, 21 nt, synthetic construct
  source 1..21: mol_type = other RNA
ttttcagtttgcttcacaact

SEQ ID NO: 926 - RNA, 21 nt, synthetic construct
  source 1..21: mol_type = other RNA
tgtttcagtttgcttcacaac

SEQ ID NO: 927 - RNA, 21 nt, synthetic construct
  source 1..21: mol_type = other RNA
tagtttcagtttgcttcacaa

SEQ ID NO: 928 - RNA, 21 nt, synthetic construct
  source 1..21: mol_type = other RNA
tcagtttcagtttgcttcaca

SEQ ID NO: 929 - RNA, 21 nt, synthetic construct
  source 1..21: mol_type = other RNA
tgcagtttcagtttgcttcac

SEQ ID NO: 930 - RNA, 21 nt, synthetic construct
  source 1..21: mol_type = other RNA
ttgcagtttcagtttgcttca

SEQ ID NO: 931 - RNA, 21 nt, synthetic construct
  source 1..21: mol_type = other RNA
tctgcagtttcagtttgcttc

SEQ ID NO: 932 - RNA, 21 nt, synthetic construct
  source 1..21: mol_type = other RNA
tgctgcagtttcagtttgctt

SEQ ID NO: 933 - RNA, 21 nt, synthetic construct
  source 1..21: mol_type = other RNA
tgaacttctgttgtctccaat

SEQ ID NO: 934 - RNA, 21 nt, synthetic construct
  source 1..21: mol_type = other RNA
tagggcagtcaatttccggga

SEQ ID NO: 935 - RNA, 21 nt, synthetic construct
  source 1..21: mol_type = other RNA
tataaagggcagtcaatttcc

SEQ ID NO: 936 - RNA, 21 nt, synthetic construct
  source 1..21: mol_type = other RNA
ttttgtataaagggcagtcaa

SEQ ID NO: 937 - RNA, 21 nt, synthetic construct
  source 1..21: mol_type = other RNA
tttacagcatcatccagcttg

SEQ ID NO: 938 - RNA, 21 nt, synthetic construct
  source 1..21: mol_type = other RNA
ttcattatctcatcaacattt

SEQ ID NO: 939 - RNA, 21 nt, synthetic construct
  source 1..21: mol_type = other RNA
tagtatcagtcattatctcat

SEQ ID NO: 940 - RNA, 21 nt, synthetic construct
  source 1..21: mol_type = other RNA
tattttcagagatgcacagaa

SEQ ID NO: 941 - RNA, 21 nt, synthetic construct
  source 1..21: mol_type = other RNA
ttattttcagagatgcacaga

SEQ ID NO: 942 - RNA, 21 nt, synthetic construct
  source 1..21: mol_type = other RNA
tttattttcagagatgcacag

SEQ ID NO: 943 - RNA, 21 nt, synthetic construct
  source 1..21: mol_type = other RNA
tcttattttcagagatgcaca

SEQ ID NO: 944 - RNA, 21 nt, synthetic construct
  source 1..21: mol_type = other RNA
tccttattttcagagatgcac

SEQ ID NO: 945 - RNA, 21 nt, synthetic construct
  source 1..21: mol_type = other RNA
ttccttattttcagagatgca

SEQ ID NO: 946 - RNA, 21 nt, synthetic construct
  source 1..21: mol_type = other RNA
tttccttattttcagagatgc

SEQ ID NO: 947 - RNA, 21 nt, synthetic construct
  source 1..21: mol_type = other RNA
ttttccttattttcagagatg

SEQ ID NO: 948 - RNA, 21 nt, synthetic construct
  source 1..21: mol_type = other RNA
tattttccttattttcagaga

SEQ ID NO: 949 - RNA, 21 nt, synthetic construct
  source 1..21: mol_type = other RNA
tcattttccttattttcagag

SEQ ID NO: 950 - RNA, 21 nt, synthetic construct
  source 1..21: mol_type = other RNA
taggcttgacatccgggtttc

SEQ ID NO: 951 - RNA, 21 nt, synthetic construct
  source 1..21: mol_type = other RNA
tgctgcaggcttgacatccgg

SEQ ID NO: 952 - RNA, 21 nt, synthetic construct
  source 1..21: mol_type = other RNA
tctactggctgcaggcttgac

SEQ ID NO: 953 - RNA, 21 nt, synthetic construct
  source 1..21: mol_type = other RNA
tgctcggacaaggccgaagga

SEQ ID NO: 954 - RNA, 21 nt, synthetic construct
  source 1..21: mol_type = other RNA
tgatgagcgcctctgtttgct

SEQ ID NO: 955 - RNA, 21 nt, synthetic construct
  source 1..21: mol_type = other RNA
tgatgtggctctgtggatgag

SEQ ID NO: 956 - RNA, 21 nt, synthetic construct
  source 1..21: mol_type = other RNA
tagcatggctgtattcaaaat

SEQ ID NO: 957 - RNA, 21 nt, synthetic construct
  source 1..21: mol_type = other RNA
taagcatggctgtattcaaaa

SEQ ID NO: 958 - RNA, 21 nt, synthetic construct
  source 1..21: mol_type = other RNA
taaagcatggctgtattcaaa

SEQ ID NO: 959 - RNA, 21 nt, synthetic construct
  source 1..21: mol_type = other RNA
tgaaagcatggctgtattcaa

SEQ ID NO: 960 - RNA, 21 nt, synthetic construct
  source 1..21: mol_type = other RNA
tggaaagcatggctgtattca

SEQ ID NO: 961 - RNA, 21 nt, synthetic construct
  source 1..21: mol_type = other RNA
ttggaaagcatggctgtattc

SEQ ID NO: 962 - RNA, 21 nt, synthetic construct
  source 1..21: mol_type = other RNA
tctggaaagcatggctgtatt

SEQ ID NO: 963 - RNA, 21 nt, synthetic construct
  source 1..21: mol_type = other RNA
tctctgtaactgcctggaaag

SEQ ID NO: 964 - RNA, 21 nt, synthetic construct
  source 1..21: mol_type = other RNA
tactctgtaactgcctggaaa

SEQ ID NO: 965 - RNA, 21 nt, synthetic construct
  source 1..21: mol_type = other RNA
taactctgtaactgcctggaa

SEQ ID NO: 966 - RNA, 21 nt, synthetic construct
  source 1..21: mol_type = other RNA
taaactctgtaactgcctgga

SEQ ID NO: 967 - RNA, 21 nt, synthetic construct
  source 1..21: mol_type = other RNA
taaaactctgtaactgcctgg

SEQ ID NO: 968 - RNA, 21 nt, synthetic construct
  source 1..21: mol_type = other RNA
ttaaaactctgtaactgcctg

SEQ ID NO: 969 - RNA, 21 nt, synthetic construct
  source 1..21: mol_type = other RNA
tataaaactctgtaactgcct

SEQ ID NO: 970 - RNA, 21 nt, synthetic construct
  source 1..21: mol_type = other RNA
taactgtatcttttgcataaa

SEQ ID NO: 971 - RNA, 21 nt, synthetic construct
  source 1..21: mol_type = other RNA
tcctttgatgtcaactgtatc

SEQ ID NO: 972 - RNA, 21 nt, synthetic construct
  source 1..21: mol_type = other RNA
tacctttgatgtcaactgtat

SEQ ID NO: 973 - RNA, 21 nt, synthetic construct
  source 1..21: mol_type = other RNA
taacctttgatgtcaactgta

SEQ ID NO: 974 - RNA, 21 nt, synthetic construct
  source 1..21: mol_type = other RNA
tgaacctttgatgtcaactgt

SEQ ID NO: 975 - RNA, 21 nt, synthetic construct
  source 1..21: mol_type = other RNA
tagaacctttgatgtcaactg

SEQ ID NO: 976 - RNA, 21 nt, synthetic construct
  source 1..21: mol_type = other RNA
tgagaacctttgatgtcaact

SEQ ID NO: 977 - RNA, 21 nt, synthetic construct
  source 1..21: mol_type = other RNA
ttgagaacctttgatgtcaac

SEQ ID NO: 978 - RNA, 21 nt, synthetic construct
  source 1..21: mol_type = other RNA
tttgagaacctttgatgtcaa

SEQ ID NO: 979 - RNA, 21 nt, synthetic construct
  source 1..21: mol_type = other RNA
tagttaacaatgccagaaata

SEQ ID NO: 980 - RNA, 21 nt, synthetic construct
  source 1..21: mol_type = other RNA
taagttaacaatgccagaaat

SEQ ID NO: 981 - RNA, 21 nt, synthetic construct
  source 1..21: mol_type = other RNA
ttaagttaacaatgccagaaa

SEQ ID NO: 982 - RNA, 21 nt, synthetic construct
  source 1..21: mol_type = other RNA
tctaagttaacaatgccagaa

SEQ ID NO: 983 - RNA, 21 nt, synthetic construct
  source 1..21: mol_type = other RNA
ttctaagttaacaatgccaga

SEQ ID NO: 984 - RNA, 21 nt, synthetic construct
  source 1..21: mol_type = other RNA
tctctaagttaacaatgccag

SEQ ID NO: 985 - RNA, 21 nt, synthetic construct
  source 1..21: mol_type = other RNA
ttctctaagttaacaatgcca

SEQ ID NO: 986 - RNA, 21 nt, synthetic construct
  source 1..21: mol_type = other RNA
ttcacaggcttctctaagtta

SEQ ID NO: 987 - RNA, 21 nt, synthetic construct
  source 1..21: mol_type = other RNA
ttggcagccaaagagcaaatc

SEQ ID NO: 988 - RNA, 21 nt, synthetic construct
  source 1..21: mol_type = other RNA
ttatgatggcagccaaagagc

SEQ ID NO: 989 - RNA, 21 nt, synthetic construct
  source 1..21: mol_type = other RNA
tggtattttatgatggcagcc

SEQ ID NO: 990 - RNA, 21 nt, synthetic construct
  source 1..21: mol_type = other RNA
tgaggtattttatgatggcag

SEQ ID NO: 991 - RNA, 21 nt, synthetic construct
  source 1..21: mol_type = other RNA
ttgaggtattttatgatggca

SEQ ID NO: 992 - RNA, 21 nt, synthetic construct
  source 1..21: mol_type = other RNA
tttgaggtattttatgatggc

SEQ ID NO: 993 - RNA, 21 nt, synthetic construct
  source 1..21: mol_type = other RNA
ttttgaggtattttatgatgg

SEQ ID NO: 994 - RNA, 21 nt, synthetic construct
  source 1..21: mol_type = other RNA
tctttgaggtattttatgatg

SEQ ID NO: 995 - RNA, 21 nt, synthetic construct
  source 1..21: mol_type = other RNA
ttctttgaggtattttatgat

SEQ ID NO: 996 - RNA, 21 nt, synthetic construct
  source 1..21: mol_type = other RNA
tttctttgaggtattttatga

SEQ ID NO: 997 - RNA, 21 nt, synthetic construct
  source 1..21: mol_type = other RNA
tttccaagttgaattctttga

SEQ ID NO: 998 - RNA, 21 nt, synthetic construct
  source 1..21: mol_type = other RNA
tgagcatcttttccaagttga

SEQ ID NO: 999 - RNA, 21 nt, synthetic construct
  source 1..21: mol_type = other RNA
tagctgtttaaaattctcagg

SEQ ID NO: 1000 - RNA, 21 nt, synthetic construct
  source 1..21: mol_type = other RNA
ttagctgtttaaaattctcag

SEQ ID NO: 1001 - RNA, 21 nt, synthetic construct
  source 1..21: mol_type = other RNA
tatagctgtttaaaattctca

SEQ ID NO: 1002 - RNA, 21 nt, synthetic construct
  source 1..21: mol_type = other RNA
tgatagctgtttaaaattctc

SEQ ID NO: 1003 - RNA, 21 nt, synthetic construct
  source 1..21: mol_type = other RNA
ttgatagctgtttaaaattct

SEQ ID NO: 1004 - RNA, 21 nt, synthetic construct
  source 1..21: mol_type = other RNA
tgattccttaatgttgttcca

SEQ ID NO: 1005 - RNA, 21 nt, synthetic construct
  source 1..21: mol_type = other RNA
tagattccttaatgttgttcc

SEQ ID NO: 1006 - RNA, 21 nt, synthetic construct
  source 1..21: mol_type = other RNA
tcagattccttaatgttgttc

SEQ ID NO: 1007 - RNA, 21 nt, synthetic construct
  source 1..21: mol_type = other RNA
tccagattccttaatgttgtt

SEQ ID NO: 1008 - RNA, 21 nt, synthetic construct
  source 1..21: mol_type = other RNA
ttccagattccttaatgttgt

SEQ ID NO: 1009 - RNA, 21 nt, synthetic construct
  source 1..21: mol_type = other RNA
ttatcagtctgattctgtagg

SEQ ID NO: 1010 - RNA, 21 nt, synthetic construct
  source 1..21: mol_type = other RNA
tatatcagtctgattctgtag

SEQ ID NO: 1011 - RNA, 21 nt, synthetic construct
  source 1..21: mol_type = other RNA
tcatatcagtctgattctgta

SEQ ID NO: 1012 - RNA, 21 nt, synthetic construct
  source 1..21: mol_type = other RNA
ttcatatcagtctgattctgt

SEQ ID NO: 1013 - RNA, 21 nt, synthetic construct
  source 1..21: mol_type = other RNA
tttcatatcagtctgattctg

SEQ ID NO: 1014 - RNA, 21 nt, synthetic construct
  source 1..21: mol_type = other RNA
ttttcatatcagtctgattct

SEQ ID NO: 1015 - RNA, 21 nt, synthetic construct
  source 1..21: mol_type = other RNA
tgttttcatatcagtctgatt

SEQ ID NO: 1016 - RNA, 21 nt, synthetic construct
  source 1..21: mol_type = other RNA
tttggttttcatatcagtctg

SEQ ID NO: 1017 - RNA, 21 nt, synthetic construct
  source 1..21: mol_type = other RNA
tagtgtggtctaaaacccaca

SEQ ID NO: 1018 - RNA, 21 nt, synthetic construct
  source 1..21: mol_type = other RNA
ttagtgtggtctaaaacccac

SEQ ID NO: 1019 - RNA, 21 nt, synthetic construct
  source 1..21: mol_type = other RNA
tttagtgtggtctaaaaccca

SEQ ID NO: 1020 - RNA, 21 nt, synthetic construct
  source 1..21: mol_type = other RNA
taacttccgtctcccaaatga

SEQ ID NO: 1021 - RNA, 21 nt, synthetic construct
  source 1..21: mol_type = other RNA
tatttaaggagtggctgggtc

SEQ ID NO: 1022 - RNA, 21 nt, synthetic construct
  source 1..21: mol_type = other RNA
tcttccgatacagcatcaagc

SEQ ID NO: 1023 - RNA, 21 nt, synthetic construct
  source 1..21: mol_type = other RNA
tacttccgatacagcatcaag

SEQ ID NO: 1024 - RNA, 21 nt, synthetic construct
  source 1..21: mol_type = other RNA
taacttccgatacagcatcaa

SEQ ID NO: 1025 - RNA, 21 nt, synthetic construct
  source 1..21: mol_type = other RNA
tgaacttccgatacagcatca

SEQ ID NO: 1026 - RNA, 21 nt, synthetic construct
  source 1..21: mol_type = other RNA
tagaacttccgatacagcatc

SEQ ID NO: 1027 - RNA, 21 nt, synthetic construct
  source 1..21: mol_type = other RNA
tgagaacttccgatacagcat

SEQ ID NO: 1028 - RNA, 21 nt, synthetic construct
  source 1..21: mol_type = other RNA
tggagaacttccgatacagca

SEQ ID NO: 1029 - RNA, 21 nt, synthetic construct
  source 1..21: mol_type = other RNA
ttttctatctgaccaaacaca

SEQ ID NO: 1030 - RNA, 21 nt, synthetic construct
  source 1..21: mol_type = other RNA
tattttctatctgaccaaaca

SEQ ID NO: 1031 - RNA, 21 nt, synthetic construct
  source 1..21: mol_type = other RNA
tgattttctatctgaccaaac

SEQ ID NO: 1032 - RNA, 21 nt, synthetic construct
  source 1..21: mol_type = other RNA
ttgattttctatctgaccaaa

SEQ ID NO: 1033 - RNA, 21 nt, synthetic construct
  source 1..21: mol_type = other RNA
tatgattttctatctgaccaa

SEQ ID NO: 1034 - RNA, 21 nt, synthetic construct
  source 1..21: mol_type = other RNA
tgatgattttctatctgacca

SEQ ID NO: 1035 - RNA, 21 nt, synthetic construct
  source 1..21: mol_type = other RNA
ttctctatgtcgggcaattta

SEQ ID NO: 1036 - RNA, 21 nt, synthetic construct
  source 1..21: mol_type = other RNA
ttgtgataaatgctacagagt

SEQ ID NO: 1037 - RNA, 21 nt, synthetic construct
  source 1..21: mol_type = other RNA
tttgtgataaatgctacagag

SEQ ID NO: 1038 - RNA, 21 nt, synthetic construct
  source 1..21: mol_type = other RNA
ttttgtgataaatgctacaga

SEQ ID NO: 1039 - RNA, 21 nt, synthetic construct
  source 1..21: mol_type = other RNA
tagaactcttgggtagaacat

SEQ ID NO: 1040 - RNA, 21 nt, synthetic construct
  source 1..21: mol_type = other RNA
tgaagaactcttgggtagaac

SEQ ID NO: 1041 - RNA, 21 nt, synthetic construct
  source 1..21: mol_type = other RNA
tagaagaactcttgggtagaa

SEQ ID NO: 1042 - RNA, 21 nt, synthetic construct
  source 1..21: mol_type = other RNA
taagaagaactcttgggtaga

SEQ ID NO: 1043 - RNA, 21 nt, synthetic construct
  source 1..21: mol_type = other RNA
tcaagaagaactcttgggtag

SEQ ID NO: 1044 - RNA, 21 nt, synthetic construct
  source 1..21: mol_type = other RNA
ttcaagaagaactcttgggta

SEQ ID NO: 1045 - RNA, 21 nt, synthetic construct
  source 1..21: mol_type = other RNA
tatcaagaagaactcttgggt

SEQ ID NO: 1046 - RNA, 21 nt, synthetic construct
  source 1..21: mol_type = other RNA
tagttttgacaatcaagaaga

SEQ ID NO: 1047 - RNA, 21 nt, synthetic construct
  source 1..21: mol_type = other RNA
taattctgactttaggtgata

SEQ ID NO: 1048 - RNA, 21 nt, synthetic construct
  source 1..21: mol_type = other RNA
taaattctgactttaggtgat

SEQ ID NO: 1049 - RNA, 21 nt, synthetic construct
  source 1..21: mol_type = other RNA
tgaaattctgactttaggtga

SEQ ID NO: 1050 - RNA, 21 nt, synthetic construct
  source 1..21: mol_type = other RNA
ttgaaattctgactttaggtg

SEQ ID NO: 1051 - RNA, 21 nt, synthetic construct
  source 1..21: mol_type = other RNA
tttgaaattctgactttaggt

SEQ ID NO: 1052 - RNA, 21 nt, synthetic construct
  source 1..21: mol_type = other RNA
tagcaggtattattgcttgaa

SEQ ID NO: 1053 - RNA, 21 nt, synthetic construct
  source 1..21: mol_type = other RNA
tgggaattaacagcaggtatt

SEQ ID NO: 1054 - RNA, 21 nt, synthetic construct
  source 1..21: mol_type = other RNA
tgtgggaattaacagcaggta

SEQ ID NO: 1055 - RNA, 21 nt, synthetic construct
  source 1..21: mol_type = other RNA
tgtccggagcaagtctgactg

SEQ ID NO: 1056 - RNA, 21 nt, synthetic construct
  source 1..21: mol_type = other RNA
tccactggactgaggagttca

SEQ ID NO: 1057 - RNA, 21 nt, synthetic construct
  source 1..21: mol_type = other RNA
taagtaatgctccactggact

SEQ ID NO: 1058 - RNA, 21 nt, synthetic construct
  source 1..21: mol_type = other RNA
tttaagtaatgctccactgga

SEQ ID NO: 1059 - RNA, 21 nt, synthetic construct
  source 1..21: mol_type = other RNA
tggaagtcagaaaggtcttta

SEQ ID NO: 1060 - RNA, 21 nt, synthetic construct
  source 1..21: mol_type = other RNA
tttaaagggaagtcagaaagg

SEQ ID NO: 1061 - RNA, 21 nt, synthetic construct
  source 1..21: mol_type = other RNA
tattaaagggaagtcagaaag

SEQ ID NO: 1062 - RNA, 21 nt, synthetic construct
  source 1..21: mol_type = other RNA
ttattaaagggaagtcagaaa

SEQ ID NO: 1063 - RNA, 21 nt, synthetic construct
  source 1..21: mol_type = other RNA
tttattaaagggaagtcagaa

SEQ ID NO: 1064 - RNA, 21 nt, synthetic construct
  source 1..21: mol_type = other RNA
ttttattaaagggaagtcaga

SEQ ID NO: 1065 - RNA, 21 nt, synthetic construct
  source 1..21: mol_type = other RNA
tgcattcggatctcgtcaata

SEQ ID NO: 1066 - RNA, 21 nt, synthetic construct
  source 1..21: mol_type = other RNA
ttcttgcaaatgcattcggat

SEQ ID NO: 1067 - RNA, 21 nt, synthetic construct
  source 1..21: mol_type = other RNA
tttcttgcaaatgcattcgga

SEQ ID NO: 1068 - RNA, 21 nt, synthetic construct
  source 1..21: mol_type = other RNA
ttttcttgcaaatgcattcgg

SEQ ID NO: 1069 - RNA, 21 nt, synthetic construct
  source 1..21: mol_type = other RNA
tatttcttgcaaatgcattcg

SEQ ID NO: 1070 - RNA, 21 nt, synthetic construct
  source 1..21: mol_type = other RNA
ttatttcttgcaaatgcattc

SEQ ID NO: 1071 - RNA, 21 nt, synthetic construct
  source 1..21: mol_type = other RNA
tttcgtatttcttgcaaatgc

SEQ ID NO: 1072 - RNA, 21 nt, synthetic construct
  source 1..21: mol_type = other RNA
tctcctgtcctgatactgtca

SEQ ID NO: 1073 - RNA, 21 nt, synthetic construct
  source 1..21: mol_type = other RNA
tactcctgtcctgatactgtc

SEQ ID NO: 1074 - RNA, 21 nt, synthetic construct
  source 1..21: mol_type = other RNA
taactcctgtcctgatactgt

SEQ ID NO: 1075 - RNA, 21 nt, synthetic construct
  source 1..21: mol_type = other RNA
taaactcctgtcctgatactg

SEQ ID NO: 1076 - RNA, 21 nt, synthetic construct
  source 1..21: mol_type = other RNA
ttaaactcctgtcctgatact

SEQ ID NO: 1077 - RNA, 21 nt, synthetic construct
  source 1..21: mol_type = other RNA
tataaactcctgtcctgatac

SEQ ID NO: 1078 - RNA, 21 nt, synthetic construct
  source 1..21: mol_type = other RNA
tcataaactcctgtcctgata

SEQ ID NO: 1079 - RNA, 21 nt, synthetic construct
  source 1..21: mol_type = other RNA
tagttctttatttctatcata

SEQ ID NO: 1080 - RNA, 21 nt, synthetic construct
  source 1..21: mol_type = other RNA
tgagttctttatttctatcat

SEQ ID NO: 1081 - RNA, 21 nt, synthetic construct
  source 1..21: mol_type = other RNA
tagagttctttatttctatca

SEQ ID NO: 1082 - RNA, 21 nt, synthetic construct
  source 1..21: mol_type = other RNA
tcagagttctttatttctatc

SEQ ID NO: 1083 - RNA, 21 nt, synthetic construct
  source 1..21: mol_type = other RNA
tgcagagttctttatttctat

SEQ ID NO: 1084 - RNA, 21 nt, synthetic construct
  source 1..21: mol_type = other RNA
ttcagttggtatacaagatac

SEQ ID NO: 1085 - RNA, 21 nt, synthetic construct
  source 1..21: mol_type = other RNA
ttttgtgcttccaacctttac

SEQ ID NO: 1086 - RNA, 21 nt, synthetic construct
  source 1..21: mol_type = other RNA
tcacagcttttgtgcttccaa

SEQ ID NO: 1087 - RNA, 21 nt, synthetic construct
  source 1..21: mol_type = other RNA
ttcacagcttttgtgcttcca

SEQ ID NO: 1088 - RNA, 21 nt, synthetic construct
  source 1..21: mol_type = other RNA
tctcacagcttttgtgcttcc

SEQ ID NO: 1089 - RNA, 21 nt, synthetic construct
  source 1..21: mol_type = other RNA
tgctcacagcttttgtgcttc

SEQ ID NO: 1090 - RNA, 21 nt, synthetic construct
  source 1..21: mol_type = other RNA
tggctcacagcttttgtgctt

SEQ ID NO: 1091 - RNA, 21 nt, synthetic construct
  source 1..21: mol_type = other RNA
tcggctcacagcttttgtgct

SEQ ID NO: 1092 - RNA, 21 nt, synthetic construct
  source 1..21: mol_type = other RNA
tgcggctcacagcttttgtgc

SEQ ID NO: 1093 - RNA, 21 nt, synthetic construct
  source 1..21: mol_type = other RNA
tggagagtgaaagcggctcac

SEQ ID NO: 1094 - RNA, 21 nt, synthetic construct
  source 1..21: mol_type = other RNA
taggagagtgaaagcggctca

SEQ ID NO: 1095 - RNA, 21 nt, synthetic construct
  source 1..21: mol_type = other RNA
taaggagagtgaaagcggctc

SEQ ID NO: 1096 - RNA, 21 nt, synthetic construct
  source 1..21: mol_type = other RNA
taaaggagagtgaaagcggct

SEQ ID NO: 1097 - RNA, 21 nt, synthetic construct
  source 1..21: mol_type = other RNA
taaaaggagagtgaaagcggc

SEQ ID NO: 1098 - RNA, 21 nt, synthetic construct
  source 1..21: mol_type = other RNA
ttaaaaggagagtgaaagcgg

SEQ ID NO: 1099 - RNA, 21 nt, synthetic construct
  source 1..21: mol_type = other RNA
ttcaagccattcagcactgca

SEQ ID NO: 1100 - RNA, 21 nt, synthetic construct
  source 1..21: mol_type = other RNA
tatcaagccattcagcactgc

SEQ ID NO: 1101 - RNA, 21 nt, synthetic construct
  source 1..21: mol_type = other RNA
taatcaagccattcagcactg

SEQ ID NO: 1102 - RNA, 21 nt, synthetic construct
  source 1..21: mol_type = other RNA
taaatcaagccattcagcact

SEQ ID NO: 1103 - RNA, 21 nt, synthetic construct
  source 1..21: mol_type = other RNA
taaaatcaagccattcagcac

SEQ ID NO: 1104 - RNA, 21 nt, synthetic construct
  source 1..21: mol_type = other RNA
ttagaaaatcaagccattcag

SEQ ID NO: 1105 - RNA, 21 nt, synthetic construct
  source 1..21: mol_type = other RNA
tctagaaaatcaagccattca

SEQ ID NO: 1106 - RNA, 21 nt, synthetic construct
  source 1..21: mol_type = other RNA
ttctagaaaatcaagccattc

SEQ ID NO: 1107 - RNA, 21 nt, synthetic construct
  source 1..21: mol_type = other RNA
tctctagaaaatcaagccatt

SEQ ID NO: 1108 - RNA, 21 nt, synthetic construct
  source 1..21: mol_type = other RNA
ttctctagaaaatcaagccat

SEQ ID NO: 1109 - RNA, 21 nt, synthetic construct
  source 1..21: mol_type = other RNA
tttctctagaaaatcaagcca

SEQ ID NO: 1110 - RNA, 21 nt, synthetic construct
  source 1..21: mol_type = other RNA
ttttctctagaaaatcaagcc

SEQ ID NO: 1111 - RNA, 21 nt, synthetic construct
  source 1..21: mol_type = other RNA
ttgaatttctctagaaaatca

SEQ ID NO: 1112 - RNA, 21 nt, synthetic construct
  source 1..21: mol_type = other RNA
ttcactgaatttctctagaaa

SEQ ID NO: 1113 - RNA, 21 nt, synthetic construct
  source 1..21: mol_type = other RNA
tgagtgataatgttcactgaa

SEQ ID NO: 1114 - RNA, 21 nt, synthetic construct
  source 1..21: mol_type = other RNA
taggagtgataatgttcactg

SEQ ID NO: 1115 - RNA, 21 nt, synthetic construct
  source 1..21: mol_type = other RNA
tacagttgctaggtgatgcac

SEQ ID NO: 1116 - RNA, 21 nt, synthetic construct
  source 1..21: mol_type = other RNA
tgggagaaaatgcagtcaaca

SEQ ID NO: 1117 - RNA, 21 nt, synthetic construct
  source 1..21: mol_type = other RNA
tctccttgcttagcgaccttg

SEQ ID NO: 1118 - RNA, 21 nt, synthetic construct
  source 1..21: mol_type = other RNA
tgcagtaatctccttgcttag

SEQ ID NO: 1119 - RNA, 21 nt, synthetic construct
  source 1..21: mol_type = other RNA
ttgagtcctctgataaatctg

SEQ ID NO: 1120 - RNA, 21 nt, synthetic construct
  source 1..21: mol_type = other RNA
tttactctctctgagtcctct

SEQ ID NO: 1121 - RNA, 21 nt, synthetic construct
  source 1..21: mol_type = other RNA
tttatcattactctctctgag

SEQ ID NO: 1122 - RNA, 21 nt, synthetic construct
  source 1..21: mol_type = other RNA
tattatcattactctctctga

SEQ ID NO: 1123 - RNA, 21 nt, synthetic construct
  source 1..21: mol_type = other RNA
taattatcattactctctctg

SEQ ID NO: 1124 - RNA, 21 nt, synthetic construct
  source 1..21: mol_type = other RNA
ttaattatcattactctctct

SEQ ID NO: 1125 - RNA, 21 nt, synthetic construct
  source 1..21: mol_type = other RNA
tatgtaggagctctttccacc

SEQ ID NO: 1126 - RNA, 21 nt, synthetic construct
  source 1..21: mol_type = other RNA
tgttttatgtaggagctcttt

SEQ ID NO: 1127 - RNA, 21 nt, synthetic construct
  source 1..21: mol_type = other RNA
ttgttttatgtaggagctctt

SEQ ID NO: 1128 - RNA, 21 nt, synthetic construct
  source 1..21: mol_type = other RNA
tcttgttttatgtaggagctc

SEQ ID NO: 1129 - RNA, 21 nt, synthetic construct
  source 1..21: mol_type = other RNA
tacttgttttatgtaggagct

SEQ ID NO: 1130 - RNA, 21 nt, synthetic construct
  source 1..21: mol_type = other RNA
taacttgttttatgtaggagc

SEQ ID NO: 1131 - RNA, 21 nt, synthetic construct
  source 1..21: mol_type = other RNA
tcaacttgttttatgtaggag

SEQ ID NO: 1132 - RNA, 21 nt, synthetic construct
  source 1..21: mol_type = other RNA
tgcaacttgttttatgtagga

SEQ ID NO: 1133 - RNA, 21 nt, synthetic construct
  source 1..21: mol_type = other RNA
ttgcaacttgttttatgtagg

SEQ ID NO: 1134 - RNA, 21 nt, synthetic construct
  source 1..21: mol_type = other RNA
tatgcaacttgttttatgtag

SEQ ID NO: 1135 - RNA, 21 nt, synthetic construct
  source 1..21: mol_type = other RNA
tcttgtgaaaatgccatccac

SEQ ID NO: 1136 - RNA, 21 nt, synthetic construct
  source 1..21: mol_type = other RNA
tccttgtgaaaatgccatcca

SEQ ID NO: 1137 - RNA, 21 nt, synthetic construct
  source 1..21: mol_type = other RNA
ttccttgtgaaaatgccatcc

SEQ ID NO: 1138 - RNA, 21 nt, synthetic construct
  source 1..21: mol_type = other RNA
tatccttgtgaaaatgccatc

SEQ ID NO: 1139 - RNA, 21 nt, synthetic construct
  source 1..21: mol_type = other RNA
tcatccttgtgaaaatgccat

SEQ ID NO: 1140 - RNA, 21 nt, synthetic construct
  source 1..21: mol_type = other RNA
tattgtctgcagcacccatcc

SEQ ID NO: 1141 - RNA, 21 nt, synthetic construct
  source 1..21: mol_type = other RNA
tatattgtctgcagcacccat

SEQ ID NO: 1142 - RNA, 21 nt, synthetic construct
  source 1..21: mol_type = other RNA
ttatattgtctgcagcaccca

SEQ ID NO: 1143 - RNA, 21 nt, synthetic construct
  source 1..21: mol_type = other RNA
tatatattgtctgcagcaccc

SEQ ID NO: 1144 - RNA, 21 nt, synthetic construct
  source 1..21: mol_type = other RNA
ttatatattgtctgcagcacc

SEQ ID NO: 1145 - RNA, 21 nt, synthetic construct
  source 1..21: mol_type = other RNA
tatatatattgtctgcagcac

SEQ ID NO: 1146 - RNA, 21 nt, synthetic construct
  source 1..21: mol_type = other RNA
ttcagttcttccataaatgta

SEQ ID NO: 1147 - RNA, 21 nt, synthetic construct
  source 1..21: mol_type = other RNA
tttatttctgctgtgtcagtc

SEQ ID NO: 1148 - RNA, 21 nt, synthetic construct
  source 1..21: mol_type = other RNA
tcaaggactgtgatgttgctt

SEQ ID NO: 1149 - RNA, 21 nt, synthetic construct
  source 1..21: mol_type = other RNA
tagataaccaaggactgtgat

SEQ ID NO: 1150 - RNA, 21 nt, synthetic construct
  source 1..21: mol_type = other RNA
tctagttcatccaagataacc

SEQ ID NO: 1151 - RNA, 21 nt, synthetic construct
  source 1..21: mol_type = other RNA
tttcctagttcatccaagata

SEQ ID NO: 1152 - RNA, 21 nt, synthetic construct
  source 1..21: mol_type = other RNA
tgtgtagcataggcaatggca

SEQ ID NO: 1153 - RNA, 21 nt, synthetic construct
  source 1..21: mol_type = other RNA
tacatctctgatgaaatactc

SEQ ID NO: 1154 - RNA, 21 nt, synthetic construct
  source 1..21: mol_type = other RNA
ttcacatctctgatgaaatac

SEQ ID NO: 1155 - RNA, 21 nt, synthetic construct
  source 1..21: mol_type = other RNA
tcagggttaaggatttcacat

SEQ ID NO: 1156 - RNA, 21 nt, synthetic construct
  source 1..21: mol_type = other RNA
tataatgggtgacaaacaggg

SEQ ID NO: 1157 - RNA, 21 nt, synthetic construct
  source 1..21: mol_type = other RNA
tactggcggataatgggtgac

SEQ ID NO: 1158 - RNA, 21 nt, synthetic construct
  source 1..21: mol_type = other RNA
ttagttcacaaactggcggat

SEQ ID NO: 1159 - RNA, 21 nt, synthetic construct
  source 1..21: mol_type = other RNA
ttttctagttcacaaactggc

SEQ ID NO: 1160 - RNA, 21 nt, synthetic construct
  source 1..21: mol_type = other RNA
tttcatcctcactgaccaaga

SEQ ID NO: 1161 - RNA, 21 nt, synthetic construct
  source 1..21: mol_type = other RNA
tgtaaaggaaggtgacaaaat

SEQ ID NO: 1162 - RNA, 21 nt, synthetic construct
  source 1..21: mol_type = other RNA
tggtaaaggaaggtgacaaaa

SEQ ID NO: 1163 - RNA, 21 nt, synthetic construct
  source 1..21: mol_type = other RNA
tatttggtaaaggaaggtgac

SEQ ID NO: 1164 - RNA, 21 nt, synthetic construct
  source 1..21: mol_type = other RNA
ttatttggtaaaggaaggtga

SEQ ID NO: 1165 - RNA, 21 nt, synthetic construct
  source 1..21: mol_type = other RNA
tttatttggtaaaggaaggtg

SEQ ID NO: 1166 - RNA, 21 nt, synthetic construct
  source 1..21: mol_type = other RNA
tgttatttggtaaaggaaggt

SEQ ID NO: 1167 - RNA, 21 nt, synthetic construct
  source 1..21: mol_type = other RNA
tattcctctagttatttggta

SEQ ID NO: 1168 - RNA, 21 nt, synthetic construct
  source 1..21: mol_type = other RNA
ttccttgctgcaattcctcta

SEQ ID NO: 1169 - RNA, 21 nt, synthetic construct
  source 1..21: mol_type = other RNA
tataactccttgctgcaattc

SEQ ID NO: 1170 - RNA, 21 nt, synthetic construct
  source 1..21: mol_type = other RNA
ttccataactccttgctgcaa

SEQ ID NO: 1171 - RNA, 21 nt, synthetic construct
  source 1..21: mol_type = other RNA
tatccataactccttgctgca

SEQ ID NO: 1172 - RNA, 21 nt, synthetic construct
  source 1..21: mol_type = other RNA
tttaatccataactccttgct

SEQ ID NO: 1173 - RNA, 21 nt, synthetic construct
  source 1..21: mol_type = other RNA
tctgctagtttagccacattt

SEQ ID NO: 1174 - RNA, 21 nt, synthetic construct
  source 1..21: mol_type = other RNA
tacttgtgagctgctttcttc

SEQ ID NO: 1175 - RNA, 21 nt, synthetic construct
  source 1..21: mol_type = other RNA
tgcaaaatacttgagtctctt

SEQ ID NO: 1176 - RNA, 21 nt, synthetic construct
  source 1..21: mol_type = other RNA
ttgcaaaatacttgagtctct

SEQ ID NO: 1177 - RNA, 21 nt, synthetic construct
  source 1..21: mol_type = other RNA
tttgcaaaatacttgagtctc

SEQ ID NO: 1178 - RNA, 21 nt, synthetic construct
  source 1..21: mol_type = other RNA
ttttgcaaaatacttgagtct

SEQ ID NO: 1179 - RNA, 21 nt, synthetic construct
  source 1..21: mol_type = other RNA
tctttgcaaaatacttgagtc

SEQ ID NO: 1180 - RNA, 21 nt, synthetic construct
  source 1..21: mol_type = other RNA
tcatcgtccataactttgcaa

SEQ ID NO: 1181 - RNA, 21 nt, synthetic construct
  source 1..21: mol_type = other RNA
tgtttcttccatgttgaactc

SEQ ID NO: 1182 - RNA, 21 nt, synthetic construct
  source 1..21: mol_type = other RNA
ttgtttcttccatgttgaact

SEQ ID NO: 1183 - RNA, 21 nt, synthetic construct
  source 1..21: mol_type = other RNA
tgtgtttcttccatgttgaac

SEQ ID NO: 1184 - RNA, 21 nt, synthetic construct
  source 1..21: mol_type = other RNA
ttgtgtttcttccatgttgaa

SEQ ID NO: 1185 - RNA, 21 nt, synthetic construct
  source 1..21: mol_type = other RNA
tctgtgtttcttccatgttga

SEQ ID NO: 1186 - RNA, 21 nt, synthetic construct
  source 1..21: mol_type = other RNA
tagaagtctgtgtttcttcca

SEQ ID NO: 1187 - RNA, 21 nt, synthetic construct
  source 1..21: mol_type = other RNA
taagaagagaagtctgtgttt

SEQ ID NO: 1188 - RNA, 21 nt, synthetic construct
  source 1..21: mol_type = other RNA
ttttgttcacaaatgtagtct

SEQ ID NO: 1189 - RNA, 21 nt, synthetic construct
  source 1..21: mol_type = other RNA
ttggagagttattttgtacag

SEQ ID NO: 1190 - RNA, 21 nt, synthetic construct
  source 1..21: mol_type = other RNA
tctggagagttattttgtaca

SEQ ID NO: 1191 - RNA, 21 nt, synthetic construct
  source 1..21: mol_type = other RNA
taagataggctgttactggag

SEQ ID NO: 1192 - RNA, 21 nt, synthetic construct
  source 1..21: mol_type = other RNA
tatgctcacatgtcacacaaa

SEQ ID NO: 1193 - RNA, 21 nt, synthetic construct
  source 1..21: mol_type = other RNA
ttttatgctcacatgtcacac

SEQ ID NO: 1194 - RNA, 21 nt, synthetic construct
  source 1..21: mol_type = other RNA
tgtcataattttatgctcaca

SEQ ID NO: 1195 - RNA, 21 nt, synthetic construct
  source 1..21: mol_type = other RNA
tggaatataccatggtcataa

SEQ ID NO: 1196 - RNA, 21 nt, synthetic construct
  source 1..21: mol_type = other RNA
taggaatataccatggtcata

SEQ ID NO: 1197 - RNA, 21 nt, synthetic construct
  source 1..21: mol_type = other RNA
tctgtttccaataggaatata

SEQ ID NO: 1198 - RNA, 21 nt, synthetic construct
  source 1..21: mol_type = other RNA
ttctctgtttccaataggaat

SEQ ID NO: 1199 - RNA, 21 nt, synthetic construct
  source 1..21: mol_type = other RNA
taaacctctctgtttccaata

SEQ ID NO: 1200 - RNA, 21 nt, synthetic construct
  source 1..21: mol_type = other RNA
tagaaaagttaggaagacaga

SEQ ID NO: 1201 - RNA, 21 nt, synthetic construct
  source 1..21: mol_type = other RNA
tacgtagaaaagttaggaaga

SEQ ID NO: 1202 - RNA, 21 nt, synthetic construct
  source 1..21: mol_type = other RNA
ttacgtagaaaagttaggaag

SEQ ID NO: 1203 - RNA, 21 nt, synthetic construct
  source 1..21: mol_type = other RNA
tagagtgtttatacgtagaaa

SEQ ID NO: 1204 - RNA, 21 nt, synthetic construct
  source 1..21: mol_type = other RNA
taagagtgtttatacgtagaa

SEQ ID NO: 1205 - RNA, 21 nt, synthetic construct
  source 1..21: mol_type = other RNA
ttcaagagtgtttatacgtag

SEQ ID NO: 1206 - RNA, 21 nt, synthetic construct
  source 1..21: mol_type = other RNA
tggaagtctattcaagagtgt

SEQ ID NO: 1207 - RNA, 21 nt, synthetic construct
  source 1..21: mol_type = other RNA
taaagtggaagtctattcaag

SEQ ID NO: 1208 - RNA, 21 nt, synthetic construct
  source 1..21: mol_type = other RNA
tcaaagtggaagtctattcaa

SEQ ID NO: 1209 - RNA, 21 nt, synthetic construct
  source 1..21: mol_type = other RNA
tttacaaagtggaagtctatt

SEQ ID NO: 1210 - RNA, 21 nt, synthetic construct
  source 1..21: mol_type = other RNA
ttaattacaaagtggaagtct

SEQ ID NO: 1211 - RNA, 21 nt, synthetic construct
  source 1..21: mol_type = other RNA
tctaattacaaagtggaagtc

SEQ ID NO: 1212 - RNA, 21 nt, synthetic construct
  source 1..21: mol_type = other RNA
tttaaggctttactggactta

SEQ ID NO: 1213 - RNA, 21 nt, synthetic construct
  source 1..21: mol_type = other RNA
tatattctgccacttaaggct

SEQ ID NO: 1214 - RNA, 21 nt, synthetic construct
  source 1..21: mol_type = other RNA
tggaattatattctgccactt

SEQ ID NO: 1215 - RNA, 21 nt, synthetic construct
  source 1..21: mol_type = other RNA
ttggatagattcctgccagat

SEQ ID NO: 1216 - RNA, 21 nt, synthetic construct
  source 1..21: mol_type = other RNA
tgttcaatggatagattcctg

SEQ ID NO: 1217 - RNA, 21 nt, synthetic construct
  source 1..21: mol_type = other RNA
tggtggataaactggttgcat

SEQ ID NO: 1218 - RNA, 21 nt, synthetic construct
  source 1..21: mol_type = other RNA
tgttcttggtggataaactgg

SEQ ID NO: 1219 - RNA, 21 nt, synthetic construct
  source 1..21: mol_type = other RNA
tttcttatgttcttggtggat

SEQ ID NO: 1220 - RNA, 21 nt, synthetic construct
  source 1..21: mol_type = other RNA
tcttattccttctgtgctcta

SEQ ID NO: 1221 - RNA, 21 nt, synthetic construct
  source 1..21: mol_type = other RNA
ttgaccttattccttctgtgc

SEQ ID NO: 1222 - RNA, 21 nt, synthetic construct
  source 1..21: mol_type = other RNA
tatgaccttattccttctgtg

SEQ ID NO: 1223 - RNA, 21 nt, synthetic construct
  source 1..21: mol_type = other RNA
ttaaactgcttaatcctatga

SEQ ID NO: 1224 - RNA, 21 nt, synthetic construct
  source 1..21: mol_type = other RNA
taacaatctttaaactgctta